(19) United States
(12) Patent Application Publication
(10) Pub. No.: US 2023/0132281 A1
(43) Pub. Date: Apr. 27, 2023

(54) RNA SEQUENCING TO DIAGNOSE SEPSIS

(71) Applicant: RHODE ISLAND HOSPITAL, Providence, RI (US)

(72) Inventors: Sean F. MONAGHAN, East Greenwich, RI (US); Alger M. FREDERICKS, East Providence, RI (US)

(21) Appl. No.: 17/760,490

(22) PCT Filed: Feb. 16, 2021
(86) PCT No.: PCT/US2021/018218

§ 371 Date: Aug. 10, 2022

Related U.S. Application Data
(60) Provisional application No. 62/976,873, filed on Feb. 14, 2020.

(51) Int. Cl. C12Q 1/689; C12Q 1/6883; G16B 30/00; G16B 40/30

(52) U.S. Cl. CPC C12Q 1/689; C12Q 1/6883; G16B 30/00; G16B 40/30

(57) ABSTRACT

Deep RNA sequencing is a technology that provides an initial diagnostic for sepsis that can also monitor the indicia of treatment and recovery (bacterial counts reduce, physiology returns to steady-state). The invention can be used for many other hospital conditions, particularly those needing an intensive care unit stay with the attendant risk of bacterial infection, such as trauma, stroke, myocardial infarction, or major surgery.

### STATEMENT REGARDING FEDERALLY SPONSORED RESEARCH OR DEVELOPMENT

This invention was made with government support under GM103652 awarded by National Institutes of Health. The government has certain rights in the invention.

### FIELD OF THE INVENTION

This invention generally relates to chemical analysis of biological material, using nucleic acid products used in the analysis of nucleic acids, e.g., primers or probes for diseases caused by alterations of genetic material.

### REFERENCE TO RELATED APPLICATIONS

This patent matter claims priority to provisional patent application U.S. Ser. No. 62/976,873, filed Feb. 14, 2020.

### BACKGROUND OF THE INVENTION

Sepsis is a life-threatening organ dysfunction due to a dysregulated host response to infection. Despite declining age-standardized incidence and mortality, sepsis remains a significant cause of health loss worldwide. Rudd et al., The Lancet, 395(10219), 200-211 (Jan. 18, 2020). Sepsis is treatable, and timely implementation of targeted interventions improves outcomes.

Sepsis is diagnosed clinically by the presence of acute infection and new organ dysfunction. Singer et al., JAMA, 315, 801-810 (February 2016). Unlike the previous concepts of septicemia or blood poisoning, the current definition of sepsis extends across bacterial, fungal, viral, and parasitic pathogens. The definition focuses on the host response as the major source of morbidity and mortality. Bone et al., Chest, 101, 1644-1655 (1992). Globally, there were about 48.9 million cases of sepsis in 2017, with about 11.0 million total sepsis-related deaths worldwide, representing 19.7% (18-2-21-4). This number may be a substantial undercount. Rudd et al., The Lancet, 395(10219), 200-211 (Jan. 18, 2020). Sepsis results from an underlying infection, so sepsis is an intermediate cause of health loss. Because, according to the principles of the International Classification of Diseases (ICD), causes of death are assigned based on the underlying disorder that triggers the chain of events leading to death rather than intermediate causes, sepsis, when reported as the cause of death, are considered miscoded.

Thus, the global burden of sepsis is more significant than previously appreciated. There is substantial variation in sepsis incidence and mortality according to Healthcare Access and Quality Index (HAQ Index), Lancet, 390, 231-266 (2017)), with the highest burden in places that cannot prevent, identify, or treat sepsis. Further research is needed to understand these disparities and developing policies and practices targeting their amelioration. More robust infection-prevention measures should be assessed and implemented in areas with the highest incidence of sepsis and among populations on which sepsis has the most significant impact. The impact of sepsis is especially severe among children, so more than half of all sepsis cases worldwide in 2017 occurred among children, many of them neonates.

Physicians diagnose sepsis using clinical judgment under one or more clinical scores. The systemic inflammatory response syndrome (SIRS) approach assesses an inflammatory state affecting the whole body, which is the body's response to an infectious or non-infectious challenge. Jui et al. (American College of Emergency Physicians), Ch. 146: Septic Shock. in Tintinalli et al. (eds.). Tintinalli's Emergency Medicine: A Comprehensive Study Guide, 7th edition, (New York: McGraw-Hill, 2011). pp. 1003-14. Sepsis has both pro-inflammatory and anti-inflammatory components. The qSOFA approach simplifies the SOFA score by including only its three clinical criteria and by including any altered mentation. Singer et al., JAMA, 315, 801-810 (February 2016). qSOFA can easily and quickly be repeated serially on patients.

A culture of the bacterial infection confirms a diagnosis of sepsis. A culture diagnosis can be delayed by forty-eight hours and sometimes cannot be performed successfully. Clinical judgment sometimes misses sepsis.

Biomarkers are being developed for sepsis, but no reliable biomarkers exist. A 2013 review concluded moderate-quality evidence exists to support the use of the procalcitonin level as a method to distinguish sepsis from non-infectious causes of SIRS. Still, he level alone could not definitively make the diagnosis. Wacker et al., The Lancet Infectious Diseases. 13(5), 426-35 (May 2013). A 2012 systematic review found that soluble urokinase-type plasminogen activator receptor (SuPAR) is a nonspecific marker of inflammation and does not accurately diagnose sepsis. Backes et al. Intensive Care Medicine, 38(9): 1418-28 (September 2012).

There remains a need in the medical art for a better diagnosis of sepsis.

### SUMMARY OF THE INVENTION

The concept of diagnostics is analogous to using a fishing lure to find a single protein, gene, or RNA sequence. The invention provides an improved concept, using a fishing net to obtain all the RNA data in a sample, and use computational biology to better sort through all the data (fish) to identify patients with sepsis and the bacteria causing the immune response. The invention provides an initial diagnostic for sepsis that can also monitor the indicia of treatment and recovery (bacterial counts reduce, physiology returns to steady-state). The invention can be used for many other hospital conditions, particularly those needing an intensive care unit stay with the attendant risk of bacterial infection, such as trauma, stroke, myocardial infarction, or major surgery.

In the first embodiment, the invention provides unmapped bacterial RNA reads to identify bacteria that cause sepsis. In the second embodiment, the invention provides unmapped viral reads to identify sepsis or viral reactivation. In the third embodiment, the invention provides the use of unmapped B/T V(D)J to identify sepsis. In the fourth embodiment, the invention provides Principal Component Analysis of RNA splicing entropy to identify sepsis. In the fifth embodiment, the invention provides RNA lariats to identify sepsis. In the sixth embodiment, the invention provides a Principal Component Analysis of gene expression, alternative RNA splicing, or alternative transcription start and end to identify sepsis.

In producing the listed embodiments, one of ordinary skill in the molecular biological art uses one or more of the following steps.

The first step is for one of ordinary skill in the molecular biological art to obtain RNA sequencing from a body sample. In the seventh embodiment, the body sample is a bodily fluid sample. In the eighth embodiment, the bodily fluid sample is blood. In the ninth embodiment, the target is 100,000,000 reads/sample.

The second step is for one to align the RNA sequencing data (reads) to the genome of interest. In the tenth embodiment, the reads from a human sample are aligned to a human genome. In the eleventh embodiment, the reads from a mouse sample are aligned to a mouse genome.

The third step is to select the un-mapped reads and analyze the reads using a Read Origin Protocol (ROP).

In the first embodiment (above), the next step is to identify bacteria that are present in the sample. From the ROP, one of ordinary skill in the molecular biological art identifies bacteria that are present in the sample. In the twelfth embodiment, one of ordinary skill in the molecular biological art or medical art uses the identified bacteria to list potential causative organisms of sepsis (product).

In the second embodiment (above), from the ROP, the next step is to identify the viruses present in the sample. In the thirteenth embodiment, one uses the virus identified with PCA to identify likely sepsis samples.

In the third embodiment (above), from the ROP, the next step is to identify the T/B cell epitopes present in the samples. In the fourteenth embodiment, one uses the T/B cell epitopes identified with PCA to identify likely sepsis samples.

Alternatively (or in combination), in the third step, one selects the mapped reads and then uses a program that enables detection and quantification of alternative RNA splicing events to identity gene expression, RNA splicing events, alternative transcription start/end, or RNA splicing entropy. In a fifteenth embodiment, the program that enables detection and quantification of alternative RNA splicing events is Whippet. In the sixteenth embodiment, one uses the gene expression changes, RNA splicing events, and alternative transcription start/end with PCA to identify likely sepsis samples. In the seventeenth embodiment, one uses the RNA splicing entropy identified with PCA to identify likely sepsis samples.

In the fifth embodiment, from the gene expression, RNA splicing events, alternative transcription start/end, or RNA splicing entropy, the next step is for one to identify RNA lariats from the mapped reads. In the eighteenth embodiment, one uses the RNA lariats with PCA to identify likely sepsis samples.

In the nineteenth embodiment, the invention provides an output product with five plots comprising bacterial RNA reads, viral reads, B/T V(D)J epitopes, RNA splicing entropy, and RNA lariat embodiments described above and a list of likely bacteria causing the infection.

RNA sequencing data be used in several ways. (1) Identification of biomarkers. Rather than need to pick a subset to test for, RNA sequencing data can identify genes with increased expression that would correlate to biomarkers of interest. (2) Identification of new biomarkers. RNA sequencing data allows for analysis of processes such as RNA splicing. The method of RNA splicing entropy can be quantified and grouped according to a Principal Component Analysis into sick or not sick. RNA lariats can also be identified in sequencing data and used as a potential biomarker. All biomarkers can be followed over time to assess for resolution of the sepsis. (3) Use of un-mapped reads in sepsis. RNA sequencing typically aligns with the genome of reference (i.e., the human genome). Reads that are not aligned to the human genome are discarded (the percentage of un-mapped reads could itself be a biomarker). These un-mapped reads could be of two major potential interests. (4) Identification of the microbe causing the infection. The unmapped reads can be referenced to the genome of disease-causing microbes (bacteria, viruses, fungi, etc.) to identify the causative organism and start treatment earlier. Serial measurements can also assess the effectiveness of treatment.

The results presented show that mice exposed to trauma separated from controls using PCA. Similarly, mice that did not survive fourteen days post exposure clustered closely together on PCA. These results show a substantial difference in global pre-mRNA processing entropy in mice exposed to trauma vs. controls, and that pre-mRNA processing entropy is useful in predicting mortality.

### BRIEF DESCRIPTION OF THE DRAWINGS

FIG. 1 is a chart showing Principal Component Analysis of samples in the blood. Three mice exposed to the trauma model were compared to three mice in the control group (total n=6). When plotting the first two principal components against each other, the exposed mice separated from the control mice. Samples clustered based on tissue type and ARDS status on the Principal Component Analysis plot, suggesting that splicing entropy can be a biomarker for ARDS status. The first two principal components plotted against each other. The percentages in parentheses represent the percent variability explained by the principal component. Circles represent control mice; squares represent mice exposed to hemorrhage followed by cecal ligation and puncture.

FIG. 2 is a chart showing a Principal Component Analysis of the survival study. A total of ten mice exposed to trauma were part of the survival experiment. A mortality rate of 30% was observed, which is consistent with previous studies using this model. When plotting the first two principal components against each other, the mice who did not survive closely clustered together. The first two principal components are plotted against each other. The percentages represent the percent variability explained by the principal component. The squares represent mice that died on or before 14 days post CLP, circles represent mice that survived.

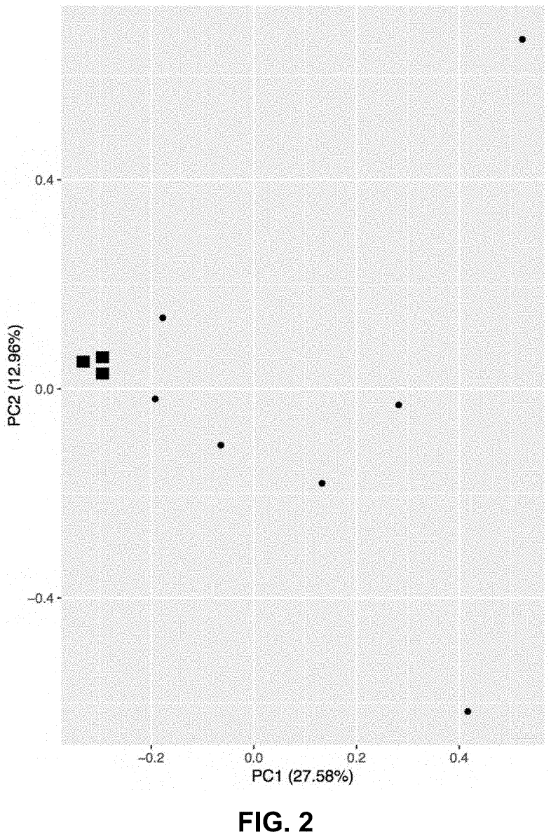

### DETAILED DESCRIPTION OF THE INVENTION

### Industrial Applicability

Despite being the cause of death in 1 out of 5 people in the world, there is not a single standard test to diagnose sepsis. Despite declining age-standardized incidence and mortality, sepsis remains a significant cause of health loss worldwide. Rudd et al., The Lancet, 395(10219), 200-211 (Jan. 18, 2020). Sepsis patients undergo the physiology common to patients in the intensive care unit: hypotension, tachycardia, hyperthermia, and hypoxia.

Delays in treatment for sepsis is known to impact mortality. Early identification of the differences between clinically similar patients would allow for earlier interventions (surgery, antibiotics). Using RNA sequencing technology combined with computation biology techniques to understand RNA biology the differences in these two patients could be identified. Earlier prediction of complications would also allow for triage of patients to facilities equipped to deal with them and allow for better discussions regarding expected mortality and morbidity.

Currently it takes days to get a final diagnosis for bacterial pathogen, since culturing of the bacteria is needed. Confirming bacteremia is currently done microbial blood culture, but the turnaround time can lead to a delay in diagnosis. Biron et al., Biomarker Insights, 10(Suppl 4), 7-17 (Sep. 15, 2015). Procalcitonin (PCT) has been shown to correlate more closely to onset and treatment of sepsis than C-reactive protein (CRP). Vijayan et al., J. Intensive Care (Aug. 3, 2017). Much work has been done with PCT as a predictor of sepsis before symptom onset. Dolin et al., Shock, 49(4), 364-70 (April 2018). PCT has low specificity for sepsis, and is elevated in cancers, autoimmune diseases, and other physiological stressors. Bloos & Reinhart, Virulence, 5(1), 154-60 (Jan. 1, 2014).

RNA sequencing data can identify the bacteria more quickly than culture. The drop in the cost of sequencing has refocused genetic analyses from DNA to RNA sequencing. Methods to analyze this data have improved. Stark et al., Nature Reviews Genetics (2019). Compared to DNA, RNA undergoes dynamic changes by transcription and post-transcriptional processing, providing unique insight into cellular activity. RNA reflects a broader source of infectious etiologies, given that both DNA and RNA viruses have RNA genetic material, whether in the genome or by transcription of mRNA. Patients with trauma who die or have complications are expected to have different changes in expression, alternative RNA splicing, and alternative transcription start/end compared to patients who survive and do not have a complication. The differences seen in RNA biology may correlate with injury severity or predict outcomes. This invention should help direct care in trauma patients when RNA sequencing speeds increase to allow for results that are available when needed for patients in the ICU (within one hour).

RNA sequencing data related to other processes (RNA splicing entropy, gene expression, viral counts, lariat counts, etc.) will provide a signature that can identify patients with sepsis. A better understanding of RNA biology in the clinical scenario of critically ill sepsis patients can have a broad impact on biomedical science. When the information in RNA sequencing data can identify patients who have not resolved the immune response to the initial sepsis, outcomes can improve.

The number of unmapped reads aligning to viral pathogenic genomes can be a biomarker of critical illness. Patients with late death should have different gene expression, alternative RNA splicing (including RNA splicing entropy), and alternative transcription start/end as compared to patients with an early death. the genes with increased alternative RNA splicing (including RNA splicing entropy), and alternative transcription start/end are expected to be different in the patients who died late compared to those who died early. These identified genes provide insight into proteins not considered in trauma patients as potential biomarkers or targets of therapeutic intervention, but point to pathological mechanism not appreciated or unclear.

Moreover, RNA biology before the trauma should be able to predict survivors. Mice that survive to fourteen days should have less RNA biology changes compared to mice at the early time point. This are done across three distinct background mice to account for the heterogeneity of humans and the comparability of the two most common immunological/genetic mouse model strains used. As it relates to comparing samples across mouse strains, since gene expression, RNA splicing, and alternative transcription start/end are all basic molecular functions, the results remain similar across the multiple strains.

Identification of B and T cell epitopes from the unmapped reads could be a biomarker for sepsis. Critical illness decreases the diversity of these epitopes. A resolution could signal an improvement in clinical status. Losing some epitopes could indicate immune suppression seen in critical illness.

Alternative transcription start and end is another biological process potentially influenced by sepsis. Current technology now allows us to identify changes in transcription with RNA sequencing data. Hardwick et al., Frontiers in Genetics, 10, 709 (2019); Cass & Xiao X, Cell Systems, 9(4), 23, 393-400.e6 (October 2019). The genes that have increased difference in alternative transcription start/end could be disease treatment targets. A change to the start or end of the RNA is likely to change the ultimate endpoint of that transcript. Understanding the changes in transcription start and end would better describe the ultimate result of proteins since that were thought to be transcribed and translated could have been transcribed (with changes in the start or end) which lead to nonsense mediated decay or the translation of an alternative isoform.

Genes with significant alternative splicing and high entropy in the mouse after trauma may be target for intervention. This invention can better diagnose sepsis and the microbe causing the disease. Emergency room and critical care physicians can use the invention.

### Solution: RNAs as Biomarkers of Critical Illness

While proteins have traditionally been used to reflect inflammatory load, RNAs are more specific to certain etiologies and clinical outcomes.

High through-put sequencing technologies allows for coding and non-coding RNAs (ncRNA) as markers of disease risk and progression. Next-generation sequencing (NGS) quantifies RNAs by sequencing of complementary DNA (cDNA), allowing transcriptomic analysis of mRNAs, ribosomal RNAs (rRNA), and ncRNAs. Kukurba & Montgomery, Cold Spring Harb. Protoc., 2015(11), 951-69 (Apr. 13, 2015).

Coding and non-coding RNAs have been studied as biomarkers. Less attention has been on the portion of data produced (9-20%) via RNA-sequencing that is consistently discarded when it cannot be mapped to a reference genome. Mangul et al., ROP: Dumpster diving in RNA-sequencing to find the source of 1 trillion reads across diverse adult human tissues. Genome Biol., 19 (Feb. 15, 2018).

The discovery of serum-stable circulating miRNAs allows the use of cell-free miRNAs as biomarkers of disease. Benz et al., Int. J. Mol. Sci., 17(1) (Jan. 9, 2016); Wang et al., J. Cell Physiol., 231(1), 25-30 (2016). Elevated miR-133a levels in serum correlate to poorer prognosis in ICU patients. Tacke et al., Crit. Care Med., 42(5), 1096-104 (May 2014). Groups of miRNAs delineate between different infectious etiologies, such as S. aureus and E. coli. Wu et al., PLoS One, 8(10) (2013). The lack of standardization in measuring circulating miRNA expression affects reproducibility between analyses and limited its clinical applicability. Lee et al., Mol. Diagn. Ther., 21(3), 259-68 (June 2017).

Physiologic stress induces viral reactivation by impairing the immune response and upregulating cell cycle progression pathways such as MAPK and NF-κB. Walton et al., PLoS One, 9(6), e98819 (Jun. 11, 2014); Traylen et al., Future Virol., 6(4), 451-63 (April 2011). Secretion of pro-inflammatory cytokines, such as TNF-α, has been shown to play a role in reactivating latent cytomegalovirus (CMV) in patients that had undergone recent stress even absent systemic inflammation. Prosch et al., Virology, 272(2), 357-65 (Jul. 5, 2000). A combination of inflammatory challenges and immune cell dysregulation has been shown to contribute to an environment that both promotes viral reactivation and maintains viremia. Walton et al., PLoS One, 9(6), e98819 (Jun. 11, 2014).

In a traumatic shock EXAMPLE, C57BL6 mice were treated by sequential hemorrhagic shock followed by cecal ligation and puncture, which induces sepsis. RNA was extracted from cellular component of lung and immune cells in blood after discarding plasma and serum. Samples were collected from both healthy and critically ill mice and sequenced via NGS at Gene Wiz in South Plainfield, N.J., USA. Reads were aligned to mm9 genome using STAR and then unmapped reads were mapped to viral genomes via ROP. Dobin et al., Bioinformatics, 29(1), 15-21 (January 2013). Mangul et al., Genome Biol., 19 (Feb. 15, 2018). Two-sample t tests were conducted to compare number of viral reads in healthy versus critically ill mouse lung and blood.

### Definitions

For convenience, the meaning of some terms and phrases used in the specification, examples, and appended claims, are listed below. Unless stated otherwise or implicit from context, these terms and phrases have the meanings below. These definitions are to aid in describing particular embodiments and are not intended to limit the claimed invention. Unless otherwise defined, all technical and scientific terms have the same meaning as commonly understood by one of ordinary skill in the art to which this invention belongs. For any apparent discrepancy between the meaning of a term in the art and a definition provided in this specification, the meaning provided in this specification shall prevail.

“Acute respiratory distress syndrome (ARDS)” has the medical art-defined meaning. ARDS is a type of respiratory failure characterized by rapid onset of widespread inflammation in the lungs. Symptoms include shortness of breath, rapid breathing, and bluish skin coloration. Causes may include sepsis, pancreatitis, trauma, pneumonia, and aspiration.

“Alternative splicing (AS)” has the molecular biological art-defined meaning. RNA splicing is a basic molecular function that occurs in all cells directly after RNA transcription, but before protein translation, in which introns are removed and exons are joined. Alternative splicing or alternative RNA splicing, or differential splicing, is a regulated process during gene expression that results in a single gene coding for multiple proteins. Exons of a gene can be included within or excluded from the final, processed messenger RNA (mRNA) produced from that gene. The proteins translated from alternatively spliced mRNAs can contain differences in their amino acid sequence and, often, in their biological functions.

“Aldo/keto reductase gene” has the molecular biological art-defined meaning.

“Base R” is an R-based computer program.

“Mann Whitney U tests” has the statistical art-defined meaning. The Mann-Whitney U test (also called the Mann-Whitney-Wilcoxon (MWW), Wilcoxon rank-sum test, or Wilcoxon-Mann-Whitney test) is a nonparametric test of the null hypothesis that it is equally likely that a randomly selected value from one population is less than or greater than a randomly selected value from a second population. This test can be used to investigate whether two independent samples were selected from populations having the same distribution.

“mountainClimber” is a cumulative-sum-based approach to identify alternative transcription start (ATS) and alternative polyadenylation (APA) as change points. Unlike many existing methods, mountainClimber runs on a single sample and identifies multiple ATS or APA sites anywhere in the transcript. Cass & Xiao X, “mountainClimber identifies alternative transcription start and polyadenylation sites in RNA-Seq.” Cell Systems, 9(4), 23, 393-400.e6 (October 2019).

“Next Generation Sequencing (NGS)” has the molecular biological art-defined meaning. NGS technology is typically characterized by being highly scalable, allowing the entire genome to be sequenced at once. Usually, this is accomplished by fragmenting the genome into small pieces, randomly sampling for a fragment, and sequencing it using one of a variety of technologies.

“Principal Component Analysis (PCA)” has the computer-art and molecular biological art-defined meaning. Principal component analysis is a statistical procedure that uses an orthogonal transformation to convert a set of observations of possibly correlated variables (entities each of which takes on various numerical values) into a set of values of linearly uncorrelated variables called principal components.

“Read origin protocol (ROP)” has the computer-art meaning of is a computational protocol that aims to discover the source of all reads, including those originating from repeat sequences, recombinant B and T cell receptors, and microbial communities. The Read Origin Protocol was developed to determine what the unmapped reads represented. Mangul al., “ROP: Dumpster diving in RNA-sequencing to find the source of 1 trillion reads across diverse adult human tissues.” Genome Biology 19, 36 (2018). Recent development of Read Origin Protocol (ROP) has demonstrated that unmapped reads align to bacterial, viral, fungal, and B/T rearrangement genomes.

“Read” has the molecular biological art-defined meaning of reading sequencing results to determine nucleotide base structure.

“Sepsis” has the medical art-defined meaning of a life-threatening condition that arises when the body's response to infection injures its tissues and organs. Bone et al., “Definitions for sepsis and organ failure and guidelines for the use of innovative therapies in sepsis.” Chest, 101, 1644-1655 (1992); Singer et al., “The third international consensus definitions for sepsis and septic shock (Sepsis-3).” JAMA, 315, 801-810 (February 2016).

“STAR aligner” is the Spliced Transcripts Alignment to a Reference (STAR), a fast RNA-seq read mapper, with support for splice-junction and fusion read detection. STAR aligns reads by finding the Maximal Mappable Prefix (MMP) hits between reads (or read pairs) and the genome, using a Suffix Array index. Different parts of a read can be mapped to different genomic positions, corresponding to splicing or RNA-fusions. The genome index includes known splice-junctions from annotated gene models, allowing for sensitive detection of spliced reads. STAR performs local alignment, automatically soft clipping ends of reads with high mismatches. Dobin et al., STAR: Ultrafast universal RNA-seq aligner. Bioinformatics, 29(1), 15-21 (January 2013).

“V(D)J recombination” has the molecular biological art-defined meaning. V(D)J recombination occurs in developing lymphocytes during the early stages of T and B cell maturation, involves somatic recombination, and results in the highly diverse repertoire of antibodies/immunoglobulins and T cell receptors (TCRs) found in B cells and T cells, respectively.

“Whippet” (OMICS_29617) is a program that enables detection and quantification of alternative RNA splicing events of any complexity that has computational requirements compatible with a laptop computer. Whippet is a program that applies the concept of lightweight algorithms to event-level splicing quantification by RNAseq. The software can facilitate the analysis of simple to complex AS events that function in normal and disease physiology. Alternative splicing events with high entropy are identified using Whippet. Sterne-Weiler et al., Molecular Cell, 72, 187-200.e186 (2018).

### Guidance from the Prior Art

A person of ordinary skill in the art of can use these patents, patent applications, and scientific references as guidance to predictable results when making and using the invention:
Ashburner et al., Gene ontology: tool for the unification of biology. The Gene Ontology Consortium. Nature Genetics, 25, 25-29 (2000).
Ayala et al., Shock-induced neutrophil mediated priming for acute lung injury in mice: divergent effects of TLR-4 and TLR-4/FasL deficiency. The American Journal of Pathology, 161, 2283-2294 (2002).
Benz et al., Circulating microRNAs as biomarkers for sepsis. Int. J. Mol. Sci., 17(1) (Jan. 9, 2016).
Biron et al., Biomarkers for Sepsis, What Is and What Might Be? Biomarker Insights, 10(Suppl 4), 7-17 (Sep. 15, 2015).
Bloos & Reinhart, Rapid diagnosis of sepsis. Virulence, 5(1), 154-60 (Jan. 1, 2014).
Carithers et al., A novel approach to high-quality postmortem tissue procurement: The GTEx Project. Biopreservation and Biobanking, 13(5), 311-9 (2015).
Cass & Xiao X, mountainClimber identifies alternative transcription start and polyadenylation sites in RNA-Seq. Cell Systems, 9(4), 23, 393-400.e6 (October 2019).
Chang et al., High-throughput binding analysis determines the binding specificity of ASF/SF2 on alternatively spliced human pre-mRNAs. Combinatorial Chemistry & High Throughput Screening, 13(3), 242-52 (2010).
Charles et al., The components of the immune system. Immunobiol. Immune Syst. Health Dis. 5th Ed. (2001).
Chikani et al., Racial/ethnic disparities in rates of traumatic injury in Arizona, 2011-2012. Public Health Reports, 131(5), 704-10 (2016).
Dobin et al., STAR: Ultrafast universal RNA-seq aligner. Bioinformatics, 29(1), 15-21 (January 2013).
Dolin et al., A novel combination of biomarkers to herald the onset of sepsis prior to the manifestation of symptoms. Shock, 49(4), 364-70 (April 2018).
Duggal et al., Innate and adaptive immune dysregulation in critically ill ICU patients. Sci. Rep., 8(1), 1-11 (Jul. 5, 2018).
Elias & Dias, Microenvironment changes (in pH) affect VEGF alternative splicing. Cancer Microenvironment, 1(1), 131-9 (2008).
Fink & Warren, Strategies to improve drug development for sepsis. Nature Reviews Drug Discovery, 13(10), 741-58 (2014).
Fredericks et al., RNA-binding proteins: Splicing factors and disease. Biomolecules, 5(2), 893-909 (2015).
Gultyaev et al., P element temperature-specific transposition, a model for possible regulation of mobile elements activity by pre-mRNA secondary structure. TSitologiia i Genetika, 48(6), 40-4 (2014).
Hardwick et al., Getting the entire message: Progress in isoform sequencing. Frontiers in Genetics, 10, 709 (2019).
Iacobellis et al., Perforated appendicitis, assessment with multidetector computed tomography. Seminars in Ultrasound, CT, and MR, 37(1), 31-6 (2016).
Kasim et al., Shutdown of achaete-scute homolog-1 expression by heterogeneous nuclear ribonucleoprotein (hnRNP)-A2/B1 in hypoxia. The Journal of Biological Chemistry, 289(39), 26973-88 (2014).
Kukurba & Montgomery, RNA sequencing and analysis. Cold Spring Harb. Protoc., 2015(11), 951-69 (Apr. 13, 2015).
Lee et al., The importance of standardization on analyzing circulating RNA. Mol. Diagn. Ther., 21(3), 259-68 (June 2017).
Lemieux et al., A function for the hnRNP A1/A2 proteins in transcription elongation. PloS One, 10(5), e0126654 (2015).
Lomas-Neira et al., Blockade of endothelial growth factor, angiopoietin-2, reduces indices of ARDS and mortality in mice resulting from the dual-insults of hemorrhagic shock and sepsis. Shock, 45(2), 157-65 (2016).
Mahen et al., mRNA secondary structures fold sequentially but exchange rapidly in vivo. PLoS Biology, 8(2), e1000307 (2010).
Mangul et al., ROP: Dumpster diving in RNA-sequencing to find the source of 1 trillion reads across diverse adult human tissues. Genome Biol., 19 (Feb. 15, 2018).
Monaghan et al. Mechanisms of indirect acute lung injury: A novel role for the coinhibitory receptor, programmed death-1. Annals of Surgery, 255(1), 158-64 (2012).
Monaghan et al., Changes in the process of alternative RNA splicing results in soluble B and T lymphocyte attenuator with biological and clinical implications in critical illness. Mol Med., 24(1), 32 (Jun. 18, 2018).
Monaghan et al., Novel anti-inflammatory mechanism in critically ill: Soluble programmed cell death receptor-1 (sPD-1). Journal of the American College of Surgeons, 213(3), S54-S5 (2011).
Monaghan et al., Programmed death 1 expression as a marker for immune and physiological dysfunction in the critically ill surgical patient. Shock, 38(2), 117-22 (2012).
Monaghan et al., Soluble programmed cell death receptor-1 (sPD-1), a potential biomarker with anti-inflammatory properties in human and experimental acute respiratory distress syndrome (ARDS). J. Transl. Med., 14(1), 312 (2016).
Pan et al., Deep surveying of alternative splicing complexity in the human transcriptome by high-throughput sequencing. Nature Genetics, 40(12), 1413-5 (2008).
Prösch et al., A Novel Link between Stress and Human Cytomegalovirus (HCMV) Infection: Sympathetic Hyperactivity Stimulates HCMV Activation. Virology, 272(2), 357-65 (Jul. 5, 2000).
Rhodes et al., The surviving sepsis campaign bundles and outcome, results from the International Multicentre Prevalence Study on Sepsis (the IMPreSS study). Intensive Care Medicine, 41(9), 1620-8 (2015).
Ritchie et al., Entropy measures quantify global splicing disorders in cancer. PLoS Computational Biology, 4(3), e1000011 (2008).
Sterne-Weiler et al., Efficient and accurate quantitative profiling of alternative splicing patterns of any complexity on a laptop. Molecular Cell, 72(1), 187-200.e6 (2018).
Swanson et al., What human sperm RNA-Seq tells us about the microbiome. Journal of Assisted Reproduction and Genetics (Feb. 4, 2020). This study was designed to assess the capacity of human sperm RNA-seq data to gauge the diversity of the associated microbiome within the ejaculate. Semen samples were collected, and semen parameters evaluated at time of collection. Sperm RNA was isolated and subjected to RNA-seq. Microbial composition was determined by aligning sequencing reads not mapped to the human genome to the NCBI RefSeq bacterial, viral and archaeal genomes following RNA-Seq. Analysis of microbial assignments utilized phyloseq and vegan. Microbial composition within each sample was characterized as a function of microbial associated RNAs. Bacteria known to be associated with the male reproductive tract were present at similar levels in all samples representing eleven genera from four phyla with one exception, an outlier. Shannon diversity index (p<0.001) and beta diversity (unweighted UniFrac distances, p=9.99 e-4; beta dispersion, p=0.006) indicated the outlier was significantly different from all other samples. The outlier sample exhibited a dramatic increase in Streptococcus. Multiple testing indicated two operational taxonomic units, S. agalactiae and S. dysgalactiae (p=0.009), were present. These results provide a first look at the microbiome as a component of human sperm RNA sequencing that has sufficient sensitivity to identify contamination or potential pathogenic bacterial colonization at least among the known contributors.
Tacke et al., Levels of circulating miR-133a are elevated in sepsis and predict mortality in critically ill patients. Crit. Care Med., 42(5):1096-104 (May 2014).
The Gene Ontology Resource: 20 years and still GOing strong. Nucleic Acids Research, 47, D330-d338 (2019).
Tompkins, Genomics of injury: The Glue Grant experience. The journal of Trauma and Acute Care Surgery, 78(4), 671-86 (2015).
Traylen et al., Virus reactivation: A panoramic view in human infections. Future Virol., 6(4), 451-63 (April 2011).
Vijayan et al., Procalcitonin: A promising diagnostic marker for sepsis and antibiotic therapy. J. Intensive Care, Aug. 3, 2017.
Walton et al., Reactivation of Multiple Viruses in Patients with Sepsis. PLoS One, 9(6), e98819 (Jun. 11, 2014).
Wang et al., MicroRNA as biomarkers and diagnostics. J. Cell Physiol., 231(1), 25-30 (2016).
Wu et al., Profiling circulating microRNA expression in experimental sepsis using cecal ligation and puncture. PLoS One, 8(10) (2013).
Zander & Farver, Pulmonary pathology e-book: A volume in foundations in diagnostic pathology series. (Elsevier Health Sciences, 2016).
Rhee et al. Increasing trauma deaths in the United States. Annals of Surgery 260, 13 (2014)
R Core Team. R: A language and environment for statistical computing. (R Foundation for Statistical Computing, 2017).
Xiao et al. A genomic storm in critically injured humans. J Exp Med 208, 2581-2590 (2011).
Wickham, ggplot2: elegant graphics for data analysis. (Springer New York, 2009).
Dong, Du, & Gardner, An interactive web-based dashboard to track COVID-19 in real time. The Lancet, Infectious Diseases (2020).
Sethuraman, Jeremiah, & Ryo A, (2020) Interpreting Diagnostic Tests for SARS-CoV-2. JAMA.
Bouadma et al. (2020) Immune Alterations in a Patient with SARS-CoV-2-Related Acute Respiratory Distress Syndrome. Journal of Clinical Immunology, 1-11.
Fredericks et al. (2020) Alternative RNA splicing and alternative transcription start/end in acute respiratory distress syndrome. Intensive Care Medicine.
Sterne-Weiler et al. (2018) Efficient and accurate quantitative profiling of alternative splicing patterns of any complexity on a laptop. Molecular Cell, 72: 187-200.e186.
Beigel et al., (2020) Remdesivir for the treatment of Covid-19—Preliminary Report. New England Journal of Medicine.
Singer et al. (2016) The third international consensus definitions for sepsis and septic shock (Sepsis-3). JAMA, 315: 801-810.
Ferguson et al. (2012) The Berlin definition of ARDS: An expanded rationale, justification, and supplementary material. Intensive Care Medicine, 38: 1573-1582.
Andrews (2014) A quality control tool for high throughput sequence data. FastQC.
Dobin et al. (2013) STAR: ultrafast universal RNA-seq aligner. Bioinformatics (Oxford, England), 29: 15-21.
Boratyn et al. (2019) Magic-BLAST, an accurate RNA-seq aligner for long and short reads. BMC Bioinformatics, 20: 405.
Wood, Lu, & Langmead, (2019) Improved metagenomic analysis with Kraken 2. Genome Biology, 20: 257.
Mi et al. (2013) Large-scale gene function analysis with the PANTHER classification system. Nature Protocols, 8: 1551-1566.
Fleige & Pfaffl (2006) RNA integrity and the effect on the real-time qRT-PCR performance. Molecular Aspects of Medicine, 27: 126-139.
SEQC Consortium (2014) A comprehensive assessment of RNA-seq accuracy, reproducibility and information content by the Sequencing Quality Control Consortium. Nature Biotechnology, 32: 903-914.
Kujawski et al. (2020) Clinical and virologic characteristics of the first 12 patients with coronavirus disease 2019 (COVID-19) in the United States. Nature Medicine.
Chen et al. (2020) Detectable 2019-nCoV viral RNA in blood is a strong indicator for the further clinical severity. Emerging Microbes & Infections 9: 469-473.
Fang et al. (2020) Comparisons of viral shedding time of SARS-CoV-2 of different samples in ICU and non-ICU patients. The Journal of Infection.
Huang et al. (2020) Clinical features of patients infected with 2019 novel coronavirus in Wuhan, China. Lancet (London, England) 395: 497-506.
Gordon et al., A SARS-CoV-2 protein interaction map reveals targets for drug repurposing. Nature (Apr. 30, 2020).
Lai, Wang, & Hsueh (2020) Co-infections among patients with COVID-19: The need for combination therapy with non-anti-SARS-CoV-2 agents? Journal of Microbiology, Immunology, and Infection, 53: 505-512.
Feng et al., (2020) COVID-19 with different severity: A multi-center study of clinical features. American Journal of Respiratory and Critical Care Medicine.
Sharifipour et al. (2020) Evaluation of bacterial co-infections of the respiratory tract in COVID-19 patients admitted to ICU. BMC Infectious Diseases, 20: 646.
Poland, Ovsyannikova, & Kennedy (2020) SARS-CoV-2 immunity: review and applications to phase 3 vaccine candidates. Lancet (London, England).
The RECOVERY Collaborative Group (2020) Dexamethasone in hospitalized patients with Covid-19—Preliminary report. New England Journal of Medicine.
Prescott & Rice (2020) Corticosteroids in COVID-19 ARDS: Evidence and hope during the pandemic. JAMA, 324: 1292-1295.
Waterer & Rello (2020) Steroids and COVID-19: We need a precision approach, not one size fits all. infectious diseases and therapy.
Bellesi et al., (2020) Increased CD95 (Fas) and PD-1 expression in peripheral blood T lymphocytes in COVID-19 patients. British Journal of Haematology.
Robilotti et al. (2020) Determinants of COVID-19 disease severity in patients with cancer. Nature Medicine, 26: 1218-1223.
Vivarelli et al. (2020) Cancer Management during COVID-19 pandemic: is immune checkpoint inhibitors-based immunotherapy harmful or beneficial? Cancers, 12.
Hadjadj et al. (2020) Impaired type I interferon activity and inflammatory responses in severe COVID-19 patients. Science (New York, N.Y., USA) 369: 718-724.
Lei et al. (2020) Activation and evasion of type I interferon responses by SARS-CoV-2. Nature Commun., 11: 3810.
Al-Samkari et al. (2020) COVID and coagulation: Bleeding and thrombotic manifestations of SARS-CoV2 Infection. Blood
Rossignol, Gagnon, & Klagsbrun (2000) Genomic organization of human neuropilin-1 and neuropilin-2 genes: identification and distribution of splice variants and soluble isoforms. Genomics 70: 211-222.
Daly et al. (2020) Neuropilin-1 is a host factor for SARS-CoV-2 infection. Science (New York, N.Y., USA).
Ackermann et al. (2020) Pulmonary vascular endothelialitis, thrombosis, and angiogenesis in Covid-19. The New England Journal of Medicine, 383: 120-128.
Tian et al. (2020) Predictors of mortality in hospitalized COVID-19 patients: A systematic review and meta-analysis. Journal of Medical Virology.
Zhang et al. (2020) D-dimer levels on admission to predict in-hospital mortality in patients with Covid-19. Journal of thrombosis and haemostasis: JTH, 18: 1324-1329.
McElvaney et al. (2020) A linear prognostic score based on the ratio of interleukin-6 to interleukin-10 predicts outcomes in COVID-19. EbioMedicine, 61: 103026.

### Materials and Methods

Mouse strains. Mice are purchased from The Jackson Laboratory. C57BL/6J, the most popular mouse model used, exhibits a Th1/more pro-inflammatory phenotype. C57BL/6J is also the background of numerous knock out animals. BALB/cJ is also another commonly used mouse and can be the background of analyses with knockout animals, but has more of a Th1/anti-inflammatory predominant repose phenotype. The CAST mouse is derived from wild mouse and genetically different from common laboratory mice. Using these three strains adjusts for the heterogeneity seen in humans.

Mouse model of sepsis; cecal ligation and puncture (CLP). A mouse model of hemorrhagic shock followed by the induction of sepsis by cecal ligation and puncture induces severe sepsis. Lomas-Neira et al., Shock, 45(2), 157-65 (2016)); Monaghan et al., Mol Med., 24(1), 32 (Jun. 18, 2018); Wu et al., PLoS One, 8(10) (2013); Monaghan et al., Annals of Surgery, 255, 158-164 (2012). Anesthetized, restrained mice in supine position catheters are inserted into both femoral arteries. Mice are bled over a 5-10-minute period to a mean blood pressure of 30 mmHg (±5 mmHg) and kept stable for 90 minutes. To achieve this level of hypotension, the mice have one mL of blood withdrawn. One mL of blood is approximately 50% of their blood volume so this correlates to class 4 hemorrhagic shock in humans. Mice are resuscitated intravenously (IV) with Ringers lactate at four times drawn blood volume. Sham hemorrhage are performed as a control in which femoral arteries ligated, but no blood are drawn to mimic the tissue destruction. The following day, sepsis is induced as a secondary challenge by cecal ligation and puncture. The timing of this secondary challenged is based on previous findings that hemorrhagic shock followed twenty-four hours by the induction of sepsis produced results in line with critical illness such as altering PaO₂ to FIO₂ ratios. The mouse model uses a double hit of hemorrhagic shock followed by cecal ligation and puncture correlates to a missed bowel injury in humans after hemorrhagic shock. This mouse model correlates with an injury severity score (ISS) of twenty-five. The dual challenge of hemorrhagic shock followed by septic shock is in line with the sepsis patients who are critically ill. Sometimes patients present with bleeding from wounds and a bowel injury that is missed upon initial assessment.

Sample sizes for these assays are based upon results from the inventor's previous work looking at the alternative splicing of sPD-1 and an effect size of Cohen's d=2.85 standard deviations difference between groups was calculated. With such a large effect size, power analysis poorly justifies sample size since, if the effect size is tenable, it would be exceedingly rare for assays of any sample size to fail to reach statistical significance. However, small sample sizes provide poor point estimates and may be very unstable. the inventors chose a sample size of six mice per group based on feasibility and hoping to provide a reasonable point estimate for each group.

Mice of both sexes are used, because there are significant sex differences in the response to bleeding from trauma. Deitch et al., Annals of Surgery, 246(3), 447-53; discussion 53-5 (2007).

Human subjects. Patients are recruited from the Trauma Intensive Care Unit (TICU) at Rhode Island Hospital with Institutional Review Board approval and consent. The patient population at Rhode Island Hospital (a level 1 trauma center) is sufficient for this EXAMPLE. Over 3700 trauma patients were admitted to the hospital in 2018. The TICU admitted 765 patients in 2018. This would cause over 3000 patients admitted to the intensive care unit over the 4-year project. Using the advanced technology of the hospital's electronic health records (EPIC) combined with the mandated trauma registry there are streamlined efforts to recruit and retain patients. Since the mouse model correlates to an injury severity score (ISS) of twenty-five, the goal are to ensure that the average ISS for all the patients is twenty-five. Minimal risk to the patient are maintained since there is no direct benefit; the blood collected are less than 50 mL over an 8-week period and not collected more than twice a week. Blood samples from patients are taken on admission (25 mL) and during the TICU stay when a complication is developed (25 mL). This should cause the maximum for the initial 8-week period after the trauma. When the patient is recovered, at least 8 weeks after the last blood draw, a final blood draw 50 mL of are done in the outpatient setting. A power analysis was done based upon previous results from human patients. The effect size of Cohen's d=0.8 using a power of 80% and alpha of 0.05 the inventors calculated a sample size of twenty-six per group. The mortality of patients in the TICU is 5%. To enroll twenty-six patients who die after trauma, the inventors need 520 TICU patients (26/0.05=520). No enrollment is planned in the last six months to ensure adequate follow up, data collection and analysis. Fourteen % of patients in the TICU have complications after trauma. Due to the correlation to the mouse model of an ISS of twenty-five, the average ISS for the enrolled patients are targeted at twenty-five. This causes the recruitment of some patients who are not used, however the samples are banked and not sent for RNA sequencing. After twenty-six patients who die and twenty-six patients with a complication are enrolled and the entire set of patients has an average ISS of twenty-five then recruitment will conclude.

Where patients are being recruited, variables such as age, weight, and medical co-morbidities are collected and compared across groups. If these variables are different (t test or rank sum), these factors are adjusted for in the analysis by regression.

In the human studies, both sexes are recruited and analyzed in the GTEx data set. Age, weight, and other health problems are constant in the mouse assays.

Sample collection and sequencing. Mouse blood and lung samples were obtained as described. Monaghan et al., Annals of Surgery, 255, 158-164 (2012). Data for humans was obtained from GTEx by their protocols. RNA was extracted using the MasterPure Complete DNA/RNA Purification kit (epicenter, Madison Wis., USA) followed by the Globin Clear Kit (ThermoScientific, Waltham, Mass., USA). RNA was then sent to Genewiz (South Plainfield, N.J., USA) for sequencing as 1400 ng RNA in forty μL of fluid.

The GTEx Project was supported by the Common Fund of the Office of the Director of the National Institutes of Health, and by NCI, NHGRI, NHLBI, NIDA, NIMH, and NINDS and the data used for the analyses were obtained from the GTEx Portal and dbGaP accession number phs000424.v6.p1.

Cloud based computing. All computational biology work are performed on cloud-based computing by Lifespan-RI Hospital approved and supported Microsoft Azure environment. This server manages all large data sets from RNA sequencing. An intentional decision was made to use cloud-based computing for this project. Due to the depth of sequencing that is needed for RNA splicing analysis (100 million reads vs. forty million), more data is generated from both sequencing and analysis (a small study generated one terabyte of sequencing data and another terabyte from the alignment to the genome). With such a large amount of data predicted available for the EXAMPLE, the ability to expand and contract the storage space and computing power in the cloud is the ideal choice. This server stores and analyzes data from both mouse and human samples. Since RNA sequencing data is always identifiable, the data from humans are treated as though it is protected health information (PHI), even though none of the typical identifiers (such as name, date of birth, etc.) are associated with the data. The server was created in collaboration with the Information Technology department at Rhode Island Hospital to ensure data security. The cloud server is only accessible through a hospital virtual desktop and data are saved only to the Azure server or a hospital computer. Data are encrypted while stored, and when in transit to or from the hospital. Any link to typical identifiers (name, date of birth, etc.) are kept separate from the sequencing data. The cloud-based server allows for large data analysis with computing and storage needs changing on a per-use basis. The Azure server is Linux based and uses programming in R and Python. The following pipeline encompasses the typical analysis: differential expression, RNA analysis is done with Whippet. This also includes an entropy measure, and genes of interest undergo GO term analysis. Genes with alternative transcription start and end sites identified through Whippet are correlated with findings from the mountainClimber analysis.

Computational analysis and statistics. RNA sequencing data from the mouse was first checked for quality using FASTQC. RNA-sequencing data collected from the GTEx consortium and the mouse ARDS model was analyzed with the Whippet software for differential gene processing. Alternative transcription events are those events identified by Whippet as ‘tandem transcription start site,’ ‘tandem alternative polyadenylation site,’ ‘alternative first exon,’ and ‘alternative last exon.’ Alternative RNA splicing events are those events labeled ‘core exon,’ ‘alternative acceptor splice site,’ ‘alternative donor splice site,’ and ‘retained intron.’ Alternative mRNA processing events where determined by a log 2 fold change of greater than 1.5+/−0.2. Statistical significance was calculated by the chi-square p-value of a contingency table based on 1000 simulations of the probability of each result.

Gene ontology (GO) was assessed using The Gene Ontology Resource Knowledgebase. Ashburner et al., Nature Genetics, 25, 25-29 (2000); The Gene Ontology Resource: 20 years and still GOing strong. Nucleic Acids Research, 47, D330-d338 (2019). Genes from the analyses were entered and outputs displayed. Outputs from gene ontology do not correlate with actual increase or decrease in a gene's expression but are related to expected based upon the set of genes entered.

Blood sample collection. Blood samples are collected on day 0 of ICU admission. Clinical data including COVID specific therapies was collected prospectively from the electronic medical record and participants were followed until hospital discharge or death. Ordinal scale can be collected as previously described by Beigel et al., (2020) New England Journal of Medicine; along with sepsis and associated SOFA score [See Singer et al., (2016) The Third International Consensus Definitions for Sepsis and Septic Shock (Sepsis-3). JAMA, 315: 801-810], and the diagnosis of ARDS [See Ferguson et al. (2012) The Berlin definition of ARDS: An expanded rationale, justification, and supplementary material. Intensive Care Medicine, 38: 1573-1582].

RNA extraction and sequencing. Whole blood can be collected in PAXgene tubes (Qiagen, Germantown, Md.) and sent to Genewiz (South Plainfield, N.J., USA) for RNA extraction, ribosomal RNA depletion and sequencing. Sequencing can be done on Illumina HiSeq machines to provide 150 base pair, paired-end reads. Libraries were prepared to have three samples per lane. Each lane provided 350 million reads ensuring each sample had >100 million reads.

Computational Biology and Statistical Analysis. All computational analysis can be done blinded to the clinical data. The data can be assessed for quality control using FastQC [Andrews (2014) A quality control tool for high throughput sequence data. FastQC]. RNA sequencing data can be aligned to the human genome utilizing the STAR aligner [Dobin et al. (2013) Bioinformatics (Oxford, England), 29: 15-21]. Reads that aligned to the human genome can be separated and referred to as ‘mapped’ reads. Reads that do not align to the human genome, which are typically discarded during standard RNA sequencing analysis, were kept and identified as ‘unmapped’ reads. The unmapped reads then aligns to the releavant comparator and counted per sample using Magic-BLAST [Boratyn et al. (2019) BMC Bioinformatics, 20: 405]. The unmapped reads were further analyzed with Kraken2 [Wood, Lu, & Langmead, (2019) Genome Biology, 20: 257] using the PlusPFP index to identify other bacterial, fungal, archaeal and viral pathogens [see Kraken 2/Bracken Refseq indexes maintained by BenLangmead. It uses Kutay B. Sezginel's modified version of the minimal GitHub pages theme].

Reads that align to the human genome, the mapped reads, also can undergo analysis for gene expression, alternative RNA splicing, and alternative transcription start/end via Whippet [Sterne-Weiler et al., (2018) Molecular Cell, 72: 187-200.e186]. When comparisons are made between groups (died vs. survived) differential gene expression can be set with thresholds of both p<0.05 and +/−1.5 log 2 fold change. Alternative splicing was defined as core exon, alternative acceptor splice site, alternative donor splice site, retained intron, alternative first exon and alternative last exon. Alternative transcription start/end events can be defined as tandem transcription start site and tandem alternative polyadenylation site. Alternative RNA splicing and alternative transcription start/end events can be compared between groups [Sterne-Weiler et al., (2018) Molecular Cell, 72: 187-200.e186]. Significance was set at great than 2 log 2 fold change as previously described [Fredericks et al., (2020) Intensive Care Medicine]. Genes identified from the analysis of mapped reads can be evaluated by GO enrichment analysis (PANTHER Overrepresentation released 20200728) [Mi et al. (2013) Nature Protocols, 8: 1551-1566].

Whippet can be used to generate an entropy value for every identified alternative splicing and transcription event of each gene. These entropy values are created without the need for groups used in the gene expression analysis. To visualize this data a principal component analysis (PCA) can be conducted to reduce the dimensionality of the dataset and to obtain an unsupervised overview of trends in entropy values among the samples. Raw entropy values from all samples can be concatenated into one matrix and missing values were replaced with column means. Mortality can be overlaid onto the PCA plot to assess the ability of these raw entropy values to predict this outcome in this sample set. This analysis was done in R (version 3.6.3).

The following EXAMPLES are provided to illustrate the invention and should not be considered to limit its scope.

### Example 1

### Unmapped Bacterial Reads to Identify Bacteria Causing Sepsis

Because bacterial infections are a common cause of morbidity in trauma patients, unmapped reads that align with bacteria are useful for the diagnosis and treatment of trauma patients. Unmapped reads from RNA sequencing data provide a valuable tool for the trauma patient. The decrease in the number of bacterial reads in the blood may be due to increased immune response. Some bacteria keep constant levels between groups, which signifies a virulent pathogen.

The technique of RNA sequencing has resulted in creating massive amounts of data. The first step with public RNA sequencing data is usually to align the reads to the reference genome of interest. RNA sequences that do not align with the reference genome (10-30%) are usually discarded when they cannot be mapped.

The inventors use a mouse model of hemorrhagic shock followed by cecal ligation and puncture. The inventors isolate RNA from blood and lung samples and had the RNA sequenced using standard techniques. They compare RNA from the test mice to sham controls. They analyze the RNA data that did not map to the mouse genome. Unmapped reads aligned to common bacterial pathogens, including Acinetobacter baumannii, Escherichia coli, Klebsiella pneumoniae, Pseudomonas aeruginosa, Staphylococcus aureus, Streptococcus agalactiae, Streptococcus pneumoniae, and Streptococcus pyogenes. The inventors also identify specific genes with high read counts.

In one assay, the blood samples from the test mice exposed to trauma had fewer reads mapping to bacteria (365,974) as compared to the control mice (902,063, p=0.02). In the lung, the bacteria counts were similar. Despite an overall decrease in mapped bacterial RNA reads in the test mice, the three Streptococcus species and Staphylococcus aureus had a similar number of reads mapping between the test mice and the control mice. The most common RNA read mapped to aldo/keto reductase gene from group B strep (82793634[uid]). There was more expression of this gene in the blood of mice after trauma (15,096) compared to controls (3671, p=0.006). This difference was not seen in the lung compartment (13,691 vs. 15,996, p=0.24). In the blood of the test mice, most of the identified bacterial sequences were reduced in counts compared to the blood of the control mice (43 vs. 16).

### Example 2

### Unmapped Viral Reads to Identify Sepsis or Viral Reactivation

Unmapped data have been aligned to regions in the genomes of viruses. In critical illness, not only does the percentage of unmapped reads suggest a biomarker, but also the alignment of unmapped reads to some viral genomes. The percentage of unmapped reads in these organs during periods of critical illness can be a biomarker of severity and outcomes.

To assess the impact of critical illness on unmapped reads and their composition, the inventors expose mice (e.g., C57BL6 mice) to sequential treatment of hemorrhagic shock followed by sepsis. This treatment produces indirect acute respiratory distress syndrome (ARDS). RNA is extracted from lung and blood samples and sequenced via next-generation RNA-sequencing. Reads are aligned to the mm9 reference genome. The sources of unmapped reads were aligned by Read Origin Protocol (ROP). Changes in the viral signature of the unmapped reads are different when comparing blood to the lung.

In a second assay, the blood samples of critically ill mice averaged 31.9 million reads versus 32.1 million reads in healthy mice, and lung samples of critically ill mice averaged 33 million reads versus 33.7 million reads in healthy mice. The blood of critically ill mice had an average of 1.5 million unmapped reads (4.74%), more than the average 52,000 unmapped reads (0.16%) in the blood of healthy mice (p=0.000082). The lungs of critically ill mice had, on average, 194,331 unmapped reads (0.58%), which was more than the average 130,480 unmapped reads (0.39%) seen in the lungs of healthy mice (p=0.031665). In blood samples, unmapped reads from critically ill mice were less likely to be viral than healthy mice (average 3480 in critically ill vs. 4866 in healthy, p=0.025955). In lung samples, unmapped reads from critically ill mice were more likely to be viral than those from healthy mice (average 6959 in critically ill vs. 3877 in healthy, p=0.031959). The results were notable for higher viral loads in lungs of critically ill mice, showing that viral RNA loads can be a biomarker of critical illness.

Human correlates can translate into a clinical setting.

### Example 3

### Unmapped B/T V(D)J Use to Identify Sepsis

In immune systems, V(D)J recombination allows for a diversity of antibodies in B cells and T cell receptors in T cells. During critical illness, the variety of these recombination events reduces, but recovers. RNA sequencing better characterizes V(D)J recombination events. RNA sequencing shows more diversity in critical illness compared to what was described previously. B and T cell composition could prove to be an important marker in critical illness and predicting outcomes of sepsis.

The inventors subject mice (e.g., C57BL6 mice) to sequential treatments of hemorrhagic shock followed by sepsis. This treatment induces acute respiratory distress syndrome (ARDS). Lung and blood samples are collected. RNA from the samples are sequenced by next-generation sequencing. Reads from critically ill and healthy mice are aligned to GRCm38 annotation and then mapped to the V(D)J annotation by Read Origin Protocol (ROP).

In a third assay, the inventors recovered ˜thirty million reads were recovered from RNA-seq data generated from lung tissue of critically ill mice and healthy controls. Alignment with STAR aligner showed an average of 7.77% unaligned reads in the healthy control, and 8.78% unaligned reads in the samples extracted from critically ill mice. Unmapped reads then underwent a secondary alignment to assay for V(D)J recombinants. Healthy mice have an average of 629 recombinant epitopes, whereas critically ill mice had an average of only 208 recombinant epitopes. Assays were done in triplicate with littermates.

Analysis of unmapped reads shows that critical illness inhibits the generation of B cell and T cell epitopes by the immune system during critical illness. Although the percentage of unmapped reads between healthy mice and critically ill mice was not significant, the composition of B and T cell epitopes differs vastly in critically ill mice.

### Example 4

### Principal Component Analysis of RNA Splicing Entropy to Identify Sepsis

Next Generation Sequencing is useful for the diagnosis and treatment of diseases.

The effect of alternative RNA splicing before translation has not been studied much, especially in the critically ill patient. Previous work showed an association between cancer and the level of global alternative splicing entropy. Elias & Dias, Cancer Microenvironment, 1(1), 131-9 (2008); Ritchie et al., PLoS Computational Biology, 4(3), e1000011 (2008). RNA splicing entropy is correlated with acute respiratory distress syndrome (ARDS) across multiple tissues. Evaluating splicing entropy can provide insights about biological processes and gene targets in the critical illness setting.

The inventors induce a mouse model of ARDS by subjecting mice to hemorrhagic shock, followed by cecal ligation and puncture. Blood and lung samples are collected from three mice undergoing ARDS and three sham controls. RNA is purified.

Next-generation RNA sequencing is performed. Alternative splicing (AS) entropy levels are determined using Whippet (v 0.11) on Julia (v 0.6.4). Principal Component Analysis (PCA) is conducted using base R (v 3.4.0). Alternative splicing events with a proportion of spliced in values between 0.05 and 0.95 are analyzed. A threshold of 1.5 is applied to determine the percentage of high entropy events. Proportions of high entropy events across tissues and experimental groups are compared using Mann Whitney U tests.

In a fourth assay, Principal Component Analysis of the blood samples was performed. Samples clustered based on tissue type and ARDS status on a Principal Component Analysis plot This result suggested that splicing entropy can serve as a biomarker for ARDS status. The inventors observed differential levels of splicing entropy across tissue types, with the most entropy in the lung.

### Example 5

### RNA Lariats to Identify Sepsis

This EXAMPLE demonstrates the collecting of RNA sequencing data from a complex tissue (blood), rather than a cell line, and uses computational biology techniques to analyze the data.

RNA splicing occurs directly after DNA transcription, but before protein translation. RNA splicing by a two-step esterification process with the formation of an intermediary lariat formed by the intron and joining of the 5′ and 3′ splice sites. Introns typically degrade rapidly.

The biology of lariats has recently been identified as important as it relates to viral biology. The DBR1 gene encodes for the only RNA debranching enzyme. Mutations of DBR1 increase susceptibility to HSV1 and increase viral brainstem infections in humans. Assessing the RNA lariat counts in the critically ill trauma patients could predict poor outcomes or prolonged immune suppression. The inventers undertook the mouse model of critical illness (CLP). Assessing for the resolution or return to a healthy level of lariat counts could be a marker to identify immune suppression or those patients at risk for a complication.

The identification of lariats from RNA sequencing data has been difficult. However, the William G. Fairbrother laboratory created a method to count lariats from RNA sequencing data. Taggart et al., Nature Structural & Molecular Biology, 19, 719-721 (2012).

In a fifth assay, the preliminary data suggests that in the critically ill mouse, the typical metabolism of RNA lariats is changed, resulting in an accumulation of lariats in the blood. The inventors found that the blood of mice with the critical illness have higher lariat counts compared to the control mice.

### Example 6

### Traumatic Shock

Lungs from healthy mice had an average of 3877 viral reads. Lungs from critically ill mice had on average 6956 viral reads. Blood from healthy mice had 4866 viral reads. Blood from critically ill mice had 3480 viral reads. Lungs from critically ill mice were more likely to have unmapped reads originating from viral genomes when compared to lungs from healthy mice (0.36% in critically ill, 0.21% in healthy; p-value=0.032). This could be due to critical illness leading to a compromised immune response that allows for viral reactivation and a higher viral load in lungs of critically ill mice. Traylen et al., Future Virol., 6(4), 451-63 (April 2011).

Blood of healthy mice were more likely to have unmapped reads originating from viral genomes than blood of critically ill mice (0.05% in critically ill, 0.11% in healthy; p-value=0.026). There are several explanations for why healthy mice could have increased viral loads in the blood compared to critically ill mice. Mature lymphocytes are constantly recirculating through blood and lymphatic organs. Charles et al., Immunobiol. Immune Syst. Health Dis. 5th Ed. (2001). In critical illness, the release of pro-inflammatory mediators may compound the intensity of immune surveillance, as documented in patients with systemic inflammatory response syndrome (SIRS). Duggal et al., Science Reports, 8(1), 1-11 (Jul. 5, 2018).

Change in leukocyte populations in critically ill mice may lead to a higher number of RNA-producing polymorphonucleocytes (PMN) in blood, which reduces the total viral RNA signal in critically ill mouse blood. Therefore, steps are taken to enrich for lymphocytes and monocytes to reduce RNA reads from PMNs.

This traumatic shock EXAMPLE demonstrated an association between critical illness and higher viral loads in mouse lung, lending promise to the clinical use of viral loads as a marker of critical illness.

### Example 7

### Processing RNA Sequencing Data to Aid in the Care of Sepsis Patients

More should be known about RNA biology, specifically alternative RNA splicing, in the sepsis population.

Over 90% of human genes with multiple exons require alternative splicing events to produce functional proteins. Pan et al., Nature Genetics 40, 1413-1415 ((2008). RNA splicing creates a large natural source of variation of the transcribed gene to the produced protein product. RNA splicing is under exquisite control under normal conditions. Fever, hypothermia, and osmotic stress from fluid shifts can influence RNA splicing in vitro and change RNA splicing, altering protein expression. Gultyaev et al., TSitologiia i Genetika, 48, 40-44 (2014); Lemieux et al., PloS One 10, e0126654 (2015); Mahen et al., PLoS Biology 8, e1000307 (2010). Acidosis influences RNA splicing. Elias & Dias, Cancer Microenvironment, 1 131-139 (2008). Hypoxia also influences RNA splicing. Romero-Garcia et al., Experimental Lung Research 40, 12-21 (2014); Kasim et al., The Journal of Biological Chemistry, 289, 26973-26988 (2014). The effects of physiologic stress on RNA splicing should be better known. The pathological significance of changes induced RNA splicing process and proteins should be better understood.

This EXAMPLE shows the use of deep RNA sequencing data using computational biology methods (RNA splicing entropy, lariat counts, viral identification, and B and T cell epitope creation) and apply these methods to three distinct data sets: mouse of different strains undergoing sepsis, deceased sepsis patients who participated in the GTEx project, and human sepsis patients.

RNA splicing entropy after sepsis. RNA splicing is a basic molecular function in all cells. This EXAMPLE uses the global index/marker of RNA splicing called ‘RNA splicing entropy’ a calculation of the precision of RNA splicing typically occurring. The entropy and thus the disorder, is maximal when the probability of all events P (xi) is equally likely and the outcome is most uncertain. This calculation are done for each type of alternative splicing event: skipped exon, retained intron, alternative donor (3′ splice site), and alternative acceptor (5′ splice site). The alternative splicing events with high entropy are identified using Whippet.

A lower percentage of RNA slicing entropy may predict increased mortality or more complications, particularly infections, in patients with sepsis. Previous work on cancer samples has shown that RNA splicing entropy is increased in the tumor compared to the healthy tissue in many cancer types. From the preliminary data in mice with and without ARDS after sepsis, RNA splicing entropy is less in the blood, 7.7% vs 10.7%, p=0.1. RNA splicing entropy was calculated for total white blood cell components of mice with critical illness caused by hemorrhage and cecal ligation and puncture and compared to controls. The RNA from blood and the lungs of mice was extracted, processed and then subjected to deep RNA sequencing.

Obtaining this data demonstrates the ability to isolate RNA samples from the target organ tissues of interest in the mouse model system. This EXAMPLE demonstrates the ability to process the complex data using computational biology and custom scripts that result from RNA sequencing. This preliminary data suggests that the process of RNA splicing in critical illness is different compared to the controls. changes in RNA splicing entropy may be a reflection/response to or a mechanism driving pathological processes that drive mortality and morbidity in patients with sepsis. Genes with significant alternative splicing and high entropy in the mouse after sepsis may be target for intervention. These genes of interest are identified using machine-learning techniques and compared across both humans and mice.

Assessment of viral activity after sepsis. In the initial assessment of RNA sequencing data, the reads are aligned to the genome of the species the sample came from. The unmapped reads can account for up to 20% of the data and this data is typically discarded. From this Read Origin Protocol analysis of multiple data sets (including GTEx data), the inventors found their protocol accounted for 99.9% of all reads. The data typically discarded was then analyzed in a seven-step process. Two of those steps are of particular interest because of the relevance to critical care: Viral reads and B and T cell receptor rearrangement.

Identification of viruses after sepsis is a marker of immune suppression since there is data suggesting sepsis re-activates herpes infections. Cook et al., Critical Care Medicine, 31, 1923-1929 ((2003)). Much current research is focused on these mechanisms and interventions. Viral counts could correlate with immune suppression or complications. This is important because of the re-activation data. RNA sequencing data from the lungs of control mice showed fewer viral reads (3877) compared to mice after sepsis (6956, p=0.032). In the blood the opposite was true. Control had 4866 counts versus sepsis with 3480 counts (p=0.026). This difference between tissue types could be due to a multitude of reasons, such as latent infections, like CMV, in the lung. Because blood is the most accessible tissue type, the efforts for the human samples should focus on the blood.

Assessment of immune cell epitopes after sepsis. During critical illness, the immune system is activated and likely creating new receptors to respond to challenges/pathogens. These epitopes come from lymphocytes, known to be reduced in sepsis with resolution to normal levels linked to recovery. Heffernan et al., Critical Care, 16, R12 (2012). While the count of lymphocytes themselves is useful, measuring the number and diversity of the epitopes could provide further insights into immune suppression after sepsis.

In the mouse model, preliminary data shows fewer epitopes in the lung of mice after sepsis, compared to control. This demonstrates the ability to analyze data from a mouse model and characterize B and T cell epitopes via computational methods. Like lymphocytes, the production of epitopes may reduce. Recovery should correlate with a return to normal immune state.

The above-described methods to assess for immune suppression in sepsis patients by analysis of RNA sequencing data to understand RNA biology are applied to these samples.

For analysis of RNA splicing entropy, lariat counts, viral identification, and B and T cell epitope creation in the mouse model, using pilot data, using forty mice (twenty critically ill, twenty healthy controls) should have 80% power to detect a difference at a two-tailed alpha of 0.05. This method is used for each of the three mouse variants.

At the time points of twenty-four hours after cecal ligation and puncture and fourteen days after cecal ligation and puncture, mice are sacrificed and organs procured. Organs to be collected are brain, lung, heart, kidney, liver, spleen, and blood. RNA from these samples are isolated as described below. The time point of twenty-four hours after CLP is selected as that is the time of most significant organ dysfunction. The time point of fourteen days is selected, since this is the point at which a mouse would be considered a survivor after this challenge.

RNA from blood samples in the mouse are processed using the MasterPure Complete RNA Purification (epicenter, Madison Wis., USA) kit for mice. Due to the high concentration of globin RNA in blood samples, these samples can then be further processed with the GLOBINclear Kit (epicenter, Madison Wis., USA). From blood one of skill in the molecular biological art can get 30-50 nanograms per microliter, with a total blood volume isolated from the mouse of about one mL. RNA from lung, heart, brain, kidney, liver, and spleen samples are extracted using MasterPure Complete RNA Purification kit for mice. After RNA samples are processed, the RNA was sequenced using standard techniques, for example by Deep RNA sequencing with a goal of 100,000,000 reads per sample. All samples should require at least 1400 nanograms of RNA for deep sequencing.

Human samples. Patients are recruited under Institutional Review Board approval and after consent is obtained. Blood samples are obtained from pre-existing catheters to minimize the risk. Blood samples are collected on admission and serially while the patient is in the intensive care unit. Samples are collected in PAXgene tubes and stored in an −80 C freezer until isolation of RNA for sequencing is needed. RNA sequencing are done in batches to minimize cost. For this experiment, it is expected 300 sepsis patients are recruited (average of 100 the first three years to allow analysis over the final two years of the project).

Control samples are obtained from healthy patients undergoing routine laboratory analysis at outpatient facilities. Blood from these patients are collected in PAXgene tubes and stored in an −80 C freezer until isolation of RNA for sequencing is needed. RNA sequencing are done in batches to minimize cost. Healthy controls are matched to sepsis patients based upon demographic/clinical data. Recruitment aims for 300 patients total (average 100 each year over the first three years). Sample size calculations for the recruitment of humans was done based upon initial results from the mice assays. Preliminary data from humans with sepsis shows more variation compared to the mice data. These differences from humans are accounted for by several things such as age, sex, medical co-morbidities, and variations in the timing of collection from the point of the sepsis.

RNA from blood samples from humans are processed using the MasterPure Complete RNA Purification (epicenter, Madison Wis., USA) kit for humans. Due to the high concentration of globin RNA in blood samples, these samples can then be further processed with the GLOBINclear Kit (epicenter, Madison Wis., USA). All samples require at least 1400 nanograms of RNA for deep sequencing, e.g., by Deep RNA sequencing with a goal of 100,000,000 reads per sample.

Genotype Tissue Expression (GTEx). The GTEx data has over 500 patients included with at least one sample that has undergone RNA sequencing. Extensive clinical data is available on these participants. The data can stratify the patients into early deaths (<36 hours) and late deaths (>36 hours). This classification and comparison between the groups was done as it highlights a population who could be intervened upon. The patients who die later die because of immune suppression leading to complications from sepsis. Earlier identification of immune suppression could change outcomes. The GTEx samples have been collected and undergone RNA sequencing. This sequencing data are analyzed as described above.

Innovativeness. RNA sequencing technology affords an avenue to bring precision medicine to sepsis patients. The inventors used blood samples from sepsis patients, process them and obtain RNA sequencing data of similar quality to that of cell lines or solid tissue samples. Monaghan et al., Shock, 47, 100 (2017). RNA sequencing allows for understanding not only the gene expression but also RNA biology. RNA is unstable compared to DNA. Kara & Zacharias, Biopolymers, 101, 418-427 (2014). RNA is influenced by the specific cellular environment (altered in sepsis).

Conceptual Innovation. Past work on sepsis and molecular mechanisms has been focused on gene transcription and protein expression. The process of alternative RNA splicing also can influence the expression of a protein independent of the gene expression. Chang et al., Combinatorial Chemistry & High Throughput Screening, 13, 242-252 (2010); Fredericks et al., Biomolecules, 5, 893-909 (2015).

By comparing findings in mice to humans using the publicly available RNA sequencing data from GTEx and human samples from the Intensive Care Unit, the inventors can establish the nature/type of RNA splicing common across species.

By determining the temporal relationship of changes in RNA splicing entropy, RNA lariats, viral identification, and B and T cell epitope creation with developing complications/mortality, the inventors can establish whether RNA biology can provide insight to immune suppression after sepsis.

Assessing information in the unmapped reads (viral and B/T cell epitopes) to determine clinical significance is using data that is typically discarded. This is similar to the use of lymphocyte counts to predict sepsis outcomes. Heffernan et al., Critical Care, 16, R12 (2012).

Technical innovation. RNA are isolated from complex tissues from both mice and humans. The isolate RNA are of high enough quality to allow for deep RNA sequencing. This analysis has only previously been done on cell line or cancer samples.

The inventors can use a series of analytical algorithms; initially, using the STAR aligner, then Whippet to assess and characterize splicing events and splicing entropy. This analysis are done across GTEx data, mice with sepsis and humans with sepsis.

The inventors can use the Read Origin Protocol as a basis. The inventors can modify as appropriate to assess viral content and B/T cell epitopes in data obtained from mouse models of sepsis, GTEx, and humans with sepsis.

The inventors can apply the scripts used previously to calculate lariat counts from RNA sequencing data. Taggart et al., Nature Structural & Molecular Biology, 19, 719-721 (2012). The RNA sequencing data is obtained from mouse models of sepsis, GTEx, and humans with sepsis.

Assaying the large amount of data that comes from RNA sequencing is commonly not successful due to several reasons. The analyses have biases for which controls are not in place. the large data should produce a statistically significant result but is it biologically and clinically significant. Using multiple biologic outputs (RNA splicing entropy, lariat counts, viral identification, and B and T cell epitope creation) across three samples (GTEx, mouse model, and humans) will mitigate.

By assaying RNA splicing entropy, lariat counts, viral identification, and B and T cell epitope creation, one of ordinary skill in the molecular biological art can identify patients with this prolonged immune suppression.

Analyzing data already collected, such as using the GTEx data, and data like the unmapped reads from RNA sequencing supports creativity. This data would typically be ignored, but with the proper clinical relevance, the data can be reanalyzed and potentially find new biomarkers. The lymphocyte count on a complete blood count with differential, a potential biomarker in the sepsis population. Heffernan et al., Critical Care, 16, R12 (2012).

Analysis of RNA sequencing data can provide one marker of the severity of the critical illness.

Evaluating RNA biology and outcomes after sepsis. Next generation RNA sequencing allows for the analysis of the RNA and assessment of not only gene expression but also other biological processes (alternative splicing, changes in transcription start and end). Correlating genomic information from high throughput sequencing technologies about a patient on arrival to the hospital with outcomes such as death and complications like infection should improve care. Since RNA is not as stable as DNA, assessing RNA are more sensitive to the physiologic stress in sepsis. The inventors can assess how the physiologic stress of sepsis influences RNA biology and alters proteins. Assaying RNA biology in critical care sepsis patients should translate to other patients with critical care after diseases.

By high throughput RNA sequencing the inventors can assay gene expression and the RNA processing events of alternative transcription start/end and alternative RNA splicing of from leukocytes in the blood. All three of these biological processes influence protein expression via generation of the RNA (gene expression), changing the beginning and end of the RNA (alternative transcription start/end), and changing the isoforms that are expressed (alternative RNA splicing). The combination of these three modalities creates a ‘transcriptomic phenotype’ and better identifies expressed proteins in the sepsis population as compared to the typical use of gene expression alone. compared to DNA, RNA is more influenced by the physiologic derangements seen in sepsis such as hypoxia and acidosis in cell culture. Elias & Dias, Cancer Microenvironment, 1(1), 131-9 (2008); Kasim et al., The Journal of Biological Chemistry, 289(39), 26973-88 (2014).

In an intensive care unit, monitoring of physiology correlates to improved clinical outcome. Clinicians do not monitor how this physiology impacts RNA biology. Using high throughput sequencing, the inventors assay RNA biology in sepsis patients. The understanding of RNA biology at the time of injury should predict mortality, complications, and other outcomes in sepsis patients. Three aims are tested using a mouse model of sepsis, data from GTEx of sepsis patients, and blood from sepsis patients with correlation to outcomes.

Aim 1: Identify changes in RNA biology (gene expression, alternative transcription start/end, and alternative RNA splicing) in the blood before and after a pre-clinical mouse model of sepsis and compare to controls.

Aim 2: Using the data available from the Genotype Tissue Expression (GTEx) project correlate findings in the mouse model to these sepsis patients (81 patients).

Aim 3: Enroll critically ill sepsis patients and identify aspects of RNA biology that identify and predict outcomes (mortality, infection).

These analyses use data from high throughput sequencing and cloud computing to establish findings of RNA biology that correlate and predict outcomes in sepsis patients. This data comes from an ancestrally diverse sepsis population and can be applied to sepsis patients across the country and to multiple critically ill patient populations.

New technology has come that allows for analysis of all genes, not just those identified by the technology at the time. Tompkins, The Journal of Trauma and Acute Care Surgery, 78(4), 671-86 (2015). With RNA sequencing technology, particularly at the depth proposed (80-100 million reads) needed for RNA biology assessment, the inventors can assess all genes transcribed, not just those identified as important with older technology. The analysis of all transcribed genes allows for the identification of genes that may be important for trauma, that in the past were overlooked, likely due to low transcription levels. with RNA sequencing technology the inventors can assay RNA biology (alternative transcription start/end and alternative RNA splicing), for a complete understanding of what genes are ultimately translated to functional proteins. Hardwick et al., Frontiers in Genetics, 10, 709 (2019).

Over 90% of human genes with multiple exons require alternative splicing events to produce functional proteins, creating a potentially large natural source of variation of the transcribed gene to the produced protein product. Pan et al., Nature Genetics, 40(12), 1413-5 (2008). Splicing is under exquisite control under normal conditions. Some conditions common in trauma, such as fever, hypothermia, and osmotic stress from fluid shifts can influence RNA splicing in vitro and change RNA splicing, altering protein expression. Gultyaev et al., TSitologiia i Genetika, 48(6), 40-4 (2014); Lemieux et al., PloS One, 10(5), e0126654 (2015); Mahen et al., PLoS Biology, 8(2), e1000307 (2010).

Using a mouse model of trauma caused by hemorrhage followed by cecal ligation and puncture, the inventors reported that alternative RNA splicing results in expression of varied isoforms of an immune modulating protein (programmed cell death receptor-1, PD-1). Preliminary data on RNA splicing entropy indicate that global RNA splicing is modified in the mouse model of trauma. Ritchie et al., PLoS Computational Biology, 4(3), e1000011 (2008). Increased RNA splicing entropy is also present in other pathologic conditions, such as cancers, as compared to normal tissue. Ritchie et al., PLoS Computational Biology, 4(3), e1000011 (2008). Increased entropy is characteristic of disease states and could be a marker of critical illness after sepsis.

Sepsis patients are a good population in which to assay critical illness and generalize the findings to other patients. A population of sepsis patients is an ideal group to assay genomic factors as previous research has been hindered by lack of racial and ethnic diversity. Multiple factors cause minorities to avoid healthcare. Chikani et al., Public Health Reports, 131(5), 704-10 (2016). By assaying sepsis patients, the inventors can collect data from a diverse population that is more in line with the general population and not the population that seeks healthcare. The findings are more generalizable, especially among an ancestrally diverse population.

Protocols for sepsis have improved outcomes. Rhodes et al., Intensive Care Medicine, 41(9), 1620-8 (2015). Sepsis can cause critical illness in a young population. The response to sepsis should not be influenced by co-morbidities associated with an increasingly aged population, but the inventors can collect co-morbidities to assess if there is an impact.

Genomic medicine is an ideal target for sepsis patients but is limited by sequencing technologies. Although genomic medicine is typically defined as using genomic information about an individual patient as part of their clinical care, this definition cannot be applied to sepsis patients or any critically ill patients.

Next generation RNA sequencing takes about 18 hours on an Illumina machine, but this does not include time for data analysis. Since the data are delayed until the outcome of the patient is known, data analysis can be blinded to allow for more robust conclusions. through this work, the efficiencies in computation biology can be elucidated so that when the sequencing technology speeds up, the analysis are quick enough to have a clinically relevant time frame (less than one hour) from sample acquisition to actionable result.

Thus, there is value in understanding of how stressors associated with sepsis can affect RNA biology (RNA splicing (and entropy) and alternative transcription start/end) and how changes in the RNA biology leads to altered protein product expression, contributing to potential dysfunction at a cell and tissue level.

Innovation. Past work focusing on trauma and molecular mechanisms has been focused on gene transcription and protein expression. The process of alternative RNA splicing and alternative transcription start/end both have the potential to influence the expression of a protein independent of the gene expression. Chang et al., Combinatorial Chemistry & High Throughput Screening, 13(3), 242-52 (2010); Fredericks et al., Biomolecules, 5(2), 893-909 (2015). By comparing findings in mice to humans using the publicly available RNA sequencing data from GTEx and human samples from the Trauma Intensive Care Unit the inventors can establish the nature/type of RNA biology that is common across species.

In determining the temporal relationship of changes in RNA biology with developing complications/mortality, the inventors can establish whether RNA biology can provide insight to immune suppression after sepsis.

Knowledge of RNA biology in the critically ill is useful because previous work on this process has focused largely on chronic diseases and genetic diseases.

The combination of gene expression, RNA splicing, and transcription start/end create a ‘transcriptomic phenotype’ that can be followed during the patients hospital stay.

RNA are isolated from complex tissues from both mice and humans. The isolate RNA are of high enough quality to allow for deep RNA sequencing. This analysis has only previously been done on cell line or cancer samples.

The inventors can use a series of analytical algorithms using the STAR aligner, then Whippet, to assess and characterize RNA biology. Results from Whippet are compared to mountainClimber to ensure accurate data as it pertains to alternative transcription start and end. This analysis are done across GTEx data, mice with sepsis and humans with sepsis.

Using multiple biologic outputs (alternative RNA splicing, including entropy, alternative transcription start/end) across three different samples (GTEx, mouse model, and humans in the trauma intensive care unit) should mitigate some of the potential flaws.

Preliminary data regarding trauma. In a small cohort of trauma patients from GTEx, three patients form the early death cohort (<48 hours) were compared to six patients from the late death cohort (>/=48 hours). In this comparison, 524 genes are significantly increased in the late death versus the early death. In the late death group, 2331 genes are decreased compared to the early death group. The GO terms associated with the genes that decreased expression in the late group compared to the early group are valid based upon previous research. The terms with a decrease in expected representation in the GO terms reference mitochondrial biology. This decrease in GO terms likely represents that genes are increased in expression at the early death time point. Mitochondrial molecular patterns have been a component of the early response to trauma and those genes would be increased in the early group.(37, 38) anemia occurs during trauma. In the late group, genes associated with erythrocyte development are over-represented, suggesting increase expression in the late death group compared to the early death group. These few GO terms and correlation to phenotypes of trauma, suggest use of early versus late death is a valid clinical tool. This preliminary data shows the ability to access, manage, and analyze GTEx data with clinically significant groups using novel computational biology techniques. Using GO terms allows us to prove clinical relevance. This project aims to obtain and analyze all the trauma samples from GTEx. The inventors can also use similar computational approaches with the prospectively collected data from trauma patients.

Multiple alternative RNA splicing events and alternative transcription start and events are detected, but there are fewer that are significant. Using the same cohort as above, this preliminary date from GTEx data, alternative splicing and alternative transcription events are characterized using Whippet. Multiple events were identified to be alternative RNA splicing and alternative transcription start/end in the blood samples. When comparing the groups there were only significant differences when assessing alternative RNA splicing and not alternative transcription start and end. This data confirms that alternative RNA splicing is an active process during trauma and could predict mortality and outcomes in trauma patients. genes with changes in splicing, and potentially transcription start/end could identify novel targets. The combination of gene expression, splicing and transcription start/end could alter what proteins were thought to have increased gene expression and subsequent protein transcription have altered processing resulting in new isoforms or changes in transcription. These findings highlight the ability to access GTEx data, categorize the samples in a clinically relevant manner, and process the RNA sequencing data with advanced computational methods, such as Whippet.

RNA splicing, specifically RNA splicing entropy shows differences after trauma. From the preliminary data in mice with and without, the inventors can show that in the blood there is less RNA splicing entropy, 7.7% versus 10.7%, p=0.1. RNA splicing entropy was calculated using Whippet. The percentage of each type of splicing event with an entropy of >1.5 (Alternative Donor, Alternative Acceptor, Retained Intron, and Skipped Exon). Using the mouse model of trauma, RNA splicing entropy was calculated for total white blood cell components of mice after trauma caused by hemorrhage with cecal ligation and puncture (n=3) and compared to controls (n=3). The RNA from blood was extracted, processed and then subjected to deep RNA sequencing. This preliminary data suggests that the process of RNA splicing in critical illness is different compared to the controls. changes in RNA splicing entropy may be a reflection/response to or a mechanism driving pathological processes that drive mortality and morbidity in patients with trauma. Obtaining this data demonstrates the ability to isolate RNA samples from the target organ tissues of interest in the mouse model system. This EXAMPLE demonstrates the ability to process the complex data using computational biology and custom scripts that result from RNA sequencing.

The trauma patients in the intensive care unit provide an ancestrally diverse population and adequate numbers to correlate mortality and other complications. The trauma intensive care unit admits over 750 patients a year with 20% of those patients coming from an ancestrally diverse background. The enrollment is in line with the general population, even though underrepresented minorities seek medical care at a reduced rate. One aspect to this invention is the correlation of the RNA sequencing data to mortality and complications.

This EXAMPLE shows the importance of not only predicting mortality, but also using RNA sequencing data to predict complications as patients with complications had a higher mortality (7.7%). Mortality could be influenced. This data shows the trauma center has the volume of patients in the intensive care unit to have an appropriately powered study.

Over four years, 520 patients can be enrolled based on sample size calculations, with fewer than the 3000 expected admissions proving feasibility.

TABLE 1

| Aim | Suggested Type of Research | Application |
|---|---|---|
| 1 | Integration of other data types, | A model organism (mouse |
| | such as environmental data, family | after trauma) will provide |
| | history, transcriptomics, | the basis for other |
| | epigenomics, functional data, or | analyses in humans after |
| | model organism data to improve | trauma. Multiple strains |
| | assessment of clinical validity or | will mimic the diverse |
| | clinical utility of genomic | human population. |
| | information. | |
| 2 | Assessment of improved | GTEx data are re-analyzed |
| | approaches for reanalyzing patient | using modern approaches and |
| | genomic data and understanding | a unique population (early |
| | its impact on clinical care. | versus late trauma deaths) |
| 3 | Evaluation of modern approaches | Trauma patients will provide |
| | to interpreting genomic data in | an ancestrally diverse |
| | ancestrally diverse populations in | population to assay this |
| | clinical settings | clinical genomic date. |

This approach uses RNA sequencing data from a mouse model of trauma, re-analysis of existing genomic data in GTEx about early versus late trauma deaths, and samples from ancestrally diverse critically ill trauma patients uniquely suited to provide clinical information applicable across many clinical scenarios; particularly critically ill patients with cancer, sepsis, stroke, or myocardial infarction. The analysis of the RNA data from next generation sequencing technology create a ‘transcriptomic phenotype’ for each trauma patient. Understanding the RNA biology at the time of injury can predict outcomes (mortality and complications) in trauma patients. The method to test the three aims, the expected result, and the potential impact are summarized in TABLE 2.

TABLE 2

| Aim | Method | Result | Impact |
|---|---|---|---|
| 1 | Mouse model of | Changes in RNA biology | These findings provide the |
| | trauma, assessing | predict mortality after the | foundation for predicting |
| | blood before | mouse model of trauma. | mortality and complications |
| | trauma, after | The results seen at 24 | in critically ill trauma |
| | trauma, and in | hours differ from those | patients. Data seen at 24 |
| | survivors | identified at 14 days. | hours and 14 days correlate |
| | | | with patients who die early |
| | | | versus late. |
| 2 | 81 deceased | Changes in RNA biology | This are the foundation for |
| | trauma patients | are identified in early | analysis of RNA data from |
| | from GTEx, 23 | versus late trauma deaths | trauma patients during their |
| | early deaths and | and these correlate with | hospital stay. |
| | 58 late deaths | mouse data. | |
| 3 | Critically ill trauma | Changes in RNA biology | Using RNA sequencing data |
| | patients assessing | on admission predict | predict mortality and |
| | blood on | complications and | complications and enhance |
| | admission and | mortality, changes over | care of trauma patients with |
| | throughout course | the hospital course | applicability to all intensive |
| | | correlate with long-term | care unit patients. |
| | | outcomes. | |

Aim 1: Identify changes in RNA biology (gene expression, alternative transcription start/end, and alternative RNA splicing) in the blood before and after a pre-clinical mouse model of trauma and compare to controls.

Rationale: to determine if altered RNA biology in its various forms can predict outcomes, RNA sequencing data must be collected at various time points during the traumatic injury. The inventors can establish the equivalency of such a pre-clinical animal model to what is encountered clinically. The inventors previously used a mouse model of hemorrhagic shock followed my septic shock by cecal ligation and puncture (CLP). Monaghan et al., J. Transl. Med., 14(1), 312 (2016). This mouse model mimics a trauma patient with hemorrhagic shock from an extremity injury who then had a missed bowel injury resulting in severe critical illness. Using this mouse model, the inventors can obtain blood at the initial injury and assess if changes in RNA biology, to predict mortality from the severe trauma model. Using a mouse model allows for acquisition of blood samples at multiple time points (twenty-four hours after injury and in those mice that survived). The inventors can first assess if RNA biology in the blood can predict mortality, if changes in RNA biology are seen twenty-four hours after injury, and how these correlate to the RNA biology of survivors at fourteen days.

Test 1: Assess RNA sequencing data and identify genes with changes in expression, alternative RNA splicing, and alternative transcription start/end to develop the ‘transcriptomic phenotype’ from shed blood in the mouse model of trauma to predict outcomes. Mice (8-12 weeks old) undergo hemorrhagic shock followed by CLP to mimic the critical illness that a trauma would undergo after hemorrhagic shock from an extremity injury complicated by a missed small bowel injury. Mice are used from the background of C57BL/6J, BALB/cJ, and CAST to simulate the heterogeneity of humans. Each group has twenty-four (twelve sham and twelve trauma) mice for each strain based upon statistical calculations. C57BL/6J mice have a 30% survival at fourteen days. The shed blood from the hemorrhage component are collected. Although this blood is collected before the effects of hemorrhage, this time point can mimic an early time point in trauma, since the mice have undergone anesthesia and isolation/catheter insertion of the artery. RNA are isolated, sequenced and analyzed as described. The mice that survive to fourteen days can also be sacrificed and used in Test 2.

Test 2: Assess RNA sequencing data and identify genes with changes in expression, alternative RNA splicing, and alternative transcription start/end to develop the ‘transcriptomic phenotype’ from the blood of mice at twenty-four hours and fourteen days after trauma. Mice (8-12 weeks old) undergo hemorrhagic shock followed by CLP to mimic a severe trauma. Mice are used from the background of C57BL/6J, BALB/cJ, and CAST. Mice are sacrificed at twenty-four hours after CLP. Mice that survive to fourteen days are also sacrificed to assess RNA biology at that point among the survivors. Appropriate controls for each type of background mice undergo sham procedures. Based upon previous work, six mice are needed for each group. After mice are sacrificed (CO₂ overdose followed by direct cardiac puncture) at either twenty-four hours or fourteen days after CLP blood are harvested. RNA from blood samples in the mouse are processed.

Human samples. Through collaboration with the military, soldiers in combat areas could be consented to donate blood before deployment. This blood would then undergo RNA sequencing and be compared to samples collected if there was an unfortunate traumatic injury. Many previous efforts using animal models to treat diseases such as sepsis failed to translate to humans. Fink & Warren, Nature Reviews Drug Discovery, 13(10), 741-58 (2014). The inventors previously studied conditions in mice with correlation to humans. Monaghan et al., J. Transl. Med., 14(1), 312 (2016); Monaghan et al., Molecular Medicine, 24(1), 32 (2018); Monaghan et al., Journal of the American College of Surgeons, 213(3), S54-S5 (2011); Monaghan et al. Annals of Surgery 255(1), 158-64 (2012). Trauma research may have better translatable results because of the timing of the disease. In trauma, the time of the event is known. This timing correlates with the induced trauma in the mouse. In sepsis, the time point at which sepsis started in the mouse is known. However, in humans, the time at which sepsis starts is impossible to know, as exemplified by inability to understand when an appendix may perforate. Iacobellis et al., Seminars in Ultrasound, CT, and MR, 37(1), 31-6 (2016). This is limited because it is a controlled traumatic challenge and should produce very consistent response to trauma. In humans, no trauma is the same. The number of humans needed to detect a difference is more since the traumas are not similar. Humans have more heterogeneity adjusted for by using multiple mouse strains. The inventors can account for differences in trauma by using the Injury Severity Score. The ISS of this challenge on the mouse is twenty-five, and this is the target average ISS of patients enrolled.

Aim 2: Using the data available from the Genotype Tissue Expression (GTEx) project correlate findings in the mouse model to these trauma patients (81 patients).

Rationale. Using the GTEx data, the inventors can assess RNA biology in the blood of trauma patients. The GTEx data has over 500 patients included with at least one sample that has undergone RNA sequencing. The patients in the GTEx data set have extensive clinical data available. Unfortunately, all patients in this data set are deceased. This should be considered in interpretation of the data. To adjust for the fact all patients are deceased, the inventors use the time to procurement of the RNA from the death of the patient as a variable due to adjust for RNA degradation and other metrics as suggested by the GTEx consortium.(50) Trauma patients are selected (n=81) and identified as early (<48 hours) versus late death (>/=48 hours). The inventors can compare RNA biology between trauma patients who died early versus late and compare it to findings in a mouse model of mice who died early (twenty-four hours) versus survivors (fourteen days)

Test 1: Assess RNA sequencing data and identify genes with changes in expression, alternative RNA splicing, and alternative transcription start/end to develop the ‘transcriptomic phenotype’ the blood of deceased trauma patients and compare among early and late deaths. There are 81 unique trauma patients in the data set with blood samples. These patients are aged 20-68, in line with the age of typical trauma patients. The GTEx samples have been collected and undergone RNA sequencing. RNA sequencing data are aligned to the human genome with STAR. RNA Splicing events are assessed using Whippet and characterized into one of the five alternative splicing events: skipped exon, retained intron, mutually exclusive exon, alternative 3′ splice site, and alternative 5′ splice site. Entropy calculation are completed using Whippet. Alternative transcription events from Whippet are compared to outputs from mountainClimber.

Test 2: Correlation of changes in expression, alternative RNA splicing, and alternative transcription start/end (the ‘transcriptomic phenotype’) in the blood of humans to the mouse samples. From mouse model (Aim 1) changes in expression, alternative RNA splicing, and alternative transcription are identified and these are compared to findings in the human GTEx data (Aim 2, Test 1). The mouse model data are taken from mice at twenty-four hours after CLP and at fourteen days after CLP. This data are compared to the human data of early (<48 hours) and late (>/=48 hours) death. The identical genetic background of laboratory mice (despite coming from three strains) allows for assumptions to be made about significance of changes at a higher resolution, due to the certainty of the genetic model. Simultaneously it creates uncertainty about the validity of findings, due to a lack of comparability to humans that experience conditions outside of the laboratory. Human data is plagued by an equal and opposite effect as data derived from animal models. The homogeneity of the mouse model is replaced with heterogeneity due to factors such as age, sex, co-morbidities, and differences in the trauma. By coupling the certainty provided by the homogeneity of the mouse model, and the uncertainty provided by the heterogeneity of the human model, the inventors create a powerful tool with the potential to validate results from mouse analyses in humans. Comparing events across species can identify RNA biology events and genes that are important at both the early and late time point. These findings are compared to those found in the prospective collected data from trauma patients.

Human samples. In this sample set, all the patients are dead. Since RNA is unstable compared to DNA, adjustments in the comparisons between groups during the analysis must be made for the time it took for samples to be collected and RNA isolated. The mouse work is comparing to mice that are alive but were sacrificed. The GTEx consortium, to adjust for problems associated with deceased donors, has described multiple methods. Carithers et al., Biopreservation and Biobanking, 13(5), 311-9(2015).

Aim 3: Enroll critically ill trauma patients and identify aspects of RNA biology that identify and predict outcomes (mortality, infection).

Rationale: A current challenge with the data from the animal models is ensuring translation to humans. This aim allows for complete translation of mouse data to humans. The human population of interest are patients admitted to the Trauma Intensive Care Unit (TICU).

Test 1: Assess RNA sequencing data and identify genes with changes in expression, alternative RNA splicing, and alternative transcription start/end in the blood can be prospectively detected and use this ‘transcriptomic phenotype’ in trauma patients on arrival and be correlated to mortality. Trauma patients are recruited from the trauma intensive care unit, which has an average of over 750 patients, admitted each year (over the last three years) and an average injury severity score (ISS) of 13, but the goal are to enroll patients with an average ISS of 25 to mimic the mouse model. Blood are collected in PAXgene tubes and stored at −80 C after informed consent is obtained. Samples are collected serially while in the ICU. Blood samples from patients are taken on admission (25 mL) and during the TICU stay when a complication is developed (25 mL). This causes the maximum for the initial 8-week period after the trauma. When the patient is recovered, at least 8 weeks after the last blood draw, a final blood draw 50 mL of are done, potentially in the outpatient setting. Patients who survive the trauma are compared to patients who died. Clinical information for the trauma patients are collected from the trauma registry. The trauma registry is a database required as part of verification by the American College of Surgeons to be a trauma center. The data are standardized across the entire recruitment period. RNA are isolated using the PAXgene RNA Kit. RNA was sequenced (goal 80 to 100 million reads). RNA sequencing data are aligned to the human genome using the STAR aligner. Changes in expression, alternative RNA splicing, alternative transcription start/end, and RNA splicing entropy are identified with Whippet. Alternative transcription findings are correlated with mountainClimber.

Test 2: Assess RNA sequencing data and identify genes with changes in expression, alternative RNA splicing, and alternative transcription start/end in the blood can be prospectively detected in trauma patients on arrival and use the ‘transcriptomic phenotype’ to correlate to outcomes and complications. Patients from the trauma intensive care unit identify differences in RNA biology between the healthy controls and trauma patients will predict outcomes and complications. Outcomes and complications are recorded from the medical record and are defined in the trauma registry (and decided by trained coders). The trauma registry will also provide some demographic data; such as injury severity score to better quantify and adjust for the severity of the trauma across patients. Outcomes to follow and use as potential for prediction include mortality, hospital length of stay, intensive care unit length of stay, ventilator free days, and discharge disposition. Complications to be recorded again are taken from the trauma registry and will include items such as infections (pneumonia, surgical site infections, urinary tract infection, bacteremia, sepsis), unplanned return to the operating room, unplanned return to the intensive care unit, tracheostomy, and feeding tube placement.

Human samples: In this sample set, all the patients are critically ill. Consenting patient who are critically ill requires a proxy and this can sometimes be difficult in the unexpected nature of trauma. The inventors have past success in consenting these patients. Human heterogeneity may make finding a significant difference between two groups difficult. Drastic difference (trauma patients in the intensive care unit survive versus die and those with complications) should allow for the identification of differences in RNA biology (‘transcriptomic phenotype’). All samples for this assay come from living patients.

### Example 8

### Survival Assay

All the test mice have the traumatic injury. They are maintained for fourteen days. At fourteen days all mice are sacrificed. The survival rate at fourteen days for the double hit model is 30%. The rate goes up to 70%. Monaghan et al. Annals of Surgery 255(1), 158-64 (2012). These estimates result in an effect size of h=0.823. A sample size of twenty-four per group during analysis would exceed 80% power at a 2-tailed alpha of 0.05 by a chi-square test of independent proportions. for survival analyses the inventors will use twenty-four mice per group. This are done to ensure enough power to detect if RNA splicing at the initial challenge can predict survivors. Sham mice are operated (8 from each mouse background strain) at this time to procure samples at the 14-day time point.

RNA isolation and sequencing. RNA data from GTEx is extracted and sequenced per their protocols. RNA from mouse blood samples are processed using the MasterPure Complete RNA Purification (epicenter, Madison Wis., USA) kit for mice. Due to the high concentration of globin RNA in blood samples, these samples will then be further processed with the GLOBINclear Kit (epicenter, Madison Wis., USA). From blood the inventors can get approximately 30-50 nanogram per microliter, with a total blood volume isolated from the mouse of about one mL. After RNA samples are processed, they are sequenced. All samples will require at least 1400 nanograms of RNA for deep sequencing. Each sample are sent out (due to advancing technologies, costs of sequencing change frequently, therefore outside facility are chosen based upon cost during sample send out) for Deep RNA sequencing with a goal of 80 million to 100 million reads per sample.

Blood from trauma patients and healthy human control samples are collected using the PAXgene tubes (PreAnalytiX, Switzerland) and isolated using the PAXgene RNA kit (PreAnalytiX, Switzerland). Since it is impossible to predict the patients who will die or have a complication on admission to the ICU, banked samples are used since the cost to perform RNA sequencing on the blood of all TICU patients at Rhode Island Hospital is impossible.

Assessment of clinical information. Clinical data relevant to the patient samples are collected from the trauma registry and the electronic medical record. This will allow for collection of endpoints such as mortality, ICU length of stay, hospital length of stay, ventilator days, renal failure, ARDS, pneumonia and other infectious complications. Besides data in the chart, the inventors will also perform functional assessments at follow up after discharge. These would be based upon previous work in critical illness and use the 36-item short form (SF-36). The assessment are done at the 8+ week follow up.

### Example 9

### Alternative RNA Splicing and Alternative Transcription Start/End in Acute Respiratory Distress Syndrome

The objective of this EXAMPLE is to use RNA sequencing data and analysis to identify novel gene targets in sepsis.

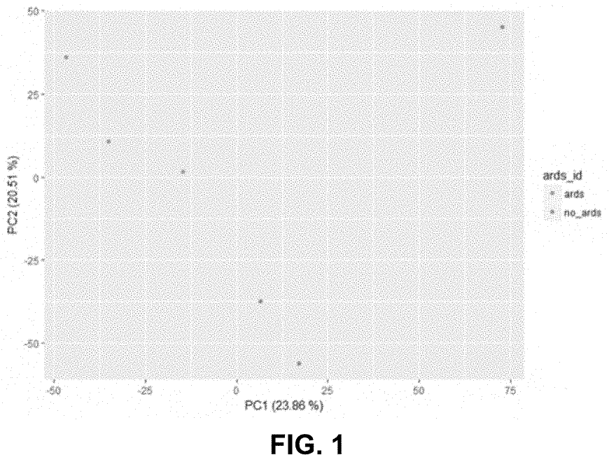

Alternatively spliced RNA arise from co/post-transcriptional events facilitated by the spliceosome, introns are removed to form the mature RNA from which protein isoforms are translated. Alternatively transcribed genes are the product of changes in promoter usage, polyadenylation signals, and RNA polymerase II interactions with DNA which can lead to changes in isoform usage similar to alternative splicing events. These are identified from the analysis of RNA sequencing data. Significant differentially alternatively transcribed genes and alternative spliced genes were identified and were overlapped with genes reported as ARDS related. See, Reilly et al., American Journal of Respiratory and Critical Care Medicine (2017). Of 89 reported ARDS related genes, 38 were confirmed in at least one differential category confirming that the use of humans and mice with DAD/ARDS is appropriate and robust (p=1.25 e-14). Eleven previously reported genes were present in all categories. These eleven genes were evaluated for the change in alternative splicing and alternative transcription GO term enrichment analysis was performed on the eleven overlapping genes, revealing twenty significant biological processes including ontology related to aging, and response to abiotic/environmental stimuli. See FIG. 1. 1639 genes show overlap in alternative splicing and alternative transcription not previously in the literature. These genes were assessed for directionality alternative splicing and alternative transcription and GO terms (TABLE 3, TABLE 4).

Assaying the underlying changes in RNA processing (alternative splicing and alternative transcription start/end) not expands basic knowledge only of pathogenicity, but also provides additional targets for therapeutics. The most enriched GO term from the alternative splicing set, carboxy-terminal domain protein kinase complex (GO:0032806) refers to phosphorylation of the CTD of RNA polymerase II, which is vital in regulating transcription and RNA processing. RNA polymerase complex binding (GO:0000993), and transport of the SLBP Independent/Dependent mature mRNA (R-HSA-159227; R-HSA-159230) are among the most enriched. Alternative pre-mRNA splicing may have the dominate role in isoform usage in genes where expressions levels do not change, whereas alternative transcription may regulate isoform usage in genes that are more dynamically expressed during critical illness. Alternative splicing and alternative transcription may have separate roles in DAD/ARDS by regulating different genes to perform distinctive functions.

In this analysis of RNA sequencing data from deceased patients with ARDS identified by DAD and a clinically relevant mouse model of ARDS, novel genes are identified.

Overview. The inventors used RNA sequencing to identify changes in mRNA processing events (RNA splicing and transcription start/end sites) can be studied with RNA sequencing data. The inventors' strategy was to use the contrast how the processing of mRNA changes in lung and blood of patients with ARDS and compare to the lung and blood of a mouse model of ARDS.

Data. For this EXAMPLE, two main approaches were taken to obtain samples. The first was to use a validated mouse model of ARDS. Ayala et al., The American Journal of Pathology, 161, 2283-2294 (2002); Monaghan et al., Molecular Medicine (Cambridge, Mass., USA), 24, 32 (2018). All experiments were done according to guidelines from the National Institutes of Health (Bethesda, Md.). For the mouse model of ARDS, C57BL/6 male mice (The Jackson Laboratory, Bar Harbor, Me., USA) between 10 and 12 weeks of age were used. ARDS was induced in the mice by hemorrhage (non-lethal shock) followed by cecal ligation and puncture (CLP). The control group was sham hemorrhage followed by sham CLP.

The second approach was to identify patients in the GTEx Project with ARDS. All patients in the GTEx projects used in this EXAMPLE are deceased. A pathologist, blinded to the specimen ID and history, identified diffuse alveolar damage in lung samples from patients in GTEx. Most cases of clinical ARDS will have diffuse alveolar damage (DAD) morphologically. Zander & Farver, Pulmonary pathology e-book: A volume in foundations in diagnostic pathology series. (Elsevier Health Sciences, 2016). Classic DAD was identified based histologic features (For full description, please see supplement). Patients with evidence of diffuse alveolar damage in the lung and a corresponding blood and lung sample that had undergone RNA sequencing were placed in the ARDS group. Patients who had no evidence of diffuse alveolar damage in the pathology sample and a blood and lung sample with RNA sequencing were placed in the control group. Most cases of clinical acute lung injury (ALI) and acute respiratory distress syndrome (ARDS) will have diffuse alveolar damage (DAD) morphologically, which is divided into 2 phases: the acute/exudative phase and the organizing/proliferative phase. Other histologic patterns encountered in a clinical setting of ALI/ARDS include diffuse alveolar hemorrhage, acute eosinophilic pneumonia (AEP), and the acute fibrinous and organizing pneumonia (AFOP). Eight patterns of acute lung injury are evaluated in this EXAMPLE. Zander & Farver, Pulmonary pathology e-book: A volume in foundations in diagnostic pathology series. (Elsevier Health Sciences, 2016). Classic DAD are was graded 1-4 based on the histologic features. Other patterns of injury were scored using a semiquantitative system for extent and histologic characteristics. For extent, grade was assigned: grade 1 (1 point): up to 10% tissue involved, grade 2 (2 points): 11-30% tissue involved, grade 3 (3 points): 31-50% tissue involved and grade 4 (4 points): >50% tissue involved. Histologic characteristics including intra-alveolar fibrin (1 point), cellular alveolar debris (I point), type II pneumocyte hyperplasia (1 point) and capillaritis/vasculitis. Total points 6 or higher were considered as DAD. Despite this complex method for categorizing diffuse alveolar damage, using this to diagnose ARDS is a major limitation. DAD could be present in other pulmonary diseases. The value RNA sequencing data from the lungs and blood of patients can provide biologic insights despite these limitations.

Results. Alternative splicing events were observed at 2-fold higher abundance as compared to alternative transcription events, yet significant alternative transcription events between groups were observed at a 6-fold higher prevalence (p=2.2 e-16). Eighty-two alternative transcription events were common across all ARDS tissues (human and mouse, blood and lung, p=2.72 e-16). No significant alternative splicing events were detected across all four tissues. As alternative splicing is species and tissue specific, it is unlikely to find an event that occurs in lung tissue and blood tissue in both human and mouse. GO term analysis was also performed on the significant differentially processing events.

The full list is TABLE 3 below.

TABLE 3
Complete list of GO Terms from Significantly Alternative Splicing and Alternative
Transcription Start/End Events Alternative Splicing n = 2362

| GO Term | logFC |
|---|---|
| Amine ligand-binding receptors (R-HSA-375280) | −6.64385619 |
| Amine-derived hormones (R-HSA-209776) | −6.64385619 |
| axonemal dynein complex (GO:0005858) | −6.64385619 |
| bitter taste receptor activity (GO:0033038) | −6.64385619 |
| calcium-independent cell-cell adhesion via plasma membrane | −6.64385619 |
| cell-adhesion molecules (GO:0016338) | |
| catecholamine binding (GO:1901338) | −6.64385619 |
| chondrocyte morphogenesis (GO:0090171) | −6.64385619 |
| chondrocyte morphogenesis involved in endochondral bone | −6.64385619 |
| morphogenesis (GO:0003414) | |
| connexin complex (GO:0005922) | −6.64385619 |
| Defective C1GALT1C1 causes Tn polyagglutination syndrome | −6.64385619 |
| (TNPS) (R-HSA-5083632) | |
| Defective GALNT12 causes colorectal cancer 1 (CRCS1) (R- | −6.64385619 |
| HSA-5083636) | |
| Defective GALNT3 causes familial hyperphosphatemic tumoral | −6.64385619 |
| calcinosis (HFTC) (R-HSA-5083625) | |
| delayed rectifier potassium channel activity (GO:0005251) | −6.64385619 |
| detection of chemical stimulus involved in sensory perception | −6.64385619 |
| (GO:0050907) | |
| detection of chemical stimulus involved in sensory perception of | −6.64385619 |
| bitter taste (GO:0001580) | |
| detection of chemical stimulus involved in sensory perception of | −6.64385619 |
| smell (GO:0050911) | |
| detection of chemical stimulus involved in sensory perception of | −6.64385619 |
| taste (GO:0050912) | |
| Eicosanoid ligand-binding receptors (R-HSA-391903) | −6.64385619 |
| FGFR2 ligand binding and activation (R-HSA-190241) | −6.64385619 |
| G protein-coupled serotonin receptor activity (GO:0004993) | −6.64385619 |
| G protein-coupled serotonin receptor signaling pathway | −6.64385619 |
| (GO:0098664) | |
| GABA receptor complex (GO:1902710) | −6.64385619 |
| GABA-A receptor complex (GO:1902711) | −6.64385619 |
| growth plate cartilage chondrocyte morphogenesis | −6.64385619 |
| (GO:0003429) | |
| growth plate cartilage morphogenesis (GO:0003422) | −6.64385619 |
| ligand-gated anion channel activity (GO:0099095) | −6.64385619 |
| odorant binding (GO:0005549) | −6.64385619 |
| olfactory receptor activity (GO:0004984) | −6.64385619 |
| piRNA metabolic process (GO:0034587) | −6.64385619 |
| positive regulation of peptidyl-serine phosphorylation of STAT | −6.64385619 |
| protein (GO:0033141) | |
| regulation of circadian sleep/wake cycle (GO:0042749) | −6.64385619 |
| serotonin receptor activity (GO:0099589) | −6.64385619 |
| serotonin receptor signaling pathway (GO:0007210) | −6.64385619 |
| taste receptor activity (GO:0008527) | −6.64385619 |
| Creation of C4 and C2 activators (R-HSA-166786) | −5.058893689 |
| immunoglobulin complex, circulating (GO:0042571) | −5.058893689 |
| Olfactory Signaling Pathway (R-HSA-381753) | −5.058893689 |
| Classical antibody-mediated complement activation (R-HSA- | −4.64385619 |
| 173623) | |
| G protein-coupled amine receptor activity (GO:0008227) | −4.64385619 |
| sensory perception of smell (GO:0007608) | −4.64385619 |
| detection of stimulus involved in sensory perception | −4.321928095 |
| (GO:0050906) | |
| transmitter-gated ion channel activity involved in regulation of | −4.321928095 |
| postsynaptic membrane potential (GO:1904315) | |
| Class C/3 (Metabotropic glutamate/pheromone receptors) (R- | −4.058893689 |
| HSA-420499) | |
| sensory perception of chemical stimulus (GO:0007606) | −4.058893689 |
| detection of chemical stimulus (GO:0009593) | −3.836501268 |
| immunoglobulin complex (GO:0019814) | −3.836501268 |
| keratin filament (GO:0045095) | −3.64385619 |
| transmitter-gated channel activity (GO:0022835) | −3.64385619 |
| transmitter-gated ion channel activity (GO:0022824) | −3.64385619 |
| complement activation, classical pathway (GO:0006958) | −3.473931188 |
| Keratinization (R-HSA-6805567) | −3.473931188 |
| Phase 2 - plateau phase (R-HSA-5576893) | −3.473931188 |
| Digestion and absorption (R-HSA-8963743) | −3.321928095 |
| exogenous drug catabolic process (GO:0042738) | −3.321928095 |
| neurotransmitter receptor activity involved in regulation of | −3.321928095 |
| postsynaptic membrane potential (GO:0099529) | |
| regulation of mesonephros development (GO:0061217) | −3.321928095 |
| keratinization (GO:0031424) | −3.184424571 |
| postsynaptic neurotransmitter receptor activity (GO:0098960) | −3.184424571 |
| sodium channel complex (GO:0034706) | −3.184424571 |
| Collagen chain trimerization (R-HSA-8948216) | −3.058893689 |
| Digestion (R-HSA-8935690) | −3.058893689 |
| extracellular matrix structural constituent conferring tensile | −3.058893689 |
| strength (GO:0030020) | |
| G protein-coupled neurotransmitter receptor activity | −3.058893689 |
| (GO:0099528) | |
| G protein-coupled receptor activity (GO:0004930) | −3.058893689 |
| Initial triggering of complement (R-HSA-166663) | −3.058893689 |
| Phase 0 - rapid depolarisation (R-HSA-5576892) | −3.058893689 |
| complement activation (GO:0006956) | −2.943416472 |
| immunoglobulin receptor binding (GO:0034987) | −2.943416472 |
| neurotransmitter receptor activity (GO:0030594) | −2.943416472 |
| steroid hydroxylase activity (GO:0008395) | −2.943416472 |
| Beta defensins (R-HSA-1461957) | −2.836501268 |
| voltage-gated potassium channel activity (GO:0005249) | −2.836501268 |
| humoral immune response mediated by circulating | −2.736965594 |
| immunoglobulin (GO:0002455) | |
| neuron fate specification (GO:0048665) | −2.736965594 |
| neuropeptide receptor binding (GO:0071855) | −2.736965594 |
| oxidoreductase activity, acting on paired donors, with | −2.736965594 |
| incorporation or reduction of molecular oxygen, reduced flavin or | |
| flavoprotein as one donor, and incorporation of one atom of | |
| oxygen (GO:0016712) | |
| calcium-dependent cell-cell adhesion via plasma membrane cell | −2.64385619 |
| adhesion molecules (GO:0016339) | |
| Formation of the cornified envelope (R-HSA-6809371) | −2.64385619 |
| G alpha (s) signalling events (R-HSA-418555) | −2.64385619 |
| gap junction (GO:0005921) | −2.64385619 |
| extracellular ligand-gated ion channel activity (GO:0005230) | −2.556393349 |
| phagocytosis, recognition (GO:0006910) | −2.556393349 |
| sensory perception of bitter taste (GO:0050913) | −2.556393349 |
| cornification (GO:0070268) | −2.473931188 |
| NCAM1 interactions (R-HSA-419037) | −2.473931188 |
| Voltage gated Potassium channels (R-HSA-1296072) | −2.473931188 |
| CD22 mediated BCR regulation (R-HSA-5690714) | −2.395928676 |
| sodium channel activity (GO:0005272) | −2.395928676 |
| cornified envelope (GO:0001533) | −2.321928095 |
| Scavenging of heme from plasma (R-HSA-2168880) | −2.251538767 |
| Defensins (R-HSA-1461973) | −2.184424571 |
| detection of visible light (GO:0009584) | −2.184424571 |
| potassium channel activity (GO:0005267) | −2.184424571 |
| Complement cascade (R-HSA-166658) | −2.120294234 |
| integral component of postsynaptic specialization membrane | −2.120294234 |
| (GO:0099060) | |
| sensory perception of taste (GO:0050909) | −2.120294234 |
| voltage-gated cation channel activity (GO:0022843) | −2.120294234 |
| hormone activity (GO:0005179) | −2.058893689 |
| chloride channel complex (GO:0034707) | −2 |
| Class A/1 (Rhodopsin-like receptors) (R-HSA-373076) | −2 |
| collagen trimer (GO:0005581) | −2 |
| GPCR ligand binding (R-HSA-500792) | −2 |
| regulation of catecholamine secretion (GO:0050433) | −2 |
| Regulation of Complement cascade (R-HSA-977606) | −2 |
| regulation of dopamine secretion (GO:0014059) | −2 |
| cardiac muscle cell action potential involved in contraction | −1.943416472 |
| (GO:0086002) | |
| phospholipase C-activating G protein-coupled receptor signaling | −1.943416472 |
| pathway (GO:0007200) | |
| intermediate filament (GO:0005882) | −1.888968688 |
| keratinocyte differentiation (GO:0030216) | −1.888968688 |
| sensory perception (GO:0007600) | −1.888968688 |
| transmission of nerve impulse (GO:0019226) | −1.888968688 |
| detection of stimulus (GO:0051606) | −1.836501268 |
| intrinsic component of postsynaptic specialization membrane | −1.836501268 |
| (GO:0098948) | |
| integral component of postsynaptic membrane (GO:0099055) | −1.785875195 |
| neuropeptide signaling pathway (GO:0007218) | −1.785875195 |
| potassium channel complex (GO:0034705) | −1.785875195 |
| sulfotransferase activity (GO:0008146) | −1.785875195 |
| antigen binding (GO:0003823) | −1.736965594 |
| homophilic cell adhesion via plasma membrane adhesion | −1.736965594 |
| molecules (GO:0007156) | |
| neuropeptide receptor activity (GO:0008188) | −1.736965594 |
| regulation of complement activation (GO:0030449) | −1.736965594 |
| Potassium Channels (R-HSA-1296071) | −1.689659879 |
| axoneme part (GO:0044447) | −1.64385619 |
| intrinsic component of postsynaptic membrane (GO:0098936) | −1.64385619 |
| T cell receptor complex (GO:0042101) | −1.64385619 |
| voltage-gated potassium channel complex (GO:0008076) | −1.64385619 |
| peptide receptor activity (GO:0001653) | −1.59946207 |
| cell fate specification (GO:0001708) | −1.556393349 |
| cilium movement (GO:0003341) | −1.556393349 |
| detection of light stimulus (GO:0009583) | −1.556393349 |
| FCGR activation (R-HSA-2029481) | −1.556393349 |
| integral component of postsynaptic density membrane | −1.556393349 |
| (GO:0099061) | |
| membrane depolarization (GO:0051899) | −1.556393349 |
| voltage-gated channel activity (GO:0022832) | −1.556393349 |
| voltage-gated ion channel activity (GO:0005244) | −1.556393349 |
| extracellular matrix component (GO:0044420) | −1.514573173 |
| G protein-coupled peptide receptor activity (GO:0008528) | −1.514573173 |
| ligand-gated channel activity (GO:0022834) | −1.514573173 |
| ligand-gated ion channel activity (GO:0015276) | −1.514573173 |
| positive regulation of synapse assembly (GO:0051965) | −1.514573173 |
| transmembrane signaling receptor activity (GO:0004888) | −1.514573173 |
| Class B/2 (Secretin family receptors) (R-HSA-373080) | −1.473931188 |
| ion gated channel activity (GO:0022839) | −1.473931188 |
| cytokine activity (GO:0005125) | −1.434402824 |
| epidermal cell differentiation (GO:0009913) | −1.434402824 |
| extracellular matrix structural constituent (GO:0005201) | −1.434402824 |
| growth factor activity (GO:0008083) | −1.434402824 |
| receptor ligand activity (GO:0048018) | −1.434402824 |
| receptor regulator activity (GO:0030545) | −1.434402824 |
| regulation of humoral immune response (GO:0002920) | −1.434402824 |
| serine-type endopeptidase inhibitor activity (GO:0004867) | −1.434402824 |
| Assembly of collagen fibrils and other multimeric structures (R- | −1.395928676 |
| HSA-2022090) | |
| Collagen biosynthesis and modifying enzymes (R-HSA-1650814) | −1.395928676 |
| G protein-coupled receptor signaling pathway (GO:0007186) | −1.395928676 |
| gated channel activity (GO:0022836) | −1.395928676 |
| Peptide ligand-binding receptors (R-HSA-375276) | −1.395928676 |
| signaling receptor activator activity (GO:0030546) | −1.395928676 |
| humoral immune response (GO:0006959) | −1.358453971 |
| integral component of synaptic membrane (GO:0099699) | −1.358453971 |
| Antimicrobial peptides (R-HSA-6803157) | −1.321928095 |
| ion channel complex (GO:0034702) | −1.321928095 |
| multicellular organismal signaling (GO:0035637) | −1.321928095 |
| cation channel complex (GO:0034703) | −1.286304185 |
| cell-cell adhesion via plasma-membrane adhesion molecules | −1.286304185 |
| (GO:0098742) | |
| detection of external stimulus (GO:0009581) | −1.286304185 |
| ligand-gated cation channel activity (GO:0099094) | −1.286304185 |
| monooxygenase activity (GO:0004497) | −1.286304185 |
| potassium ion transmembrane transporter activity (GO:0015079) | −1.286304185 |
| Role of LAT2/NTAL/LAB on calcium mobilization (R-HSA- | −1.286304185 |
| 2730905) | |
| B cell mediated immunity (GO:0019724) | −1.251538767 |
| cation channel activity (GO:0005261) | −1.251538767 |
| immunoglobulin mediated immune response (GO:0016064) | −1.251538767 |
| intrinsic component of synaptic membrane (GO:0099240) | −1.251538767 |
| potassium ion transmembrane transport (GO:0071805) | −1.251538767 |
| regulation of postsynaptic membrane potential (GO:0060078) | −1.251538767 |
| postsynaptic specialization membrane (GO:0099634) | −1.217591435 |
| regulation of amine transport (GO:0051952) | −1.217591435 |
| detection of abiotic stimulus (GO:0009582) | −1.184424571 |
| nervous system process (GO:0050877) | −1.184424571 |
| phagocytosis, engulfment (GO:0006911) | −1.184424571 |
| action potential (GO:0001508) | −1.152003093 |
| cardiac conduction (GO:0061337) | −1.152003093 |
| channel activity (GO:0015267) | −1.152003093 |
| GPCR downstream signalling (R-HSA-388396) | −1.152003093 |
| ion channel activity (GO:0005216) | −1.152003093 |
| passive transmembrane transporter activity (GO:0022803) | −1.152003093 |
| Signaling by GPCR (R-HSA-372790) | −1.152003093 |
| signaling receptor activity (GO:0038023) | −1.152003093 |
| transmembrane transporter complex (GO:1902495) | −1.152003093 |
| actin-mediated cell contraction (GO:0070252) | −1.120294234 |
| adenylate cydase-activating G protein-coupled receptor signaling | −1.120294234 |
| pathway (GO:0007189) | |
| G protein-coupled receptor signaling pathway, coupled to cyclic | −1.120294234 |
| nucleotide second messenger (GO:0007187) | |
| serine-type endopeptidase activity (GO:0004252) | −1.089267338 |
| synapse assembly (GO:0007416) | −1.089267338 |
| transporter complex (GO:1990351) | −1.089267338 |
| basement membrane (GO:0005604) | −1.058893689 |
| digestion (GO:0007586) | −1.058893689 |
| heparin binding (GO:0008201) | −1.058893689 |
| intermediate filament cytoskeleton (GO:0045111) | −1.058893689 |
| potassium ion transport (GO:0006813) | −1.058893689 |
| regulation of synapse assembly (GO:0051963) | −1.058893689 |
| sensory perception of light stimulus (GO:0050953) | −1.058893689 |
| Unclassified (UNCLASSIFIED) | −1.058893689 |
| adenylate cyclase-modulating G protein-coupled receptor | −1.029146346 |
| signaling pathway (GO:0007188) | |
| Collagen formation (R-HSA-1474290) | −1.029146346 |
| epidermis development (GO:0008544) | −1.029146346 |
| extracellular matrix (GO:0031012) | −1.029146346 |
| intrinsic component of presynaptic membrane (GO:0098889) | −1.029146346 |
| molecular transducer activity (GO:0060089) | −1.029146346 |
| skin development (GO:0043588) | −1.029146346 |
| visual perception (GO:0007601) | −1.029146346 |
| Binding and Uptake of Ligands by Scavenger Receptors (R-HSA- | −1 |
| 2173782) | |
| Golgi lumen (GO:0005796) | −0.971430848 |
| antimicrobial humoral response (GO:0019730) | −0.943416472 |
| cAMP-mediated signaling (GO:0019933) | −0.943416472 |
| Cardiac conduction (R-HSA-5576891) | −0.915935735 |
| anchored component of membrane (GO:0031225) | −0.888968688 |
| collagen-containing extracellular matrix (GO:0062023) | −0.888968688 |
| sodium ion transmembrane transporter activity (GO:0015081) | −0.888968688 |
| plasma membrane invagination (GO:0099024) | −0.836501268 |
| postsynaptic membrane (GO:0045211) | −0.836501268 |
| cell recognition (GO:0008037) | −0.810966176 |
| sensory perception of sound (GO:0007605) | −0.810966176 |
| system process (GO:0003008) | −0.810966176 |
| anterograde trans-synaptic signaling (GO:0098916) | −0.785875195 |
| chemical synaptic transmission (GO:0007268) | −0.785875195 |
| cyclic-nucleotide-mediated signaling (GO:0019935) | −0.785875195 |
| Degradation of the extracellular matrix (R-HSA-1474228) | −0.785875195 |
| sensory perception of mechanical stimulus (GO:0050954) | −0.785875195 |
| trans-synaptic signaling (GO:0099537) | −0.785875195 |
| glycosaminoglycan binding (GO:0005539) | −0.76121314 |
| immunoglobulin production (GO:0002377) | −0.76121314 |
| Neuronal System (R-HSA-112316) | −0.76121314 |
| serine-type peptidase activity (GO:0008236) | −0.76121314 |
| defense response to bacterium (GO:0042742) | −0.713118852 |
| hydrolase activity, acting on acid phosphorus-nitrogen bonds | −0.689659879 |
| (GO:0016825) | |
| serine hydrolase activity (GO:0017171) | −0.689659879 |
| cell fate commitment (GO:0045165) | −0.666576266 |
| synaptic signaling (GO:0099536) | −0.666576266 |
| inner ear development (GO:0048839) | −0.64385619 |
| ear development (GO:0043583) | −0.621488377 |
| metal ion transmembrane transporter activity (GO:0046873) | −0.621488377 |
| sensory organ morphogenesis (GO:0090596) | −0.621488377 |
| epithelial cell differentiation (GO:0030855) | −0.59946207 |
| integral component of plasma membrane (GO:0005887) | −0.59946207 |
| synaptic membrane (GO:0097060) | −0.59946207 |
| lymphocyte mediated immunity (GO:0002449) | −0.577766999 |
| Muscle contraction (R-HSA-397014) | −0.577766999 |
| G alpha (I) signalling events (R-HSA-418594) | −0.556393349 |
| intrinsic component of plasma membrane (GO:0031226) | −0.556393349 |
| regionalization (GO:0003002) | −0.556393349 |
| monovalent inorganic cation transmembrane transporter activity | −0.535331733 |
| (GO:0015077) | |
| pattern specification process (GO:0007389) | −0.535331733 |
| receptor complex (GO:0043235) | −0.535331733 |
| extracellular matrix organization (GO:0030198) | −0.514573173 |
| adaptive immune response (GO:0002250) | −0.473931188 |
| plasma membrane receptor complex (GO:0098802) | −0.473931188 |
| inorganic cation transmembrane transporter activity | −0.434402824 |
| (GO:0022890) | |
| plasma membrane protein complex (GO:0098797) | −0.434402824 |
| regulation of membrane potential (GO:0042391) | −0.434402824 |
| sensory organ development (GO:0007423) | −0.434402824 |
| calcium ion binding (GO:0005509) | −0.415037499 |
| external side of plasma membrane (GO:0009897) | −0.415037499 |
| inorganic molecular entity transmembrane transporter activity | −0.377069649 |
| (GO:0015318) | |
| animal organ morphogenesis (GO:0009887) | −0.358453971 |
| cation transmembrane transporter activity (GO:0008324) | −0.358453971 |
| epithelium development (GO:0060429) | −0.340075442 |
| cell adhesion (GO:0007155) | −0.321928095 |
| cell surface (GO:0009986) | −0.321928095 |
| DNA-binding transcription factor activity, RNA polymerase II- | −0.321928095 |
| specific (GO:0000981) | |
| biological adhesion (GO:0022610) | −0.304006187 |
| plasma membrane part (GO:0044459) | −0.304006187 |
| ion transmembrane transporter activity (GO:0015075) | −0.286304185 |
| DNA-binding transcription factor activity (GO:0003700) | −0.251538767 |
| integral component of membrane (GO:0016021) | −0.234465254 |
| intrinsic component of membrane (GO:0031224) | −0.234465254 |
| plasma membrane (GO:0005886) | −0.234465254 |
| tissue development (GO:0009888) | −0.234465254 |
| cell periphery (GO:0071944) | −0.217591435 |
| extracellular region (GO:0005576) | −0.120294234 |
| multicellular organismal process (GO:0032501) | −0.120294234 |
| membrane part (GO:0044425) | −0.089267338 |
| cellular component (GO:0005575) | 0.097610797 |
| membrane (GO:0016020) | 0.124328135 |
| biological process (GO:0008150) | 0.137503524 |
| response to stimulus (GO:0050896) | 0.137503524 |
| cation binding (GO:0043169) | 0.150559677 |
| regulation of transcription by RNA polymerase II (GO:0006357) | 0.150559677 |
| biological regulation (GO:0065007) | 0.163498732 |
| cell surface receptor signaling pathway (GO:0007166) | 0.163498732 |
| cellular response to stimulus (GO:0051716) | 0.163498732 |
| metal ion binding (GO:0046872) | 0.163498732 |
| molecular_function (GO:0003674) | 0.163498732 |
| regulation of biological process (GO:0050789) | 0.163498732 |
| cell (GO:0005623) | 0.176322773 |
| cell part (GO:0044464) | 0.176322773 |
| regulation of cellular process (GO:0050794) | 0.176322773 |
| regulation of multicellular organismal development (GO:2000026) | 0.176322773 |
| regulation of multicellular organismal process (GO:0051239) | 0.176322773 |
| positive regulation of multicellular organismal process | 0.189033824 |
| (GO:0051240) | |
| regulation of cell differentiation (GO:0045595) | 0.201633861 |
| regulation of cell population proliferation (GO:0042127) | 0.201633861 |
| regulation of developmental process (GO:0050793) | 0.201633861 |
| membrane protein complex (GO:0098796) | 0.214124805 |
| immune response (GO:0006955) | 0.22650853 |
| regulation of anatomical structure morphogenesis (GO:0022603) | 0.22650853 |
| response to endogenous stimulus (GO:0009719) | 0.22650853 |
| cellular response to endogenous stimulus (GO:0071495) | 0.23878686 |
| regulation of transcription, DNA-templated (GO:0006355) | 0.23878686 |
| cellular process (GO:0009987) | 0.250961574 |
| regulation of biological guality (GO:0065008) | 0.250961574 |
| regulation of localization (GO:0032879) | 0.250961574 |
| regulation of nucleic acid-templated transcription (GO:1903506) | 0.250961574 |
| regulation of RNA biosynthetic process (GO:2001141) | 0.250961574 |
| regulation of transport (GO:0051049) | 0.250961574 |
| response to hormone (GO:0009725) | 0.250961574 |
| transition metal ion binding (GO:0046914) | 0.250961574 |
| binding (GO:0005488) | 0.263034406 |
| cellular homeostasis (GO:0019725) | 0.263034406 |
| homeostatic process (GO:0042592) | 0.263034406 |
| ion binding (GO:0043167) | 0.263034406 |
| multi-organism process (GO:0051704) | 0.263034406 |
| regulation of cellular component movement (GO:0051270) | 0.263034406 |
| positive regulation of protein phosphorylation (GO:0001934) | 0.275007047 |
| positive regulation of transcription by RNA polymerase II | 0.275007047 |
| (GO:0045944) | |
| positive regulation of response to stimulus (GO:0048584) | 0.286881148 |
| enzyme linked receptor protein signaling pathway (GO:0007167) | 0.298658316 |
| lipid binding (GO:0008289) | 0.298658316 |
| positive regulation of phosphate metabolic process | 0.298658316 |
| (GO:0045937) | |
| positive regulation of phosphorus metabolic process | 0.298658316 |
| (GO:0010562) | |
| positive regulation of phosphorylation (GO:0042327) | 0.298658316 |
| regulation of cell motility (GO:2000145) | 0.298658316 |
| regulation of locomotion (GO:0040012) | 0.298658316 |
| regulation of RNA metabolic process (GO:0051252) | 0.298658316 |
| cellular response to chemical stimulus (GO:0070887) | 0.310340121 |
| cellular response to hormone stimulus (GO:0032870) | 0.310340121 |
| cytoplasmic region (GO:0099568) | 0.310340121 |
| regulation of cell migration (GO:0030334) | 0.310340121 |
| regulation of response to stimulus (GO:0048583) | 0.310340121 |
| response to oxygen-containing compound (GO:1901700) | 0.310340121 |
| Transport of small molecules (R-HSA-382551) | 0.310340121 |
| zinc ion binding (GO:0008270) | 0.310340121 |
| actin filament-based process (GO:0030029) | 0.321928095 |
| negative regulation of nucleic acid-templated transcription | 0.321928095 |
| (GO:1903507) | |
| negative regulation of RNA biosynthetic process (GO:1902679) | 0.321928095 |
| negative regulation of transcription by RNA polymerase II | 0.321928095 |
| (GO:0000122) | |
| negative regulation of transcription, DNA-templated | 0.321928095 |
| (GO:0045892) | |
| regulation of cell communication (GO:0010646) | 0.321928095 |
| regulation of cellular biosynthetic process (GO:0031326) | 0.321928095 |
| regulation of cellular macromolecule biosynthetic process | 0.321928095 |
| (GO:2000112) | |
| regulation of nucleobase-containing compound metabolic | 0.321928095 |
| process (GO:0019219) | |
| regulation of signaling (GO:0023051) | 0.321928095 |
| positive regulation of biological process (GO:0048518) | 0.333423734 |
| regulation of biosynthetic process (GO:0009889) | 0.333423734 |
| regulation of cell activation (GO:0050865) | 0.333423734 |
| regulation of cell projection organization (GO:0031344) | 0.333423734 |
| regulation of leukocyte activation (GO:0002694) | 0.333423734 |
| regulation of macromolecule biosynthetic process (GO:0010556) | 0.333423734 |
| regulation of plasma membrane bounded cell projection | 0.333423734 |
| organization (GO:0120035) | |
| cytoskeleton (GO:0005856) | 0.344828497 |
| Hemostasis (R-HSA-109582) | 0.344828497 |
| negative regulation of cellular process (GO:0048523) | 0.344828497 |
| negative regulation of RNA metabolic process (GO:0051253) | 0.344828497 |
| organic acid metabolic process (GO:0006082) | 0.344828497 |
| positive regulation of transcription, DNA-templated | 0.344828497 |
| (GO:0045893) | |
| regulation of gene expression (GO:0010468) | 0.344828497 |
| regulation of nitrogen compound metabolic process | 0.344828497 |
| (GO:0051171) | |
| regulation of primary metabolic process (GO:0080090) | 0.344828497 |
| response to organic substance (GO:0010033) | 0.344828497 |
| small molecule biosynthetic process (GO:0044283) | 0.344828497 |
| cellular response to organic substance (GO:0071310) | 0.35614381 |
| cytoskeletal part (GO:0044430) | 0.35614381 |
| intracellular (GO:0005622) | 0.35614381 |
| intracellular part (GO:0044424) | 0.35614381 |
| negative regulation of biological process (GO:0048519) | 0.35614381 |
| negative regulation of catalytic activity (GO:0043086) | 0.35614381 |
| negative regulation of molecular function (GO:0044092) | 0.35614381 |
| organelle (GO:0043226) | 0.35614381 |
| oxoacid metabolic process (GO:0043436) | 0.35614381 |
| positive regulation of cell communication (GO:0010647) | 0.35614381 |
| positive regulation of cell motility (GO:2000147) | 0.35614381 |
| positive regulation of cellular component movement | 0.35614381 |
| (GO:0051272) | |
| positive regulation of cellular process (GO:0048522) | 0.35614381 |
| positive regulation of locomotion (GO:0040017) | 0.35614381 |
| positive regulation of signaling (GO:0023056) | 0.35614381 |
| regulation of macromolecule metabolic process (GO:0060255) | 0.35614381 |
| regulation of protein phosphorylation (GO:0001932) | 0.35614381 |
| activation of immune response (GO:0002253) | 0.367371066 |
| cellular response to oxygen-containing compound (GO:1901701) | 0.367371066 |
| cytokine-mediated signaling pathway (GO:0019221) | 0.367371066 |
| immune response-activating cell surface receptor signaling | 0.367371066 |
| pathway (GO:0002429) | |
| immune system process (GO:0002376) | 0.367371066 |
| lipid metabolic process (GO:0006629) | 0.367371066 |
| negative regulation of cell communication (GO:0010648) | 0.367371066 |
| negative regulation of nucleobase-containing compound | 0.367371066 |
| metabolic process (GO:0045934) | |
| negative regulation of response to stimulus (GO:0048585) | 0.367371066 |
| negative regulation of signaling (GO:0023057) | 0.367371066 |
| oxidoreductase activity (GO:0016491) | 0.367371066 |
| protein dimerization activity (GO:0046983) | 0.367371066 |
| regulation of cellular metabolic process (GO:0031323) | 0.367371066 |
| regulation of metabolic process (GO:0019222) | 0.367371066 |
| regulation of signal transduction (GO:0009966) | 0.367371066 |
| actin cytoskeleton (GO:0015629) | 0.378511623 |
| cellular response to nitrogen compound (GO:1901699) | 0.378511623 |
| cellular response to organonitrogen compound (GO:0071417) | 0.378511623 |
| localization (GO:0051179) | 0.378511623 |
| negative regulation of apoptotic process (GO:0043066) | 0.378511623 |
| negative regulation of cell death (GO:0060548) | 0.378511623 |
| negative regulation of programmed cell death (GO:0043069) | 0.378511623 |
| positive regulation of gene expression (GO:0010628) | 0.378511623 |
| positive regulation of intracellular signal transduction | 0.378511623 |
| (GO:1902533) | |
| positive regulation of protein modification process (GO:0031401) | 0.378511623 |
| positive regulation of signal transduction (GO:0009967) | 0.378511623 |
| regulation of cell adhesion (GO:0030155) | 0.378511623 |
| regulation of response to external stimulus (GO:0032101) | 0.378511623 |
| carbohydrate metabolic process (GO:0005975) | 0.389566812 |
| carboxylic acid metabolic process (GO:0019752) | 0.389566812 |
| cellular response to drug (GO:0035690) | 0.389566812 |
| cytoskeleton organization (GO:0007010) | 0.389566812 |
| Generic Transcription Pathway (R-HSA-212436) | 0.389566812 |
| immune response-regulating cell surface receptor signaling | 0.389566812 |
| pathway (GO:0002768) | |
| positive regulation of immune system process (GO:0002684) | 0.389566812 |
| positive regulation of nucleic acid-templated transcription | 0.389566812 |
| (GO:1903508) | |
| positive regulation of RNA biosynthetic process (GO:1902680) | 0.389566812 |
| positive regulation of transport (GO:0051050) | 0.389566812 |
| protein binding (GO:0005515) | 0.389566812 |
| regulation of Wnt signaling pathway (GO:0030111) | 0.389566812 |
| small molecule catabolic process (GO:0044282) | 0.389566812 |
| carbohydrate derivative biosynthetic process (GO:1901137) | 0.40053793 |
| carbohydrate derivative metabolic process (GO:1901135) | 0.40053793 |
| cytoskeletal protein binding (GO:0008092) | 0.40053793 |
| hydrolase activity (GO:0016787) | 0.40053793 |
| intracellular organelle (GO:0043229) | 0.40053793 |
| negative regulation of cellular biosynthetic process | 0.40053793 |
| (GO:0031327) | |
| negative regulation of signal transduction (GO:0009968) | 0.40053793 |
| positive regulation of cell migration (GO:0030335) | 0.40053793 |
| regulation of apoptotic process (GO:0042981) | 0.40053793 |
| response to abiotic stimulus (GO:0009628) | 0.40053793 |
| response to inorganic substance (GO:0010035) | 0.40053793 |
| actin cytoskeleton organization (GO:0030036) | 0.411426246 |
| endoplasmic reticulum (GO:0005783) | 0.411426246 |
| in utero embryonic development (GO:0001701) | 0.411426246 |
| membrane-bounded organelle (GO:0043227) | 0.411426246 |
| negative regulation of biosynthetic process (GO:0009890) | 0.411426246 |
| negative regulation of cellular macromolecule biosynthetic | 0.411426246 |
| process (GO:2000113) | |
| negative regulation of immune system process (GO:0002683) | 0.411426246 |
| negative regulation of macromolecule biosynthetic process | 0.411426246 |
| (GO:0010558) | |
| negative regulation of nitrogen compound metabolic process | 0.411426246 |
| (GO:0051172) | |
| plasma membrane bounded cell projection assembly | 0.411426246 |
| (GO:0120031) | |
| positive regulation of immune response (GO:0050778) | 0.411426246 |
| positive regulation of RNA metabolic process (GO:0051254) | 0.411426246 |
| regulation of cell death (GO:0010941) | 0.411426246 |
| regulation of cell-cell adhesion (GO:0022407) | 0.411426246 |
| regulation of immune system process (GO:0002682) | 0.411426246 |
| regulation of programmed cell death (GO:0043067) | 0.411426246 |
| response to light stimulus (GO:0009416) | 0.411426246 |
| transmembrane receptor protein tyrosine kinase signaling | 0.411426246 |
| pathway (GO:0007169) | |
| transport vesicle (GO:0030133) | 0.411426246 |
| alcohol metabolic process (GO:0006066) | 0.422233001 |
| antigen receptor-mediated signaling pathway (GO:0050851) | 0.422233001 |
| cell projection assembly (GO:0030031) | 0.422233001 |
| heterocyclic compound binding (GO:1901363) | 0.422233001 |
| immune response-activating signal transduction (GO:0002757) | 0.422233001 |
| nucleic acid binding (GO:0003676) | 0.422233001 |
| organic cyclic compound binding (GO:0097159) | 0.422233001 |
| positive regulation of biosynthetic process (GO:0009891) | 0.422233001 |
| positive regulation of cell projection organization (GO:0031346) | 0.422233001 |
| positive regulation of cellular biosynthetic process (GO:0031328) | 0.422233001 |
| positive regulation of macromolecule metabolic process | 0.422233001 |
| (GO:0010604) | |
| positive regulation of nitrogen compound metabolic process | 0.422233001 |
| (GO:0051173) | |
| regulation of actin cytoskeleton organization (GO:0032956) | 0.422233001 |
| regulation of molecular function (GO:0065009) | 0.422233001 |
| regulation of neuron death (GO:1901214) | 0.422233001 |
| regulation of phosphate metabolic process (GO:0019220) | 0.422233001 |
| regulation of phosphorus metabolic process (GO:0051174) | 0.422233001 |
| regulation of phosphorylation (GO:0042325) | 0.422233001 |
| response to nitrogen compound (GO:1901698) | 0.422233001 |
| response to peptide hormone (GO:0043434) | 0.422233001 |
| small molecule metabolic process (GO:0044281) | 0.422233001 |
| cellular lipid metabolic process (GO:0044255) | 0.432959407 |
| coagulation (GO:0050817) | 0.432959407 |
| cytoplasm (GO:0005737) | 0.432959407 |
| establishment of localization (GO:0051234) | 0.432959407 |
| immune response-regulating signaling pathway (GO:0002764) | 0.432959407 |
| negative regulation of cellular metabolic process (GO:0031324) | 0.432959407 |
| phosphoric ester hydrolase activity (GO:0042578) | 0.432959407 |
| positive regulation of cellular metabolic process (GO:0031325) | 0.432959407 |
| positive regulation of macromolecule biosynthetic process | 0.432959407 |
| (GO:0010557) | |
| positive regulation of metabolic process (GO:0009893) | 0.432959407 |
| positive regulation of nucleobase-containing compound | 0.432959407 |
| metabolic process (GO:0045935) | |
| regulation of hydrolase activity (GO:0051336) | 0.432959407 |
| regulation of supramolecular fiber organization (GO:1902903) | 0.432959407 |
| response to organonitrogen compound (GO:0010243) | 0.432959407 |
| transport (GO:0006810) | 0.432959407 |
| blood coagulation (GO:0007596) | 0.443606651 |
| cellular amino acid metabolic process (GO:0006520) | 0.443606651 |
| cellular component organization (GO:0016043) | 0.443606651 |
| negative regulation of macromolecule metabolic process | 0.443606651 |
| (GO:0010605) | |
| positive regulation of cellular protein metabolic process | 0.443606651 |
| (GO:0032270) | |
| protein homodimerization activity (GO:0042803) | 0.443606651 |
| regulation of immune response (GO:0050776) | 0.443606651 |
| response to stress (GO:0006950) | 0.443606651 |
| vesicle (GO:0031982) | 0.443606651 |
| Axon guidance (R-HSA-422475) | 0.454175893 |
| cell cortex (GO:0005938) | 0.454175893 |
| hemostasis (GO:0007599) | 0.454175893 |
| intracellular signal transduction (GO:0035556) | 0.454175893 |
| negative regulation of gene expression (GO:0010629) | 0.454175893 |
| negative regulation of metabolic process (GO:0009892) | 0.454175893 |
| organelle assembly (GO:0070925) | 0.454175893 |
| positive regulation of cytokine production (GO:0001819) | 0.454175893 |
| positive regulation of protein metabolic process (GO:0051247) | 0.454175893 |
| purine-containing compound metabolic process (GO:0072521) | 0.454175893 |
| regulation of protein modification process (GO:0031399) | 0.454175893 |
| response to peptide (GO:1901652) | 0.454175893 |
| cellular component organization or biogenesis (GO:0071840) | 0.464668267 |
| cellular response to cytokine stimulus (GO:0071345) | 0.464668267 |
| cofactor binding (GO:0048037) | 0.464668267 |
| endoplasmic reticulum part (GO:0044432) | 0.464668267 |
| extracellular exosome (GO:0070062) | 0.464668267 |
| extracellular organelle (GO:0043230) | 0.464668267 |
| extracellular vesicle (GO:1903561) | 0.464668267 |
| intracellular membrane-bounded organelle (GO:0043231) | 0.464668267 |
| nuclear division (GO:0000280) | 0.464668267 |
| phospholipid binding (GO:0005543) | 0.464668267 |
| positive regulation of cell adhesion (GO:0045785) | 0.464668267 |
| regulation of cellular component size (GO:0032535) | 0.464668267 |
| regulation of intracellular signal transduction (GO:1902531) | 0.464668267 |
| regulation of MAP kinase activity (GO:0043405) | 0.464668267 |
| regulation of proteolysis (GO:0030162) | 0.464668267 |
| response to extracellular stimulus (GO:0009991) | 0.464668267 |
| response to radiation (GO:0009314) | 0.464668267 |
| catalytic activity (GO:0003824) | 0.475084883 |
| cell death (GO:0008219) | 0.475084883 |
| endomembrane system (GO:0012505) | 0.475084883 |
| hydrolase activity, acting on ester bonds (GO:0016788) | 0.475084883 |
| identical protein binding (GO:0042802) | 0.475084883 |
| membrane microdomain (GO:0098857) | 0.475084883 |
| membrane region (GO:0098589) | 0.475084883 |
| microtubule-based process (GO:0007017) | 0.475084883 |
| programmed cell death (GO:0012501) | 0.475084883 |
| regulation of catalytic activity (GO:0050790) | 0.475084883 |
| regulation of stress-activated MAPK cascade (GO:0032872) | 0.475084883 |
| regulation of vesicle-mediated transport (GO:0060627) | 0.475084883 |
| response to antibiotic (GO:0046677) | 0.475084883 |
| response to cytokine (GO:0034097) | 0.475084883 |
| response to nutrient levels (GO:0031667) | 0.475084883 |
| anion binding (GO:0043168) | 0.485426827 |
| cell-cell signaling by wnt (GO:0198738) | 0.485426827 |
| enzyme regulator activity (GO:0030234) | 0.485426827 |
| extrinsic component of membrane (GO:0019898) | 0.485426827 |
| GTPase activity (GO:0003924) | 0.485426827 |
| membrane raft (GO:0045121) | 0.485426827 |
| microtubule cytoskeleton organization (GO:0000226) | 0.485426827 |
| organelle fission (GO:0048285) | 0.485426827 |
| oxidation-reduction process (GO:0055114) | 0.485426827 |
| positive regulation of cellular component biogenesis | 0.485426827 |
| (GO:0044089) | |
| positive regulation of protein kinase activity (GO:0045860) | 0.485426827 |
| positive regulation of protein serine/threonine kinase activity | 0.485426827 |
| (GO:0071902) | |
| regulation of cellular component organization (GO:0051128) | 0.485426827 |
| regulation of cellular protein metabolic process (GO:0032268) | 0.485426827 |
| regulation of protein metabolic process (GO:0051246) | 0.485426827 |
| regulation of Ras protein signal transduction (GO:0046578) | 0.485426827 |
| regulation of stress-activated protein kinase signaling cascade | 0.485426827 |
| (GO:0070302) | |
| RNA Polymerase II Transcription (R-HSA-73857) | 0.485426827 |
| Wnt signaling pathway (GO:0016055) | 0.485426827 |
| carbohydrate derivative binding (GO:0097367) | 0.495695163 |
| catalytic activity, acting on a protein (GO:0140096) | 0.495695163 |
| negative regulation of cellular component organization | 0.495695163 |
| (GO:0051129) | |
| nucleus (GO:0005634) | 0.495695163 |
| protein complex oligomerization (GO:0051259) | 0.495695163 |
| regulation of peptide transport (GO:0090087) | 0.495695163 |
| regulation of small GTPase mediated signal transduction | 0.495695163 |
| (GO:0051056) | |
| secretory vesicle (GO:0099503) | 0.495695163 |
| cellular response to abiotic stimulus (GO:0071214) | 0.50589093 |
| cellular response to environmental stimulus (GO:0104004) | 0.50589093 |
| cytoplasmic part (GO:0044444) | 0.50589093 |
| establishment or maintenance of cell polarity (GO:0007163) | 0.50589093 |
| Fatty acid metabolism (R-HSA-8978868) | 0.50589093 |
| leukocyte mediated immunity (GO:0002443) | 0.50589093 |
| Metabolism of amino acids and derivatives (R-HSA-71291) | 0.50589093 |
| organonitrogen compound metabolic process (GO:1901564) | 0.50589093 |
| positive regulation of kinase activity (GO:0033674) | 0.50589093 |
| positive regulation of response to external stimulus | 0.50589093 |
| (GO:0032103) | |
| purine nucleotide metabolic process (GO:0006163) | 0.50589093 |
| regulation of cytoskeleton organization (GO:0051493) | 0.50589093 |
| regulation of leukocyte differentiation (GO:1902105) | 0.50589093 |
| anchoring junction (GO:0070161) | 0.516015147 |
| cellular response to peptide hormone stimulus (GO:0071375) | 0.516015147 |
| cellular response to tumor necrosis factor (GO:0071356) | 0.516015147 |
| cytoplasmic vesicle membrane (GO:0030659) | 0.516015147 |
| microtubule (GO:0005874) | 0.516015147 |
| negative regulation of cellular protein metabolic process | 0.516015147 |
| (GO:0032269) | |
| negative regulation of protein metabolic process (GO:0051248) | 0.516015147 |
| post-translational protein modification (GO:0043687) | 0.516015147 |
| purine ribonucleotide metabolic process (GO:0009150) | 0.516015147 |
| regulation of cellular component biogenesis (GO:0044087) | 0.516015147 |
| regulation of leukocyte cell-cell adhesion (GO:1903037) | 0.516015147 |
| regulation of lipid metabolic process (GO:0019216) | 0.516015147 |
| regulation of protein transport (GO:0051223) | 0.516015147 |
| response to virus (GO:0009615) | 0.516015147 |
| activation of protein kinase activity (GO:0032147) | 0.526068812 |
| cell cortex part (GO:0044448) | 0.526068812 |
| cellular response to molecule of bacterial origin (GO:0071219) | 0.526068812 |
| Golgi apparatus (GO:0005794) | 0.526068812 |
| Golgi apparatus part (GO:0044431) | 0.526068812 |
| guanyl nucleotide binding (GO:0019001) | 0.526068812 |
| guanyl ribonucleotide binding (GO:0032561) | 0.526068812 |
| membrane organization (GO:0061024) | 0.526068812 |
| metabolic process (GO:0008152) | 0.526068812 |
| microtubule binding (GO:0008017) | 0.526068812 |
| organic substance metabolic process (GO:0071704) | 0.526068812 |
| positive regulation of cytoskeleton organization (GO:0051495) | 0.526068812 |
| positive regulation of hemopoiesis (GO:1903708) | 0.526068812 |
| positive regulation of immune effector process (GO:0002699) | 0.526068812 |
| positive regulation of protein transport (GO:0051222) | 0.526068812 |
| regulation of cell projection assembly (GO:0060491) | 0.526068812 |
| regulation of cytokine production (GO:0001817) | 0.526068812 |
| regulation of protein serine/threonine kinase activity | 0.526068812 |
| (GO:0071900) | |
| response to tumor necrosis factor (GO:0034612) | 0.526068812 |
| transcription factor complex (GO:0005667) | 0.526068812 |
| DNA conformation change (GO:0071103) | 0.5360529 |
| immune effector process (GO:0002252) | 0.5360529 |
| Metabolism (R-HSA-1430728) | 0.5360529 |
| monosaccharide metabolic process (GO:0005996) | 0.5360529 |
| organic cyclic compound catabolic process (GO:1901361) | 0.5360529 |
| phosphatase activity (GO:0016791) | 0.5360529 |
| positive regulation of molecular function (GO:0044093) | 0.5360529 |
| positive regulation of transferase activity (GO:0051347) | 0.5360529 |
| primary metabolic process (GO:0044238) | 0.5360529 |
| protein-containing complex (GO:0032991) | 0.5360529 |
| protein-DNA complex assembly (GO:0065004) | 0.5360529 |
| proteolysis (GO:0006508) | 0.5360529 |
| regulation of cellular localization (GO:0060341) | 0.5360529 |
| regulation of defense response (GO:0031347) | 0.5360529 |
| regulation of myeloid cell differentiation (GO:0045637) | 0.5360529 |
| regulation of plasma membrane bounded cell projection | 0.5360529 |
| assembly (GO:0120032) | |
| regulation of protein kinase activity (GO:0045859) | 0.5360529 |
| ribonucleotide metabolic process (GO:0009259) | 0.5360529 |
| small molecule binding (GO:0036094) | 0.5360529 |
| TCF dependent signaling in response to WNT (R-HSA-201681) | 0.5360529 |
| tubulin binding (GO:0015631) | 0.5360529 |
| vesicle membrane (GO:0012506) | 0.5360529 |
| adherens junction (GO:0005912) | 0.545968369 |
| cellular component assembly (GO:0022607) | 0.545968369 |
| cofactor metabolic process (GO:0051186) | 0.545968369 |
| dephosphorylation (GO:0016311) | 0.545968369 |
| Disorders of transmembrane transporters (R-HSA-5619115) | 0.545968369 |
| drug binding (GO:0008144) | 0.545968369 |
| Gene expression (Transcription) (R-HSA-74160) | 0.545968369 |
| MAPK cascade (GO:0000165) | 0.545968369 |
| microtubule-based transport (GO:0099111) | 0.545968369 |
| nucleoside phosphate metabolic process (GO:0006753) | 0.545968369 |
| nudeoside-triphosphatase activity (GO:0017111) | 0.545968369 |
| organelle part (GO:0044422) | 0.545968369 |
| positive regulation of catalytic activity (GO:0043085) | 0.545968369 |
| positive regulation of cell-cell adhesion (GO:0022409) | 0.545968369 |
| positive regulation of cellular component organization | 0.545968369 |
| (GO:0051130) | |
| protein dephosphorylation (GO:0006470) | 0.545968369 |
| protein metabolic process (GO:0019538) | 0.545968369 |
| regulation of establishment of protein localization (GO:0070201) | 0.545968369 |
| regulation of phosphatase activity (GO:0010921) | 0.545968369 |
| regulation of protein localization (GO:0032880) | 0.545968369 |
| regulation of protein polymerization (GO:0032271) | 0.545968369 |
| secretion (GO:0046903) | 0.545968369 |
| secretory granule (GO:0030141) | 0.545968369 |
| Signaling by Receptor Tyrosine Kinases (R-HSA-9006934) | 0.545968369 |
| Signaling by WNT (R-HSA-195721) | 0.545968369 |
| T cell activation (GO:0042110) | 0.545968369 |
| cell adhesion molecule binding (GO:0050839) | 0.555816155 |
| cellular response to peptide (GO:1901653) | 0.555816155 |
| clarthin-coated vesicle (GO:0030136) | 0.555816155 |
| hydrolase activity, acting on acid anhydrides (GO:0016817) | 0.555816155 |
| hydrolase activity, acting on acid anhydrides, in phosphorus- | 0.555816155 |
| containing anhydrides (GO:0016818) | |
| intracellular non-membrane-bounded organelle (GO:0043232) | 0.555816155 |
| lipid biosynthetic process (GO:0008610) | 0.555816155 |
| nitrogen compound metabolic process (GO:0006807) | 0.555816155 |
| non-membrane-bounded organelle (GO:0043228) | 0.555816155 |
| protein binding, bridging (GO:0030674) | 0.555816155 |
| pyrophosphatase activity (GO:0016462) | 0.555816155 |
| regulation of carbohydrate metabolic process (GO:0006109) | 0.555816155 |
| regulation of cysteine-type endopeptidase activity (GO:2000116) | 0.555816155 |
| regulation of kinase activity (GO:0043549) | 0.555816155 |
| regulation of response to stress (GO:0080134) | 0.555816155 |
| cellular response to external stimulus (GO:0071496) | 0.565597176 |
| cytoplasmic vesicle (GO:0031410) | 0.565597176 |
| early endosome membrane (GO:0031901) | 0.565597176 |
| endocytic vesicle (GO:0030139) | 0.565597176 |
| GTP binding (GO:0005525) | 0.565597176 |
| immune system development (GO:0002520) | 0.565597176 |
| intracellular vesicle (GO:0097708) | 0.565597176 |
| leukocyte differentiation (GO:0002521) | 0.565597176 |
| membrane lipid metabolic process (GO:0006643) | 0.565597176 |
| negative regulation of phosphate metabolic process | 0.565597176 |
| (GO:0045936) | |
| negative regulation of phosphorus metabolic process | 0.565597176 |
| (GO:0010563) | |
| nucleobase-containing small molecule metabolic process | 0.565597176 |
| (GO:0055086) | |
| nucleotide metabolic process (GO:0009117) | 0.565597176 |
| perinuclear region of cytoplasm (GO:0048471) | 0.565597176 |
| Platelet degranulation (R-HSA-114608) | 0.565597176 |
| positive regulation of cell death (GO:0010942) | 0.565597176 |
| positive regulation of establishment of protein localization | 0.565597176 |
| (GO:1904951) | |
| positive regulation of hydrolase activity (GO:0051345) | 0.565597176 |
| positive regulation of Wnt signaling pathway (GO:0030177) | 0.565597176 |
| protein phosphorylation (GO:0006468) | 0.565597176 |
| purine nucleoside binding (GO:0001883) | 0.565597176 |
| purine ribonucleoside binding (GO:0032550) | 0.565597176 |
| regulation of lymphocyte differentiation (GO:0045619) | 0.565597176 |
| regulation of nuclear division (GO:0051783) | 0.565597176 |
| regulation of T cell activation (GO:0050863) | 0.565597176 |
| regulation of transferase activity (GO:0051338) | 0.565597176 |
| response to insulin (GO:0032868) | 0.565597176 |
| ribose phosphate metabolic process (GO:0019693) | 0.565597176 |
| signal transduction by protein phosphorylation (GO:0023014) | 0.565597176 |
| cellular metabolic process (GO:0044237) | 0.575312331 |
| cellular response to biotic stimulus (GO:0071216) | 0.575312331 |
| cellular response to radiation (GO:0071478) | 0.575312331 |
| condensed chromosome (GO:0000793) | 0.575312331 |
| export from cell (GO:0140352) | 0.575312331 |
| macromolecule metabolic process (GO:0043170) | 0.575312331 |
| Metabolism of lipids (R-HSA-556833) | 0.575312331 |
| negative regulation of cytokine production (GO:0001818) | 0.575312331 |
| nucleoside binding (GO:0001882) | 0.575312331 |
| positive regulation of apoptotic process (GO:0043065) | 0.575312331 |
| positive regulation of programmed cell death (GO:0043068) | 0.575312331 |
| protein kinase regulator activity (GO:0019887) | 0.575312331 |
| protein localization to plasma membrane (GO:0072659) | 0.575312331 |
| regulation of GTPase activity (GO:0043087) | 0.575312331 |
| regulation of leukocyte mediated immunity (GO:0002703) | 0.575312331 |
| regulation of microtubule-based process (GO:0032886) | 0.575312331 |
| response to decreased oxygen levels (GO:0036293) | 0.575312331 |
| response to hypoxia (GO:0001666) | 0.575312331 |
| Rho GTPase cycle (R-HSA-194840) | 0.575312331 |
| ribonucleoside binding (GO:0032549) | 0.575312331 |
| cellular protein modification process (GO:0006464) | 0.584962501 |
| endoplasmic reticulum membrane (GO:0005789) | 0.584962501 |
| Glycerophospholipid biosynthesis (R-HSA-1483206) | 0.584962501 |
| hematopoietic or lymphoid organ development (GO:0048534) | 0.584962501 |
| Immune System (R-HSA-168256) | 0.584962501 |
| intracellular organelle part (GO:0044446) | 0.584962501 |
| macromolecule modification (GO:0043412) | 0.584962501 |
| negative regulation of apoptotic signaling pathway (GO:2001234) | 0.584962501 |
| negative regulation of organelle organization (GO:0010639) | 0.584962501 |
| negative regulation of phosphorylation (GO:0042326) | 0.584962501 |
| nuclear outer membrane-endoplasmic reticulum membrane | 0.584962501 |
| network (GO:0042175) | |
| phosphate-containing compound metabolic process | 0.584962501 |
| (GO:0006796) | |
| positive regulation of endopeptidase activity (GO:0010950) | 0.584962501 |
| protein kinase activity (GO:0004672) | 0.584962501 |
| protein modification process (GO:0036211) | 0.584962501 |
| protein-containing complex binding (GO:0044877) | 0.584962501 |
| protein-DNA complex subunit organization (GO:0071824) | 0.584962501 |
| purine nucleotide biosynthetic process (GO:0006164) | 0.584962501 |
| purine ribonucleotide biosynthetic process (GO:0009152) | 0.584962501 |
| regulation of cellular ketone metabolic process (GO:0010565) | 0.584962501 |
| regulation of cysteine-type endopeptidase activity involved in | 0.584962501 |
| apoptotic process (GO:0043281) | |
| Response to elevated platelet cytosolic Ca2+ (R-HSA-76005) | 0.584962501 |
| response to oxygen levels (GO:0070482) | 0.584962501 |
| transcription corepressor activity (GO:0003714) | 0.584962501 |
| catabolic process (GO:0009056) | 0.59454855 |
| cellular component biogenesis (GO:0044085) | 0.59454855 |
| cellular response to extracellular stimulus (GO:0031668) | 0.59454855 |
| cellular response to toxic substance (GO:0097237) | 0.59454855 |
| chromosome segregation (GO:0007059) | 0.59454855 |
| cortical cytoskeleton (GO:0030863) | 0.59454855 |
| Cytokine Signaling in Immune system (R-HSA-1280215) | 0.59454855 |
| Fc-epsilon receptor signaling pathway (GO:0038095) | 0.59454855 |
| glycerolipid metabolic process (GO:0046486) | 0.59454855 |
| hemopoiesis (GO:0030097) | 0.59454855 |
| kinase regulator activity (GO:0019207) | 0.59454855 |
| microtubule cytoskeleton (GO:0015630) | 0.59454855 |
| negative regulation of protein phosphorylation (GO:0001933) | 0.59454855 |
| organic substance catabolic process (GO:1901575) | 0.59454855 |
| organic substance transport (GO:0071702) | 0.59454855 |
| organonitrogen compound biosynthetic process (GO:1901566) | 0.59454855 |
| organophosphate metabolic process (GO:0019637) | 0.59454855 |
| phosphorus metabolic process (GO:0006793) | 0.59454855 |
| Platelet activation, signaling and aggregation (R-HSA-76002) | 0.59454855 |
| positive regulation of GTPase activity (GO:0043547) | 0.59454855 |
| purine-containing compound biosynthetic process (GO:0072522) | 0.59454855 |
| RAF/MAP kinase cascade (R-HSA-5673001) | 0.59454855 |
| Signaling by Nuclear Receptors (R-HSA-9006931) | 0.59454855 |
| apoptotic process (GO:0006915) | 0.604071324 |
| bounding membrane of organelle (GO:0098588) | 0.604071324 |
| chromatin binding (GO:0003682) | 0.604071324 |
| coenzyme binding (GO:0050662) | 0.604071324 |
| cysteine-type peptidase activity (GO:0008234) | 0.604071324 |
| DNA recombination (GO:0006310) | 0.604071324 |
| Golgi membrane (GO:0000139) | 0.604071324 |
| lymphocyte differentiation (GO:0030098) | 0.604071324 |
| MAPK1/MAPK3 signaling (R-HSA-5684996) | 0.604071324 |
| organonitrogen compound catabolic process (GO:1901565) | 0.604071324 |
| positive regulation of cell cycle process (GO:0090068) | 0.604071324 |
| positive regulation of defense response (GO:0031349) | 0.604071324 |
| positive regulation of DNA-binding transcription factor activity | 0.604071324 |
| (GO:0051091) | |
| Post-translational protein modification (R-HSA-597592) | 0.604071324 |
| purine ribonucleotide binding (GO:0032555) | 0.604071324 |
| ribonucleotide binding (GO:0032553) | 0.604071324 |
| transferase activity (GO:0016740) | 0.604071324 |
| actin filament (GO:0005884) | 0.613531653 |
| aromatic compound catabolic process (GO:0019439) | 0.613531653 |
| cytosolic ribosome (GO:0022626) | 0.613531653 |
| Golgi stack (GO:0005795) | 0.613531653 |
| Interferon Signaling (R-HSA-913531) | 0.613531653 |
| isomerase activity (GO:0016853) | 0.613531653 |
| negative regulation of intracellular signal transduction | 0.613531653 |
| (GO:1902532) | |
| organelle membrane (GO:0031090) | 0.613531653 |
| organelle organization (GO:0006996) | 0.613531653 |
| positive regulation of canonical Wnt signaling pathway | 0.613531653 |
| (GO:0090263) | |
| positive regulation of cell cycle (GO:0045787) | 0.613531653 |
| positive regulation of peptidase activity (GO:0010952) | 0.613531653 |
| positive regulation of T cell activation (GO:0050870) | 0.613531653 |
| purine nucleotide binding (GO:0017076) | 0.613531653 |
| regulation of small molecule metabolic process (GO:0062012) | 0.613531653 |
| secretion by cell (GO:0032940) | 0.613531653 |
| Signaling by the B Cell Receptor (BCR)(R-HSA-983705) | 0.613531653 |
| adenyl ribonucleotide binding (GO:0032559) | 0.622930351 |
| biosynthetic process (GO:0009058) | 0.622930351 |
| cellular macromolecule metabolic process (GO:0044260) | 0.622930351 |
| cellular nitrogen compound catabolic process (GO:0044270) | 0.622930351 |
| generation of precursor metabolites and energy (GO:0006091) | 0.622930351 |
| intracellular receptor signaling pathway (GO:0030522) | 0.622930351 |
| molecular adaptor activity (GO:0060090) | 0.622930351 |
| nucleoside phosphate binding (GO:1901265) | 0.622930351 |
| nucleotide binding (GO:0000166) | 0.622930351 |
| phosphorylation (GO:0016310) | 0.622930351 |
| phosphotransferase activity, alcohol group as acceptor | 0.622930351 |
| (GO:0016773) | |
| positive regulation of leukocyte cell-cell adhesion (GO:1903039) | 0.622930351 |
| protein localization to membrane (GO:0072657) | 0.622930351 |
| purine ribonucleoside triphosphate binding (GO:0035639) | 0.622930351 |
| regulation of DNA-binding transcription factor activity | 0.622930351 |
| (GO:0051090) | |
| regulation of endocytosis (GO:0030100) | 0.622930351 |
| ribonucleotide biosynthetic process (GO:0009260) | 0.622930351 |
| T cell differentiation (GO:0030217) | 0.622930351 |
| vesicle-mediated transport (GO:0016192) | 0.622930351 |
| adenyl nucleotide binding (GO:0030554) | 0.632268215 |
| centriole (GO:0005814) | 0.632268215 |
| coated vesicle membrane (GO:0030662) | 0.632268215 |
| early endosome (GO:0005769) | 0.632268215 |
| kinase activity (GO:0016301) | 0.632268215 |
| macromolecule localization (GO:0033036) | 0.632268215 |
| MAPK family signaling cascades (R-HSA-5683057) | 0.632268215 |
| organic substance biosynthetic process (GO:1901576) | 0.632268215 |
| regulation of dephosphorylation (GO:0035303) | 0.632268215 |
| Signaling by Interleukins (R-HSA-449147) | 0.632268215 |
| ATP binding (GO:0005524) | 0.641546029 |
| ATPase activity (GO:0016887) | 0.641546029 |
| cellular biosynthetic process (GO:0044249) | 0.641546029 |
| cellular protein metabolic process (GO:0044267) | 0.641546029 |
| cellular response to nutrient levels (GO:0031669) | 0.641546029 |
| cytoplasmic vesicle part (GO:0044433) | 0.641546029 |
| heterocycle catabolic process (GO:0046700) | 0.641546029 |
| Innate Immune System (R-HSA-168249) | 0.641546029 |
| methylation (GO:0032259) | 0.641546029 |
| negative regulation of protein modification process | 0.641546029 |
| (GO:0031400) | |
| nucleoside phosphate biosynthetic process (GO:1901293) | 0.641546029 |
| regulation of adaptive immune response (GO:0002819) | 0.641546029 |
| regulation of organelle organization (GO:0033043) | 0.641546029 |
| ribose phosphate biosynthetic process (GO:0046390) | 0.641546029 |
| transferase activity, transferring acyl groups (GO:0016746) | 0.641546029 |
| cellular response to insulin stimulus (GO:0032869) | 0.650764559 |
| coenzyme metabolic process (GO:0006732) | 0.650764559 |
| COPII-coated ER to Golgi transport vesicle (GO:0030134) | 0.650764559 |
| cytoplasmic side of plasma membrane (GO:0009898) | 0.650764559 |
| cytosolic part (GO:0044445) | 0.650764559 |
| Estrogen-dependent gene expression (R-HSA-9018519) | 0.650764559 |
| Fc epsilon receptor (FCERI)signaling (R-HSA-2454202) | 0.650764559 |
| monocarboxylic acid catabolic process (GO:0072329) | 0.650764559 |
| negative regulation of transferase activity (GO:0051348) | 0.650764559 |
| organic cyclic compound biosynthetic process (GO:1901362) | 0.650764559 |
| phosphatidylinositol binding (GO:0035091) | 0.650764559 |
| protein domain specific binding (GO:0019904) | 0.650764559 |
| Ras guanyl-nucleotide exchange factor activity (GO:0005088) | 0.650764559 |
| regulation of apoptotic signaling pathway (GO:2001233) | 0.650764559 |
| regulation of binding (GO:0051098) | 0.650764559 |
| Rho GTPase binding (GO:0017048) | 0.650764559 |
| vacuolar lumen (GO:0005775) | 0.650764559 |
| whole membrane (GO:0098805) | 0.650764559 |
| cell leading edge (GO:0031252) | 0.659924558 |
| cellular catabolic process (GO:0044248) | 0.659924558 |
| coated vesicle (GO:0030135) | 0.659924558 |
| Disease (R-HSA-1643685) | 0.659924558 |
| enzyme activator activity (GO:0008047) | 0.659924558 |
| hexose metabolic process (GO:0019318) | 0.659924558 |
| membrane fusion (GO:0061025) | 0.659924558 |
| Metabolism of carbohydrates (R-HSA-71387) | 0.659924558 |
| Metabolism of proteins (R-HSA-392499) | 0.659924558 |
| microtubule organizing center (GO:0005815) | 0.659924558 |
| negative regulation of catabolic process (GO:0009895) | 0.659924558 |
| negative regulation of kinase activity (GO:0033673) | 0.659924558 |
| nucleotide biosynthetic process (GO:0009165) | 0.659924558 |
| organic cyclic compound metabolic process (GO:1901360) | 0.659924558 |
| protein heterooligomerization (GO:0051291) | 0.659924558 |
| regulation of hemopoiesis (GO:1903706) | 0.659924558 |
| regulation of microtubule cytoskeleton organization | 0.659924558 |
| (GO:0070507) | |
| regulation of multi-organism process (GO:0043900) | 0.659924558 |
| B cell activation (GO:0042113) | 0.669026766 |
| EPH-Ephrin signaling (R-HSA-2682334) | 0.669026766 |
| glucose metabolic process (GO:0006006) | 0.669026766 |
| lymphocyte activation (GO:0046649) | 0.669026766 |
| maintenance of location (GO:0051235) | 0.669026766 |
| microbody part (GO:0044438) | 0.669026766 |
| microtubule organizing center part (GO:0044450) | 0.669026766 |
| nuclear transcription factor complex (GO:0044798) | 0.669026766 |
| peroxisomal part (GO:0044439) | 0.669026766 |
| regulation of mitotic nuclear division (GO:0007088) | 0.669026766 |
| regulation of protein dephosphorylation (GO:0035304) | 0.669026766 |
| RNA Polymerase I Transcription (R-HSA-73864) | 0.669026766 |
| transferase activity, transferring phosphorus-containing groups | 0.669026766 |
| (GO:0016772) | |
| Adaptive Immune System (R-HSA-1280218) | 0.678071905 |
| ESR-mediated signaling (R-HSA-8939211) | 0.678071905 |
| GTPase activator activity (GO:0005096) | 0.678071905 |
| inclusion body (GO:0016234) | 0.678071905 |
| negative regulation of protein kinase activity (GO:0006469) | 0.678071905 |
| positive regulation of proteolysis (GO:0045862) | 0.678071905 |
| Processing of DNA double-strand break ends (R-HSA-5693607) | 0.678071905 |
| protein-containing complex assembly (GO:0065003) | 0.678071905 |
| protein-containing complex subunit organization (GO:0043933) | 0.678071905 |
| regulation of protein complex assembly (GO:0043254) | 0.678071905 |
| Selenoamino acid metabolism (R-HSA-2408522) | 0.678071905 |
| sister chromatid segregation (GO:0000819) | 0.678071905 |
| transcription factor binding (GO:0008134) | 0.678071905 |
| transcription initiation from RNA polymerase II promoter | 0.678071905 |
| (GO:0006367) | |
| cell cycle arrest (GO:0007050) | 0.687060688 |
| cellular aromatic compound metabolic process (GO:0006725) | 0.687060688 |
| cytoplasmic side of membrane (GO:0098562) | 0.687060688 |
| cytosol (GO:0005829) | 0.687060688 |
| Formation of a pool of free 40S subunits (R-HSA-72689) | 0.687060688 |
| kinase binding (GO:0019900) | 0.687060688 |
| neuron projection cytoplasm (GO:0120111) | 0.687060688 |
| phospholipid metabolic process (GO:0006644) | 0.687060688 |
| recycling endosome (GO:0055037) | 0.687060688 |
| regulation of cellular response to stress (GO:0080135) | 0.687060688 |
| regulation of reactive oxygen species metabolic process | 0.687060688 |
| (GO:2000377) | |
| aromatic compound biosynthetic process (GO:0019438) | 0.695993813 |
| ATP metabolic process (GO:0046034) | 0.695993813 |
| cell activation (GO:0001775) | 0.695993813 |
| cell-substrate junction (GO:0030055) | 0.695993813 |
| cellular carbohydrate metabolic process (GO:0044262) | 0.695993813 |
| cellular localization (GO:0051641) | 0.695993813 |
| cellular response to leukemia inhibitory factor (GO:1990830) | 0.695993813 |
| cytoplasmic ribonucleoprotein granule (GO:0036464) | 0.695993813 |
| glycerophospholipid metabolic process (GO:0006650) | 0.695993813 |
| GTPase regulator activity (GO:0030695) | 0.695993813 |
| negative regulation of cellular catabolic process (GO:0031330) | 0.695993813 |
| negative regulation of DNA-binding transcription factor activity | 0.695993813 |
| (GO:0043433) | |
| negative regulation of intrinsic apoptotic signaling pathway | 0.695993813 |
| (GO:2001243) | |
| nuclease activity (GO:0004518) | 0.695993813 |
| protein localization (GO:0008104) | 0.695993813 |
| regulation of DNA repair (GO:0006282) | 0.695993813 |
| regulation of nucleotide metabolic process (GO:0006140) | 0.695993813 |
| response to leukemia inhibitory factor (GO:1990823) | 0.695993813 |
| response to reactive oxygen species (GO:0000302) | 0.695993813 |
| RUNX1 regulates transcription of genes involved in differentiation | 0.695993813 |
| of HSCs (R-HSA-8939236) | |
| small GTPase mediated signal transduction (GO:0007264) | 0.695993813 |
| transcription coregulator activity (GO:0003712) | 0.695993813 |
| transferase activity, transferring acyl groups other than amino- | 0.695993813 |
| acyl groups (GO:0016747) | |
| transport along microtubule (GO:0010970) | 0.695993813 |
| cell cycle (GO:0007049) | 0.704871964 |
| cell-substrate adherens junction (GO:0005924) | 0.704871964 |
| endosome (GO:0005768) | 0.704871964 |
| hormone receptor binding (GO:0051427) | 0.704871964 |
| macromolecule biosynthetic process (GO:0009059) | 0.704871964 |
| macromolecule methylation (GO:0043414) | 0.704871964 |
| microbody (GO:0042579) | 0.704871964 |
| mitotic DNA integrity checkpoint (GO:0044774) | 0.704871964 |
| negative regulation of protein binding (GO:0032091) | 0.704871964 |
| nitrogen compound transport (GO:0071705) | 0.704871964 |
| non-canonical Wnt signaling pathway (GO:0035567) | 0.704871964 |
| pattern recognition receptor signaling pathway (GO:0002221) | 0.704871964 |
| peptidyl-amino acid modification (GO:0018193) | 0.704871964 |
| peptidyl-serine modification (GO:0018209) | 0.704871964 |
| peroxisome (GO:0005777) | 0.704871964 |
| positive regulation of organelle organization (GO:0010638) | 0.704871964 |
| protein phosphatase binding (GO:0019903) | 0.704871964 |
| regulation of cell cycle G1/S phase transition (GO:1902806) | 0.704871964 |
| regulation of protein binding (GO:0043393) | 0.704871964 |
| response to interleukin-1 (GO:0070555) | 0.704871964 |
| stress-activated MAPK cascade (GO:0051403) | 0.704871964 |
| cellular amide metabolic process (GO:0043603) | 0.713695815 |
| cellular macromolecule localization (GO:0070727) | 0.713695815 |
| cellular nitrogen compound metabolic process (GO:0034641) | 0.713695815 |
| cellular protein localization (GO:0034613) | 0.713695815 |
| DNA metabolic process (GO:0006259) | 0.713695815 |
| enzyme binding (GO:0019899) | 0.713695815 |
| focal adhesion (GO:0005925) | 0.713695815 |
| heterocycle metabolic process (GO:0046483) | 0.713695815 |
| intrinsic component of organelle membrane (GO:0031300) | 0.713695815 |
| methyltransferase activity (GO:0008168) | 0.713695815 |
| nucleobase-containing compound catabolic process | 0.713695815 |
| (GO:0034655) | |
| positive regulation of endocytosis (GO:0045807) | 0.713695815 |
| protein kinase binding (GO:0019901) | 0.713695815 |
| regulation of response to biotic stimulus (GO:0002831) | 0.713695815 |
| regulation of response to DNA damage stimulus (GO:2001020) | 0.713695815 |
| regulation of symbiosis, encompassing mutualism through | 0.713695815 |
| parasitism (GO:0043903) | |
| ribonucleoprotein granule (GO:0035770) | 0.713695815 |
| RNA modification (GO:0009451) | 0.713695815 |
| secretory granule membrane (GO:0030667) | 0.713695815 |
| SH3 domain binding (GO:0017124) | 0.713695815 |
| signal transduction by p53 class mediator (GO:0072331) | 0.713695815 |
| Signaling by Rho GTPases (R-HSA-194315) | 0.713695815 |
| Signaling by TGF-beta family members (R-HSA-9006936) | 0.713695815 |
| Sphingolipid metabolism (R-HSA-428157) | 0.713695815 |
| transcription by RNA polymerase II (GO:0006366) | 0.713695815 |
| transferase activity, transferring one-carbon groups | 0.713695815 |
| (GO:0016741) | |
| vesicle lumen (GO:0031983) | 0.713695815 |
| apoptotic signaling pathway (GO:0097190) | 0.722466024 |
| cell cycle process (GO:0022402) | 0.722466024 |
| cellular response to interleukin-1 (GO:0071347) | 0.722466024 |
| chromosome organization (GO:0051276) | 0.722466024 |
| COPI-dependent Golgi-to-ER retrograde traffic (R-HSA-6811434) | 0.722466024 |
| cytoplasmic vesicle lumen (GO:0060205) | 0.722466024 |
| exocytosis (GO:0006887) | 0.722466024 |
| extrinsic apoptotic signaling pathway (GO:0097191) | 0.722466024 |
| gene silencing by RNA (GO:0031047) | 0.722466024 |
| heterocycle biosynthetic process (GO:0018130) | 0.722466024 |
| intracellular organelle lumen (GO:0070013) | 0.722466024 |
| lipid modification (GO:0030258) | 0.722466024 |
| maintenance of location in cell (GO:0051651) | 0.722466024 |
| membrane-enclosed lumen (GO:0031974) | 0.722466024 |
| Metabolism of nucleotides (R-HSA-15869) | 0.722466024 |
| mitochondrion (GO:0005739) | 0.722466024 |
| mitotic DNA damage checkpoint (GO:0044773) | 0.722466024 |
| Mitotic Prophase (R-HSA-68875) | 0.722466024 |
| negative regulation of cellular protein localization (GO:1903828) | 0.722466024 |
| nucleobase-containing compound biosynthetic process | 0.722466024 |
| (GO:0034654) | |
| nudeoside-triphosphatase regulator activity (GO:0060589) | 0.722466024 |
| organelle lumen (GO:0043233) | 0.722466024 |
| organelle outer membrane (GO:0031968) | 0.722466024 |
| outer membrane (GO:0019867) | 0.722466024 |
| positive regulation of mitotic cell cycle (GO:0045931) | 0.722466024 |
| Ras GTPase binding (GO:0017016) | 0.722466024 |
| Ras protein signal transduction (GO:0007265) | 0.722466024 |
| regulation of cell cycle (GO:0051726) | 0.722466024 |
| regulation of cellular protein localization (GO:1903827) | 0.722466024 |
| regulation of DNA metabolic process (GO:0051052) | 0.722466024 |
| regulation of purine nucleotide metabolic process (GO:1900542) | 0.722466024 |
| small GTPase binding (GO:0031267) | 0.722466024 |
| cellular macromolecule biosynthetic process (GO:0034645) | 0.731183242 |
| Cellular Senescence (R-HSA-2559583) | 0.731183242 |
| chromatin (GO:0000785) | 0.731183242 |
| DNA biosynthetic process (GO:0071897) | 0.731183242 |
| establishment of localization in cell (GO:0051649) | 0.731183242 |
| Fc receptor signaling pathway (GO:0038093) | 0.731183242 |
| Golgi subcompartment (GO:0098791) | 0.731183242 |
| Golgi-associated vesicle membrane (GO:0030660) | 0.731183242 |
| guanyl-nucleotide exchange factor activity (GO:0005085) | 0.731183242 |
| Nonsense Mediated Decay (NMD) enhanced by the Exon | 0.731183242 |
| Junction Complex (EJC)(R-HSA-975957) | |
| Nonsense-Mediated Decay (NMD) (R-HSA-927802) | 0.731183242 |
| nuclear chromosome (GO:0000228) | 0.731183242 |
| organelle subcompartment (GO:0031984) | 0.731183242 |
| organophosphate biosynthetic process (GO:0090407) | 0.731183242 |
| positive regulation of NF-kappaB transcription factor activity | 0.731183242 |
| (GO:0051092) | |
| protein serine/threonine kinase activity (GO:0004674) | 0.731183242 |
| regulated exocytosis (GO:0045055) | 0.731183242 |
| regulation of G1/S transition of mitotic cell cycle (GO:2000045) | 0.731183242 |
| regulation of viral process (GO:0050792) | 0.731183242 |
| respiratory chain complex (GO:0098803) | 0.731183242 |
| Ub-specific processing proteases (R-HSA-5689880) | 0.731183242 |
| ubiguitin protein ligase activity (GO:0061630) | 0.731183242 |
| cellular component disassembly (GO:0022411) | 0.739848103 |
| chromatin organization (GO:0006325) | 0.739848103 |
| cytoskeleton-dependent intracellular transport (GO:0030705) | 0.739848103 |
| gene silencing (GO:0016458) | 0.739848103 |
| Golgi-associated vesicle (GO:0005798) | 0.739848103 |
| HDR through Homologous Recombination (HRR) or Single | 0.739848103 |
| Strand Annealing (SSA) (R-HSA-5693567) | |
| interaction with host (GO:0051701) | 0.739848103 |
| L13a-mediated translational silencing of Ceruloplasmin | 0.739848103 |
| expression (R-HSA-156827) | |
| leukocyte activation (GO:0045321) | 0.739848103 |
| mitochondrial membrane organization (GO:0007006) | 0.739848103 |
| mRNA binding (GO:0003729) | 0.739848103 |
| negative regulation of NF-kappaB transcription factor activity | 0.739848103 |
| (GO:0032088) | |
| nuclear chromosome part (GO:0044454) | 0.739848103 |
| nucleobase-containing compound metabolic process | 0.739848103 |
| (GO:0006139) | |
| phosphatase binding (GO:0019902) | 0.739848103 |
| positive regulation of response to DNA damage stimulus | 0.739848103 |
| (GO:2001022) | |
| respirasome (GO:0070469) | 0.739848103 |
| response to oxidative stress (GO:0006979) | 0.739848103 |
| secretory granule lumen (GO:0034774) | 0.739848103 |
| ubiguitin-like protein ligase activity (GO:0061659) | 0.739848103 |
| Vesicle-mediated transport (R-HSA-5653656) | 0.739848103 |
| catalytic activity, acting on DNA (GO:0140097) | 0.748461233 |
| cellular nitrogen compound biosynthetic process (GO:0044271) | 0.748461233 |
| cellular response to hypoxia (GO:0071456) | 0.748461233 |
| cellular response to oxygen levels (GO:0071453) | 0.748461233 |
| Chaperonin-mediated protein folding (R-HSA-390466) | 0.748461233 |
| chromatin remodeling (GO:0006338) | 0.748461233 |
| establishment of protein localization to peroxisome | 0.748461233 |
| (GO:0072663) | |
| GTPase binding (GO:0051020) | 0.748461233 |
| integral component of organelle membrane (GO:0031301) | 0.748461233 |
| ligase activity (GO:0016874) | 0.748461233 |
| mitotic cell cycle checkpoint (GO:0007093) | 0.748461233 |
| modification of morphology or physiology of other organism | 0.748461233 |
| involved in symbiotic interaction (GO:0051817) | |
| Neddylation (R-HSA-8951664) | 0.748461233 |
| negative regulation of mitotic cell cycle (GO:0045930) | 0.748461233 |
| nuclear chromatin (GO:0000790) | 0.748461233 |
| peroxisomal transport (GO:0043574) | 0.748461233 |
| phosphatidylinositol metabolic process (GO:0046488) | 0.748461233 |
| Phospholipid metabolism (R-HSA-1483257) | 0.748461233 |
| positive regulation of I-kappaB kinase/NF-kappaB signaling | 0.748461233 |
| (GO:0043123) | |
| positive regulation of protein catabolic process (GO:0045732) | 0.748461233 |
| positive regulation of response to biotic stimulus (GO:0002833) | 0.748461233 |
| protein localization to peroxisome (GO:0072662) | 0.748461233 |
| protein targeting to peroxisome (GO:0006625) | 0.748461233 |
| regulation of cytokine-mediated signaling pathway (GO:0001959) | 0.748461233 |
| regulation of I-kappaB kinase/NF-kappaB signaling | 0.748461233 |
| (GO:0043122) | |
| regulation of organelle assembly (GO:1902115) | 0.748461233 |
| regulation of protein localization to nucleus (GO:1900180) | 0.748461233 |
| ribonuclease activity (GO:0004540) | 0.748461233 |
| Transcriptional regulation by RUNX2 (R-HSA-8878166) | 0.748461233 |
| Wnt signaling pathway, planar cell polarity pathway | 0.748461233 |
| (GO:0060071) | |
| Biosynthesis of the N-glycan precursor (dolichol lipid-linked | 0.757023247 |
| oligosaccharide, LLO)and transfer to a nascent protein (R-HSA- | |
| 446193) | |
| cell division (GO:0051301) | 0.757023247 |
| cellular response to decreased oxygen levels (GO:0036294) | 0.757023247 |
| cytoskeleton-dependent cytokinesis (GO:0061640) | 0.757023247 |
| DNA-binding transcription factor binding (GO:0140297) | 0.757023247 |
| DNA-templated transcription, initiation (GO:0006352) | 0.757023247 |
| electron transport chain (GO:0022900) | 0.757023247 |
| homeostasis of number of cells (GO:0048872) | 0.757023247 |
| Homology Directed Repair (R-HSA-5693538) | 0.757023247 |
| inner mitochondrial membrane protein complex (GO:0098800) | 0.757023247 |
| magnesium ion binding (GO:0000287) | 0.757023247 |
| maintenance of protein location (GO:0045185) | 0.757023247 |
| myeloid cell differentiation (GO:0030099) | 0.757023247 |
| nuclear part (GO:0044428) | 0.757023247 |
| nucleic acid-templated transcription (GO:0097659) | 0.757023247 |
| oxidoreductase activity, acting on NAD(P)H (GO:0016651) | 0.757023247 |
| positive regulation of neuron death (GO:1901216) | 0.757023247 |
| positive regulation of protein complex assembly (GO:0031334) | 0.757023247 |
| protein ubiquitination (GO:0016567) | 0.757023247 |
| regulation of cell cycle process (GO:0010564) | 0.757023247 |
| regulation of generation of precursor metabolites and energy | 0.757023247 |
| (GO:0043467) | |
| regulation of innate immune response (GO:0045088) | 0.757023247 |
| regulation of phagocytosis (GO:0050764) | 0.757023247 |
| regulation of response to cytokine stimulus (GO:0060759) | 0.757023247 |
| response to hydrogen peroxide (GO:0042542) | 0.757023247 |
| RNA polymerase II transcription factor complex (GO:0090575) | 0.757023247 |
| transcription, DNA-templated (GO:0006351) | 0.757023247 |
| amide transport (GO:0042886) | 0.765534746 |
| cellular protein-containing complex assembly (GO:0034622) | 0.765534746 |
| fatty acid catabolic process (GO:0009062) | 0.765534746 |
| GTP hydrolysis and joining of the 60S ribosomal subunit (R-HSA- | 0.765534746 |
| 72706) | |
| Hedgehog ‘on’ state (R-HSA-5632684) | 0.765534746 |
| negative regulation of cell cycle (GO:0045786) | 0.765534746 |
| positive regulation of binding (GO:0051099) | 0.765534746 |
| positive regulation of catabolic process (GO:0009896) | 0.765534746 |
| positive regulation of cellular protein localization (GO:1903829) | 0.765534746 |
| positive regulation of innate immune response (GO:0045089) | 0.765534746 |
| positive regulation of multi-organism process (GO:0043902) | 0.765534746 |
| protein alkylation (GO:0008213) | 0.765534746 |
| Protein folding (R-HSA-391251) | 0.765534746 |
| protein methylation (GO:0006479) | 0.765534746 |
| protein targeting to membrane (GO:0006612) | 0.765534746 |
| RNA biosynthetic process (GO:0032774) | 0.765534746 |
| Signaling by Hedgehog (R-HSA-5358351) | 0.765534746 |
| spindle assembly (GO:0051225) | 0.765534746 |
| SRP-dependent cotranslational protein targeting to membrane | 0.765534746 |
| (GO:0006614) | |
| SRP-dependent cotranslational protein targeting to membrane | 0.765534746 |
| (R-HSA-1799339) | |
| tertiary granule (GO:0070820) | 0.765534746 |
| ubiquitin-protein transferase activity (GO:0004842) | 0.765534746 |
| vacuole (GO:0005773) | 0.765534746 |
| Cell death signalling via NRAGE, NRIF and NADE (R-HSA- | 0.773996325 |
| 204998) | |
| cellular response to starvation (GO:0009267) | 0.773996325 |
| cellular response to stress (GO:0033554) | 0.773996325 |
| chromosomal part (GO:0044427) | 0.773996325 |
| endomembrane system organization (GO:0010256) | 0.773996325 |
| endosomal part (GO:0044440) | 0.773996325 |
| establishment of protein localization to membrane (GO:0090150) | 0.773996325 |
| intrinsic apoptotic signaling pathway (GO:0097193) | 0.773996325 |
| MHC class II antigen presentation (R-HSA-2132295) | 0.773996325 |
| Mitochondrial biogenesis (R-HSA-1592230) | 0.773996325 |
| mitochondrial outer membrane (GO:0005741) | 0.773996325 |
| mitochondrial transmembrane transport (GO:1990542) | 0.773996325 |
| nuclear lumen (GO:0031981) | 0.773996325 |
| nucleic acid metabolic process (GO:0090304) | 0.773996325 |
| nucleic acid phosphodiester bond hydrolysis (GO:0090305) | 0.773996325 |
| peptide transport (GO:0015833) | 0.773996325 |
| protein autophosphorylation (GO:0046777) | 0.773996325 |
| protein-containing complex localization (GO:0031503) | 0.773996325 |
| response to starvation (GO:0042594) | 0.773996325 |
| Signaling by ROBO receptors (R-HSA-376176) | 0.773996325 |
| ubiquitin-like protein transferase activity (GO:0019787) | 0.773996325 |
| ATPase activity, coupled (GO:0042623) | 0.782408565 |
| cellular response to antibiotic (GO:0071236) | 0.782408565 |
| chromosome (GO:0005694) | 0.782408565 |
| cotranslational protein targeting to membrane (GO:0006613) | 0.782408565 |
| DDX58/IFIH1-mediated induction of interferon-alpha/beta (R- | 0.782408565 |
| HSA-168928) | |
| endoplasmic reticulum-Golgi intermediate compartment | 0.782408565 |
| membrane (GO:0033116) | |
| establishment of protein localization (GO:0045184) | 0.782408565 |
| establishment of protein localization to endoplasmic reticulum | 0.782408565 |
| (GO:0072599) | |
| HATs acetylate histones (R-HSA-3214847) | 0.782408565 |
| lamellipodium (GO:0030027) | 0.782408565 |
| mitochondrial part (GO:0044429) | 0.782408565 |
| mitotic sister chromatid segregation (GO:0000070) | 0.782408565 |
| Organelle biogenesis and maintenance (R-HSA-1852241) | 0.782408565 |
| positive regulation of apoptotic signaling pathway (GO:2001235) | 0.782408565 |
| positive regulation of protein binding (GO:0032092) | 0.782408565 |
| positive regulation of protein ubiguitination (GO:0031398) | 0.782408565 |
| PPARA activates gene expression (R-HSA-1989781) | 0.782408565 |
| protein stabilization (GO:0050821) | 0.782408565 |
| Protein ubiquitination (R-HSA-8852135) | 0.782408565 |
| regulation of chromatin organization (GO:1902275) | 0.782408565 |
| Regulation of expression of SLITs and ROBOs (R-HSA- | 0.782408565 |
| 9010553) | |
| Regulation of lipid metabolism by Peroxisome proliferator- | 0.782408565 |
| activated receptor alpha (PPARalpha) (R-HSA-400206) | |
| regulation of mitotic cell cycle (GO:0007346) | 0.782408565 |
| regulation of protein catabolic process (GO:0042176) | 0.782408565 |
| RHO GTPase Effectors (R-HSA-195258) | 0.782408565 |
| RNA polymerase II-specific DNA-binding transcription factor | 0.782408565 |
| binding (GO:0061629) | |
| Transcriptional regulation by RUNX1 (R-HSA-8878171) | 0.782408565 |
| Cap-dependent Translation Initiation (R-HSA-72737) | 0.790772038 |
| cell division site part (GO:0032155) | 0.790772038 |
| chaperone binding (GO:0051087) | 0.790772038 |
| Cilium Assembly (R-HSA-5617833) | 0.790772038 |
| endosome membrane (GO:0010008) | 0.790772038 |
| Eukaryotic Translation Initiation (R-HSA-72613) | 0.790772038 |
| lysosome (GO:0005764) | 0.790772038 |
| lytic vacuole (GO:0000323) | 0.790772038 |
| mitochondrial membrane (GO:0031966) | 0.790772038 |
| negative regulation of gene expression, epigenetic | 0.790772038 |
| (GO:0045814) | |
| organelle localization (GO:0051640) | 0.790772038 |
| peptidyl-lysine methylation (GO:0018022) | 0.790772038 |
| peptidyl-serine phosphorylation (GO:0018105) | 0.790772038 |
| peptidyl-threonine modification (GO:0018210) | 0.790772038 |
| protein folding (GO:0006457) | 0.790772038 |
| protein localization to endoplasmic reticulum (GO:0070972) | 0.790772038 |
| protein modification by small protein conjugation (GO:0032446) | 0.790772038 |
| protein targeting to ER (GO:0045047) | 0.790772038 |
| protein transport (GO:0015031) | 0.790772038 |
| Rab GTPase binding (GO:0017137) | 0.790772038 |
| regulation of stem cell differentiation (GO:2000736) | 0.790772038 |
| RNA phosphodiester bond hydrolysis (GO:0090501) | 0.790772038 |
| Signaling by NOTCH (R-HSA-157118) | 0.790772038 |
| toll-like receptor signaling pathway (GO:0002224) | 0.790772038 |
| transcription coactivator activity (GO:0003713) | 0.790772038 |
| unfolded protein binding (GO:0051082) | 0.790772038 |
| vacuolar part (GO:0044437) | 0.790772038 |
| antigen processing and presentation of peptide or polysaccharide | 0.799087306 |
| antigen via MHC class II (GO:0002504) | |
| Beta-catenin independent WNT signaling (R-HSA-3858494) | 0.799087306 |
| cell activation involved in immune response (GO:0002263) | 0.799087306 |
| coenzyme biosynthetic process (GO:0009108) | 0.799087306 |
| cofactor biosynthetic process (GO:0051188) | 0.799087306 |
| envelope (GO:0031975) | 0.799087306 |
| histone methylation (GO:0016571) | 0.799087306 |
| Interleukin-12 family signaling (R-HSA-447115) | 0.799087306 |
| leukocyte activation involved in immune response (GO:0002366) | 0.799087306 |
| mitochondrial envelope (GO:0005740) | 0.799087306 |
| negative regulation of chromosome organization (GO:2001251) | 0.799087306 |
| nuclear envelope (GO:0005635) | 0.799087306 |
| nuclear-transcribed mRNA catabolic process, nonsense- | 0.799087306 |
| mediated decay (GO:0000184) | |
| nucleoside monophosphate metabolic process (GO:0009123) | 0.799087306 |
| organelle envelope (GO:0031967) | 0.799087306 |
| organelle inner membrane (GO:0019866) | 0.799087306 |
| oxidoreductase complex (GO:1990204) | 0.799087306 |
| protein modification by small protein conjugation or removal | 0.799087306 |
| (GO:0070647) | |
| recombinational repair (GO:0000725) | 0.799087306 |
| regulation of intrinsic apoptotic signaling pathway (GO:2001242) | 0.799087306 |
| response to ionizing radiation (GO:0010212) | 0.799087306 |
| stress-activated protein kinase signaling cascade (GO:0031098) | 0.799087306 |
| vesicle fusion (GO:0006906) | 0.799087306 |
| vesicle organization (GO:0016050) | 0.799087306 |
| Antigen processing: Ubiquitination & Proteasome degradation | 0.807354922 |
| (R-HSA-983168) | |
| axon cytoplasm (GO:1904115) | 0.807354922 |
| cellular response to oxidative stress (GO:0034599) | 0.807354922 |
| cellular response to reactive oxygen species (GO:0034614) | 0.807354922 |
| centrosome (GO:0005813) | 0.807354922 |
| condensed chromosome kinetochore (GO:0000777) | 0.807354922 |
| condensed chromosome, centromeric region (GO:0000779) | 0.807354922 |
| cullin-RING ubiquitin ligase complex (GO:0031461) | 0.807354922 |
| establishment of organelle localization (GO:0051656) | 0.807354922 |
| glycerolipid biosynthetic process (GO:0045017) | 0.807354922 |
| histone deacetylase binding (GO:0042826) | 0.807354922 |
| innate immune response-activating signal transduction | 0.807354922 |
| (GO:0002758) | |
| intrinsic component of endoplasmic reticulum membrane | 0.807354922 |
| (GO:0031227) | |
| macromolecule catabolic process (GO:0009057) | 0.807354922 |
| mitochondrial membrane part (GO:0044455) | 0.807354922 |
| mitochondrial transport (GO:0006839) | 0.807354922 |
| negative regulation of cell cycle phase transition (GO:1901988) | 0.807354922 |
| negative regulation of translation (GO:0017148) | 0.807354922 |
| peptide metabolic process (GO:0006518) | 0.807354922 |
| phosphoprotein binding (GO:0051219) | 0.807354922 |
| positive regulation of cellular catabolic process (GO:0031331) | 0.807354922 |
| positive regulation of proteolysis involved in cellular protein | 0.807354922 |
| catabolic process (GO:1903052) | |
| protein localization to organelle (GO:0033365) | 0.807354922 |
| protein serine/threonine phosphatase activity (GO:0004722) | 0.807354922 |
| regulation of catabolic process (GO:0009894) | 0.807354922 |
| regulation of intracellular transport (GO:0032386) | 0.807354922 |
| regulation of protein ubiguitination (GO:0031396) | 0.807354922 |
| RNA metabolic process (GO:0016070) | 0.807354922 |
| structural constituent of ribosome (GO:0003735) | 0 807354922 |
| antigen processing and presentation of exogenous peptide | 0.815575429 |
| antigen via MHC class II (GO:0019886) | |
| DNA damage checkpoint (GO:0000077) | 0.815575429 |
| DNA integrity checkpoint (GO:0031570) | 0.815575429 |
| double-strand break repair via homologous recombination | 0.815575429 |
| (GO:0000724) | |
| energy derivation by oxidation of organic compounds | 0.815575429 |
| (GO:0015980) | |
| heat shock protein binding (GO:0031072) | 0.815575429 |
| intracellular transport (GO:0046907) | 0.815575429 |
| N-methyltransferase activity (GO:0008170) | 0.815575429 |
| negative regulation of cellular amide metabolic process | 0.815575429 |
| (GO:0034249) | |
| negative regulation of mitotic cell cycle phase transition | 0.815575429 |
| (GO:1901991) | |
| nucleoplasm (GO:0005654) | 0.815575429 |
| p75 NTR receptor-mediated signalling (R-HSA-193704) | 0.815575429 |
| positive regulation of protein localization to nucleus | 0.815575429 |
| (GO:1900182) | |
| regulation of cellular catabolic process (GO:0031329) | 0.815575429 |
| regulation of chromosome segregation (GO:0051983) | 0.815575429 |
| regulation of gene silencing (GO:0060968) | 0.815575429 |
| response to unfolded protein (GO:0006986) | 0.815575429 |
| RNA methylation (GO:0001510) | 0.815575429 |
| ruffle membrane (GO:0032587) | 0.815575429 |
| activation of innate immune response (GO:0002218) | 0.82374936 |
| antigen processing and presentation of peptide antigen via MHC | 0.82374936 |
| class II (GO:0002495) | |
| cadherin binding (GO:0045296) | 0.82374936 |
| cell cycle checkpoint (GO:0000075) | 0.82374936 |
| cellular response to unfolded protein (GO:0034620) | 0.82374936 |
| cytoplasmic stress granule (GO:0010494) | 0.82374936 |
| DNA Double-Strand Break Repair (R-HSA-5693532) | 0.82374936 |
| endoplasmic reticulum-Golgi intermediate compartment | 0.82374936 |
| (GO:0005793) | |
| intracellular protein transport (GO:0006886) | 0.82374936 |
| mitochondrial inner membrane (GO:0005743) | 0.82374936 |
| mitochondrion organization (GO:0007005) | 0.82374936 |
| myeloid leukocyte activation (GO:0002274) | 0.82374936 |
| negative regulation of cell cycle process (GO:0010948) | 0.82374936 |
| nuclear membrane (GO:0031965) | 0.82374936 |
| phagocytic vesicle membrane (GO:0030670) | 0.82374936 |
| positive regulation of cellular protein catabolic process | 0.82374936 |
| (GO:1903364) | |
| protein C-terminus binding (GO:0008022) | 0.82374936 |
| regulation of protein stability (GO:0031647) | 0.82374936 |
| signal transduction in response to DNA damage (GO:0042770) | 0.82374936 |
| trans-Golgi network (GO:0005802) | 0.82374936 |
| transcriptional repressor complex (GO:0017053) | 0.82374936 |
| cellular response to UV (GO:0034644) | 0.831877241 |
| Deubiquitination (R-HSA-5688426) | 0.831877241 |
| gene expression (GO:0010467) | 0.831877241 |
| interspecies interaction between organisms (GO:0044419) | 0.831877241 |
| late endosome (GO:0005770) | 0.831877241 |
| lipid phosphorylation (GO:0046834) | 0.831877241 |
| microtubule cytoskeleton organization involved in mitosis | 0.831877241 |
| (GO:1902850) | |
| mitochondrial respirasome (GO:0005746) | 0.831877241 |
| N-acyltransferase activity (GO:0016410) | 0.831877241 |
| negative regulation of protein catabolic process (GO:0042177) | 0.831877241 |
| nucleotidyltransferase activity (GO:0016779) | 0.831877241 |
| organelle transport along microtubule (GO:0072384) | 0.831877241 |
| P-body (GQ:0000932) | 0.831877241 |
| positive regulation of DNA metabolic process (GO:0051054) | 0.831877241 |
| positive regulation of histone modification (GO:0031058) | 0.831877241 |
| positive regulation of macroautophagy (GO:0016239) | 0.831877241 |
| positive regulation of mitochondrion organization (GO:0010822) | 0.831877241 |
| positive regulation of protein modification by small protein | 0.831877241 |
| conjugation or removal (GO:1903322) | |
| positive regulation of translation (GO:0045727) | 0.831877241 |
| protein catabolic process (GO:0030163) | 0.831877241 |
| regulation of cell cycle phase transition (GO:1901987) | 0.831877241 |
| regulation of cellular amine metabolic process (GO:0033238) | 0.831877241 |
| regulation of proteolysis involved in cellular protein catabolic | 0.831877241 |
| process (GO:1903050) | |
| Signaling by NTRKs (R-HSA-166520) | 0.831877241 |
| spindle (GO:0005819) | 0.831877241 |
| spindle organization (GO:0007051) | 0.831877241 |
| trans-Golgi network membrane (GO:0032588) | 0.831877241 |
| Transcriptional regulation of white adipocyte differentiation (R- | 0.831877241 |
| HSA-381340) | |
| Cargo recognition for clathrin-mediated endocytosis (R-HSA- | 0.839959587 |
| 8856825) | |
| Class I MHC mediated antigen processing & presentation (R- | 0.839959587 |
| HSA-983169) | |
| cytokinesis (GO:0000910) | 0.839959587 |
| fatty acid oxidation (GO:0019395) | 0.839959587 |
| glycerophospholipid biosynthetic process (GO:0046474) | 0.839959587 |
| integral component of endoplasmic reticulum membrane | 0.839959587 |
| (GO:0030176) | |
| mitotic spindle organization (GO:0007052) | 0.839959587 |
| nuclear receptor transcription coactivator activity (GO:0030374) | 0.839959587 |
| positive regulation of cellular amide metabolic process | 0.839959587 |
| (GO:0034250) | |
| positive regulation of gene expression, epigenetic (GO:0045815) | 0.839959587 |
| positive regulation of mRNA metabolic process (GO:1903313) | 0.839959587 |
| positive regulation of ubiquitin-dependent protein catabolic | 0.839959587 |
| process (GO:2000060) | |
| protein N-linked glycosylation (GO:0006487) | 0.839959587 |
| protein targeting (GO:0006605) | 0.839959587 |
| regulation of mitochondrion organization (GO:0010821) | 0.839959587 |
| response to topologically incorrect protein (GO:0035966) | 0.839959587 |
| single-stranded RNA binding (GO:0003727) | 0.839959587 |
| Transport to the Golgi and subsequent modification (R-HSA- | 0.839959587 |
| 948021) | |
| vesicle-mediated transport to the plasma membrane | 0.839959587 |
| (GO:0098876) | |
| cellular response to DNA damage stimulus (GO:0006974) | 0.847996907 |
| Cellular responses to stress (R-HSA-2262752) | 0.847996907 |
| centrosome cycle (GO:0007098) | 0.847996907 |
| clarthin-coated pit (GO:0005905) | 0.847996907 |
| Clathrin-mediated endocytosis (R-HSA-8856828) | 0.847996907 |
| Costimulation by the CD28 family (R-HSA-388841) | 0.847996907 |
| Diseases of signal transduction (R-HSA-5663202) | 0.847996907 |
| establishment of protein localization to organelle (GO:0072594) | 0.847996907 |
| Golgi-to-ER retrograde transport (R-HSA-8856688) | 0.847996907 |
| histone lysine methylation (GO:0034968) | 0.847996907 |
| Influenza Infection (R-HSA-168254) | 0.847996907 |
| Influenza Viral RNA Transcription and Replication (R-HSA- | 0.847996907 |
| 168273) | |
| lipid oxidation (GO:0034440) | 0.847996907 |
| microtubule organizing center organization (GO:0031023) | 0.847996907 |
| organelle fusion (GO:0048284) | 0.847996907 |
| organelle membrane fusion (GO:0090174) | 0.847996907 |
| oxidative phosphorylation (GO:0006119) | 0.847996907 |
| positive regulation of chromatin organization (GO:1905269) | 0.847996907 |
| regulation of cellular protein catabolic process (GO:1903362) | 0.847996907 |
| regulation of histone modification (GO:0031056) | 0.847996907 |
| TNFR2 non-canonical NF-kB pathway (R-HSA-5668541) | 0.847996907 |
| ubiguitin-like protein ligase binding (GO:0044389) | 0.847996907 |
| viral life cycle (GO:0019058) | 0.847996907 |
| amide biosynthetic process (GO:0043604) | 0.855989697 |
| Asparagine N-linked glycosylation (R-HSA-446203) | 0.855989697 |
| Cellular responses to external stimuli (R-HSA-8953897) | 0.855989697 |
| mitotic cell cycle (GO:0000278) | 0.855989697 |
| mitotic spindle (GO:0072686) | 0.855989697 |
| modification-dependent protein catabolic process (GO:0019941) | 0.855989697 |
| nuclear receptor binding (GO:0016922) | 0.855989697 |
| peptidyl-threonine phosphorylation (GO:0018107) | 0.855989697 |
| phospholipid biosynthetic process (GO:0008654) | 0.855989697 |
| regulation of chromosome organization (GO:0033044) | 0.855989697 |
| regulation of gene expression, epigenetic (GO:0040029) | 0.855989697 |
| regulation of mRNA catabolic process (GO:0061013) | 0.855989697 |
| regulation of protein modification by small protein conjugation or | 0.855989697 |
| removal (GO:1903320) | |
| regulation of RNA splicing (GO:0043484) | 0.855989697 |
| regulation of signal transduction by p53 class mediator | 0.855989697 |
| (GO:1901796) | |
| RNA methyltransferase activity (GO:0008173) | 0.855989697 |
| S-adenosylmethionine-dependent methyltransferase activity | 0.855989697 |
| (GO:0008757) | |
| spindle pole (GO:0000922) | 0.855989697 |
| The citric acid (TCA) cycle and respiratory electron transport (R- | 0.855989697 |
| HSA-1428517) | |
| tRNA modification (GO:0006400) | 0.855989697 |
| ubiquitin protein ligase binding (GO:0031625) | 0.855989697 |
| ubiquitin-dependent protein catabolic process (GO:0006511) | 0.855989697 |
| cellular macromolecule catabolic process (GO:0044265) | 0.86393845 |
| cellular response to topologically incorrect protein (GO:0035967) | 0.86393845 |
| Death Receptor Signalling (R-HSA-73887) | 0.86393845 |
| Epigenetic regulation of gene expression (R-HSA-212165) | 0.86393845 |
| Influenza Life Cycle (R-HSA-168255) | 0.86393845 |
| microbody membrane (GO:0031903) | 0.86393845 |
| midbody (GO:0030496) | 0.86393845 |
| mitochondrial matrix (GO:0005759) | 0.86393845 |
| mitotic nuclear division (GO:0140014) | 0.86393845 |
| modification-dependent macromolecule catabolic process | 0.86393845 |
| (GO:0043632) | |
| negative regulation of autophagy (GO:0010507) | 0.86393845 |
| negative regulation of protein ubiquitination (GO:0031397) | 0.86393845 |
| nuclear-transcribed mRNA catabolic process (GO:0000956) | 0.86393845 |
| nucleus organization (GO:0006997) | 0.86393845 |
| organelle localization by membrane tethering (GO:0140056) | 0.86393845 |
| PCP/CE pathway (R-HSA-4086400) | 0.86393845 |
| peroxisomal membrane (GO:0005778) | 0.86393845 |
| positive regulation of intracellular transport (GO:0032388) | 0.86393845 |
| positive regulation of proteasomal protein catabolic process | 0.86393845 |
| (GO:1901800) | |
| protein localization to chromosome (GO:0034502) | 0.86393845 |
| protein methyltransferase activity (GO:0008276) | 0.86393845 |
| protein polyubiquitination (GO:0000209) | 0.86393845 |
| proteolysis involved in cellular protein catabolic process | 0.86393845 |
| (GO:0051603) | |
| regulation of intracellular protein transport (GO:0033157) | 0.86393845 |
| regulation of sister chromatid segregation (GO:0033045) | 0.86393845 |
| Respiratory electron transport, ATP synthesis by chemiosmotic | 0.86393845 |
| coupling, and heat production by uncoupling proteins. (R-HSA- | |
| 163200) | |
| response to UV (GO:0009411) | 0.86393845 |
| specific granule (GO:0042581) | 0.86393845 |
| tumor necrosis factor-mediated signaling pathway (GO:0033209) | 0.86393845 |
| vesicle localization (GO:0051648) | 0.86393845 |
| cellular protein catabolic process (GO:0044257) | 0.871843649 |
| cellular response to hydrogen peroxide (GO:0070301) | 0.871843649 |
| endoplasmic reticulum to Golgi vesicle-mediated transport | 0.871843649 |
| (GO:0006888) | |
| histone binding (GO:0042393) | 0.871843649 |
| Intracellular signaling by second messengers (R-HSA-9006925) | 0.871843649 |
| mitotic cell cycle process (GO:1903047) | 0.871843649 |
| myeloid cell activation involved in immune response | 0.871843649 |
| (GO:0002275) | |
| myeloid cell homeostasis (GO:0002262) | 0.871843649 |
| nuclear body (GO:0016604) | 0.871843649 |
| p53 binding (GO:0002039) | 0.871843649 |
| positive regulation of autophagy (GO:0010508) | 0.871843649 |
| protein import into nucleus (GO:0006606) | 0.871843649 |
| regulation of autophagy (GO:0010506) | 0.871843649 |
| regulation of DNA biosynthetic process (GO:2000278) | 0.871843649 |
| regulation of mitotic cell cycle phase transition (GO:1901990) | 0.871843649 |
| regulation of TOR signaling (GO:0032006) | 0.871843649 |
| Translocation of SLC2A4 (GLUT4) to the plasma membrane (R- | 0.871843649 |
| HSA-1445148) | |
| vacuolar membrane (GO:0005774) | 0.871843649 |
| azurophil granule lumen (GO:0035578) | 0.879705766 |
| chaperone-mediated protein folding (GO:0061077) | 0.879705766 |
| DNA repair (GO:0006281) | 0.879705766 |
| Formation of the ternary complex, and subsequently, the 43S | 0.879705766 |
| complex (R-HSA-72695) | |
| granulocyte activation (GO:0036230) | 0.879705766 |
| heterochromatin (GO:0000792) | 0.879705766 |
| Interleukin-12 signaling (R-HSA-9020591) | 0.879705766 |
| Interleukin-3, Interleukin-5 and GM-CSF signaling (R-HSA- | 0.879705766 |
| 512988) | |
| large ribosomal subunit (GO:0015934) | 0.879705766 |
| leukocyte degranulation (GO:0043299) | 0.879705766 |
| membrane docking (GO:0022406) | 0.879705766 |
| mRNA catabolic process (GO:0006402) | 0.879705766 |
| myeloid leukocyte mediated immunity (GO:0002444) | 0.879705766 |
| nucleolus (GO:0005730) | 0.879705766 |
| PIP3 activates AKT signaling (R-HSA-1257604) | 0.879705766 |
| positive regulation of intracellular protein transport (GO:0090316) | 0.879705766 |
| positive regulation of proteasomal ubiquitin-dependent protein | 0.879705766 |
| catabolic process (GO:0032436) | |
| protein acylation (GO:0043543) | 0.879705766 |
| protein kinase complex (GO:1902911) | 0.879705766 |
| Pyruvate metabolism and Citric Acid (TCA) cycle (R-HSA-71406) | 0.879705766 |
| regulation of cellular amide metabolic process (GO:0034248) | 0.879705766 |
| regulation of glycolytic process (GO:0006110) | 0.879705766 |
| regulation of proteasomal protein catabolic process | 0.879705766 |
| (GO:0061136) | |
| regulation of RNA stability (GO:0043487) | 0.879705766 |
| RNA catabolic process (GO:0006401) | 0.879705766 |
| steroid hormone receptor binding (GO:0035258) | 0.879705766 |
| symbiotic process (GO:0044403) | 0.879705766 |
| cell redox homeostasis (GO:0045454) | 0.887525271 |
| chromosome, centromeric region (GO:0000775) | 0.887525271 |
| cytoplasmic translation (GO:0002181) | 0.887525271 |
| double-strand break repair (GO:0006302) | 0.887525271 |
| endoplasmic reticulum organization (GO:0007029) | 0.887525271 |
| erythrocyte differentiation (GO:0030218) | 0.887525271 |
| Membrane Trafficking (R-HSA-199991) | 0.887525271 |
| Metabolism of polyamines (R-HSA-351202) | 0.887525271 |
| negative regulation of protein modification by small protein | 0.887525271 |
| conjugation or removal (GO:1903321) | |
| negative regulation of proteolysis involved in cellular protein | 0.887525271 |
| catabolic process (GO:1903051) | |
| neutrophil activation (GO:0042119) | 0.887525271 |
| neutrophil activation involved in immune response (GO:0002283) | 0.887525271 |
| neutrophil degranulation (GO:0043312) | 0.887525271 |
| Neutrophil degranulation (R-HSA-6798695) | 0.887525271 |
| neutrophil mediated immunity (GO:0002446) | 0.887525271 |
| protein localization to nucleus (GO:0034504) | 0.887525271 |
| regulation of ATP metabolic process (GO:1903578) | 0.887525271 |
| regulation of carbohydrate catabolic process (GO:0043470) | 0.887525271 |
| regulation of centrosome cycle (GO:0046605) | 0.887525271 |
| regulation of mRNA splicing, via spliceosome (GO:0048024) | 0.887525271 |
| regulation of mRNA stability (GO:0043488) | 0.887525271 |
| regulation of nucleocytoplasmic transport (GO:0046822) | 0.887525271 |
| Resolution of Sister Chromatid Cohesion (R-HSA-2500257) | 0.887525271 |
| ribosomal large subunit biogenesis (GO:0042273) | 0.887525271 |
| ribosomal subunit (GO:0044391) | 0.887525271 |
| chromosomal region (GO:0098687) | 0.895302621 |
| Circadian Clock (R-HSA-400253) | 0.895302621 |
| erythrocyte homeostasis (GO:0034101) | 0.895302621 |
| Golgi vesicle transport (GO:0048193) | 0.895302621 |
| Hedgehog ‘off’ state (R-HSA-5610787) | 0.895302621 |
| import into nucleus (GO:0051170) | 0.895302621 |
| integral component of mitochondrial inner membrane | 0.895302621 |
| (GO:0031305) | |
| intrinsic component of mitochondrial inner membrane | 0.895302621 |
| (GO:0031304) | |
| mitotic cytokinesis (GO:0000281) | 0.895302621 |
| nuclear hormone receptor binding (GO:0035257) | 0.895302621 |
| nucleobase-containing compound transport (GO:0015931) | 0.895302621 |
| organelle envelope lumen (GO:0031970) | 0.895302621 |
| protein localization to vacuole (GO:0072665) | 0.895302621 |
| regulation of antigen receptor-mediated signaling pathway | 0.895302621 |
| (GO:0050854) | |
| regulation of translation (GO:0006417) | 0.895302621 |
| regulation of ubiquitin-dependent protein catabolic process | 0.895302621 |
| (GO:2000058) | |
| RHO GTPases Activate Formins (R-HSA-5663220) | 0.895302621 |
| single-stranded DNA binding (GO:0003697) | 0.895302621 |
| small ribosomal subunit (GO:0015935) | 0.895302621 |
| viral transcription (GO:0019083) | 0.895302621 |
| aerobic respiration (GO:0009060) | 0.90303827 |
| antigen processing and presentation (GO:0019882) | 0.90303827 |
| catalytic activity, acting on atRNA (GO:0140101) | 0.90303827 |
| cell cycle G1/S phase transition (GO:0044843) | 0.90303827 |
| establishment of vesicle localization (GO:0051650) | 0.90303827 |
| G1/S transition of mitotic cell cycle (GO:0000082) | 0.90303827 |
| Golgi organization (GO:0007030) | 0.90303827 |
| kinetochore (GO:0000776) | 0.90303827 |
| mitochondrial intermembrane space (GO:0005758) | 0.90303827 |
| NAD binding (GO:0051287) | 0.90303827 |
| negative regulation of cellular protein catabolic process | 0.90303827 |
| (GO:1903363) | |
| negative regulation of proteasomal protein catabolic process | 0.90303827 |
| (GO:1901799) | |
| protein deubiquitination (GO:0016579) | 0.90303827 |
| protein K48-linked ubiquitination (GO:0070936) | 0.90303827 |
| regulation of mitotic sister chromatid segregation (GO:0033047) | 0.90303827 |
| regulation of mRNA metabolic process (GO:1903311) | 0.90303827 |
| ribosome (GO:0005840) | 0.90303827 |
| RNA binding (GO:0003723) | 0.90303827 |
| serine/threonine protein kinase complex (GO:1902554) | 0.90303827 |
| T cell receptor signaling pathway (GO:0050852) | 0.90303827 |
| Toll-Like Receptors Cascades (R-HSA-168898) | 0.90303827 |
| ABC-family proteins mediated transport (R-HSA-382556) | 0.910732662 |
| Apoptosis (R-HSA-109581) | 0.910732662 |
| catalytic activity, acting on RNA (GO:0140098) | 0.910732662 |
| endoplasmic reticulum unfolded protein response (GO:0030968) | 0.910732662 |
| endosome organization (GO:0007032) | 0.910732662 |
| ER to Golgi Anterograde Transport (R-HSA-199977) | 0.910732662 |
| G2/M Checkpoints (R-HSA-69481) | 0.910732662 |
| Homologous DNA Pairing and Strand Exchange (R-HSA- | 0.910732662 |
| 5693579) | |
| peptidyl-lysine modification (GO:0018205) | 0.910732662 |
| posttranscriptional regulation of gene expression (GO:0010608) | 0.910732662 |
| rRNA processing (R-HSA-72312) | 0.910732662 |
| Signaling by EGFR (R-HSA-177929) | 0.910732662 |
| Signaling byVEGF (R-HSA-194138) | 0.910732662 |
| tertiary granule membrane (GO:0070821) | 0.910732662 |
| Translation initiation complex formation (R-HSA-72649) | 0.910732662 |
| translational initiation (GO:0006413) | 0.910732662 |
| ubiquitin ligase complex (GO:0000151) | 0.910732662 |
| viral process (GO:0016032) | 0.910732662 |
| clathrin coat (GO:0030118) | 0.918386234 |
| Cyclin D associated events in G1 (R-HSA-69231) | 0.918386234 |
| G1 Phase (R-HSA-69236) | 0.918386234 |
| late endosome membrane (GO:0031902) | 0.918386234 |
| lysosomal membrane (GO:0005765) | 0.918386234 |
| lytic vacuole membrane (GO:0098852) | 0.918386234 |
| melanosome (GO:0042470) | 0.918386234 |
| mRNA metabolic process (GO:0016071) | 0.918386234 |
| nuclear pore (GO:0005643) | 0.918386234 |
| nucleoplasm part (GO:0044451) | 0.918386234 |
| phagocytic vesicle (GO:0045335) | 0.918386234 |
| pigment granule (GO:0048770) | 0.918386234 |
| positive regulation of nudeocytoplasmic transport (GO:0046824) | 0.918386234 |
| Programmed Cell Death (R-HSA-5357801) | 0.918386234 |
| regulation of histone methylation (GO:0031060) | 0.918386234 |
| response to endoplasmic reticulum stress (GO:0034976) | 0.918386234 |
| ribonucleoprotein complex (GO:1990904) | 0.918386234 |
| ruffle (GO:0001726) | 0.918386234 |
| viral gene expression (GO:0019080) | 0.918386234 |
| Activation of the mRNA upon binding of the cap-binding complex | 0.925999419 |
| and eIFs, and subsequent binding to 43S (R-HSA-72662) | |
| Cell Cycle (R-HSA-1640170) | 0.925999419 |
| cellular respiration (GO:0045333) | 0.925999419 |
| covalent chromatin modification (GO:0016569) | 0.925999419 |
| endosomal transport (GO:0016197) | 0.925999419 |
| peptide biosynthetic process (GO:0043043) | 0.925999419 |
| proteasomal protein catabolic process (GO:0010498) | 0.925999419 |
| proteasome-mediated ubiquitin-dependent protein catabolic | 0.925999419 |
| process (GO:0043161) | |
| protein acetylation (GO:0006473) | 0.925999419 |
| rRNA processing in the nucleus and cytosol (R-HSA-8868773) | 0.925999419 |
| Signaling by NOTCH1 (R-HSA-1980143) | 0.925999419 |
| translation regulator activity (GO:0045182) | 0.925999419 |
| tRNA processing (GO:0008033) | 0.925999419 |
| Asymmetric localization of PCP proteins (R-HSA-4608870) | 0.933572638 |
| autophagosome (GO:0005776) | 0.933572638 |
| azurophil granule (GO:0042582) | 0.933572638 |
| catalytic complex (GO:1902494) | 0.933572638 |
| Cell Cycle Checkpoints (R-HSA-69620) | 0.933572638 |
| chromosome, telomeric region (GO:0000781) | 0.933572638 |
| COPI-independent Golgi-to-ER retrograde traffic (R-HSA- | 0.933572638 |
| 6811436) | |
| COPI-mediated anterograde transport (R-HSA-6807878) | 0.933572638 |
| COPII-mediated vesicle transport (R-HSA-204005) | 0.933572638 |
| DNA-dependent ATPase activity (GO:0008094) | 0.933572638 |
| innate immune response activating cell surface receptor | 0.933572638 |
| signaling pathway (GO:0002220) | |
| Major pathway of rRNA processing in the nucleolus and cytosol | 0.933572638 |
| (R-HSA-6791226) | |
| myeloid cell development (GO:0061515) | 0.933572638 |
| ncRNA metabolic process (GO:0034660) | 0.933572638 |
| nuclear speck (GO:0016607) | 0.933572638 |
| oxidoreductase activity, acting on NAD(P)H, quinone or similar | 0.933572638 |
| compound as acceptor (GO:0016655) | |
| positive regulation of DNA biosynthetic process (GO:2000573) | 0.933572638 |
| positive regulation of intrinsic apoptotic signaling pathway | 0.933572638 |
| (GO:2001244) | |
| positive regulation of type I interferon production (GO:0032481) | 0.933572638 |
| primary lysosome (GO:0005766) | 0.933572638 |
| protein modification by small protein removal (GO:0070646) | 0.933572638 |
| regulation of cholesterol metabolic process (GO:0090181) | 0.933572638 |
| regulation of proteasomal ubiquitin-dependent protein catabolic | 0.933572638 |
| process (GO:0032434) | |
| Signaling by NTRK1 (TRKA) (R-HSA-187037) | 0.933572638 |
| SUMO E3 ligases SUMOylate target proteins (R-HSA-3108232) | 0.933572638 |
| SUMOylation (R-HSA-2990846) | 0.933572638 |
| TBC/RABGAPs (R-HSA-8854214) | 0.933572638 |
| telomere organization (GO:0032200) | 0.933572638 |
| vascular endothelial growth factor receptor signaling pathway | 0.933572638 |
| (GO:0048010) | |
| antigen processing and presentation of exogenous antigen | 0.941106311 |
| (GO:0019884) | |
| cell cycle phase transition (GO:0044770) | 0.941106311 |
| DNA helicase activity (GO:0003678) | 0.941106311 |
| histone methyltransferase activity (GO:0042054) | 0.941106311 |
| Intra-Golgi and retrograde Golgi-to-ER traffic (R-HSA-6811442) | 0.941106311 |
| iron-sulfur cluster binding (GO:0051536) | 0.941106311 |
| metal cluster binding (GO:0051540) | 0.941106311 |
| mitochondrial ATP synthesis coupled electron transport | 0.941106311 |
| (GO:0042775) | |
| mitochondrial protein complex (GO:0098798) | 0.941106311 |
| mitotic cell cycle phase transition (GO:0044772) | 0.941106311 |
| modification-dependent protein binding (GO:0140030) | 0.941106311 |
| nucleic acid transport (GO:0050657) | 0.941106311 |
| repressing transcription factor binding (GO:0070491) | 0.941106311 |
| respiratory electron transport chain (GO:0022904) | 0.941106311 |
| RNA transport (GO:0050658) | 0.941106311 |
| telomere maintenance (GO:0000723) | 0.941106311 |
| The role of GTSE1 in G2/M progression after G2 checkpoint (R- | 0.941106311 |
| HSA-8852276) | |
| ATP synthesis coupled electron transport (GO:0042773) | 0.948600847 |
| Chromatin modifying enzymes (R-HSA-3247509) | 0.948600847 |
| Chromatin organization (R-HSA-4839726) | 0.948600847 |
| establishment of RNA localization (GO:0051236) | 0.948600847 |
| fatty acid beta-oxidation (GO:0006635) | 0.948600847 |
| fibrillar center (GO:0001650) | 0.948600847 |
| G2/M transition of mitotic cell cycle (GO:0000086) | 0.948600847 |
| HDR through Homologous Recombination (HRR) (R-HSA- | 0.948600847 |
| 5685942) | |
| histone modification (GO:0016570) | 0.948600847 |
| mitochondrial respiratory chain complex assembly (GO:0033108) | 0.948600847 |
| mRNA processing (GO:0006397) | 0.948600847 |
| positive regulation of viral life cycle (GO:1903902) | 0.948600847 |
| protein import (GO:0017038) | 0.948600847 |
| regulation of telomerase activity (GO:0051972) | 0.948600847 |
| ribonucleoside monophosphate metabolic process (GO:0009161) | 0.948600847 |
| Ribosomal scanning and start codon recognition (R-HSA-72702) | 0.948600847 |
| vesicle budding from membrane (GO:0006900) | 0.948600847 |
| autophagosome assembly (GO:0000045) | 0.956056652 |
| cell cycle G2/M phase transition (GO:0044839) | 0.956056652 |
| damaged DNA binding (GO:0003684) | 0.956056652 |
| DNA Repair (R-HSA-73894) | 0.956056652 |
| DNA synthesis involved in DNA repair (GO:0000731) | 0.956056652 |
| Gene and protein expression by JAK-STAT signaling after | 0.956056652 |
| Interleukin-12 stimulation (R-HSA-8950505) | |
| M Phase (R-HSA-68886) | 0.956056652 |
| protein transmembrane transport (GO:0071806) | 0.956056652 |
| protein-containing complex disassembly (GO:0032984) | 0.956056652 |
| regulation of DNA replication (GO:0006275) | 0.956056652 |
| regulation of interferon-beta production (GO:0032648) | 0.956056652 |
| RNA localization (GO:0006403) | 0.956056652 |
| RNA processing (GO:0006396) | 0.956056652 |
| transferase complex (GO:1990234) | 0.956056652 |
| transferase complex, transferring phosphorus-containing groups | 0.956056652 |
| (GO:0061695) | |
| translation regulator activity, nucleic acid binding (GO:0090079) | 0.956056652 |
| vesicle tethering complex (GO:0099023) | 0.956056652 |
| acetyltransferase activity (GO:0016407) | 0.963474124 |
| antigen processing and presentation of exogenous peptide | 0.963474124 |
| antigen (GO:0002478) | |
| antigen processing and presentation of peptide antigen | 0.963474124 |
| (GO:0048002) | |
| Cell Cycle, Mitotic (R-HSA-69278) | 0.963474124 |
| ciliary basal body-plasma membrane docking (GO:0097711) | 0.963474124 |
| coated membrane (GO:0048475) | 0.963474124 |
| COPII-coated vesicle budding (GO:0090114) | 0.963474124 |
| cytosolic transport (GO:0016482) | 0.963474124 |
| DNA replication (GO:0006260) | 0.963474124 |
| integral component of mitochondrial membrane (GO:0032592) | 0.963474124 |
| Intrinsic Pathway for Apoptosis (R-HSA-109606) | 0.963474124 |
| membrane coat (GO:0030117) | 0.963474124 |
| mRNA transport (GO:0051028) | 0.963474124 |
| phosphatidylinositol biosynthetic process (GO:0006661) | 0.963474124 |
| Presynaptic phase of homologous DNA pairing and strand | 0.963474124 |
| exchange (R-HSA-5693616) | |
| protein sumoylation (GO:0016925) | 0.963474124 |
| regulation of cell cycle G2/M phase transition (GO:1902749) | 0.963474124 |
| regulation of macroautophagy (GO:0016241) | 0.963474124 |
| regulation of type I interferon production (GO:0032479) | 0.963474124 |
| regulation of viral transcription (GO:0046782) | 0.963474124 |
| Transcriptional Regulation by TP53 (R-HSA-3700989) | 0.963474124 |
| translation (GO:0006412) | 0.963474124 |
| Amplification of signal from unattached kinetochores via a MAD2 | 0.970853654 |
| inhibitory signal (R-HSA-141444) | |
| Amplification of signal from the kinetochores (R-HSA-141424) | 0.970853654 |
| Anchoring of the basal body to the plasma membrane (R-HSA- | 0.970853654 |
| 5620912) | |
| autophagosome organization (GO:1905037) | 0.970853654 |
| DNA geometric change (GO:0032392) | 0.970853654 |
| I-kappaB kinase/NF-kappaB signaling (GO:0007249) | 0.970853654 |
| internal protein amino acid acetylation (GO:0006475) | 0.970853654 |
| intrinsic component of mitochondrial membrane (GO:0098573) | 0.970853654 |
| nudeocytoplasmic transport (GO:0006913) | 0.970853654 |
| polysome (GO:0005844) | 0.970853654 |
| positive regulation of chromosome organization (GO:2001252) | 0.970853654 |
| posttranscriptional gene silencing by RNA (GO:0035194) | 0.970853654 |
| regulation of G2/M transition of mitotic cell cycle (GO:0010389) | 0.970853654 |
| stimulatory C-type lectin receptor signaling pathway | 0.970853654 |
| (GO:0002223) | |
| Toll Like Receptor 4 (TLR4)Cascade (R-HSA-166016) | 0.970853654 |
| transcription by RNA polymerase III (GO:0006383) | 0.970853654 |
| Transcriptional activity of SMAD2/SMAD3:SMAD4 heterotrimer | 0.970853654 |
| (R-HSA-2173793) | |
| ABC transporter disorders (R-HSA-5619084) | 0.97819563 |
| autophagy (GO:0006914) | 0.97819563 |
| Infectious disease (R-HSA-5663205) | 0.97819563 |
| intracellular protein transmembrane transport (GO:0065002) | 0.97819563 |
| nuclear chromosome, telomeric region (GO:0000784) | 0.97819563 |
| nuclear transport (GO:0051169) | 0.97819563 |
| nucleolar part (GO:0044452) | 0.97819563 |
| PI Metabolism (R-HSA-1483255) | 0.97819563 |
| postreplication repair (GO:0006301) | 0.97819563 |
| posttranscriptional gene silencing (GO:0016441) | 0.97819563 |
| process utilizing autophagic mechanism (GO:0061919) | 0.97819563 |
| Recruitment of NuMA to mitotic centrosomes (R-HSA-380320) | 0.97819563 |
| regulation of gene silencing by RNA (GO:0060966) | 0.97819563 |
| regulation of mRNA processing (GO:0050684) | 0.97819563 |
| regulation of posttranscriptional gene silencing (GO:0060147) | 0.97819563 |
| Regulation of RUNX2 expression and activity (R-HSA-8939902) | 0.97819563 |
| regulation of transcription from RNA polymerase II promoter in | 0.97819563 |
| response to stress (GO:0043618) | |
| retrograde vesicle-mediated transport, Golgi to endoplasmic | 0.97819563 |
| reticulum (GO:0006890) | |
| RNA 3′-end processing (GO:0031123) | 0.97819563 |
| Signaling by RAS mutants (R-HSA-6802949) | 0.97819563 |
| Transcriptional activation of mitochondrial biogenesis (R-HSA- | 0.97819563 |
| 2151201) | |
| tRNA metabolic process (GO:0006399) | 0.97819563 |
| cellular protein complex disassembly (GO:0043624) | 0.98550043 |
| COPII vesicle coating (GO:0048208) | 0.98550043 |
| core promoter binding (GO:0001047) | 0.98550043 |
| Interleukin-1 family signaling (R-HSA-446652) | 0.98550043 |
| Mitotic Prometaphase (R-HSA-68877) | 0.98550043 |
| N-acetyltransferase activity (GO:0008080) | 0.98550043 |
| ncRNA processing (GO:0034470) | 0.98550043 |
| nuclear envelope organization (GO:0006998) | 0.98550043 |
| phosphatase complex (GO:1903293) | 0.98550043 |
| protein serine/threonine phosphatase complex (GO:0008287) | 0.98550043 |
| regulation of DNA-templated transcription in response to stress | 0.98550043 |
| (GO:0043620) | |
| regulation of gene silencing by miRNA (GO:0060964) | 0.98550043 |
| replication fork (GO:0005657) | 0.98550043 |
| TRAF6 mediated induction of NFkB and MAP kinases upon | 0.98550043 |
| TLR7/8 or 9 activation (R-HSA-975138) | |
| vesicle targeting, rough ER to cis-Golgi (GO:0048207) | 0.98550043 |
| Activation of ATR in response to replication stress (R-HSA- | 0.992768431 |
| 176187) | |
| Cajal body (GO:0015030) | 0.992768431 |
| Cellular response to heat stress (R-HSA-3371556) | 0.992768431 |
| MyD88 dependent cascade initiated on endosome (R-HSA- | 0.992768431 |
| 975155) | |
| non-membrane spanning protein tyrosine kinase activity | 0.992768431 |
| (GO:0004715) | |
| organelle disassembly (GO:1903008) | 0.992768431 |
| regulation of translational initiation (GO:0006446) | 0.992768431 |
| ribosomal small subunit biogenesis (GO:0042274) | 0.992768431 |
| ribosome biogenesis (GO:0042254) | 0.992768431 |
| RNA helicase activity (GO:0003724) | 0.992768431 |
| SCF-dependent proteasomal ubiquitin-dependent protein | 0.992768431 |
| catabolic process (GO:0031146) | |
| TAK1 activates NFkB by phosphorylation and activation of IKKs | 0.992768431 |
| complex (R-HSA-445989) | |
| Toll Like Receptor 7/8 (TLR7/8)Cascade (R-HSA-168181) | 0.992768431 |
| TP53 Regulates Metabolic Genes (R-HSA-5628897) | 0.992768431 |
| tRNA processing (R-HSA-72306) | 0.992768431 |
| C-type lectin receptors (CLRs) (R-HSA-5621481) | 1 |
| ERAD pathway (GO:0036503) | 1 |
| helicase activity (GO:0004386) | 1 |
| Interleukin-17 signaling (R-HSA-448424) | 1 |
| internal peptidyl-lysine acetylation (GO:0018393) | 1 |
| MAPK6/MAPK4 signaling (R-HSA-5687128) | 1 |
| Mitotic Spindle Checkpoint (R-HSA-69618) | 1 |
| PKMTs methylate histone lysines (R-HSA-3214841) | 1 |
| ribonucleoprotein complex subunit organization (GO:0071826) | 1 |
| ubiquitin-dependent ERAD pathway (GO:0030433) | 1 |
| vesicle targeting, to, from or within Golgi (GO:0048199) | 1 |
| Constitutive Signaling by NOTCH1 HD + PEST Domain Mutants | 1.007195501 |
| (R-HSA-2894862) | |
| Constitutive Signaling by NOTCH1 PEST Domain Mutants (R- | 1.007195501 |
| HSA-2644606) | |
| DNA duplex unwinding (GO:0032508) | 1.007195501 |
| DNA-dependent DNA replication (GO:0006261) | 1.007195501 |
| Golgi vesicle budding (GO:0048194) | 1.007195501 |
| negative regulation of cell cycle G2/M phase transition | 1.007195501 |
| (GO:1902750) | |
| nuclear periphery (GO:0034399) | 1.007195501 |
| Oncogenic MAPK signaling (R-HSA-6802957) | 1.007195501 |
| PcG protein complex (GO:0031519) | 1.007195501 |
| peptidyl-lysine acetylation (GO:0018394) | 1.007195501 |
| regulation of G0 to G1 transition (GO:0070316) | 1.007195501 |
| Regulation of HSF1-mediated heat shock response (R-HSA- | 1.007195501 |
| 3371453) | |
| Regulation of TP53 Activity (R-HSA-5633007) | 1.007195501 |
| retrograde transport, endosome to Golgi (GO:0042147) | 1.007195501 |
| Signaling by NOTCH1 HD + PEST Domain Mutants in Cancer (R- | 1.007195501 |
| HSA-2894858) | |
| Signaling by NOTCH1 in Cancer (R-HSA-2644603) | 1.007195501 |
| Signaling by NOTCH1 PEST Domain Mutants in Cancer (R-HSA- | 1.007195501 |
| 2644602) | |
| specific granule membrane (GO:0035579) | 1.007195501 |
| Toll Like Receptor 9 (TLR9)Cascade (R-HSA-168138) | 1.007195501 |
| VEGFA-VEGFR2 Pathway (R-HSA-4420097) | 1.007195501 |
| vesicle coating (GO:0006901) | 1.007195501 |
| androgen receptor binding (GO:0050681) | 1.014355293 |
| azurophil granule membrane (GO:0035577) | 1.014355293 |
| core promoter sequence-specific DNA binding (GO:0001046) | 1.014355293 |
| Golgi to plasma membrane transport (GO:0006893) | 1.014355293 |
| histone acetylation (GO:0016573) | 1.014355293 |
| mitotic prometaphase (GO:0000236) | 1.014355293 |
| negative regulation of ubiquitin-dependent protein catabolic | 1.014355293 |
| process (GO:2000059) | |
| protein localization to mitochondrion (GO:0070585) | 1.014355293 |
| protein targeting to mitochondrion (GO:0006626) | 1.014355293 |
| Regulation of PTEN gene transcription (R-HSA-8943724) | 1.014355293 |
| ribonucleoprotein complex assembly (GO:0022618) | 1.014355293 |
| RNA splicing (GO:0008380) | 1.014355293 |
| Separation of Sister Chromatids (R-HSA-2467813) | 1.014355293 |
| vacuole organization (GO:0007033) | 1.014355293 |
| DNA-dependent DNA replication maintenance of fidelity | 1.021479727 |
| (GO:0045005) | |
| Glucose metabolism (R-HSA-70326) | 1.021479727 |
| Hedgehog ligand biogenesis (R-HSA-5358346) | 1.021479727 |
| lysosomal transport (GO:0007041) | 1.021479727 |
| MAP2K and MAPK activation (R-HSA-5674135) | 1.021479727 |
| Mitochondrial protein import (R-HSA-1268020) | 1.021479727 |
| negative regulation of G2/M transition of mitotic cell cycle | 1.021479727 |
| (GO:0010972) | |
| Negative regulation of MAPK pathway (R-HSA-5675221) | 1.021479727 |
| NOD1/2 Signaling Pathway (R-HSA-168638) | 1.021479727 |
| nuclear matrix (GO:0016363) | 1.021479727 |
| PML body (GO:0016605) | 1.021479727 |
| protein deacylation (GO:0035601) | 1.021479727 |
| regulation of DNA-dependent DNA replication (GO:0090329) | 1.021479727 |
| regulation of response to endoplasmic reticulum stress | 1.021479727 |
| (GO:1905897) | |
| tau protein binding (GO:0048156) | 1.021479727 |
| toxin transport (GO:1901998) | 1.021479727 |
| establishment of protein localization to mitochondrion | 1.028569152 |
| (GO:0072655) | |
| macromolecule deacylation (GO:0098732) | 1.028569152 |
| mitochondrial respiratory chain complex I (GO:0005747) | 1.028569152 |
| mitochondrial respiratory chain complex I assembly | 1.028569152 |
| (GO:0032981) | |
| Mitotic Anaphase (R-HSA-68882) | 1.028569152 |
| NADH dehydrogenase (quinone)activity (GO:0050136) | 1.028569152 |
| NADH dehydrogenase (ubiquinone)activity (GO:0008137) | 1.028569152 |
| NADH dehydrogenase activity (GO:0003954) | 1.028569152 |
| NADH dehydrogenase complex (GO:0030964) | 1.028569152 |
| NADH dehydrogenase complex assembly (GO:0010257) | 1.028569152 |
| post-Golgi vesicle-mediated transport (GO:0006892) | 1.028569152 |
| Regulation of TP53 Activity through Phosphorylation (R-HSA- | 1.028569152 |
| 6804756) | |
| respiratory chain complex I (GO:0045271) | 1.028569152 |
| spliceosomal complex assembly (GO:0000245) | 1.028569152 |
| Toll Like Receptor 2 (TLR2)Cascade (R-HSA-181438) | 1.028569152 |
| Toll Like Receptor TLR1:TLR2 Cascade (R-HSA-168179) | 1.028569152 |
| UCH proteinases (R-HSA-5689603) | 1.028569152 |
| vacuolar transport (GO:0007034) | 1.028569152 |
| CD28 co-stimulation (R-HSA-389356) | 1.03562391 |
| exonuclease activity (GO:0004527) | 1.03562391 |
| macroautophagy (GO:0016236) | 1.03562391 |
| Mitotic G2-G2/M phases (R-HSA-453274) | 1.03562391 |
| Mitotic Metaphase and Anaphase (R-HSA-2555396) | 1.03562391 |
| protein monoubiguitination (GO:0006513) | 1.03562391 |
| ribonucleoprotein complex biogenesis (GO:0022613) | 1.03562391 |
| Signaling by high-kinase activity BRAF mutants (R-HSA- | 1.03562391 |
| 6802948) | |
| Signaling by TGF-beta Receptor Complex (R-HSA-170834) | 1.03562391 |
| small nuclear ribonucleoprotein complex (GO:0030532) | 1.03562391 |
| tRNA binding (GO:0000049) | 1.03562391 |
| cellular response to glucose starvation (GO:0042149) | 1.042644337 |
| G2/M Transition (R-HSA-69275) | 1.042644337 |
| methyltransferase complex (GO:0034708) | 1.042644337 |
| MyD88 cascade initiated on plasma membrane (R-HSA-975871) | 1.042644337 |
| nuclear replication fork (GO:0043596) | 1.042644337 |
| Rab regulation of trafficking (R-HSA-9007101) | 1.042644337 |
| regulation of cellular amino acid metabolic process | 1.042644337 |
| (GO:0006521) | |
| regulation of cellular response to heat (GO:1900034) | 1.042644337 |
| Regulation of PLK1 Activity at G2/M Transition (R-HSA-2565942) | 1.042644337 |
| Regulation of TNFR1 signaling (R-HSA-5357905) | 1.042644337 |
| Respiratory electron transport (R-HSA-611105) | 1.042644337 |
| rRNA metabolic process (GO:0016072) | 1.042644337 |
| site of DNA damage (GO:0090734) | 1.042644337 |
| Termination of translesion DNA synthesis (R-HSA-5656169) | 1.042644337 |
| TNF signaling (R-HSA-75893) | 1.042644337 |
| Toll Like Receptor 10 (TLR10)Cascade (R-HSA-168142) | 1.042644337 |
| Toll Like Receptor 5 (TLR5)Cascade (R-HSA-168176) | 1.042644337 |
| Translation (R-HSA-72766) | 1.042644337 |
| vesicle coat (GO:0030120) | 1.042644337 |
| vesicle targeting (GO:0006903) | 1.042644337 |
| ficolin-1-rich granule (GO:0101002) | 1.049630768 |
| ficolin-1-rich granule lumen (GO:1904813) | 1.049630768 |
| Metabolism of RNA (R-HSA-8953854) | 1.049630768 |
| mRNA splicing, via spliceosome (GO:0000398) | 1.049630768 |
| nuclear-transcribed mRNA catabolic process, deadenylation- | 1.049630768 |
| dependent decay (GO:0000288) | |
| positive regulation of viral process (GO:0048524) | 1.049630768 |
| regulation of cholesterol biosynthetic process (GO:0045540) | 1.049630768 |
| regulation of sterol biosynthetic process (GO:0106118) | 1.049630768 |
| RNA splicing, via transesterification reactions (GO:0000375) | 1.049630768 |
| RNA splicing, via transesterification reactions with bulged | 1.049630768 |
| adenosine as nucleophile (GO:0000377) | |
| rRNA processing (GO:0006364) | 1.049630768 |
| site of double-strand break (GO:0035861) | 1.049630768 |
| Downstream TCR signaling (R-HSA-202424) | 1.056583528 |
| histone H4 acetylation (GO:0043967) | 1.056583528 |
| IRE1-mediated unfolded protein response (GO:0036498) | 1.056583528 |
| M phase (GO:0000279) | 1.056583528 |
| mitochondrial nucleoid (GO:0042645) | 1.056583528 |
| mitotic M phase (GO:0000087) | 1.056583528 |
| nucleoid (GO:0009295) | 1.056583528 |
| Oncogene Induced Senescence (R-HSA-2559585) | 1.056583528 |
| protein deacetylation (GO:0006476) | 1.056583528 |
| protein N-terminus binding (GO:0047485) | 1.056583528 |
| regulation of telomere maintenance via telomerase | 1.056583528 |
| (GO:0032210) | |
| Signaling by BRAF and RAF fusions (R-HSA-6802952) | 1.056583528 |
| Sm-like protein family complex (GO:0120114) | 1.056583528 |
| spliceosomal snRNP complex (GO:0097525) | 1.056583528 |
| 5′-3′ RNA polymerase activity (GO:0034062) | 1.063502942 |
| DNA-templated transcription, termination (GO:0006353) | 1.063502942 |
| Loss of Nip from mitotic centrosomes (R-HSA-380259) | 1.063502942 |
| Loss of proteins required for interphase microtubule organization | 1.063502942 |
| from the centrosome (R-HSA-380284) | |
| negative regulation of response to endoplasmic reticulum stress | 1.063502942 |
| (GO:1903573) | |
| Negative regulators of DDX58/IFIH1 signaling (R-HSA-936440) | 1.063502942 |
| NOTCH1 Intracellular Domain Regulates Transcription (R-HSA- | 1.063502942 |
| 2122947) | |
| RNA polymerase activity (GO:0097747) | 1.063502942 |
| biological phase (GO:0044848) | 1.070389328 |
| cell cycle phase (GO:0022403) | 1.070389328 |
| Cellular response to hypoxia (R-HSA-2262749) | 1.070389328 |
| H4 histone acetyltransferase complex (GO:1902562) | 1.070389328 |
| maturation of SSU-rRNA (GO:0030490) | 1.070389328 |
| mitotic cell cycle phase (GO:0098763) | 1.070389328 |
| Paradoxical activation of RAF signaling by kinase inactive BRAF | 1.070389328 |
| (R-HSA-6802955) | |
| preribosome (GO:0030684) | 1.070389328 |
| regulation of hematopoietic progenitor cell differentiation | 1.070389328 |
| (GO:1901532) | |
| Regulation of Hypoxia-inducible Factor (HIF)by oxygen (R-HSA- | 1.070389328 |
| 1234174) | |
| regulation of telomere maintenance via telomere lengthening | 1.070389328 |
| (GO:1904356) | |
| response to amino acid starvation (GO:1990928) | 1.070389328 |
| Signaling by moderate kinase activity BRAF mutants (R-HSA- | 1.070389328 |
| 6802946) | |
| SUMOylation of chromatin organization proteins (R-HSA- | 1.070389328 |
| 4551638) | |
| SWI/SNF superfamily-type complex (GO:0070603) | 1.070389328 |
| transcription elongation factor complex (GO:0008023) | 1.070389328 |
| ubiquitin-like protein binding (GO:0032182) | 1.070389328 |
| Antigen processing-Cross presentation (R-HSA-1236975) | 1.077242999 |
| Antiviral mechanism by IFN-stimulated genes (R-HSA-1169410) | 1.077242999 |
| AURKA Activation by TPX2 (R-HSA-8854518) | 1.077242999 |
| gene silencing by miRNA (GO:0035195) | 1.077242999 |
| histone methyltransferase complex (GO:0035097) | 1.077242999 |
| ISG15 antiviral mechanism (R-HSA-1169408) | 1.077242999 |
| lysosome organization (GO:0007040) | 1.077242999 |
| Lysosome Vesicle Biogenesis (R-HSA-432720) | 1.077242999 |
| lytic vacuole organization (GO:0080171) | 1.077242999 |
| Mitotic G1-G1/S phases (R-HSA-453279) | 1.077242999 |
| MyD88:Mal cascade initiated on plasma membrane (R-HSA- | 1.077242999 |
| 166058) | |
| nuclear export (GO:0051168) | 1.077242999 |
| RNA polymerase complex (GO:0030880) | 1.077242999 |
| RNA Polymerase III Abortive And Retractive Initiation (R-HSA- | 1.077242999 |
| 749476) | |
| RNA Polymerase III Transcription (R-HSA-74158) | 1.077242999 |
| Synthesis of PIPs at the plasma membrane (R-HSA-1660499) | 1.077242999 |
| Toll Like Receptor TLR6:TLR2 Cascade (R-HSA-168188) | 1.077242999 |
| Unfolded Protein Response (UPR)(R-HSA-381119) | 1.077242999 |
| anaphase (GO:0051322) | 1.084064265 |
| ATPase complex (GO:1904949) | 1.084064265 |
| AUF1 (hnRNP D0)binds and destabilizes mRNA (R-HSA- | 1.084064265 |
| 450408) | |
| G1/S DNA Damage Checkpoints (R-HSA-69615) | 1.084064265 |
| Golgi Associated Vesicle Biogenesis (R-HSA-432722) | 1.084064265 |
| histone deacetylation (GO:0016575) | 1.084064265 |
| host cell (GO:0043657) | 1.084064265 |
| host cellular component (GO:0018995) | 1.084064265 |
| mitotic anaphase (GO:0000090) | 1.084064265 |
| MyD88-independent TLR4 cascade (R-HSA-166166) | 1.084064265 |
| nuclear transcriptional repressor complex (GO:0090568) | 1.084064265 |
| Nucleotide-binding domain, leucine rich repeat containing | 1.084064265 |
| receptor (NLR)signaling pathways (R-HSA-168643) | |
| protein export from nucleus (GO:0006611) | 1.084064265 |
| regulation of autophagosome assembly (GO:2000785) | 1.084064265 |
| Regulation of TP53 Expression and Degradation (R-HSA- | 1.084064265 |
| 6806003) | |
| rRNA modification in the nucleus and cytosol (R-HSA-6790901) | 1.084064265 |
| Synthesis of active ubiquitin: roles of E1 and E2 enzymes (R- | 1.084064265 |
| HSA-8866652) | |
| TCR signaling (R-HSA-202403) | 1.084064265 |
| TNFR1-induced NFkappaB signaling pathway (R-HSA-5357956) | 1.084064265 |
| Toll Like Receptor 3 (TLR3)Cascade (R-HSA-168164) | 1.084064265 |
| TRIF(TICAM1)-mediated TLR4 signaling (R-HSA-937061) | 1.084064265 |
| ubiquitin binding (GO:0043130) | 1.084064265 |
| 90S preribosome (GO:0030686) | 1.09085343 |
| cellular response to amino acid starvation (GO:0034198) | 1.09085343 |
| Centrosome maturation (R-HSA-380287) | 1.09085343 |
| Complex I biogenesis (R-HSA-6799198) | 1.09085343 |
| double-strand break repair via nonhomologous end joining | 1.09085343 |
| (GO:0006303) | |
| Endosomal Sorting Complex Required For Transport | 1.09085343 |
| (ESCRT)(R-HSA-917729) | |
| G1/S Transition (R-HSA-69206) | 1.09085343 |
| general transcription initiation factor binding (GO:0140296) | 1.09085343 |
| histone acetyltransferase complex (GO:0000123) | 1.09085343 |
| Intra-Golgi traffic (R-HSA-6811438) | 1.09085343 |
| mitochondrial electron transport, NADH to ubiquinone | 1.09085343 |
| (GO:0006120) | |
| non-recombinational repair (GO:0000726) | 1.09085343 |
| protein targeting to vacuole (GO:0006623) | 1.09085343 |
| Recruitment of mitotic centrosome proteins and complexes (R- | 1.09085343 |
| HSA-380270) | |
| Regulation of RAS by GAPs (R-HSA-5658442) | 1.09085343 |
| regulation of vacuole organization (GO:0044088) | 1.09085343 |
| Activation of APC/C and APC/C:Cdc20 mediated degradation of | 1.097610797 |
| mitotic proteins (R-HSA-176814) | |
| Association of TriC/CCT with target proteins during biosynthesis | 1.097610797 |
| (R-HSA-390471) | |
| Cytosolic sensors of pathogen-associated DNA (R-HSA- | 1.097610797 |
| 1834949) | |
| DNA Replication (R-HSA-69306) | 1.097610797 |
| DNA strand elongation (R-HSA-69190) | 1.097610797 |
| negative regulation of telomere maintenance (GO:0032205) | 1.097610797 |
| peptidase complex (GO:1905368) | 1.097610797 |
| phagophore assembly site (GO:0000407) | 1.097610797 |
| RAB GEFs exchange GTP for GDP on RABs (R-HSA-8876198) | 1.097610797 |
| ribonucleoprotein complex export from nucleus (GO:0071426) | 1.097610797 |
| ribonucleoprotein complex localization (GO:0071166) | 1.097610797 |
| RNA polymerase II, holoenzyme (GO:0016591) | 1.097610797 |
| spliceosomal complex (GO:0005681) | 1.097610797 |
| spliceosomal tri-snRNP complex (GO:0097526) | 1.097610797 |
| Transcriptional regulation by RUNX3 (R-HSA-8878159) | 1.097610797 |
| translation factor activity, RNA binding (GO:0008135) | 1.097610797 |
| U4/U6 x U5 tri-snRNP complex (GO:0046540) | 1.097610797 |
| acetyltransferase complex (GO:1902493) | 1.10433666 |
| antigen processing and presentation of peptide antigen via MHC | 1.10433666 |
| class I (GO:0002474) | |
| AP-type membrane coat adaptor complex (GO:0030119) | 1.10433666 |
| Calnexin/calreticulin cycle (R-HSA-901042) | 1.10433666 |
| Clathrin derived vesicle budding (R-HSA-421837) | 1.10433666 |
| DNA damage response, detection of DNA damage | 1.10433666 |
| (GO:0042769) | |
| endosome to lysosome transport (GO:0008333) | 1.10433666 |
| ER-Phagosome pathway (R-HSA-1236974) | 1.10433666 |
| Hh mutants abrogate ligand secretion (R-HSA-5387390) | 1.10433666 |
| histone deacetylase complex (GO:0000118) | 1.10433666 |
| negative regulation of GO to G1 transition (GO:0070317) | 1.10433666 |
| negative regulation of type I interferon production (GO:0032480) | 1.10433666 |
| p53-Dependent G1 DNA Damage Response (R-HSA-69563) | 1.10433666 |
| p53-Dependent G1/S DNA damage checkpoint (R-HSA-69580) | 1.10433666 |
| polyubiquitin modification-dependent protein binding | 1.10433666 |
| (GO:0031593) | |
| protein acetyltransferase complex (GO:0031248) | 1.10433666 |
| PTEN Regulation (R-HSA-6807070) | 1.10433666 |
| regulation of transcription from RNA polymerase II promoter in | 1.10433666 |
| response to hypoxia (GO:0061418) | |
| RNA export from nucleus (GO:0006405) | 1.10433666 |
| TP53 Regulates Transcription of DNA Repair Genes (R-HSA- | 1.10433666 |
| 6796648) | |
| trans-Golgi Network Vesicle Budding (R-HSA-199992) | 1.10433666 |
| transcription factor TFIID complex (GO:0005669) | 1.10433666 |
| Translesion synthesis by Y family DNA polymerases bypasses | 1.10433666 |
| lesions on DNA template (R-HSA-110313) | |
| Activation of gene expression by SREBF (SREBP)(R-HSA- | 1.111031312 |
| 2426168) | |
| Activation of the pre-replicative complex (R-HSA-68962) | 1.111031312 |
| antigen processing and presentation of exogenous peptide | 1.111031312 |
| antigen via MHC class I (GO:0042590) | |
| APC/C-mediated degradation of cell cycle proteins (R-HSA- | 1.111031312 |
| 174143) | |
| COPI-coated vesicle (GO:0030137) | 1.111031312 |
| Glycolysis (R-HSA-70171) | 1.111031312 |
| histone H3 acetylation (GO:0043966) | 1.111031312 |
| maintenance of protein localization in organelle (GO:0072595) | 1.111031312 |
| Mitophagy (R-HSA-5205647) | 1.111031312 |
| negative regulation of DNA replication (GO:0008156) | 1.111031312 |
| NIK/NF-kappaB signaling (GO:0038061) | 1.111031312 |
| nuclear DNA-directed RNA polymerase complex (GO:0055029) | 1.111031312 |
| Oxygen-dependent proline hydroxylation of Hypoxia-inducible | 1.111031312 |
| Factor Alpha (R-HSA-1234176) | |
| Regulation of cholesterol biosynthesis by SREBP (SREBF)(R- | 1.111031312 |
| HSA-1655829) | |
| regulation of hematopoietic stem cell differentiation | 1.111031312 |
| (GO:1902036) | |
| Regulation of mitotic cell cycle (R-HSA-453276) | 1.111031312 |
| RNA Polymerase III Transcription Initiation From Type 2 | 1.111031312 |
| Promoter (R-HSA-76066) | |
| S Phase (R-HSA-69242) | 1.111031312 |
| SCF(Skp2)-mediated degradation of p27/p21 (R-HSA-187577) | 1.111031312 |
| Signaling by NOTCH4 (R-HSA-9013694) | 1.111031312 |
| XBP1(S)activates chaperone genes (R-HSA-381038) | 1.111031312 |
| antigen processing and presentation of exogenous peptide | 1.117695043 |
| antigen via MHC class I, TAP-dependent (GO:0002479) | |
| APC/C:Cdc20 mediated degradation of mitotic proteins (R-HSA- | 1.117695043 |
| 176409) | |
| Cul4-RING E3 ubiquitin ligase complex (GO:0080008) | 1.117695043 |
| Cyclin E associated events during G1/S transition (R-HSA- | 1.117695043 |
| 69202) | |
| DNA-directed RNA polymerase complex (GO:0000428) | 1.117695043 |
| double-stranded RNA binding (GO:0003725) | 1.117695043 |
| Macroautophagy (R-HSA-1632852) | 1.117695043 |
| Pausing and recovery of Tat-mediated HIV elongation (R-HSA- | 1.117695043 |
| 167238) | |
| positive regulation of telomere maintenance via telomerase | 1.117695043 |
| (GO:0032212) | |
| regulation of telomere maintenance (GO:0032204) | 1.117695043 |
| regulation of type I interferon-mediated signaling pathway | 1.117695043 |
| (GO:0060338) | |
| Switching of origins to a post-replicative state (R-HSA-69052) | 1.117695043 |
| Tat-mediated HIV elongation arrest and recovery (R-HSA- | 1.117695043 |
| 167243) | |
| 4 iron, 4 sulfur cluster binding (GO:0051539) | 1.124328135 |
| aminoacyl-tRNA ligase activity (GO:0004812) | 1.124328135 |
| Cyclin A:Cdk2-associated events at S phase entry (R-HSA- | 1.124328135 |
| 69656) | |
| Degradation of DVL (R-HSA-4641258) | 1.124328135 |
| DNA Damage Bypass (R-HSA-73893) | 1.124328135 |
| DNA-directed 5′-3′ RNA polymerase activity (GO:0003899) | 1.124328135 |
| HIV Life Cycle (R-HSA-162587) | 1.124328135 |
| IRE1 alpha activates chaperones (R-HSA-381070) | 1.124328135 |
| Late Phase of HIV Life Cycle (R-HSA-162599) | 1.124328135 |
| ligase activity, forming carbon-oxygen bonds (GO:0016875) | 1.124328135 |
| multi-organism localization (GO:1902579) | 1.124328135 |
| multi-organism transport (GO:0044766) | 1.124328135 |
| N-glycan trimming in the ER and Calnexin/Calreticulin cycle (R- | 1.124328135 |
| HSA-532668) | |
| Orel removal from chromatin (R-HSA-68949) | 1.124328135 |
| PERK regulates gene expression (R-HSA-381042) | 1.124328135 |
| regulation of autophagy of mitochondrion (GO:1903146) | 1.124328135 |
| Regulation of TP53 Degradation (R-HSA-6804757) | 1.124328135 |
| RNA Polymerase III Transcription Initiation From Type 1 | 1.124328135 |
| Promoter (R-HSA-76061) | |
| RNA Polymerase III Transcription Initiation From Type 3 | 1.124328135 |
| Promoter (R-HSA-76071) | |
| SUMOylation of DNA damage response and repair proteins (R- | 1.124328135 |
| HSA-3108214) | |
| SUMOylation of RNA binding proteins (R-HSA-4570464) | 1.124328135 |
| Synthesis of DNA (R-HSA-69239) | 1.124328135 |
| transport of virus (GO:0046794) | 1.124328135 |
| tRNA aminoacylation (GO:0043039) | 1.124328135 |
| amino acid activation (GO:0043038) | 1.13093087 |
| APC:Cdc20 mediated degradation of cell cycle proteins prior to | 1.13093087 |
| satisfation of the cell cycle checkpoint (R-HSA-179419) | |
| CDK-mediated phosphorylation and removal of Cdc6 (R-HSA- | 1.13093087 |
| 69017) | |
| Cleavage of Growing Transcript in the Termination Region (R- | 1.13093087 |
| HSA-109688) | |
| Energy dependent regulation of mTOR by LKB1-AMPK (R-HSA- | 1.13093087 |
| 380972) | |
| ER-nudeus signaling pathway (GO:0006984) | 1.13093087 |
| HIV elongation arrest and recovery (R-HSA-167287) | 1.13093087 |
| Interleukin-1 signaling (R-HSA-9020702) | 1.13093087 |
| mitochondrial gene expression (GO:0140053) | 1.13093087 |
| nuclear ubiguitin ligase complex (GO:0000152) | 1.13093087 |
| Pausing and recovery of HIV elongation (R-HSA-167290) | 1.13093087 |
| positive regulation of telomere maintenance (GO:0032206) | 1.13093087 |
| Regulation of APC/C activators between G1/S and early | 1.13093087 |
| anaphase (R-HSA-176408) | |
| Regulation of mRNA stability by proteins that bind AU-rich | 1.13093087 |
| elements (R-HSA-450531) | |
| ribonucleoprotein complex binding (GO:0043021) | 1.13093087 |
| RNA Polymerase II Transcription Termination (R-HSA-73856) | 1.13093087 |
| RNA Polymerase III Transcription Initiation (R-HSA-76046) | 1.13093087 |
| RUNX1 interacts with co-factors whose precise effect on RUNX1 | 1.13093087 |
| targets is not known (R-HSA-8939243) | |
| anaphase-promoting complex-dependent catabolic process | 1.137503524 |
| (GO:0031145) | |
| Assembly of the pre-replicative complex (R-HSA-68867) | 1.137503524 |
| CDT1 association with the CDC6:ORC:origin complex (R-HSA- | 1.137503524 |
| 68827) | |
| Degradation of beta-catenin by the destruction complex (R-HSA- | 1.137503524 |
| 195253) | |
| Degradation of GLI1 by the proteasome (R-HSA-5610780) | 1.137503524 |
| HDR through Single Strand Annealing (SSA)(R-HSA-5685938) | 1.137503524 |
| interleukin-1-mediated signaling pathway (GO:0070498) | 1.137503524 |
| mRNA 3′-end processing (GO:0031124) | 1.137503524 |
| mRNA Splicing - Minor Pathway (R-HSA-72165) | 1.137503524 |
| Nuclear import of Rev protein (R-HSA-180746) | 1.137503524 |
| positive regulation of telomere maintenance via telomere | 1.137503524 |
| lengthening (GO:1904358) | |
| telomeric DNA binding (GO:0042162) | 1.137503524 |
| DNA Replication Pre-lnitiation (R-HSA-69002) | 1.14404637 |
| M/G1 Transition (R-HSA-68874) | 1.14404637 |
| MAPK targets/ Nuclear events mediated by MAP kinases (R- | 1.14404637 |
| HSA-450282) | |
| mediator complex (GO:0016592) | 1.14404637 |
| Mitochondrial calcium ion transport (R-HSA-8949215) | 1.14404637 |
| mRNA export from nucleus (GO:0006406) | 1.14404637 |
| mRNA-containing ribonucleoprotein complex export from nucleus | 1.14404637 |
| (GO:0071427) | |
| Nuclear Envelope Breakdown (R-HSA-2980766) | 1.14404637 |
| nudeotide-excision repair (GO:0006289) | 1.14404637 |
| peptide N-acetyltransferase activity (GO:0034212) | 1.14404637 |
| Rab guanyl-nudeotide exchange factor activity (GO:0017112) | 1.14404637 |
| Recognition of DNA damage by PCNA-containing replication | 1.14404637 |
| complex (R-HSA-110314) | |
| Regulation of PTEN stability and activity (R-HSA-8948751) | 1.14404637 |
| ribosome binding (GO:0043022) | 1.14404637 |
| tRNA aminoacylation for protein translation (GO:0006418) | 1.14404637 |
| U2-type spliceosomal complex (GO:0005684) | 1.14404637 |
| APC/C:Cdh1 mediated degradation of Cdc20 and other | 1.150559677 |
| APC/C:Cdh1 targeted proteins in late mitosis/early G1 (R-HSA- | |
| 174178) | |
| Cdc20:Phospho-APC/C mediated degradation of Cyclin A (R- | 1.150559677 |
| HSA-174184) | |
| FBXL7 down-regulates AURKA during mitotic entry and in early | 1.150559677 |
| mitosis (R-HSA-8854050) | |
| HIV Infection (R-HSA-162906) | 1.150559677 |
| Host Interactions with Influenza Factors (R-HSA-168253) | 1.150559677 |
| MAP kinase activation (R-HSA-450294) | 1.150559677 |
| MicroRNA (miRNA)biogenesis (R-HSA-203927) | 1.150559677 |
| mRNA Splicing (R-HSA-72172) | 1.150559677 |
| Nuclear Pore Complex (NPC)Disassembly (R-HSA-3301854) | 1.150559677 |
| nucleotide-excision repair, DNA incision (GO:0033683) | 1.150559677 |
| exonuclease activity, active with either ribo- or deoxyribonucleic | 1.15704371 |
| acids and producing 5′-phosphomonoesters (GO:0016796) | |
| histone acetyltransferase activity (GO:0004402) | 1.15704371 |
| Interactions of Rev with host cellular proteins (R-HSA-177243) | 1.15704371 |
| mRNA Splicing - Major Pathway (R-HSA-72163) | 1.15704371 |
| multivesicular body sorting pathway (GO:0071985) | 1.15704371 |
| Retrograde transport at the Trans-Golgi-Network (R-HSA- | 1.15704371 |
| 6811440) | |
| transcription by RNA polymerase I (GO:0006360) | 1.15704371 |
| Transcription of the HIV genome (R-HSA-167172) | 1.15704371 |
| Degradation of GLI2 by the proteasome (R-HSA-5610783) | 1.163498732 |
| Downregulation of TGF-beta receptor signaling (R-HSA- | 1.163498732 |
| 2173788) | |
| Gap-filling DNA repair synthesis and ligation in GG-NER (R- | 1.163498732 |
| HSA-5696397) | |
| GLI3 is processed to GLI3R by the proteasome (R-HSA- | 1.163498732 |
| 5610785) | |
| peptide-lysine-N-acetyltransferase activity (GO:0061733) | 1.163498732 |
| preribosome, large subunit precursor (GO:0030687) | 1.163498732 |
| Processing of Capped Intron-Containing Pre-mRNA (R-HSA- | 1.163498732 |
| 72203) | |
| RNA Polymerase II Pre-transcription Events (R-HSA-674695) | 1.163498732 |
| Transcriptional Regulation by E2F6 (R-HSA-8953750) | 1.163498732 |
| translational elongation (GO:0006414) | 1.163498732 |
| DAP12 signaling (R-HSA-2424491) | 1.169925001 |
| Defective CFTR causes cystic fibrosis (R-HSA-5678895) | 1.169925001 |
| p53-lndependent DNA Damage Response (R-HSA-69610) | 1.169925001 |
| p53-lndependent G1/S DNA damage checkpoint (R-HSA-69613) | 1.169925001 |
| translational termination (GO:0006415) | 1.169925001 |
| tRNA processing in the nucleus (R-HSA-6784531) | 1.169925001 |
| Ubiquitin Mediated Degradation of Phosphorylated Cdc25A (R- | 1.169925001 |
| HSA-69601) | |
| Ubiquitin-dependent degradation of Cyclin D (R-HSA-75815) | 1.169925001 |
| Ubiguitin-dependent degradation of Cyclin D1 (R-HSA-69229) | 1.169925001 |
| 3′-5′ exonuclease activity (GO:0008408) | 1.176322773 |
| Base Excision Repair (R-HSA-73884) | 1.176322773 |
| Degradation of AXIN (R-HSA-4641257) | 1.176322773 |
| Host Interactions of HIV factors (R-HSA-162909) | 1.176322773 |
| intracellular transport of virus (GO:0075733) | 1.176322773 |
| Resolution of Abasic Sites (AP sites)(R-HSA-73933) | 1.176322773 |
| The role of Nef in HIV-1 replication and disease pathogenesis (R- | 1.176322773 |
| HSA-164952) | 1.182692298 |
| Deadenylation-dependent mRNA decay (R-HSA-429914) | |
| Formation of HIV-1 elongation complex containing HIV-1 Tat (R- | 1.182692298 |
| HSA-167200) | |
| Hh mutants that don't undergo autocatalytic processing are | 1.182692298 |
| degraded by ERAD (R-HSA-5362768) | |
| HIV Transcription Elongation (R-HSA-167169) | 1.182692298 |
| HIV Transcription Initiation (R-HSA-167161) | 1.182692298 |
| immunological synapse (GO:0001772) | 1.182692298 |
| ncRNA transcription (GO:0098781) | 1.182692298 |
| NS1 Mediated Effects on Host Pathways (R-HSA-168276) | 1.182692298 |
| nudeotide-excision repair, DNA incision, 5′-to lesion | 1.182692298 |
| (GO:0006296) | |
| nudeotide-sugar metabolic process (GO:0009225) | 1.182692298 |
| proteasome complex (GO:0000502) | 1.182692298 |
| Regulation of Glucokinase by Glucokinase Regulatory Protein | 1.182692298 |
| (R-HSA-170822) | |
| RNA Polymerase II HIV Promoter Escape (R-HSA-167162) | 1.182692298 |
| RNA Polymerase II Promoter Escape (R-HSA-73776) | 1.182692298 |
| RNA Polymerase II Transcription Initiation (R-HSA-75953) | 1.182692298 |
| RNA Polymerase II Transcription Initiation And Promoter | 1.182692298 |
| Clearance (R-HSA-76042) | |
| RNA Polymerase II Transcription Pre-lnitiation And Promoter | 1.182692298 |
| Opening (R-HSA-73779) | |
| Tat-mediated elongation of the HIV-1 transcript (R-HSA-167246) | 1.182692298 |
| transcription initiation from RNA polymerase I promoter | 1.182692298 |
| (GO:0006361) | |
| APC/C:Cdc20 mediated degradation of Securin (R-HSA-174154) | 1.189033824 |
| endopeptidase complex (GO:1905369) | 1.189033824 |
| Export of Viral Ribonucleoproteins from Nucleus (R-HSA- | 1.189033824 |
| 168274) | |
| Formation of HIV elongation complex in the absence of HIV Tat | 1.189033824 |
| (R-HSA-167152) | |
| histone deacetylase activity (GO:0004407) | 1.189033824 |
| Metabolism of non-coding RNA (R-HSA-194441) | 1.189033824 |
| mitochondrial large ribosomal subunit (GO:0005762) | 1.189033824 |
| mitochondrial ribosome (GO:0005761) | 1.189033824 |
| mitochondrial small ribosomal subunit (GO:0005763) | 1.189033824 |
| negative regulation of mRNA processing (GO:0050686) | 1.189033824 |
| organellar large ribosomal subunit (GO:0000315) | 1.189033824 |
| organellar ribosome (GO:0000313) | 1.189033824 |
| organellar small ribosomal subunit (GO:0000314) | 1.189033824 |
| production of miRNAs involved in gene silencing by miRNA | 1.189033824 |
| (GO:0035196) | |
| snRNA binding (GO:0017069) | 1.189033824 |
| snRNP Assembly (R-HSA-191859) | 1.189033824 |
| Cross-presentation of soluble exogenous antigens | 1.195347598 |
| (endosomes)(R-HSA-1236978) | |
| CTLA4 inhibitory signaling (R-HSA-389513) | 1.195347598 |
| Formation of RNA Pol II elongation complex (R-HSA-112382) | 1.195347598 |
| Inactivation of APC/C via direct inhibition of the APC/C complex | 1.195347598 |
| (R-HSA-141430) | |
| Inflammasomes (R-HSA-622312) | 1.195347598 |
| Inhibition of the proteolytic activity of APC/C required for the | 1.195347598 |
| onset of anaphase by mitotic spindle checkpoint components (R- | |
| HSA-141405) | |
| mitochondrial translation (GO:0032543) | 1.195347598 |
| mTOR signalling (R-HSA-165159) | 1.195347598 |
| positive requlation of viral transcription (GO:0050434) | 1.195347598 |
| precatalytic spliceosome (GO:0071011) | 1.195347598 |
| protein deacetylase activity (GO:0033558) | 1.195347598 |
| Regulation of activated PAK-2p34 by proteasome mediated | 1.195347598 |
| degradation (R-HSA-211733) | |
| regulation of DNA-templated transcription, elongation | 1.195347598 |
| (GO:0032784) | |
| RNA Polymerase II Transcription Elongation (R-HSA-75955) | 1.195347598 |
| U2-type catalytic step 2 spliceosome (GO:0071007) | 1.195347598 |
| U2-type precatalytic spliceosome (GO:0071005) | 1.195347598 |
| Autodegradation of the E3 ubiquitin ligase COP1 (R-HSA- | 1.201633861 |
| 349425) | |
| Formation of Incision Complex in GG-NER (R-HSA-5696395) | 1.201633861 |
| Regulation of Apoptosis (R-HSA-169911) | 1.201633861 |
| regulation of defense response to virus by virus (GO:0050690) | 1 201633861 |
| Rev-mediated nuclear export of HIV RNA (R-HSA-165054) | 1.201633861 |
| RNA Polymerase I Transcription Termination (R-HSA-73863) | 1.201633861 |
| SUMOylation of SUMOylation proteins (R-HSA-4085377) | 1.201633861 |
| termination of RNA polymerase I transcription (GO:0006363) | 1.201633861 |
| TGF-beta receptor signaling activates SMADs (R-HSA-2173789) | 1.201633861 |
| tRNA Aminoacylation (R-HSA-379724) | 1.201633861 |
| Vif-mediated degradation of APOBEC3G (R-HSA-180585) | 1.201633861 |
| Vpu mediated degradation of CD4 (R-HSA-180534) | 1.201633861 |
| 7-methylguanosine mRNA capping (GO:0006370) | 1.207892852 |
| catalytic step 2 spliceosome (GO:0071013) | 1.207892852 |
| Citric acid cycle (TCA cycle)(R-HSA-71403) | 1.207892852 |
| ERK/MAPK targets (R-HSA-198753) | 1.207892852 |
| interphase (GO:0051325) | 1.207892852 |
| Mitochondrial translation elongation (R-HSA-5389840) | 1.207892852 |
| Mitochondrial translation initiation (R-HSA-5368286) | 1.207892852 |
| Mitochondrial translation termination (R-HSA-5419276) | 1.207892852 |
| mitochondrial translational elongation (GO:0070125) | 1.207892852 |
| mitochondrial translational termination (GO:0070126) | 1.207892852 |
| mitotic interphase (GO:0051329) | 1.207892852 |
| nuclear DNA replication (GO:0033260) | 1.207892852 |
| SCF-beta-TrCP mediated degradation of Emil (R-HSA-174113) | 1.207892852 |
| Stabilization of p53 (R-HSA-69541) | 1.207892852 |
| 7-methylguanosine RNA capping (GO:0009452) | 1.214124805 |
| cell cycle DNA replication (GO:0044786) | 1.214124805 |
| Mitochondrial translation (R-HSA-5368287) | 1.214124805 |
| mRNA 3′-end processing (R-HSA-72187) | 1.214124805 |
| mTORCI-mediated signalling (R-HSA-166208) | 1.214124805 |
| RHO GTPases Activate WASPs and WAVEs (R-HSA-5663213) | 1.214124805 |
| RNA capping (GO:0036260) | 1.214124805 |
| CLEC7A (Dectin-1 Signaling (R-HSA-5607764) | 1.220329955 |
| Constitutive Signaling by AKT1 E17K in Cancer (R-HSA- | 1.220329955 |
| 5674400) | |
| Dectin-1 mediated noncanonical NF-kB signaling (R-HSA- | 1.220329955 |
| 5607761) | |
| NIK-->noncanonical NF-kB signaling (R-HSA-5676590) | 1.220329955 |
| RNA polymerase binding (GO:0070063) | 1.220329955 |
| tRNA transport (GO:0051031) | 1.220329955 |
| Autodegradation of Cdh1 by Cdh1:APC/C (R-HSA-174084) | 1.22650853 |
| DNA Damage Recognition in GG-NER (R-HSA-5696394) | 1.22650853 |
| exosome (RNase complex)(GO:0000178) | 1.22650853 |
| Gap-filling DNA repair synthesis and ligation in TC-NER (R-HSA- | 1.22650853 |
| 6782210) | |
| ncRNA export from nucleus (GO:0097064) | 1.22650853 |
| negative regulation of DNA-dependent DNA replication | 1.22650853 |
| (GO:2000104) | |
| Nuclear Events (kinase and transcription factor activation)(R- | 1.22650853 |
| HSA-198725) | |
| Regulation of ornithine decarboxylase (ODC)(R-HSA-350562) | 1.22650853 |
| transcription elongation from RNA polymerase II promoter | 1.22650853 |
| (GO:0006368) | |
| Activation of NF-kappaB in B cells (R-HSA-1169091) | 1.232660757 |
| DNA-templated transcription, elongation (GO:0006354) | 1.232660757 |
| Downstream signaling events of B Cell Receptor (BCR)(R-HSA- | 1.232660757 |
| 1168372) | |
| Dual incision in TC-NER (R-HSA-6782135) | 1.232660757 |
| exoribonuclease complex (GO:1905354) | 1.232660757 |
| Formation of TC-NER Pre-lncision Complex (R-HSA-6781823) | 1.232660757 |
| histone ubiquitination (GO:0016574) | 1.232660757 |
| maturation of 5.8S rRNA (GO:0000460) | 1.232660757 |
| mitotic S phase (GO:0000084) | 1.232660757 |
| Negative regulation of NOTCH4 signaling (R-HSA-9604323) | 1.232660757 |
| S phase (GO:0051320) | 1.232660757 |
| translation initiation factor activity (GO:0003743) | 1.232660757 |
| Transport of Mature Transcript to Cytoplasm (R-HSA-72202) | 1.232660757 |
| basal RNA polymerase II transcription machinery binding | 1.23878686 |
| (GO:0001099) | |
| basal transcription machinery binding (GO:0001098) | 1.23878686 |
| Dual Incision in GG-NER (R-HSA-5696400) | 1.23878686 |
| Global Genome Nucleotide Excision Repair (GG-NER)(R-HSA- | 1.23878686 |
| 5696399) | |
| INO80-type complex (GO:0097346) | 1.23878686 |
| NEP/NS2 Interacts with the Cellular Export Machinery (R-HSA- | 1.23878686 |
| 168333) | |
| Nucleotide Excision Repair (R-HSA-5696398) | 1.23878686 |
| RNA phosphodiester bond hydrolysis, exonucleolytic | 1.23878686 |
| (GO:0090503) | |
| snRNA transcription (GO:0009301) | 1.23878686 |
| snRNA transcription by RNA polymerase II (GO:0042795) | 1.23878686 |
| SUMOylation of DNA replication proteins (R-HSA-4615885) | 1.23878686 |
| Transport of Mature mRNA derived from an Intron-Containing | 1.23878686 |
| Transcript (R-HSA-159236) | |
| Extension of Telomeres (R-HSA-180786) | 1.244887059 |
| histone monoubiquitination (GO:0010390) | 1.244887059 |
| nucleotide-excision repair, preincision complex assembly | 1.244887059 |
| (GO:0006294) | |
| Regulation of TP53 Activity through Acetylation (R-HSA- | 1.244887059 |
| 6804758) | |
| regulation of transcription elongation from RNA polymerase II | 1.244887059 |
| promoter (GO:0034243) | |
| RNA Polymerase I Promoter Escape (R-HSA-73772) | 1.244887059 |
| RNA polymerase II transcribes snRNA genes (R-HSA-6807505) | 1.244887059 |
| transcription elongation from RNA polymerase I promoter | 1.244887059 |
| (GO:0006362) | |
| transcription-coupled nucleotide-excision repair (GO:0006283) | 1.244887059 |
| positive regulation of DNA-templated transcription, elongation | 1.250961574 |
| (GO:0032786) | |
| RNA polymerase core enzyme binding (GO:0043175) | 1.250961574 |
| RNA Polymerase I Transcription Initiation (R-HSA-73762) | 1.250961574 |
| Transcription-Coupled Nucleotide Excision Repair (TC-NER)(R- | 1.250961574 |
| HSA-6781827) | |
| 3′-5′-exoribonuclease activity (GO:0000175) | 1.257010618 |
| exonucleolytic catabolism of deadenylated mRNA (GO:0043928) | 1.257010618 |
| Interactions of Vpr with host cellular proteins (R-HSA-176033) | 1.257010618 |
| tRNA export from nucleus (GO:0006409) | 1.257010618 |
| tRNA-containing ribonucleoprotein complex export from nucleus | 1.257010618 |
| (GO:0071431) | |
| exoribonuclease activity, producing 5′-phosphomonoesters | 1.263034406 |
| (GO:0016896) | |
| nuclear-transcribed mRNA catabolic process, exonucleolytic | 1.263034406 |
| (GO:0000291) | |
| Regulation of RUNX3 expression and activity (R-HSA-8941858) | 1.263034406 |
| exoribonuclease activity (GO:0004532) | 1.269033146 |
| Lagging Strand Synthesis (R-HSA-69186) | 1.269033146 |
| Transport of Mature mRNA Derived from an Intronless Transcript | 1.269033146 |
| (R-HSA-159231) | |
| Transport of Mature mRNAs Derived from Intronless Transcripts | 1.269033146 |
| (R-HSA-159234) | |
| Viral Messenger RNA Synthesis (R-HSA-168325) | 1.269033146 |
| PCNA-Dependent Long Patch Base Excision Repair (R-HSA- | 1.275007047 |
| 5651801) | |
| Abortive elongation of HIV-1 transcript in the absence of Tat (R- | 1.280956314 |
| HSA-167242) | |
| proteasome regulatory particle (GO:0005838) | 1.280956314 |
| proteasome accessory complex (GO:0022624) | 1.286881148 |
| Resolution of AP sites via the multiple-nucleotide patch | 1.286881148 |
| replacement pathway (R-HSA-110373) | |
| Telomere C-strand (Lagging Strand)Synthesis (R-HSA-174417) | 1.286881148 |
| telomere maintenance via semi-conservative replication | 1.286881148 |
| (GO:0032201) | |
| mRNA Capping (R-HSA-72086) | 1.292781749 |
| RNA Pol II CTD phosphorylation and interaction with CE (R- | 1.292781749 |
| HSA-77075) | |
| RNA Pol II CTD phosphorylation and interaction with CE during | 1.292781749 |
| HIV infection (R-HSA-167160) | |
| Transport of Ribonucleoproteins into the Host Nucleus (R-HSA- | 1.292781749 |
| 168271) | |
| Formation of the Early Elongation Complex (R-HSA-113418) | 1.298658316 |
| Formation of the HIV-1 Early Elongation Complex (R-HSA- | 1.298658316 |
| 167158) | |
| MLL1 complex (GO:0071339) | 1.298658316 |
| MLL1/2 complex (GO:0044665) | 1.298658316 |
| Transport of the SLBP Dependant Mature mRNA (R-HSA- | 1.298658316 |
| 159230) | |
| Transport of the SLBP independent Mature mRNA (R-HSA- | 1.298658316 |
| 159227) | |
| Vpr-mediated nuclear import of PICs (R-HSA-180910) | 1.298658316 |
| RNA polymerase II complex binding (GO:0000993) | 1.304511042 |
| SUMOylation of ubiquitinylation proteins (R-HSA-3232142) | 1.304511042 |
| carboxy-terminal domain protein kinase complex (GO:0032806) | 1.344828497 |
| Cytosolic tRNA aminoacylation (R-HSA-379716) | 1.344828497 |
| nudeotide-excision repair, preincision complex stabilization | 1.344828497 |
| (GO:0006293) | |
| RAF activation (R-HSA-5673000) | 1.344828497 |
| Synthesis of PIPs at the early endosome membrane (R-HSA- | 1.344828497 |
| 1660516) | |
| Alternative Trascription Start/End n = 835 | |
| bitter taste receptor activity (GO:0033038) | −6.64385619 |
| regulation of peptidyl-serine phosphorylation of STAT protein | −6.64385619 |
| (GO:0033139) | |
| immunoglobulin complex (GO:0019814) | −4.058893689 |
| immunoglobulin complex, circulating (GO:0042571) | −4.058893689 |
| detection of chemical stimulus involved in sensory perception of | −3.321928095 |
| smell (GO:0050911) | |
| olfactory receptor activity (GO:0004984) | −3.321928095 |
| Classical antibody-mediated complement activation (R-HSA- | −3.184424571 |
| 173623) | |
| detection of chemical stimulus involved in sensory perception | −3.184424571 |
| (GO:0050907) | |
| detection of chemical stimulus involved in sensory perception of | −2.943416472 |
| bitter taste (GO:0001580) | |
| odorant binding (GO:0005549) | −2.943416472 |
| Olfactory Signaling Pathway (R-HSA-381753) | −2.943416472 |
| Creation of C4 and C2 activators (R-HSA-166786) | −2.836501268 |
| complement activation, classical pathway (GO:0006958) | −2.736965594 |
| CD22 mediated BCR regulation (R-HSA-5690714) | −2.64385619 |
| immunoglobulin receptor binding (GO:0034987) | −2.64385619 |
| keratin filament (GO:0045095) | −2.64385619 |
| sensory perception of smell (GO:0007608) | −2.64385619 |
| detection of chemical stimulus involved in sensory perception of | −2.556393349 |
| taste (GO:0050912) | |
| detection of stimulus involved in sensory perception | −2.556393349 |
| (GO:0050906) | |
| sensory perception of bitter taste (GO:0050913) | −2.556393349 |
| detection of chemical stimulus (GO:0009593) | −2.473931188 |
| Initial triggering of complement (R-HSA-166663) | −2.473931188 |
| sensory perception of chemical stimulus (GO:0007606) | −2.395928676 |
| humoral immune response mediated by circulating | −2.321928095 |
| immunoglobulin (GO:0002455) | |
| phagocytosis, recognition (GO:0006910) | −2.321928095 |
| complement activation (GO:0006956) | −2.251538767 |
| Scavenging of heme from plasma (R-HSA-2168880) | −2.251538767 |
| T cell receptor complex (GO:0042101) | −2.120294234 |
| Keratinization (R-HSA-6805567) | −2.058893689 |
| FCGR activation (R-HSA-2029481) | −2 |
| keratinization (GO:0031424) | −1.888968688 |
| detection of stimulus (GO:0051606) | −1.556393349 |
| G alpha (s signalling events (R-HSA-418555)) | −1.556393349 |
| antigen binding (GO:0003823) | −1.514573173 |
| keratinocyte differentiation (GO:0030216) | −1.395928676 |
| nucleosome (GO:0000786) | −1.395928676 |
| regulation of complement activation (GO:0030449) | −1.395928676 |
| Complement cascade (R-HSA-166658) | −1.358453971 |
| phaqocytosis, engulfment (GO:0006911) | −1.358453971 |
| Formation of the cornified envelope (R-HSA-6809371) | −1.321928095 |
| Regulation of Complement cascade (R-HSA-977606) | −1.321928095 |
| Antimicrobial peptides (R-HSA-6803157) | −1.286304185 |
| intermediate filament (GO:0005882) | −1.251538767 |
| immunoglobulin production (GO:0002377) | −1.184424571 |
| B cell mediated immunity (GO:0019724) | −1.152003093 |
| immunoglobulin mediated immune response (GO:0016064) | −1.152003093 |
| G protein-coupled receptor activity (GO:0004930) | −1.120294234 |
| plasma membrane invagination (GO:0099024) | −1.120294234 |
| cornification (GO:0070268) | −1.089267338 |
| humoral immune response (GO:0006959) | −1.089267338 |
| membrane invagination (GO:0010324) | −1.089267338 |
| epidermal cell differentiation (GO:0009913) | −1.058893689 |
| regulation of humoral immune response (GO:0002920) | −1.058893689 |
| B cell receptor signaling pathway (GO:0050853) | −1.029146346 |
| sensory perception (GO:0007600) | −0.915935735 |
| defense response to bacterium (GO:0042742) | −0.888968688 |
| intermediate filament cytoskeleton (GO:0045111) | −0.888968688 |
| production of molecular mediator of immune response | −0.888968688 |
| (GO:0002440) | |
| Unclassified (UNCLASSIFIED) | −0.785875195 |
| cytokine activity (GO:0005125) | −0.736965594 |
| skin development (GO:0043588) | −0.736965594 |
| G protein-coupled receptor signaling pathway (GO:0007186) | −0.666576266 |
| epidermis development (GO:0008544) | −0.64385619 |
| adaptive immune response (GO:0002250) | −0.621488377 |
| lymphocyte mediated immunity (GO:0002449) | −0.59946207 |
| GPCR downstream signalling (R-HSA-388396) | −0.577766999 |
| Signaling by GPCR (R-HSA-372790) | −0.577766999 |
| nervous system process (GO:0050877) | −0.473931188 |
| transmembrane signaling receptor activity (GO:0004888) | −0.454031631 |
| signaling receptor activity (GO:0038023) | −0.304006187 |
| system process (GO:0003008) | −0.286304185 |
| molecular transducer activity (GO:0060089) | −0.251538767 |
| cellular component (GO:0005575) | 0.084064265 |
| biological process (GO:0008150) | 0.111031312 |
| membrane (GO:0016020) | 0.111031312 |
| signal transduction (GO:0007165) | 0.124328135 |
| signaling (GO:0023052) | 0.124328135 |
| anatomical structure development (GO:0048856) | 0.137503524 |
| cell (GO:0005623) | 0.137503524 |
| cell communication (GO:0007154) | 0.137503524 |
| cell part (GO:0044464) | 0.137503524 |
| cellular developmental process (GO:0048869) | 0.137503524 |
| developmental process (GO:0032502) | 0.137503524 |
| molecular function (GO:0003674) | 0.137503524 |
| response to chemical (GO:0042221) | 0.137503524 |
| response to stimulus (GO:0050896) | 0.137503524 |
| multicellular organism development (GO:0007275) | 0.150559677 |
| system development (GO:0048731) | 0.150559677 |
| multi-organism process (GO:0051704) | 0.163498732 |
| regulation of biological process (GO:0050789) | 0.163498732 |
| biological regulation (GO:0065007) | 0.176322773 |
| cellular process (GO:0009987) | 0.176322773 |
| cellular response to stimulus (GO:0051716) | 0.176322773 |
| regulation of cellular process (GO:0050794) | 0.176322773 |
| transcription regulator activity (GO:0140110) | 0.189033824 |
| binding (GO:0005488) | 0.201633861 |
| DNA binding (GO:0003677) | 0.201633861 |
| cation binding (GO:0043169) | 0.214124805 |
| cell surface receptor signaling pathway (GO:0007166) | 0.214124805 |
| regulation of immune system process (GO:0002682) | 0.214124805 |
| extracellular exosome (GO:0070062) | 0.22650853 |
| extracellular organelle (GO:0043230) | 0.22650853 |
| metal ion binding (GO:0046872) | 0.22650853 |
| anatomical structure morphogenesis (GO:0009653) | 0.23878686 |
| extracellular vesicle (GO:1903561) | 0.23878686 |
| intracellular (GO:0005622) | 0.23878686 |
| intracellular part (GO:0044424) | 0.23878686 |
| organelle (GO:0043226) | 0.23878686 |
| chemical homeostasis (GO:0048878) | 0.250961574 |
| cytoskeletal part (GO:0044430) | 0.250961574 |
| homeostatic process (GO:0042592) | 0.250961574 |
| Innate Immune System (R-HSA-168249) | 0.250961574 |
| ion binding (GO:0043167) | 0.250961574 |
| nucleic acid binding (GO:0003676) | 0.250961574 |
| positive regulation of response to stimulus (GO:0048584) | 0.250961574 |
| regulation of transcription by RNA polymerase II (GO:0006357) | 0.250961574 |
| heterocyclic compound binding (GO:1901363) | 0.263034406 |
| intracellular organelle (GO:0043229) | 0.263034406 |
| negative regulation of developmental process (GO:0051093) | 0.263034406 |
| negative regulation of molecular function (GO:0044092) | 0.263034406 |
| negative regulation of multicellular organismal process | 0.263034406 |
| (GO:0051241) | |
| nervous system development (GO:0007399) | 0.263034406 |
| organic cyclic compound binding (GO:0097159) | 0.263034406 |
| plasma membrane region (GO:0098590) | 0.263034406 |
| protein dimerization activity (GO:0046983) | 0.263034406 |
| proteolysis (GO:0006508) | 0.263034406 |
| regulation of cell population proliferation (GO:0042127) | 0.263034406 |
| regulation of multicellular organismal development (GO:2000026) | 0.263034406 |
| secretory granule (GO:0030141) | 0.263034406 |
| transport (GO:0006810) | 0.263034406 |
| biological adhesion (GO:0022610) | 0.275007047 |
| catalytic activity (GO:0003824) | 0.275007047 |
| cell adhesion (GO:0007155) | 0.275007047 |
| cell development (GO:0048468) | 0.275007047 |
| cell projection part (GO:0044463) | 0.275007047 |
| cytoskeleton (GO:0005856) | 0.275007047 |
| establishment of localization (GO:0051234) | 0.275007047 |
| localization (GO:0051179) | 0.275007047 |
| membrane-bounded organelle (GO:0043227) | 0.275007047 |
| neuron differentiation (GO:0030182) | 0.275007047 |
| organic acid metabolic process (GO:0006082) | 0.275007047 |
| oxoacid metabolic process (GO:0043436) | 0.275007047 |
| plasma membrane bounded cell projection part (GO:0120038) | 0.275007047 |
| protein-containing complex (GO:0032991) | 0.275007047 |
| regulation of multicellular organismal process (GO:0051239) | 0.275007047 |
| regulation of response to stimulus (GO:0048583) | 0.275007047 |
| response to stress (GO:0006950) | 0.275007047 |
| secretory vesicle (GO:0099503) | 0.275007047 |
| cellular response to endogenous stimulus (GO:0071495) | 0.286881148 |
| intracellular membrane-bounded organelle (GO:0043231) | 0.286881148 |
| neurogenesis (GO:0022008) | 0.286881148 |
| neuron projection (GO:0043005) | 0.286881148 |
| nucleus (GO:0005634) | 0.286881148 |
| organonitrogen compound metabolic process (GO:1901564) | 0.286881148 |
| plasma membrane bounded cell projection (GO:0120025) | 0.286881148 |
| positive regulation of biological process (GO:0048518) | 0.286881148 |
| regulation of nervous system development (GO:0051960) | 0.286881148 |
| regulation of nitrogen compound metabolic process | 0.286881148 |
| (GO:0051171) | |
| regulation of primary metabolic process (GO:0080090) | 0.286881148 |
| response to endogenous stimulus (GO:0009719) | 0.286881148 |
| somatodendritic compartment (GO:0036477) | 0.286881148 |
| tube development (GO:0035295) | 0.286881148 |
| vesicle (GO:0031982) | 0.286881148 |
| carboxylic acid metabolic process (GO:0019752) | 0.298658316 |
| cell projection (GO:0042995) | 0.298658316 |
| cellular response to chemical stimulus (GO:0070887) | 0.298658316 |
| circulatory system development (GO:0072359) | 0.298658316 |
| cytoplasm (GO:0005737) | 0.298658316 |
| generation of neurons (GO:0048699) | 0.298658316 |
| head development (GO:0060322) | 0.298658316 |
| Immune System (R-HSA-168256) | 0.298658316 |
| metabolic process (GO:0008152) | 0.298658316 |
| Metabolism (R-HSA-1430728) | 0.298658316 |
| negative regulation of protein metabolic process (GO:0051248) | 0.298658316 |
| positive regulation of transcription by RNA polymerase II | 0.298658316 |
| (GO:0045944) | |
| protein binding (GO:0005515) | 0.298658316 |
| protein metabolic process (GO:0019538) | 0.298658316 |
| regulation of anatomical structure morphogenesis (GO:0022603) | 0.298658316 |
| regulation of biological quality (GO:0065008) | 0.298658316 |
| regulation of biosynthetic process (GO:0009889) | 0.298658316 |
| regulation of cellular biosynthetic process (GO:0031326) | 0.298658316 |
| regulation of cellular metabolic process (GO:0031323) | 0.298658316 |
| regulation of developmental process (GO:0050793) | 0.298658316 |
| regulation of macromolecule metabolic process (GO:0060255) | 0.298658316 |
| regulation of metabolic process (GO:0019222) | 0.298658316 |
| regulation of nucleic acid-templated transcription (GO:1903506) | 0.298658316 |
| regulation of response to external stimulus (GO:0032101) | 0.298658316 |
| regulation of RNA biosynthetic process (GO:2001141) | 0.298658316 |
| regulation of secretion (GO:0051046) | 0.298658316 |
| regulation of transcription, DNA-templated (GO:0006355) | 0.298658316 |
| small molecule binding (GO:0036094) | 0.298658316 |
| catalytic activity, acting on a protein (GO:0140096) | 0.310340121 |
| cell death (GO:0008219) | 0.310340121 |
| cytokine-mediated signaling pathway (GO:0019221) | 0.310340121 |
| enzyme linked receptor protein signaling pathway (GO:0007167) | 0.310340121 |
| intracellular non-membrane-bounded organelle (GO:0043232) | 0.310340121 |
| lipid metabolic process (GO:0006629) | 0.310340121 |
| negative regulation of biological process (GO:0048519) | 0.310340121 |
| negative regulation of cellular process (GO:0048523) | 0.310340121 |
| negative regulation of cellular protein metabolic process | 0.310340121 |
| (GO:0032269) | |
| negative regulation of response to stimulus (GO:0048585) | 0.310340121 |
| neuron part (GO:0097458) | 0.310340121 |
| nitrogen compound metabolic process (GO:0006807) | 0.310340121 |
| non-membrane-bounded organelle (GO:0043228) | 0.310340121 |
| organic substance metabolic process (GO:0071704) | 0.310340121 |
| positive regulation of cellular process (GO:0048522) | 0.310340121 |
| positive regulation of multicellular organismal process | 0.310340121 |
| (GO:0051240) | |
| primary metabolic process (GO:0044238) | 0.310340121 |
| programmed cell death (GO:0012501) | 0.310340121 |
| protein-containing complex subunit organization (GO:0043933) | 0.310340121 |
| regulation of cellular macromolecule biosynthetic process | 0.310340121 |
| (GO:2000112) | |
| regulation of cellular protein metabolic process (GO:0032268) | 0.310340121 |
| regulation of defense response (GO:0031347) | 0.310340121 |
| regulation of hydrolase activity (GO:0051336) | 0.310340121 |
| regulation of macromolecule biosynthetic process (GO:0010556) | 0.310340121 |
| regulation of multi-organism process (GO:0043900) | 0.310340121 |
| regulation of protein metabolic process (GO:0051246) | 0.310340121 |
| response to abiotic stimulus (GO:0009628) | 0.310340121 |
| response to organic substance (GO:0010033) | 0.310340121 |
| small molecule metabolic process (GO:0044281) | 0.310340121 |
| anion binding (GO:0043168) | 0.321928095 |
| carbohydrate derivative metabolic process (GO:1901135) | 0.321928095 |
| cellular amide metabolic process (GO:0043603) | 0.321928095 |
| cellular component biogenesis (GO:0044085) | 0.321928095 |
| cellular component organization (GO:0016043) | 0.321928095 |
| cellular component organization or biogenesis (GO:0071840) | 0.321928095 |
| cellular response to organic substance (GO:0071310) | 0.321928095 |
| enzyme regulator activity (GO:0030234) | 0.321928095 |
| leukocyte activation (GO:0045321) | 0.321928095 |
| macromolecule metabolic process (GO:0043170) | 0.321928095 |
| membrane organization (GO:0061024) | 0.321928095 |
| negative regulation of cell communication (GO:0010648) | 0.321928095 |
| negative regulation of signal transduction (GO:0009968) | 0.321928095 |
| negative regulation of signaling (GO:0023057) | 0.321928095 |
| organonitrogen compound biosynthetic process (GO:1901566) | 0.321928095 |
| organonitrogen compound catabolic process (GO:1901565) | 0.321928095 |
| positive regulation of biosynthetic process (GO:0009891) | 0.321928095 |
| positive regulation of cell population proliferation (GO:0008284) | 0.321928095 |
| positive regulation of cellular biosynthetic process (GO:0031328) | 0.321928095 |
| positive regulation of developmental process (GO:0051094) | 0.321928095 |
| positive regulation of nucleic acid-templated transcription | 0.321928095 |
| (GO:1903508) | |
| positive regulation of signaling (GO:0023056) | 0.321928095 |
| positive regulation of transcription, DNA-templated | 0.321928095 |
| (GO:0045893) | |
| protein-containing complex assembly (GO:0065003) | 0.321928095 |
| regulation of cell development (GO:0060284) | 0.321928095 |
| regulation of cell differentiation (GO:0045595) | 0.321928095 |
| regulation of gene expression (GO:0010468) | 0.321928095 |
| regulation of localization (GO:0032879) | 0.321928095 |
| regulation of molecular function (GO:0065009) | 0.321928095 |
| regulation of RNA metabolic process (GO:0051252) | 0.321928095 |
| regulation of transport (GO:0051049) | 0.321928095 |
| response to oxygen-containing compound (GO:1901700) | 0.321928095 |
| Transport of small molecules (R-HSA-382551) | 0.321928095 |
| tube morphogenesis (GO:0035239) | 0.321928095 |
| carbohydrate derivative binding (GO:0097367) | 0.333423734 |
| cellular component assembly (GO:0022607) | 0.333423734 |
| cellular metabolic process (GO:0044237) | 0.333423734 |
| cytoplasmic part (GO:0044444) | 0.333423734 |
| endoplasmic reticulum (GO:0005783) | 0.333423734 |
| export from cell (GO:0140352) | 0.333423734 |
| immune system development (GO:0002520) | 0.333423734 |
| neuron development (GO:0048666) | 0.333423734 |
| organelle part (GO:0044422) | 0.333423734 |
| organic substance catabolic process (GO:1901575) | 0.333423734 |
| positive regulation of cell communication (GO:0010647) | 0.333423734 |
| positive regulation of cellular metabolic process (GO:0031325) | 0.333423734 |
| positive regulation of gene expression (GO:0010628) | 0.333423734 |
| positive regulation of macromolecule biosynthetic process | 0.333423734 |
| (GO:0010557) | |
| positive regulation of metabolic process (GO:0009893) | 0.333423734 |
| positive regulation of nitrogen compound metabolic process | 0.333423734 |
| (GO:0051173) | |
| positive regulation of RNA biosynthetic process (GO:1902680) | 0.333423734 |
| postsynapse (GO:0098794) | 0.333423734 |
| regulation of catalytic activity (GO:0050790) | 0.333423734 |
| regulation of cell communication (GO:0010646) | 0.333423734 |
| regulation of neurogenesis (GO:0050767) | 0.333423734 |
| regulation of nucleobase-containing compound metabolic | 0.333423734 |
| process (GO:0019219) | |
| regulation of protein modification process (GO:0031399) | 0.333423734 |
| regulation of protein phosphorylation (GO:0001932) | 0.333423734 |
| regulation of signal transduction (GO:0009966) | 0.333423734 |
| regulation of signaling (GO:0023051) | 0.333423734 |
| response to nitrogen compound (GO:1901698) | 0.333423734 |
| response to organonitrogen compound (GO:0010243) | 0.333423734 |
| secretion (GO:0046903) | 0.333423734 |
| small molecule biosynthetic process (GO:0044283) | 0.333423734 |
| vesicle-mediated transport (GO:0016192) | 0.333423734 |
| amide biosynthetic process (GO:0043604) | 0.344828497 |
| catabolic process (GO:0009056) | 0.344828497 |
| cell activation (GO:0001775) | 0.344828497 |
| cellular lipid metabolic process (GO:0044255) | 0.344828497 |
| cellular response to nitrogen compound (GO:1901699) | 0.344828497 |
| cellular response to organic cyclic compound (GO:0071407) | 0.344828497 |
| cellular response to oxygen-containing compound (GO:1901701) | 0.344828497 |
| chromatin organization (GO:0006325) | 0.344828497 |
| chromosome organization (GO:0051276) | 0.344828497 |
| endomembrane system (GO:0012505) | 0.344828497 |
| hematopoietic or lymphoid organ development (GO:0048534) | 0.344828497 |
| intracellular organelle part (GO:0044446) | 0.344828497 |
| mitochondrial envelope (GO:0005740) | 0.344828497 |
| mitochondrial membrane (GO:0031966) | 0.344828497 |
| mitochondrion (GO:0005739) | 0.344828497 |
| negative regulation of macromolecule metabolic process | 0.344828497 |
| (GO:0010605) | |
| negative regulation of metabolic process (GO:0009892) | 0.344828497 |
| negative regulation of nitrogen compound metabolic process | 0.344828497 |
| (GO:0051172) | |
| nucleoside phosphate binding (GO:1901265) | 0.344828497 |
| nucleotide binding (GO:0000166) | 0.344828497 |
| positive regulation of cellular protein metabolic process | 0.344828497 |
| (GO:0032270) | |
| positive regulation of macromolecule metabolic process | 0.344828497 |
| (GO:0010604) | |
| positive regulation of nervous system development | 0.344828497 |
| (GO:0051962) | |
| positive regulation of nucleobase-containing compound | 0.344828497 |
| metabolic process (GO:0045935) | |
| positive regulation of phosphate metabolic process | 0.344828497 |
| (GO:0045937) | |
| positive regulation of phosphorus metabolic process | 0.344828497 |
| (GO:0010562) | |
| positive regulation of protein modification process (GO:0031401) | 0.344828497 |
| positive regulation of protein phosphorylation (GO:0001934) | 0.344828497 |
| positive regulation of RNA metabolic process (GO:0051254) | 0.344828497 |
| positive regulation of transport (GO:0051050) | 0.344828497 |
| protein homodimerization activity (GO:0042803) | 0.344828497 |
| regulation of apoptotic process (GO:0042981) | 0.344828497 |
| regulation of cell death (GO:0010941) | 0.344828497 |
| regulation of phosphate metabolic process (GO:0019220) | 0.344828497 |
| regulation of phosphorus metabolic process (GO:0051174) | 0.344828497 |
| regulation of programmed cell death (GO:0043067) | 0.344828497 |
| response to lipid (GO:0033993) | 0.344828497 |
| secretion by cell (GO:0032940) | 0.344828497 |
| synapse part (GO:0044456) | 0.344828497 |
| cell morphogenesis involved in differentiation (GO:0000904) | 0.35614381 |
| cellular protein metabolic process (GO:0044267) | 0.35614381 |
| cellular protein modification process (GO:0006464) | 0.35614381 |
| cytoplasmic vesicle membrane (GO:0030659) | 0.35614381 |
| dendrite (GO:0030425) | 0.35614381 |
| dendritic tree (GO:0097447) | 0.35614381 |
| drug binding (GO:0008144) | 0.35614381 |
| endoplasmic reticulum part (GO:0044432) | 0.35614381 |
| envelope (GO:0031975) | 0.35614381 |
| Generic Transcription Pathway (R-HSA-212436) | 0.35614381 |
| identical protein binding (GO:0042802) | 0.35614381 |
| macromolecule modification (GO:0043412) | 0.35614381 |
| microtubule-based process (GO:0007017) | 0.35614381 |
| negative regulation of cellular metabolic process (GO:0031324) | 0.35614381 |
| neuron projection development (GO:0031175) | 0.35614381 |
| organelle envelope (GO:0031967) | 0.35614381 |
| organic cyclic compound biosynthetic process (GO:1901362) | 0.35614381 |
| organic cyclic compound metabolic process (GO:1901360) | 0.35614381 |
| positive regulation of protein metabolic process (GO:0051247) | 0.35614381 |
| positive regulation of signal transduction (GO:0009967) | 0.35614381 |
| protein complex oligomerization (GO:0051259) | 0.35614381 |
| protein modification process (GO:0036211) | 0.35614381 |
| regulation of phosphorylation (GO:0042325) | 0.35614381 |
| regulation of vesicle-mediated transport (GO:0060627) | 0.35614381 |
| vesicle membrane (GO:0012506) | 0.35614381 |
| biosynthetic process (GO:0009058) | 0.367371066 |
| cellular aromatic compound metabolic process (GO:0006725) | 0.367371066 |
| cellular macromolecule metabolic process (GO:0044260) | 0.367371066 |
| cellular nitrogen compound metabolic process (GO:0034641) | 0.367371066 |
| chromatin (GO:0000785) | 0.367371066 |
| cytoplasmic region (GO:0099568) | 0.367371066 |
| exocytosis (GO:0006887) | 0.367371066 |
| macromolecule biosynthetic process (GO:0009059) | 0.367371066 |
| Metabolism of proteins (R-HSA-392499) | 0.367371066 |
| microtubule cytoskeleton (GO:0015630) | 0.367371066 |
| microtubule organizing center (GO:0005815) | 0.367371066 |
| mitochondrial matrix (GO:0005759) | 0.367371066 |
| mitochondrial part (GO:0044429) | 0.367371066 |
| negative regulation of protein modification process | 0.367371066 |
| (GO:0031400) | |
| organelle organization (GO:0006996) | 0.367371066 |
| organic substance biosynthetic process (GO:1901576) | 0.367371066 |
| organic substance transport (GO:0071702) | 0.367371066 |
| positive regulation of cell differentiation (GO:0045597) | 0.367371066 |
| positive regulation of establishment of protein localization | 0.367371066 |
| (GO:1904951) | |
| positive regulation of phosphorylation (GO:0042327) | 0.367371066 |
| positive regulation of response to external stimulus | 0.367371066 |
| (GO:0032103) | |
| Post-translational protein modification (R-HSA-597592) | 0.367371066 |
| presynapse (GO:0098793) | 0.367371066 |
| purine nucleotide binding (GO:0017076) | 0.367371066 |
| purine ribonucleoside triphosphate binding (GO:0035639) | 0.367371066 |
| purine ribonucleotide binding (GO:0032555) | 0.367371066 |
| regulated exocytosis (GO:0045055) | 0.367371066 |
| regulation of innate immune response (GO:0045088) | 0.367371066 |
| regulation of intracellular signal transduction (GO:1902531) | 0.367371066 |
| regulation of protein complex assembly (GO:0043254) | 0.367371066 |
| response to hormone (GO:0009725) | 0.367371066 |
| response to peptide (GO:1901652) | 0.367371066 |
| ribonucleotide binding (GO:0032553) | 0.367371066 |
| RNA Polymerase II Transcription (R-HSA-73857) | 0.367371066 |
| cellular biosynthetic process (GO:0044249) | 0.378511623 |
| cellular catabolic process (GO:0044248) | 0.378511623 |
| cellular nitrogen compound biosynthetic process (GO:0044271) | 0.378511623 |
| cellular response to cytokine stimulus (GO:0071345) | 0.378511623 |
| cytoplasmic vesicle (GO:0031410) | 0.378511623 |
| Golgi apparatus part (GO:0044431) | 0.378511623 |
| hemopoiesis (GO:0030097) | 0.378511623 |
| intracellular vesicle (GO:0097708) | 0.378511623 |
| lipid binding (GO:0008289) | 0.378511623 |
| monocarboxylic acid metabolic process (GO:0032787) | 0.378511623 |
| negative regulation of intracellular signal transduction | 0.378511623 |
| (GO:1902532) | |
| organelle membrane (GO:0031090) | 0.378511623 |
| positive regulation of cellular component biogenesis | 0.378511623 |
| (GO:0044089) | |
| positive regulation of intracellular signal transduction | 0.378511623 |
| (GO:1902533) | |
| aromatic compound biosynthetic process (GO:0019438) | 0.389566812 |
| ATP binding (GO:0005524) | 0.389566812 |
| cell-cell junction (GO:0005911) | 0.389566812 |
| cellular macromolecule biosynthetic process (GO:0034645) | 0.389566812 |
| cellular response to lipid (GO:0071396) | 0.389566812 |
| chromosome (GO:0005694) | 0.389566812 |
| Cytokine Signaling in Immune system (R-HSA-1280215) | 0.389566812 |
| cytoplasmic vesicle part (GO:0044433) | 0.389566812 |
| Gene expression (Transcription)(R-HSA-74160) | 0.389566812 |
| heterocycle biosynthetic process (GO:0018130) | 0.389566812 |
| heterocycle metabolic process (GO:0046483) | 0.389566812 |
| lipid biosynthetic process (GO:0008610) | 0.389566812 |
| macromolecule localization (GO:0033036) | 0.389566812 |
| negative regulation of cell death (GO:0060548) | 0.389566812 |
| negative regulation of nucleic acid-templated transcription | 0.389566812 |
| (GO:1903507) | |
| negative regulation of RNA biosynthetic process (GO:1902679) | 0.389566812 |
| negative regulation of transcription by RNA polymerase II | 0.389566812 |
| (GO:0000122) | |
| nucleobase-containing small molecule metabolic process | 0.389566812 |
| (GO:0055086) | |
| nucleoside phosphate metabolic process (GO:0006753) | 0.389566812 |
| plasma membrane bounded cell projection organization | 0.389566812 |
| (GO:0120036) | |
| positive regulation of apoptotic process (GO:0043065) | 0.389566812 |
| positive regulation of molecular function (GO:0044093) | 0.389566812 |
| positive regulation of multi-organism process (GO:0043902) | 0.389566812 |
| positive regulation of protein transport (GO:0051222) | 0.389566812 |
| regulation of cellular component movement (GO:0051270) | 0.389566812 |
| regulation of cellular component organization (GO:0051128) | 0.389566812 |
| regulation of response to stress (GO:0080134) | 0.389566812 |
| ribonucleoprotein complex (GO:1990904) | 0.389566812 |
| synapse (GO:0045202) | 0.389566812 |
| vacuolar part (GO:0044437) | 0.389566812 |
| adenyl nucleotide binding (GO:0030554) | 0.40053793 |
| adenyl ribonucleotide binding (GO:0032559) | 0.40053793 |
| amide transport (GO:0042886) | 0.40053793 |
| cell junction (GO:0030054) | 0.40053793 |
| cell morphogenesis (GO:0000902) | 0.40053793 |
| cell projection organization (GO:0030030) | 0.40053793 |
| cellular component morphogenesis (GO:0032989) | 0.40053793 |
| chromatin binding (GO:0003682) | 0.40053793 |
| chromosomal part (GO:0044427) | 0.40053793 |
| cytoskeleton organization (GO:0007010) | 0.40053793 |
| endoplasmic reticulum membrane (GO:0005789) | 0.40053793 |
| gene expression (GO:0010467) | 0.40053793 |
| negative regulation of apoptotic process (GO:0043066) | 0.40053793 |
| negative regulation of biosynthetic process (GO:0009890) | 0.40053793 |
| negative regulation of programmed cell death (GO:0043069) | 0.40053793 |
| negative regulation of transcription, DNA-templated | 0.40053793 |
| (GO:0045892) | |
| nitrogen compound transport (GO:0071705) | 0.40053793 |
| nuclear envelope (GO:0005635) | 0.40053793 |
| nucleic acid metabolic process (GO:0090304) | 0.40053793 |
| nucleobase-containing compound metabolic process | 0.40053793 |
| (GO:0006139) | |
| nucleolus (GO:0005730) | 0.40053793 |
| nucleotide metabolic process (GO:0009117) | 0.40053793 |
| organophosphate metabolic process (GO:0019637) | 0.40053793 |
| peptide transport (GO:0015833) | 0.40053793 |
| perinuclear region of cytoplasm (GO:0048471) | 0.40053793 |
| positive regulation of catalytic activity (GO:0043085) | 0.40053793 |
| positive regulation of cell death (GO:0010942) | 0.40053793 |
| positive regulation of cell development (GO:0010720) | 0.40053793 |
| positive regulation of hydrolase activity (GO:0051345) | 0.40053793 |
| positive regulation of neurogenesis (GO:0050769) | 0.40053793 |
| positive regulation of programmed cell death (GO:0043068) | 0.40053793 |
| protein catabolic process (GO:0030163) | 0.40053793 |
| protein-containing complex binding (GO:0044877) | 0.40053793 |
| regulation of cytokine production (GO:0001817) | 0.40053793 |
| regulation of cytoskeleton organization (GO:0051493) | 0.40053793 |
| regulation of locomotion (GO:0040012) | 0.40053793 |
| regulation of neuron differentiation (GO:0045664) | 0.40053793 |
| regulation of peptide transport (GO:0090087) | 0.40053793 |
| response to cytokine (GO:0034097) | 0.40053793 |
| response to inorganic substance (GO:0010035) | 0.40053793 |
| RNA binding (GO:0003723) | 0.40053793 |
| vacuole (GO:0005773) | 0.40053793 |
| Vesicle-mediated transport (R-HSA-5653656) | 0.40053793 |
| apoptotic process (GO:0006915) | 0.411426246 |
| axon part (GO:0033267) | 0.411426246 |
| bounding membrane of organelle (GO:0098588) | 0.411426246 |
| cell cycle process (GO:0022402) | 0.411426246 |
| Cell Cycle, Mitotic (R-HSA-69278) | 0.411426246 |
| centrosome (GO:0005813) | 0.411426246 |
| cytoskeletal protein binding (GO:0008092) | 0.411426246 |
| DNA metabolic process (GO:0006259) | 0.411426246 |
| glutamatergic synapse (GO:0098978) | 0.411426246 |
| Golgi apparatus (GO:0005794) | 0.411426246 |
| intracellular organelle lumen (GO:0070013) | 0.411426246 |
| intrinsic component of organelle membrane (GO:0031300) | 0.411426246 |
| lysosome (GO:0005764) | 0.411426246 |
| lytic vacuole (GO:0000323) | 0.411426246 |
| macromolecule catabolic process (GO:0009057) | 0.411426246 |
| membrane-enclosed lumen (GO:0031974) | 0.411426246 |
| microtubule cytoskeleton organization (GO:0000226) | 0.411426246 |
| negative regulation of cellular biosynthetic process | 0.411426246 |
| (GO:0031327) | |
| negative regulation of gene expression (GO:0010629) | 0.411426246 |
| negative regulation of macromolecule biosynthetic process | 0.411426246 |
| (GO:0010558) | |
| negative regulation of nucleobase-containing compound | 0.411426246 |
| metabolic process (GO:0045934) | |
| negative regulation of organelle organization (GO:0010639) | 0.411426246 |
| negative regulation of phosphate metabolic process | 0.411426246 |
| (GO:0045936) | |
| negative regulation of phosphorus metabolic process | 0.411426246 |
| (GO:0010563) | |
| negative regulation of RNA metabolic process (GO:0051253) | 0.411426246 |
| neuron projection morphogenesis (GO:0048812) | 0.411426246 |
| nuclear outer membrane-endoplasmic reticulum membrane | 0.411426246 |
| network (GO:0042175) | |
| nucleobase-containing compound biosynthetic process | 0.411426246 |
| (GO:0034654) | |
| organelle lumen (GO:0043233) | 0.411426246 |
| phosphate-containing compound metabolic process | 0.411426246 |
| (GO:0006796) | |
| phosphorus metabolic process (GO:0006793) | 0.411426246 |
| positive regulation of catabolic process (GO:0009896) | 0.411426246 |
| positive regulation of cellular component movement | 0.411426246 |
| (GO:0051272) | |
| positive regulation of cytokine production (GO:0001819) | 0.411426246 |
| positive regulation of GTPase activity (GO:0043547) | 0.411426246 |
| protein localization (GO:0008104) | 0.411426246 |
| protein localization to organelle (GO:0033365) | 0.411426246 |
| protein transport (GO:0015031) | 0.411426246 |
| protein ubiquitination (GO:0016567) | 0.411426246 |
| regulation of cell motility (GO:2000145) | 0.411426246 |
| regulation of cellular component biogenesis (GO:0044087) | 0.411426246 |
| regulation of establishment of protein localization (GO:0070201) | 0.411426246 |
| regulation of protein serine/threonine kinase activity | 0.411426246 |
| (GO:0071900) | |
| regulation of transferase activity (GO:0051338) | 0.411426246 |
| RNA metabolic process (GO:0016070) | 0.411426246 |
| transferase activity (GO:0016740) | 0.411426246 |
| transmembrane receptor protein tyrosine kinase signaling | 0.411426246 |
| pathway (GO:0007169) | |
| axon (GO:0030424) | 0.422233001 |
| cellular macromolecule localization (GO:0070727) | 0.422233001 |
| cellular protein localization (GO:0034613) | 0.422233001 |
| cytosol (GO:0005829) | 0.422233001 |
| Disease (R-HSA-1643685) | 0.422233001 |
| establishment of protein localization (GO:0045184) | 0.422233001 |
| extrinsic component of membrane (GO:0019898) | 0.422233001 |
| negative regulation of cell cycle (GO:0045786) | 0.422233001 |
| negative regulation of cellular component organization | 0.422233001 |
| (GO:0051129) | |
| negative regulation of cellular macromolecule biosynthetic | 0.422233001 |
| process (GO:2000113) | |
| negative regulation of protein phosphorylation (GO:0001933) | 0.422233001 |
| nuclear chromosome (GO:0000228) | 0.422233001 |
| nuclear chromosome part (GO:0044454) | 0.422233001 |
| plasma membrane bounded cell projection morphogenesis | 0.422233001 |
| (GO:0120039) | |
| positive regulation of locomotion (GO:0040017) | 0.422233001 |
| positive regulation of response to biotic stimulus (GO:0002833) | 0.422233001 |
| protein targeting (GO:0006605) | 0.422233001 |
| regulation of actin filament-based process (GO:0032970) | 0.422233001 |
| regulation of GTPase activity (GO:0043087) | 0.422233001 |
| regulation of hemopoiesis (GO:1903706) | 0.422233001 |
| regulation of protein kinase activity (GO:0045859) | 0.422233001 |
| regulation of protein localization (GO:0032880) | 0.422233001 |
| regulation of protein transport (GO:0051223) | 0.422233001 |
| cell cycle (GO:0007049) | 0.432959407 |
| Cell Cycle (R-HSA-1640170) | 0.432959407 |
| cell projection morphogenesis (GO:0048858) | 0.432959407 |
| cell-cell signaling by wnt (GO:0198738) | 0.432959407 |
| cellular localization (GO:0051641) | 0.432959407 |
| cellular protein catabolic process (GO:0044257) | 0.432959407 |
| establishment of localization in cell (GO:0051649) | 0.432959407 |
| glycerolipid metabolic process (GO:0046486) | 0.432959407 |
| Golgi membrane (GO:0000139) | 0.432959407 |
| intracellular protein transport (GO:0006886) | 0.432959407 |
| intracellular signal transduction (GO:0035556) | 0.432959407 |
| Metabolism of lipids (R-HSA-556833) | 0.432959407 |
| nuclear chromatin (GO:0000790) | 0.432959407 |
| nuclear part (GO:0044428) | 0.432959407 |
| phospholipid metabolic process (GO:0006644) | 0.432959407 |
| positive regulation of cell cycle (GO:0045787) | 0.432959407 |
| positive regulation of cell motility (GO:2000147) | 0.432959407 |
| positive regulation of cellular catabolic process (GO:0031331) | 0.432959407 |
| postsynaptic specialization (GO:0099572) | 0.432959407 |
| protein modification by small protein conjugation (GO:0032446) | 0.432959407 |
| protein modification by small protein conjugation or removal | 0.432959407 |
| (GO:0070647) | |
| proteolysis involved in cellular protein catabolic process | 0.432959407 |
| (GO:0051603) | |
| regulation of cell projection organization (GO:0031344) | 0.432959407 |
| regulation of cellular localization (GO:0060341) | 0.432959407 |
| regulation of kinase activity (GO:0043549) | 0.432959407 |
| regulation of mitotic cell cycle (GO:0007346) | 0.432959407 |
| regulation of neuron projection development (GO:0010975) | 0.432959407 |
| regulation of plasma membrane bounded cell projection | 0.432959407 |
| organization (GO:0120035) | |
| RNA processing (GO:0006396) | 0.432959407 |
| Wnt signaling pathway (GO:0016055) | 0.432959407 |
| Axon guidance (R-HSA-422475) | 0.443606651 |
| catalytic complex (GO:1902494) | 0.443606651 |
| cell projection assembly (GO:0030031) | 0.443606651 |
| cellular macromolecule catabolic process (GO:0044265) | 0.443606651 |
| cellular response to hormone stimulus (GO:0032870) | 0.443606651 |
| Cellular responses to external stimuli (R-HSA-8953897) | 0.443606651 |
| Class I MHC mediated antigen processing & presentation (R- | 0.443606651 |
| HSA-983169) | |
| covalent chromatin modification (GO:0016569) | 0.443606651 |
| endocytic vesicle (GO:0030139) | 0.443606651 |
| integral component of organelle membrane (GO:0031301) | 0.443606651 |
| intracellular transport (GO:0046907) | 0.443606651 |
| modification-dependent protein catabolic process (GO:0019941) | 0.443606651 |
| negative regulation of phosphorylation (GO:0042326) | 0.443606651 |
| nucleic acid-templated transcription (GO:0097659) | 0.443606651 |
| phosphorylation (GO:0016310) | 0.443606651 |
| plasma membrane bounded cell projection assembly | 0.443606651 |
| (GO:0120031) | |
| positive regulation of cellular component organization | 0.443606651 |
| (GO:0051130) | |
| positive regulation of innate immune response (GO:0045089) | 0.443606651 |
| protein kinase binding (GO:0019901) | 0.443606651 |
| regulation of cell cycle phase transition (GO:1901987) | 0.443606651 |
| regulation of cell migration (GO:0030334) | 0.443606651 |
| regulation of protein catabolic process (GO:0042176) | 0.443606651 |
| RNA biosynthetic process (GO:0032774) | 0.443606651 |
| transcription, DNA-templated (GO:0006351) | 0.443606651 |
| ubiquitin-dependent protein catabolic process (GO:0006511) | 0.443606651 |
| whole membrane (GO:0098805) | 0.443606651 |
| actin cytoskeleton (GO:0015629) | 0.454175893 |
| actin filament-based process (GO:0030029) | 0.454175893 |
| enzyme activator activity (GO:0008047) | 0.454175893 |
| enzyme binding (GO:0019899) | 0.454175893 |
| interspecies interaction between organisms (GO:0044419) | 0.454175893 |
| kinase binding (GO:0019900) | 0.454175893 |
| late endosome (GO:0005770) | 0.454175893 |
| modification-dependent macromolecule catabolic process | 0.454175893 |
| (GO:0043632) | |
| negative regulation of catabolic process (GO:0009895) | 0.454175893 |
| nuclear lumen (GO:0031981) | 0.454175893 |
| organophosphate biosynthetic process (GO:0090407) | 0.454175893 |
| positive regulation of neuron differentiation (GO:0045666) | 0.454175893 |
| positive regulation of transferase activity (GO:0051347) | 0.454175893 |
| protein localization to membrane (GO:0072657) | 0.454175893 |
| regulation of apoptotic signaling pathway (GO:2001233) | 0.454175893 |
| regulation of cell adhesion (GO:0030155) | 0.454175893 |
| regulation of cell cycle (GO:0051726) | 0.454175893 |
| regulation of DNA-binding transcription factor activity | 0.454175893 |
| (GO:0051090) | |
| regulation of small GTPase mediated signal transduction | 0.454175893 |
| (GO:0051056) | |
| regulation of T cell activation (GO:0050863) | 0.454175893 |
| Signaling by Interleukins (R-HSA-449147) | 0.454175893 |
| transcription by RNA polymerase II (GO:0006366) | 0.454175893 |
| transcription coregulator activity (GO:0003712) | 0.454175893 |
| transferase activity, transferring phosphorus-containing groups | 0.454175893 |
| (GO:0016772) | |
| activation of protein kinase activity (GO:0032147) | 0.464668267 |
| Antigen processing: Ubiquitination & Proteasome degradation | 0.464668267 |
| (R-HSA-983168) | |
| cell cortex (GO:0005938) | 0.464668267 |
| cell part morphogenesis (GO:0032990) | 0.464668267 |
| Cellular responses to stress (R-HSA-2262752) | 0.464668267 |
| Metabolism of RNA (R-HSA-8953854) | 0.464668267 |
| neuron to neuron synapse (GO:0098984) | 0.464668267 |
| nuclear membrane (GO:0031965) | 0.464668267 |
| peptidyl-amino acid modification (GO:0018193) | 0.464668267 |
| posttranscriptional regulation of gene expression (GO:0010608) | 0.464668267 |
| protein phosphorylation (GO:0006468) | 0.464668267 |
| regulation of cell morphogenesis (GO:0022604) | 0.464668267 |
| regulation of cell-cell adhesion (GO:0022407) | 0.464668267 |
| regulation of leukocyte cell-cell adhesion (GO:1903037) | 0.464668267 |
| regulation of mitotic cell cycle phase transition (GO:1901990) | 0.464668267 |
| ubiguitin-protein transferase activity (GO:0004842) | 0.464668267 |
| vacuolar membrane (GO:0005774) | 0.464668267 |
| vesicle organization (GO:0016050) | 0.464668267 |
| actin cytoskeleton organization (GO:0030036) | 0.475084883 |
| cell division (GO:0051301) | 0.475084883 |
| cellular response to external stimulus (GO:0071496) | 0.475084883 |
| early endosome (GO:0005769) | 0.475084883 |
| endosome (GO:0005768) | 0.475084883 |
| glycerophospholipid metabolic process (GO:0006650) | 0.475084883 |
| histone modification (GO:0016570) | 0.475084883 |
| lytic vacuole membrane (GO:0098852) | 0.475084883 |
| negative regulation of cell cycle process (GO:0010948) | 0.475084883 |
| phosphotransferase activity, alcohol group as acceptor | 0.475084883 |
| (GO:0016773) | |
| positive regulation of cell migration (GO:0030335) | 0.475084883 |
| positive regulation of cell projection organization (GO:0031346) | 0.475084883 |
| positive regulation of protein kinase activity (GO:0045860) | 0.475084883 |
| positive regulation of proteolysis (GO:0045862) | 0.475084883 |
| protein kinase activity (GO:0004672) | 0.475084883 |
| regulation of catabolic process (GO:0009894) | 0.475084883 |
| regulation of cell cycle process (GO:0010564) | 0.475084883 |
| regulation of cellular response to stress (GO:0080135) | 0.475084883 |
| regulation of DNA metabolic process (GO:0051052) | 0.475084883 |
| regulation of intracellular transport (GO:0032386) | 0.475084883 |
| regulation of organelle organization (GO:0033043) | 0.475084883 |
| regulation of transporter activity (GO:0032409) | 0.475084883 |
| response to oxidative stress (GO:0006979) | 0.475084883 |
| supramolecular fiber organization (GO:0097435) | 0.475084883 |
| asymmetric synapse (GO:0032279) | 0.485426827 |
| cellular response to DNA damage stimulus (GO:0006974) | 0.485426827 |
| cellular response to stress (GO:0033554) | 0.485426827 |
| DNA repair (GO:0006281) | 0.485426827 |
| Golgi vesicle transport (GO:0048193) | 0.485426827 |
| kinase activity (GO:0016301) | 0.485426827 |
| lysosomal membrane (GO:0005765) | 0.485426827 |
| MAPK cascade (GO:0000165) | 0.485426827 |
| membrane region (GO:0098589) | 0.485426827 |
| mitotic cell cycle process (GO:1903047) | 0.485426827 |
| mRNA metabolic process (GO:0016071) | 0.485426827 |
| nucleoside-triphosphatase regulator activity (GO:0060589) | 0.485426827 |
| positive regulation of cell adhesion (GO:0045785) | 0.485426827 |
| positive regulation of DNA-binding transcription factor activity | 0.485426827 |
| (GO:0051091) | |
| positive regulation of neuron projection development | 0.485426827 |
| (GO:0010976) | |
| positive regulation of protein serine/threonine kinase activity | 0.485426827 |
| (GO:0071902) | |
| postsynaptic density (GO:0014069) | 0.485426827 |
| regulation of translation (GO:0006417) | 0.485426827 |
| regulation of transmembrane transporter activity (GO:0022898) | 0.485426827 |
| RNA splicing (GO:0008380) | 0.485426827 |
| transcription factor binding (GO:0008134) | 0.485426827 |
| ubiquitin-like protein transferase activity (GO:0019787) | 0.485426827 |
| autophagy (GO:0006914) | 0.495695163 |
| endosomal part (GO:0044440) | 0.495695163 |
| Golgi subcompartment (GO:0098791) | 0.495695163 |
| membrane raft (GO:0045121) | 0.495695163 |
| mitotic cell cycle (GO:0000278) | 0.495695163 |
| mRNA processing (GO:0006397) | 0.495695163 |
| negative regulation of cellular catabolic process (GO:0031330) | 0.495695163 |
| nucleoplasm (GO:0005654) | 0.495695163 |
| positive regulation of kinase activity (GO:0033674) | 0.495695163 |
| process utilizing autophagic mechanism (GO:0061919) | 0.495695163 |
| proteasome-mediated ubiquitin-dependent protein catabolic | 0.495695163 |
| process (GO:0043161) | |
| regulation of cellular catabolic process (GO:0031329) | 0.495695163 |
| RNA splicing, via transesterification reactions (GO:0000375) | 0.495695163 |
| signal transduction by protein phosphorylation (GO:0023014) | 0.495695163 |
| actin binding (GO:0003779) | 0.50589093 |
| coenzyme metabolic process (GO:0006732) | 0.50589093 |
| DNA Repair (R-HSA-73894) | 0.50589093 |
| establishment of organelle localization (GO:0051656) | 0.50589093 |
| membrane microdomain (GO:0098857) | 0.50589093 |
| mRNA splicing, via spliceosome (GO:0000398) | 0.50589093 |
| organelle outer membrane (GO:0031968) | 0.50589093 |
| organelle subcompartment (GO:0031984) | 0.50589093 |
| outer membrane (GO:0019867) | 0.50589093 |
| peptidyl-lysine modification (GO:0018205) | 0.50589093 |
| proteasomal protein catabolic process (GO:0010498) | 0.50589093 |
| regulation of cellular amide metabolic process (GO:0034248) | 0.50589093 |
| RNA splicing, via transesterification reactions with bulged | 0.50589093 |
| adenosine as nucleophile (GO:0000377) | |
| Signaling by Receptor Tyrosine Kinases (R-HSA-9006934) | 0.50589093 |
| spindle (GO:0005819) | 0.50589093 |
| trans-Golgi network (GO:0005802) | 0.50589093 |
| transcription coactivator activity (GO:0003713) | 0.50589093 |
| Transcriptional Regulation by TP53 (R-HSA-3700989) | 0.50589093 |
| cell adhesion molecule binding (GO:0050839) | 0.516015147 |
| Diseases of signal transduction (R-HSA-5663202) | 0.516015147 |
| positive regulation of cell-cell adhesion (GO:0022409) | 0.516015147 |
| positive regulation of organelle organization (GO:0010638) | 0.516015147 |
| protein polyubiquitination (GO:0000209) | 0.516015147 |
| protein serine/threonine kinase activity (GO:0004674) | 0.516015147 |
| symbiotic process (GO:0044403) | 0.516015147 |
| transferase complex (GO:1990234) | 0.516015147 |
| ubiquitin ligase complex (GO:0000151) | 0.516015147 |
| biological phase (GO:0044848) | 0.526068812 |
| cell cycle phase (GO:0022403) | 0.526068812 |
| endosome membrane (GO:0010008) | 0.526068812 |
| GTPase binding (GO:0051020) | 0.526068812 |
| Membrane Trafficking (R-HSA-199991) | 0.526068812 |
| mitotic cell cycle phase (GO:0098763) | 0.526068812 |
| organelle localization (GO:0051640) | 0.526068812 |
| phospholipid biosynthetic process (GO:0008654) | 0.526068812 |
| positive regulation of cellular protein localization (GO:1903829) | 0.526068812 |
| regulation of cellular protein localization (GO:1903827) | 0.526068812 |
| regulation of gene expression, epigenetic (GO:0040029) | 0.526068812 |
| viral process (GO:0016032) | 0.526068812 |
| DNA-binding transcription factor binding (GO:0140297) | 0.5360529 |
| glycerophospholipid biosynthetic process (GO:0046474) | 0.5360529 |
| mRNA binding (GO:0003729) | 0.5360529 |
| RNA polymerase II-specific DNA-binding transcription factor | 0.5360529 |
| binding (GO:0061629) | |
| Intracellular signaling by second messengers (R-HSA-9006925) | 0.545968369 |
| mitochondrial outer membrane (GO:0005741) | 0.545968369 |
| Neddylation (R-HSA-8951664) | 0.545968369 |
| Platelet activation, signaling and aggregation (R-HSA-76002) | 0.545968369 |
| positive regulation of leukocyte cell-cell adhesion (GO:1903039) | 0.545968369 |
| regulation of autophagy (GO:0010506) | 0.545968369 |
| anchoring junction (GO:0070161) | 0.555816155 |
| chromosomal region (GO:0098687) | 0.555816155 |
| endomembrane system organization (GO:0010256) | 0.555816155 |
| glycerolipid biosynthetic process (GO:0045017) | 0.555816155 |
| protein localization to cell periphery (GO:1990778) | 0.555816155 |
| Ras GTPase binding (GO:0017016) | 0.555816155 |
| actin filament organization (GO:0007015) | 0.565597176 |
| cadherin binding (GO:0045296) | 0.565597176 |
| cellular response to oxidative stress (GO:0034599) | 0.565597176 |
| cellular response to steroid hormone stimulus (GO:0071383) | 0.565597176 |
| molecular adaptor activity (GO:0060090) | 0.565597176 |
| nuclear speck (GO:0016607) | 0.565597176 |
| peptidyl-serine modification (GO:0018209) | 0.565597176 |
| Ras protein signal transduction (GO:0007265) | 0.565597176 |
| regulation of axonogenesis (GO:0050770) | 0.565597176 |
| regulation of cell morphogenesis involved in differentiation | 0.565597176 |
| (GO:0010769) | |
| regulation of intracellular protein transport (GO:0033157) | 0.565597176 |
| small GTPase binding (GO:0031267) | 0.565597176 |
| cell-substrate adhesion (GO:0031589) | 0.575312331 |
| DNA replication (GO:0006260) | 0.575312331 |
| double-strand break repair (GO:0006302) | 0.575312331 |
| nucleoplasm part (GO:0044451) | 0.575312331 |
| Processing of Capped Intron-Containing Pre-mRNA (R-HSA- | 0.575312331 |
| 72203) | |
| protein binding, bridging (GO:0030674) | 0.575312331 |
| regulation of leukocyte migration (GO:0002685) | 0.575312331 |
| small GTPase mediated signal transduction (GO:0007264) | 0.575312331 |
| viral life cycle (GO:0019058) | 0.575312331 |
| regulation of chromosome organization (GO:0033044) | 0.584962501 |
| adherens junction (GO:0005912) | 0.59454855 |
| mitotic cell cycle phase transition (GO:0044772) | 0.59454855 |
| nuclear body (GO:0016604) | 0.59454855 |
| positive regulation of I-kappaB kinase/NF-kappaB signaling | 0.59454855 |
| (GO:0043123) | |
| growth cone (GO:0030426) | 0.604071324 |
| protein localization to plasma membrane (GO:0072659) | 0.604071324 |
| protein stabilization (GO:0050821) | 0.604071324 |
| regulation of cell cycle G1/S phase transition (GO:1902806) | 0.604071324 |
| regulation of cytokine-mediated signaling pathway (GO:0001959) | 0.604071324 |
| regulation of mRNA catabolic process (GO:0061013) | 0.604071324 |
| regulation of mRNA metabolic process (GO:1903311) | 0.604071324 |
| regulation of mRNA stability (GO:0043488) | 0.604071324 |
| response to reactive oxygen species (GO:0000302) | 0.604071324 |
| site of polarized growth (GO:0030427) | 0.604071324 |
| ubiquitin-like protein ligase binding (GO:0044389) | 0.604071324 |
| cell cycle phase transition (GO:0044770) | 0.613531653 |
| membrane docking (GO:0022406) | 0.613531653 |
| organelle localization by membrane tethering (GO:0140056) | 0.613531653 |
| protein domain specific binding (GO:0019904) | 0.613531653 |
| regulation of I-kappaB kinase/NF-kappaB signaling | 0.613531653 |
| (GO:0043122) | |
| regulation of protein stability (GO:0031647) | 0.613531653 |
| regulation of response to cytokine stimulus (GO:0060759) | 0.613531653 |
| cell-substrate adherens junction (GO:0005924) | 0.632268215 |
| cell-substrate junction (GO:0030055) | 0.632268215 |
| focal adhesion (GO:0005925) | 0.632268215 |
| interaction with host (GO:0051701) | 0.632268215 |
| peptidyl-serine phosphorylation (GO:0018105) | 0.632268215 |
| protein C-terminus binding (GO:0008022) | 0.632268215 |
| regulation of G1/S transition of mitotic cell cycle (GO:2000045) | 0.632268215 |
| ubiquitin protein ligase binding (GO:0031625) | 0.632268215 |
| SUMO E3 ligases SUMOylate target proteins (R-HSA-3108232) | 0.641546029 |
| regulation of mRNA processing (GO:0050684) | 0.650764559 |
| cell leading edge (GO:0031252) | 0.659924558 |
| SUMOylation (R-HSA-2990846) | 0.659924558 |
| regulation of RNA splicing (GO:0043484) | 0.669026766 |
| G2/M transition of mitotic cell cycle (GO:0000086) | 0.678071905 |
| cell cycle G2/M phase transition (GO:0044839) | 0.687060688 |
| nuclear hormone receptor binding (GO:0035257) | 0.687060688 |
| stress-activated protein kinase signaling cascade (GO:0031098) | 0.695993813 |
| Golgi organization (GO:0007030) | 0.704871964 |
| positive regulation of chromosome organization (GO:2001252) | 0.704871964 |
| Clathrin-mediated endocytosis (R-HSA-8856828) | 0.722466024 |
| Death Receptor Signalling (R-HSA-73887) | 0.722466024 |
| intracellular receptor signaling pathway (GO:0030522) | 0.722466024 |
| steroid hormone mediated signaling pathway (GO:0043401) | 0.722466024 |
| chromosome, telomeric region (GO:0000781) | 0.731183242 |
| positive regulation of cell morphogenesis involved in | 0.731183242 |
| differentiation (GO:0010770) | |
| actin filament (GO:0005884) | 0.739848103 |
| lamellipodium (GO:0030027) | 0.757023247 |
| ruffle (GO:0001726) | 0.782408565 |
| Signaling by VEGF (R-HSA-194138) | 0.790772038 |
| cellular response to leukemia inhibitory factor (GO:1990830) | 0.799087306 |
| nuclear chromosome, telomeric region (GO:0000784) | 0.799087306 |
| PML body (GO:0016605) | 0.799087306 |
| response to leukemia inhibitory factor (GO:1990823) | 0.799087306 |
| regulation of telomere maintenance (GO:0032204) | 0.815575429 |
| VEGFA-VEGFR2 Pathway (R-HSA-4420097) | 0.815575429 |
| SH3 domain binding (GO:0017124) | 0.82374936 |
| regulation of cell junction assembly (GO:1901888) | 0.831877241 |
| cis-Golgi network (GO:0005801) | 0.86393845 |

TABLE 4
Direction and Tissue of Change for Genes with Significant
Alternative Splicing and Alternative Transcription Start/End

| | Alternative Transcription | | | | Alternative Splicing | | | |
|---|---|---|---|---|---|---|---|---|
| Gene | HL | ML | HB | MB | HL | ML | HB | MB |
| AACS | 0.062 | — | — | — | — | 0.058 | — | 0.426 |
| AAMDC | — | −0.149 | — | 0.070 | — | — | — | 0.369 |
| ABCB6 | — | 0.327 | — | −0.412 | — | — | — | 0.353 |
| ABCB8 | −0.007 | — | — | 0.584 | — | — | — | −0.751 |
| ABCC1 | — | — | — | −0.561 | — | — | — | 0.464 |
| ABCC2 | — | 0.340 | — | — | −0.318 | — | — | — |
| ABCG1 | — | — | — | 0.094 | — | 0.041 | — | — |
| ABHD11 | — | −0.528 | — | — | — | — | — | 0.647 |
| ABI2 | 0.000 | — | — | 0.127 | — | — | — | 0.226 |
| ABL1 | — | — | — | 0.172 | — | — | — | −0.305 |
| ABR | — | — | −0.004 | 0.299 | — | — | — | 0.073 |
| ABTB1 | — | — | — | 0.386 | — | — | — | 0.039 |
| ACAD10 | — | −0.402 | — | 0.399 | — | — | — | 0.599 |
| ACADSB | — | — | — | 0.161 | — | — | — | 0.134 |
| ACADVL | — | −0.284 | — | — | — | — | — | −0.258 |
| ACAP2 | — | −0.440 | — | −0.322 | — | 0.162 | — | — |
| ACBD5 | — | 0.178 | — | −0.530 | — | — | — | 0.591 |
| ACE | −0.190 | 0.002 | — | 0.254 | — | 0.009 | — | — |
| ACIN1 | — | 0.244 | — | 0.070 | — | — | — | 0.174 |
| ACOT7 | — | −0.071 | 0.045 | 0.030 | — | 0.024 | — | — |
| ACOX3 | — | — | — | −0.808 | — | — | — | −0.284 |
| ACSS1 | — | — | 0.025 | −0.783 | — | — | — | 0.291 |
| ACTB | — | — | −0.007 | −0.209 | — | 0.013 | — | — |
| ACTN4 | — | — | — | 0.236 | — | — | — | 0.092 |
| ACTR10 | — | — | — | 0.116 | — | — | — | 0.279 |
| ACTR1A | — | 0.039 | — | −0.015 | — | — | — | 0.072 |
| ADAL | — | −0.399 | — | — | — | 0.178 | — | — |
| ADAM17 | — | −0.192 | −0.005 | 0.135 | — | — | — | 0.240 |
| ADAM33 | — | 0.131 | — | −0.621 | — | −0.595 | — | — |
| ADAM8 | — | — | — | 0.333 | — | — | — | −0.153 |
| ADAMTS10 | — | 0.029 | — | 0.733 | — | 0.142 | — | — |
| ADARB1 | — | −0.312 | — | — | — | 0.297 | — | — |
| ADCY6 | — | 0.022 | — | −0.287 | — | — | — | 0.243 |
| ADD1 | — | 0.001 | — | −0.219 | — | 0.151 | — | — |
| ADD3 | — | — | — | 0.038 | — | — | — | −0.146 |
| ADGRE5 | — | 0.002 | — | 0.020 | — | — | — | 0.111 |
| ADK | — | 0.193 | — | −0.888 | — | 0.029 | — | — |
| ADRM1 | — | 0.108 | — | 0.390 | — | — | — | 0.293 |
| AFDN | — | — | — | −0.277 | — | 0.044 | — | — |
| AFF4 | — | — | — | −0.340 | — | — | — | 0.163 |
| AGBL2 | — | — | 0.535 | — | — | −0.237 | — | — |
| AGER | — | 0.005 | — | — | 0.003 | 0.016 | — | — |
| AGL | — | — | — | −0.655 | — | 0.454 | — | — |
| AGPAT3 | — | — | — | 0.098 | — | — | — | −0.492 |
| AGTPBP1 | — | 0.129 | — | 0.446 | — | — | — | 0.470 |
| AHCTF1 | — | — | −0.022 | 0.416 | — | — | — | 0.522 |
| AHCYL1 | — | 0.039 | — | 0.117 | — | — | — | 0.435 |
| AHCYL2 | −0.005 | −0.124 | — | 0.305 | — | 0.063 | — | 0.255 |
| AHNAK | — | −0.325 | — | 0.294 | — | −0.350 | — | 0.059 |
| AHSA1 | — | 0.184 | — | 0.348 | — | — | — | 0.067 |
| AIFM1 | — | — | 0.191 | — | — | — | — | 0.701 |
| AKAP2 | −0.001 | — | — | — | — | 0.010 | — | — |
| AKAP8 | — | — | 0.360 | 0.021 | — | — | — | 0.255 |
| AKAP8L | — | −0.041 | — | −0.301 | — | 0.112 | — | — |
| AKTIP | — | −0.457 | 0.005 | — | — | 0.065 | — | — |
| ALAS1 | — | 0.162 | — | 0.209 | — | — | — | 0.216 |
| ALDH18A1 | — | 0.247 | — | 0.563 | — | — | — | −0.556 |
| ALDH3A2 | — | — | — | −0.186 | — | 0.129 | — | — |
| ALDH3B1 | — | — | 0.003 | — | — | 0.057 | — | — |
| ALKBH6 | — | 0.417 | — | — | — | — | — | 0.496 |
| AMN1 | — | −0.235 | — | −0.584 | — | — | — | 0.505 |
| AMPD3 | −0.013 | — | — | 0.192 | — | −0.063 | — | — |
| ANAPC16 | — | −0.189 | — | 0.151 | — | — | — | 0.337 |
| ANK2 | −0.009 | −0.336 | — | — | — | 0.352 | — | — |
| ANK3 | 0.012 | 0.115 | — | −0.048 | — | 0.080 | 0.203 | 0.477 |
| ANKIB1 | −0.024 | — | — | — | — | 0.118 | — | — |
| ANKRD1 | — | 0.200 | — | — | — | −0.031 | — | — |
| ANKRD12 | — | −0.235 | — | −0.301 | — | — | — | 0.271 |
| ANKRD54 | — | 0.402 | — | — | — | — | — | 0.572 |
| ANKZF1 | — | −0.509 | 0.017 | 0.553 | — | — | — | 0.763 |
| ANO10 | — | 0.418 | — | 0.539 | — | 0.070 | — | — |
| ANTXR1 | — | 0.259 | — | — | — | 0.043 | — | — |
| ANTXR2 | — | — | — | 0.097 | — | 0.018 | — | — |
| ANXA7 | — | 0.041 | — | — | — | 0.062 | — | — |
| AP1M1 | — | — | — | 0.427 | — | — | — | 0.241 |
| AP3D1 | — | −0.140 | — | 0.082 | — | — | — | 0.272 |
| AP3M1 | — | — | — | 0.119 | — | — | — | −0.256 |
| AP4E1 | — | −0.297 | — | — | — | — | — | −0.454 |
| APEH | — | −0.096 | — | −0.275 | — | — | — | −0.222 |
| APEX2 | — | 0.184 | — | 0.083 | — | — | — | 0.845 |
| APOBEC1 | — | 0.189 | — | — | — | — | — | 0.283 |
| APOBEC3H | — | — | — | 0.142 | — | — | — | 0.211 |
| APPL2 | — | — | −0.009 | — | — | — | — | −0.792 |
| AQR | — | −0.084 | — | — | — | — | — | 0.344 |
| ARAF | — | −0.092 | — | −0.526 | — | — | — | −0.698 |
| ARAP1 | 0.017 | — | — | 0.133 | — | 0.126 | — | — |
| ARAP2 | — | — | — | −0.199 | — | — | −0.446 | — |
| ARFGAP1 | — | 0.223 | — | 0.054 | — | — | — | 0.249 |
| ARHGAP21 | — | — | — | 0.303 | — | 0.056 | — | — |
| ARHGAP25 | — | — | — | 0.017 | — | 0.176 | — | −0.185 |
| ARHGAP4 | — | — | — | −0.057 | — | 0.158 | — | 0.091 |
| ARHGEF2 | — | 0.029 | — | 0.051 | — | — | — | 0.325 |
| ARHGEF40 | — | 0.315 | — | — | — | 0.197 | — | — |
| ARID1A | −0.002 | −0.098 | — | 0.224 | — | — | — | 0.447 |
| ARID5A | — | 0.215 | — | 0.024 | — | — | — | 0.582 |
| ARL11 | — | −0.320 | — | 0.877 | — | — | — | 0.293 |
| ARL3 | — | 0.110 | — | 0.181 | — | — | — | 0.053 |
| ARMC10 | — | 0.053 | — | 0.251 | — | — | — | 0.216 |
| ARMCX3 | −0.022 | — | — | — | — | 0.260 | — | — |
| ARPC1B | — | −0.166 | — | — | — | — | — | 0.028 |
| ARRB1 | — | 0.001 | — | 0.001 | — | 0.078 | — | 0.068 |
| ARRDC1 | — | −0.385 | — | — | — | — | — | 0.103 |
| ARRDC2 | — | −0.321 | 0.167 | 0.433 | — | — | — | 0.797 |
| ARRDC3 | — | 0.085 | 0.010 | 0.265 | — | — | — | 0.347 |
| AS3MT | — | 0.323 | — | −0.367 | — | — | — | 0.121 |
| ASB1 | — | — | — | 0.372 | — | — | — | 0.034 |
| ASB3 | — | — | — | −0.764 | — | −0.150 | — | — |
| ASH2L | — | 0.177 | — | −0.552 | — | — | — | 0.279 |
| ATAD2B | — | — | — | 0.855 | — | — | — | 0.350 |
| ATAT1 | — | 0.327 | — | — | — | 0.160 | — | — |
| ATG16L1 | — | 0.681 | 0.018 | 0.034 | — | — | — | 0.702 |
| ATG2B | — | 0.262 | — | 0.610 | — | — | — | 0.295 |
| ATG4D | — | −0.240 | — | — | — | — | — | −0.332 |
| ATG7 | — | — | — | 0.019 | — | — | — | 0.282 |
| ATL3 | — | −0.399 | — | 0.282 | — | — | — | 0.327 |
| ATP11A | — | — | 0.047 | — | — | — | — | −0.197 |
| ATP11B | — | 0.325 | — | 0.417 | — | — | — | 0.067 |
| ATP13A3 | — | — | 0.014 | — | — | — | — | 0.606 |
| ATP1B2 | — | 0.128 | — | — | — | 0.114 | — | — |
| ATP2C1 | — | −0.275 | −0.125 | 0.047 | — | 0.116 | — | 0.017 |
| ATP5F1E | — | −0.276 | — | 0.396 | — | — | — | −0.126 |
| ATP5MPL | −0.004 | — | −0.028 | 0.010 | — | — | — | 0.133 |
| ATP5PB | — | 0.229 | — | — | — | — | — | 0.062 |
| ATP6AP1 | — | — | — | 0.085 | — | — | — | 0.080 |
| ATP6V1B2 | — | — | — | −0.299 | — | — | — | 0.174 |
| ATP8A1 | — | −0.284 | — | — | — | 0.054 | — | — |
| ATRAID | — | 0.652 | — | 0.324 | — | — | — | 0.410 |
| ATRIP | — | −0.237 | −0.079 | −0.272 | — | — | — | 0.815 |
| ATRX | 0.093 | — | — | 0.035 | — | −0.028 | — | — |
| ATXN2L | — | −0.582 | — | — | — | — | — | −0.163 |
| AUH | — | 0.453 | — | 0.539 | — | — | — | 0.226 |
| AUP1 | — | 0.089 | — | 0.752 | — | — | — | 0.119 |
| AZIN1 | — | — | — | 0.235 | — | 0.185 | — | — |
| B3GALNT2 | — | −0.274 | — | 0.236 | — | 0.352 | — | — |
| BAD | −0.033 | −0.167 | −0.056 | −0.203 | — | — | — | 0.225 |
| BAZ2A | 0.007 | — | — | — | — | — | — | −0.246 |
| BCAR3 | — | −0.345 | — | −0.237 | — | −0.054 | — | — |
| BCL2L1 | — | 0.008 | — | 0.031 | — | — | — | 0.117 |
| BCL9 | — | — | — | −0.126 | — | −0.375 | — | — |
| BECN1 | −0.012 | 0.323 | — | 0.024 | — | 0.089 | — | 0.019 |
| BET1L | — | — | — | −0.130 | — | — | — | 0.265 |
| BGN | — | 0.054 | — | — | — | 0.034 | — | — |
| BICD2 | — | 0.022 | — | 0.005 | — | — | — | 0.133 |
| BICDL1 | — | 0.351 | — | 0.270 | — | −0.244 | — | 0.439 |
| BIN1 | — | 0.136 | — | 0.030 | — | — | — | 0.081 |
| BIN3 | — | −0.123 | — | — | — | −0.067 | — | — |
| BIRC6 | — | — | — | 0.250 | — | — | — | 0.466 |
| BLMH | — | — | — | 0.705 | — | — | — | 0.472 |
| BMPR1B | — | — | −0.186 | — | — | −0.772 | — | — |
| BMS1 | — | 0.270 | — | — | — | — | — | 0.555 |
| BNIP3L | — | — | −0.293 | — | — | 0.064 | — | 0.345 |
| BRAT1 | — | 0.172 | — | — | — | −0.309 | — | — |
| BRCC3 | — | — | — | 0.360 | — | — | — | −0.334 |
| BRD2 | — | — | — | 0.016 | — | — | — | 0.131 |
| BRD9 | 0.009 | 0.148 | — | 0.330 | — | — | — | 0.480 |
| BSCL2 | — | — | — | 0.455 | — | 0.150 | — | — |
| BSG | — | −0.016 | — | 0.098 | — | — | — | −0.029 |
| BTBD19 | — | — | — | −0.111 | — | — | — | −0.814 |
| BTBD9 | — | 0.016 | — | −0.327 | — | 0.085 | — | — |
| BTC | — | 0.597 | — | — | — | 0.277 | — | — |
| BTF3 | — | 0.272 | — | 0.017 | — | 0.017 | — | — |
| BTLA | — | −0.047 | — | −0.242 | — | — | — | −0.339 |
| BTRC | — | — | 0.449 | −0.370 | — | 0.065 | — | 0.296 |
| C11orf1 | — | −0.225 | — | 0.290 | — | — | — | 0.073 |
| C12orf29 | — | −0.107 | — | — | — | −0.176 | — | — |
| C12orf57 | — | 0.499 | — | 0.176 | — | 0.121 | — | 0.108 |
| C16orf70 | — | — | — | 0.392 | — | — | — | 0.419 |
| C18orf21 | — | — | — | 0.164 | — | — | — | −0.230 |
| C19orf38 | — | 0.645 | — | — | — | −0.135 | — | 0.074 |
| C1orf122 | — | — | — | 0.452 | — | 0.365 | — | — |
| C1orf43 | — | 0.007 | — | 0.262 | — | — | — | −0.153 |
| C1orf61 | 0.592 | — | 0.217 | — | — | — | −0.347 | — |
| C1S | — | 0.065 | — | — | −0.649 | 0.084 | — | — |
| C20orf194 | — | 0.360 | — | −0.279 | — | 0.045 | — | — |
| C2CD2 | — | 0.030 | — | −0.161 | — | 0.192 | — | — |
| C3orf18 | −0.013 | — | 0.114 | — | — | — | −0.345 | — |
| C6orf89 | — | 0.387 | — | −0.470 | — | — | — | 0.097 |
| C8orf34 | — | 0.119 | — | — | — | 0.600 | — | — |
| C8orf82 | — | −0.191 | — | −0.402 | — | — | — | −0.289 |
| C9orf85 | — | — | — | 0.026 | — | — | — | 0.502 |
| CACNA1D | — | 0.039 | — | −0.538 | — | 0.207 | — | — |
| CACNA1E | −0.269 | — | — | — | — | — | — | 0.568 |
| CACNA2D1 | — | — | — | −0.322 | — | 0.211 | — | — |
| CADM1 | −0.010 | 0.020 | — | −0.460 | — | 0.035 | — | — |
| CALD1 | — | — | — | 0.032 | — | 0.257 | — | 0.094 |
| CALML4 | — | 0.323 | — | 0.392 | — | — | — | 0.584 |
| CAMK1 | — | 0.038 | — | — | — | 0.319 | — | — |
| CAMKK2 | — | 0.115 | — | 0.052 | — | — | — | 0.311 |
| CAMTA1 | — | −0.096 | — | 0.098 | — | −0.688 | — | — |
| CARM1 | — | −0.012 | — | 0.585 | — | 0.282 | — | — |
| CARMIL2 | 0.406 | — | — | — | — | — | — | 0.687 |
| CARS2 | — | — | — | 0.400 | — | — | — | 0.484 |
| CASC3 | — | — | — | 0.033 | — | — | — | −0.648 |
| CASC4 | −0.089 | 0.008 | — | −0.385 | — | 0.028 | — | — |
| CASP2 | −0.005 | −0.070 | 0.004 | 0.073 | — | — | — | 0.432 |
| CAV1 | −0.004 | 0.045 | — | −0.209 | — | 0.034 | — | — |
| CBX7 | — | −0.038 | — | 0.014 | — | — | — | 0.457 |
| CC2D1B | — | 0.453 | — | — | — | — | — | 0.719 |
| CCAR2 | — | — | −0.009 | — | — | — | — | 0.652 |
| CCDC107 | — | 0.030 | — | — | — | 0.103 | — | — |
| CCDC114 | — | 0.281 | — | — | — | — | 0.369 | — |
| CCDC12 | — | — | — | 0.185 | — | — | — | 0.043 |
| CCDC25 | — | 0.051 | −0.136 | — | — | — | — | 0.240 |
| CCDC33 | — | −0.426 | — | — | — | −0.686 | — | — |
| CCDC85A | — | 0.106 | — | 0.243 | — | −0.205 | — | — |
| CCDC88B | — | — | −0.069 | — | — | — | — | −0.484 |
| CCDC88C | — | −0.325 | — | — | — | 0.254 | — | — |
| CCDC9 | — | 0.096 | — | −0.730 | — | — | — | 0.350 |
| CCDC97 | — | — | — | 0.083 | — | — | — | 0.276 |
| CCNC | — | — | −0.048 | 0.141 | — | — | — | 0.493 |
| CCND3 | — | −0.446 | — | −0.011 | — | −0.114 | — | −0.060 |
| CCNG2 | −0.002 | 0.109 | — | 0.017 | — | — | — | 0.240 |
| CCNT2 | — | — | 0.039 | −0.347 | — | — | — | 0.169 |
| CCT5 | 0.021 | — | −0.547 | — | — | — | — | 0.139 |
| CD164 | −0.001 | 0.028 | — | 0.034 | — | — | — | 0.044 |
| CD200R1 | — | 0.403 | — | −0.513 | — | — | — | 0.714 |
| CD200R1L | — | 0.403 | — | −0.513 | — | — | — | 0.714 |
| CD209 | — | — | — | 0.064 | — | — | — | 0.786 |
| CD22 | — | −0.366 | — | 0.403 | — | — | — | 0.098 |
| CD226 | — | — | — | 0.069 | — | — | — | 0.029 |
| CD27 | — | 0.252 | — | −0.397 | — | — | — | −0.622 |
| CD2AP | — | 0.145 | — | −0.276 | — | — | — | 0.461 |
| CD320 | — | −0.535 | — | −0.431 | — | — | — | 0.731 |
| CD36 | — | 0.005 | — | −0.623 | −0.301 | — | — | — |
| CD44 | — | — | −0.001 | 0.030 | — | — | — | −0.038 |
| CD47 | — | 0.194 | — | 0.001 | — | 0.056 | — | 0.121 |
| CD52 | — | — | — | 0.074 | — | — | — | 0.041 |
| CD55 | −0.121 | 0.060 | — | — | 0.007 | — | — | — |
| CD59 | — | — | 0.008 | 0.382 | — | — | — | 0.044 |
| CD8A | — | — | — | −0.141 | — | — | — | 0.837 |
| CDC25B | — | — | — | −0.267 | — | — | — | 0.252 |
| CDC34 | — | — | — | 0.304 | — | — | — | 0.118 |
| CDC42BPA | — | — | — | 0.420 | — | 0.029 | — | 0.244 |
| CDCA8 | 0.152 | — | — | — | — | — | — | 0.315 |
| CDH13 | −0.016 | — | — | — | — | 0.068 | — | — |
| CDIPT | — | −0.035 | — | 0.060 | — | 0.115 | — | — |
| CDK10 | −0.009 | — | — | — | — | 0.330 | — | — |
| CDK14 | — | 0.109 | — | — | — | 0.018 | — | — |
| CDK2 | — | 0.271 | — | 0.279 | — | — | — | 0.196 |
| CDKN1A | — | −0.069 | — | −0.348 | — | −0.202 | — | — |
| CDKN2D | — | — | — | 0.028 | — | — | — | −0.067 |
| CEACAM1 | 0.019 | — | −0.044 | 0.077 | — | — | — | 0.370 |
| CEACAM3 | — | — | — | 0.077 | — | — | — | 0.370 |
| CEACAM5 | — | — | — | 0.077 | — | — | — | 0.370 |
| CEACAM6 | — | — | — | 0.077 | — | — | — | 0.370 |
| CEACAM7 | — | — | — | 0.077 | — | — | — | 0.370 |
| CEACAM8 | — | — | — | 0.077 | — | — | — | 0.370 |
| CENPC | — | 0.346 | — | 0.246 | — | 0.264 | — | 0.468 |
| CENPT | — | — | — | 0.792 | — | — | — | −0.601 |
| CEP57 | — | — | — | 0.052 | — | — | — | −0.673 |
| CEP83 | — | — | — | −0.421 | — | — | — | 0.190 |
| CEP95 | — | — | — | 0.766 | — | — | — | 0.757 |
| CEPT1 | 0.067 | — | 0.257 | — | — | — | — | −0.147 |
| CFAP20 | — | −0.075 | — | — | — | — | — | 0.570 |
| CFP | — | — | −0.010 | 0.285 | — | — | — | −0.286 |
| CGRRF1 | — | 0.279 | — | 0.274 | — | — | — | 0.763 |
| CHCHD1 | — | — | — | 0.334 | — | 0.081 | — | 0.260 |
| CHCHD2 | — | — | — | 0.195 | — | — | — | 0.028 |
| CHCHD7 | 0.008 | — | −0.016 | −0.417 | — | — | — | 0.083 |
| CHD8 | — | −0.047 | — | −0.047 | — | — | — | 0.708 |
| CHD9 | — | — | — | 0.508 | — | — | — | 0.211 |
| CHID1 | — | −0.452 | — | — | — | — | — | 0.510 |
| CHMP6 | — | 0.249 | — | 0.131 | — | — | — | 0.341 |
| CHP1 | — | −0.125 | — | −0.028 | — | — | — | 0.108 |
| CHPT1 | — | 0.175 | — | — | — | 0.025 | — | −0.659 |
| CHTF8 | — | — | — | 0.167 | — | — | — | −0.139 |
| CIC | — | 0.196 | — | — | — | — | — | 0.076 |
| CINP | — | −0.153 | — | 0.023 | — | — | — | 0.472 |
| CIR1 | 0.032 | 0.298 | 0.033 | — | — | — | — | −0.466 |
| CIRBP | — | — | 0.066 | −0.311 | — | — | — | −0.252 |
| CITED2 | — | — | — | −0.319 | — | — | — | 0.356 |
| CKAP5 | — | −0.652 | — | 0.443 | — | — | — | 0.131 |
| CKB | −0.044 | 0.016 | −0.058 | — | — | 0.092 | — | — |
| CLCN3 | — | −0.262 | — | 0.277 | — | — | — | 0.212 |
| CLCN7 | — | — | — | 0.937 | — | — | — | 0.769 |
| CLEC2D | — | — | 0.011 | — | — | 0.587 | — | — |
| CLEC4C | 0.720 | −0.111 | — | −0.455 | — | 0.257 | — | — |
| CLIP1 | −0.001 | — | — | 0.640 | — | — | — | 0.527 |
| CLK3 | — | — | −0.011 | 0.310 | — | — | — | −0.323 |
| CLK4 | — | −0.161 | — | 0.031 | — | — | — | 0.423 |
| CLTA | — | — | −0.007 | 0.112 | — | 0.016 | — | 0.028 |
| CMC2 | — | 0.057 | — | 0.479 | — | — | — | 0.452 |
| CMTM7 | — | −0.418 | — | 0.375 | — | — | — | 0.208 |
| CMTR1 | — | 0.283 | — | 0.286 | — | 0.107 | — | −0.165 |
| CNKSR2 | −0.027 | — | — | — | — | −0.609 | — | — |
| CNN3 | — | 0.030 | — | 0.322 | — | — | — | 0.422 |
| CNOT1 | 0.011 | — | 0.048 | 0.229 | — | — | — | 0.316 |
| CNOT10 | — | 0.029 | — | 0.210 | — | — | — | 0.367 |
| CNP | 0.001 | −0.332 | 0.006 | — | — | — | — | 0.099 |
| CNPY3 | — | 0.134 | — | 0.600 | — | 0.060 | — | — |
| COBL | — | 0.111 | — | — | — | 0.073 | — | — |
| COCH | −0.359 | — | −0.362 | — | — | — | 0.244 | — |
| COLEC12 | — | −0.124 | — | −0.083 | — | 0.028 | — | — |
| COMMD3 | — | — | −0.054 | −0.330 | — | — | — | 0.568 |
| COMMD4 | — | — | — | 0.443 | — | — | — | 0.290 |
| COMMD6 | — | — | — | −0.334 | — | — | — | 0.502 |
| COPS6 | — | — | — | 0.188 | — | — | — | 0.195 |
| COPS9 | — | — | −0.067 | — | — | — | — | 0.127 |
| COPZ1 | 0.017 | — | 0.026 | −0.219 | — | — | — | 0.103 |
| COQ4 | — | — | — | 0.166 | — | 0.354 | — | — |
| COX4I1 | — | 0.161 | — | — | — | 0.031 | — | — |
| COX6B1 | — | — | — | 0.008 | — | — | — | 0.005 |
| COX6B2 | — | 0.027 | — | — | — | — | — | 0.155 |
| COX7A1 | — | — | −0.269 | — | — | — | — | 0.334 |
| COX7A2L | — | — | — | 0.002 | — | — | — | 0.012 |
| CPEB3 | — | −0.062 | — | — | — | 0.572 | — | — |
| CPED1 | — | −0.222 | 0.562 | — | — | 0.034 | — | — |
| CPQ | — | 0.024 | — | −0.797 | — | 0.060 | — | — |
| CPSF3 | — | 0.165 | — | 0.120 | — | — | — | 0.420 |
| CPSF7 | −0.007 | — | 0.007 | −0.323 | — | −0.089 | — | — |
| CR1 | — | — | — | 0.388 | — | — | — | 0.387 |
| CRCP | — | — | — | 0.105 | — | — | — | 0.266 |
| CREB1 | — | −0.267 | — | — | — | — | — | 0.051 |
| CRK | — | −0.055 | — | 0.285 | — | 0.205 | — | — |
| CROCC | — | 0.617 | — | −0.856 | — | −0.462 | — | — |
| CRTC2 | — | −0.208 | — | 0.579 | — | — | — | −0.579 |
| CSDE1 | −0.016 | −0.218 | −0.005 | 0.069 | — | −0.014 | — | — |
| CSNK1G2 | — | — | — | 0.066 | — | — | — | 0.244 |
| CSPP1 | — | −0.104 | — | −0.151 | — | — | — | 0.688 |
| CTAGE1 | — | 0.104 | — | — | — | — | — | 0.236 |
| CTAGE15 | — | 0.104 | — | — | — | — | — | 0.236 |
| CTAGE4 | — | 0.104 | — | — | — | — | — | 0.236 |
| CTAGE6 | — | 0.104 | — | — | — | — | — | 0.236 |
| CTAGE8 | — | 0.104 | — | — | — | — | — | 0.236 |
| CTAGE9 | — | 0.104 | — | — | — | — | — | 0.236 |
| CTNNB1 | — | — | −0.261 | — | 0.015 | — | — | — |
| CTNND1 | — | — | — | 0.261 | — | — | — | 0.381 |
| CTSF | — | — | — | −0.072 | — | 0.179 | — | — |
| CUL9 | — | 0.263 | — | — | — | 0.319 | — | — |
| CUTA | — | — | — | 0.605 | — | 0.155 | — | 0.489 |
| CUX1 | — | — | — | 0.043 | — | — | — | 0.143 |
| CWF19L1 | — | — | — | −0.846 | — | — | — | −0.652 |
| CYB5A | — | 0.047 | — | 0.015 | — | — | — | 0.006 |
| CYBC1 | — | −0.052 | −0.010 | 0.191 | — | — | — | 0.287 |
| CYFIP1 | — | — | 0.006 | — | — | — | — | 0.436 |
| CYLD | — | 0.157 | — | −0.538 | — | −0.036 | — | 0.294 |
| CYP17A1 | −0.195 | — | −0.192 | 0.801 | — | — | −0.120 | — |
| CYP27A1 | — | — | — | −0.842 | — | — | — | 0.703 |
| CYP3A5 | −0.036 | — | −0.549 | — | — | — | 0.216 | — |
| CYP4B1 | 0.047 | — | — | — | — | −0.029 | — | — |
| CYP4F8 | — | 0.279 | — | −0.751 | — | −0.427 | | 0.780 |
| DAAM1 | — | −0.426 | — | — | — | 0.166 | — | 0.433 |
| DAB2 | — | 0.126 | — | — | — | −0.052 | — | — |
| DAG1 | −0.001 | 0.019 | — | — | — | 0.017 | — | — |
| DAZAP2 | −0.004 | — | 0.000 | 0.001 | — | — | — | 0.011 |
| DBF4 | −0.023 | 0.454 | 0.039 | 0.171 | — | — | — | 0.567 |
| DBI | — | — | −0.007 | 0.202 | — | — | — | 0.082 |
| DCAF11 | — | — | — | 0.079 | — | — | — | 0.213 |
| DCAF8 | — | 0.026 | — | −0.026 | — | — | — | 0.065 |
| DCN | — | — | — | 0.678 | — | — | −0.089 | — |
| DDX27 | — | −0.269 | — | −0.198 | — | — | — | 0.557 |
| DDX47 | — | 0.346 | −0.120 | — | — | — | — | 0.291 |
| DDX49 | — | 0.232 | — | — | — | — | — | −0.214 |
| DDX54 | — | — | — | 0.446 | — | — | — | −0.219 |
| DDX58 | — | 0.282 | — | 0.380 | — | — | — | 0.314 |
| DECR2 | — | 0.727 | — | 0.453 | — | 0.354 | — | 0.517 |
| DEF8 | — | −0.949 | — | — | — | 0.133 | — | — |
| DENND6A | — | −0.070 | — | 0.154 | — | — | — | 0.334 |
| DENND6B | — | −0.089 | — | 0.586 | — | — | — | 0.678 |
| DERA | — | — | — | 0.490 | — | — | — | 0.128 |
| DERPC | — | — | — | 0.167 | — | — | — | −0.139 |
| DGAT1 | — | — | — | −0.310 | — | — | — | −0.244 |
| DGUOK | — | −0.879 | — | — | — | 0.253 | — | 0.320 |
| DHDDS | −0.002 | 0.468 | — | 0.195 | — | 0.137 | — | — |
| DHODH | — | — | — | −0.232 | — | — | — | 0.384 |
| DHX33 | −0.003 | — | — | −0.204 | — | — | — | 0.516 |
| DHX36 | — | 0.307 | — | −0.275 | — | — | — | 0.633 |
| DIABLO | — | −0.138 | 0.685 | — | — | — | — | −0.343 |
| DIAPH3 | — | −0.383 | — | −0.112 | — | — | — | 0.216 |
| DIDO1 | — | −0.102 | — | −0.199 | — | — | — | −0.163 |
| DIXDC1 | −0.011 | 0.628 | — | — | — | 0.097 | — | — |
| DLG1 | — | — | — | 0.158 | — | — | — | 0.434 |
| DLG2 | — | −0.175 | — | — | — | — | — | 0.061 |
| DLGAP4 | — | — | — | 0.009 | — | — | — | 0.113 |
| DMKN | — | — | −0.333 | — | — | 0.212 | — | — |
| DMTN | — | 0.449 | — | 0.631 | — | 0.283 | — | — |
| DNAH8 | — | 0.102 | — | 0.180 | — | — | — | 0.801 |
| DNAJB14 | — | 0.404 | — | −0.394 | — | — | — | 0.471 |
| DNAJC11 | — | — | — | −0.537 | — | 0.262 | — | — |
| DNAJC28 | — | 0.070 | — | 0.422 | 0.284 | — | — | — |
| DNAJC5 | — | — | — | 0.238 | — | — | — | −0.199 |
| DNAJC8 | — | — | — | −0.345 | — | — | — | 0.082 |
| DNASE1L1 | — | 0.414 | — | 0.057 | — | — | — | 0.449 |
| DNM1L | −0.001 | 0.107 | 0.007 | −0.085 | — | 0.145 | — | 0.147 |
| DNMBP | — | 0.349 | — | — | — | — | — | 0.468 |
| DNMT3A | −0.676 | — | — | −0.380 | — | — | — | 0.239 |
| DOCK4 | — | 0.221 | — | — | — | 0.027 | — | — |
| DOCK7 | — | 0.296 | — | −0.057 | — | — | — | 0.753 |
| DOCK8 | — | — | −0.018 | −0.470 | — | — | — | −0.323 |
| DOCK9 | — | — | — | −0.532 | — | −0.187 | — | 0.533 |
| DOK1 | — | −0.658 | — | — | — | 0.390 | — | 0.184 |
| DOLPP1 | — | 0.277 | — | −0.163 | — | — | — | 0.263 |
| DOP1A | — | 0.327 | — | — | — | — | — | −0.606 |
| DPH2 | — | 0.334 | −0.036 | 0.596 | — | — | — | 0.478 |
| DPH5 | — | −0.095 | — | — | — | — | — | 0.637 |
| DPP8 | — | — | — | 0.219 | — | — | — | 0.356 |
| DSE | — | — | −0.008 | — | 0.276 | — | — | — |
| DST | — | — | — | 0.580 | — | — | — | 0.416 |
| DTNA | 0.356 | −0.050 | — | — | — | 0.148 | — | — |
| DUSP16 | — | 0.812 | — | — | — | 0.340 | — | — |
| DUSP22 | −0.032 | — | — | — | — | — | — | −0.333 |
| DYNC1I2 | −0.052 | 0.233 | — | 0.324 | — | — | — | 0.057 |
| DYNC1LI2 | — | 0.288 | — | 0.461 | — | 0.088 | — | — |
| DYRK4 | — | — | −0.056 | — | — | — | −0.149 | — |
| E2F6 | — | 0.132 | — | — | — | — | — | 0.728 |
| EBPL | — | — | — | 0.733 | — | 0.294 | — | 0.620 |
| ECD | — | 0.128 | — | — | — | — | — | 0.794 |
| ECHS1 | — | −0.147 | — | — | — | — | — | 0.220 |
| ECI1 | — | −0.256 | — | −0.554 | — | 0.048 | — | — |
| ECT2 | — | — | — | 0.273 | — | 0.353 | — | — |
| EDEM3 | — | — | 0.026 | 0.268 | — | 0.135 | — | — |
| EEA1 | — | −0.162 | — | −0.382 | — | — | — | 0.361 |
| EEF1D | −0.014 | — | −0.007 | — | — | — | — | 0.040 |
| EEF1G | −0.041 | — | — | — | — | — | — | 0.061 |
| EFEMP2 | — | −0.252 | — | — | — | 0.193 | — | — |
| EGFL7 | — | 0.002 | — | — | −0.015 | — | — | — |
| EGFLAM | — | 0.345 | −0.256 | — | — | 0.066 | — | — |
| EHBP1L1 | — | 0.440 | — | 0.290 | — | — | — | 0.278 |
| EHMT2 | — | 0.193 | — | — | — | — | — | 0.204 |
| EI24 | −0.012 | 0.219 | — | — | — | — | — | −0.209 |
| EIF3A | — | — | — | 0.377 | — | — | — | 0.211 |
| EIF4A2 | −0.002 | −0.104 | — | 0.098 | — | 0.134 | — | 0.085 |
| EIF4B | −0.001 | — | — | — | — | — | — | 0.262 |
| EIF4G1 | — | — | −0.015 | — | — | — | — | 0.131 |
| EIF4G3 | — | — | — | 0.304 | — | 0.027 | — | — |
| ELOB | — | — | — | 0.048 | — | 0.056 | — | — |
| ELOC | — | — | — | 0.119 | — | — | — | 0.222 |
| ELOF1 | — | −0.267 | — | 0.153 | — | — | — | 0.233 |
| ELP3 | — | 0.327 | — | 0.324 | — | — | — | 0.413 |
| EMC1 | — | — | — | −0.352 | — | — | — | −0.184 |
| EMILIN2 | — | −0.009 | — | 0.439 | — | −0.077 | — | — |
| EML1 | — | 0.088 | — | — | — | 0.065 | — | — |
| EML2 | — | 0.314 | — | 0.770 | — | — | — | 0.420 |
| EMSY | — | — | — | 0.179 | — | 0.261 | — | — |
| ENTPD4 | 0.004 | — | — | −0.122 | — | — | — | 0.557 |
| ENTR1 | — | — | — | 0.026 | — | — | — | 0.299 |
| ENY2 | — | 0.011 | — | −0.156 | — | — | — | −0.251 |
| EP300 | −0.008 | — | — | 0.250 | — | — | — | 0.199 |
| EP400 | — | 0.414 | — | 0.598 | — | — | — | 0.697 |
| EPB41 | 0.010 | 0.036 | 0.021 | 0.038 | — | −0.073 | — | — |
| EPB41L2 | — | — | — | 0.067 | — | 0.091 | — | — |
| EPN1 | — | — | — | 0.307 | — | — | — | −0.209 |
| EPRS | — | — | — | 0.653 | — | — | — | 0.286 |
| ERCC2 | — | — | — | 0.165 | — | — | — | 0.414 |
| ERG | — | 0.165 | — | −0.075 | — | — | 0.507 | — |
| ERGIC1 | — | — | — | 0.008 | — | — | — | 0.132 |
| ERLIN2 | −0.107 | — | — | — | — | — | — | −0.302 |
| ESD | — | — | — | 0.033 | — | 0.246 | — | — |
| ESPL1 | — | −0.404 | — | — | — | 0.260 | — | — |
| ESPN | 0.240 | — | — | −0.778 | — | −0.336 | — | — |
| ESR2 | −0.430 | 0.370 | −0.272 | 0.247 | 0.459 | — | — | — |
| ESYT1 | −0.013 | — | −0.030 | — | — | 0.151 | — | 0.198 |
| ESYT2 | — | — | — | 0.904 | — | — | — | 0.399 |
| ETV3 | — | −0.310 | — | −0.160 | — | — | — | 0.470 |
| ETV5 | — | — | — | −0.936 | — | 0.030 | — | — |
| EXD2 | — | — | — | 0.398 | — | — | — | 0.161 |
| EXOC6B | — | 0.090 | — | −0.190 | — | — | — | 0.803 |
| EXOC7 | 0.009 | — | — | — | — | −0.083 | — | 0.510 |
| EXOSC10 | — | 0.141 | — | — | — | — | — | 0.446 |
| EXOSC5 | — | — | — | −0.223 | — | — | — | −0.256 |
| EXOSC8 | — | 0.738 | — | −0.438 | — | — | — | 0.542 |
| EXOSC9 | — | — | — | 0.208 | — | — | — | 0.514 |
| EYA1 | — | 0.491 | — | −0.393 | — | — | — | 0.725 |
| EZH1 | — | −0.072 | −0.004 | — | — | — | — | 0.307 |
| FADS2 | — | — | — | 0.243 | — | 0.073 | — | — |
| FAIM | — | — | — | 0.499 | — | 0.092 | — | — |
| FAIM2 | — | −0.399 | — | — | — | 0.215 | — | — |
| FAM126A | — | 0.308 | — | −0.395 | — | — | — | 0.032 |
| FAM133B | — | — | — | 0.253 | — | — | — | 0.431 |
| FAM13B | — | 0.149 | — | 0.028 | — | 0.058 | — | 0.045 |
| FAM149B1 | — | 0.055 | — | −0.153 | — | — | — | 0.459 |
| FAM156A | — | 0.456 | — | 0.140 | 0.369 | — | — | −0.388 |
| FAM156B | — | 0.456 | — | 0.140 | — | — | — | −0.388 |
| FAM172A | −0.002 | — | — | 0.083 | — | 0.148 | — | — |
| FAM173A | — | 0.604 | — | −0.270 | — | — | — | −0.193 |
| FAM189B | — | 0.045 | — | — | — | — | — | 0.487 |
| FAM192A | — | 0.260 | — | — | — | 0.073 | — | −0.208 |
| FAM204A | — | — | — | 0.043 | — | — | — | −0.261 |
| FAM214B | −0.325 | −0.082 | — | — | — | — | — | 0.277 |
| FAM227A | — | −0.224 | — | — | — | 0.596 | — | — |
| FAM45A | — | −0.119 | — | — | — | — | — | 0.177 |
| FAM47E- | −0.054 | — | — | — | — | −0.400 | — | — |
| STBD1 | | | | | | | | |
| FAM53B | — | 0.006 | — | 0.112 | — | — | — | 0.049 |
| FAM86B1 | — | — | −0.227 | — | — | — | — | −0.356 |
| FAM86C1 | −0.202 | — | 0.086 | — | — | — | — | −0.356 |
| FAM91A1 | 0.009 | — | — | — | — | — | — | 0.307 |
| FAS | −0.001 | 0.455 | 0.012 | — | — | — | — | 0.464 |
| FASTK | — | — | — | 0.075 | — | — | — | 0.285 |
| FAU | — | −0.159 | — | 0.116 | — | — | — | 0.025 |
| FBLIM1 | −0.001 | — | — | — | — | 0.089 | — | — |
| FBXL2 | — | −0.016 | — | — | — | — | — | 0.026 |
| FBXL4 | — | −0.057 | — | — | — | — | — | 0.753 |
| FBXO24 | 0.365 | — | −0.707 | — | 0.597 | — | — | — |
| FBXW10 | 0.316 | 0.469 | — | — | 0.621 | — | — | — |
| FBXW2 | — | 0.034 | — | 0.271 | — | — | — | 0.055 |
| FBXW7 | — | 0.177 | — | — | — | — | — | 0.721 |
| FCGR2A | — | −0.004 | — | 0.125 | — | −0.274 | — | — |
| FCGR2B | — | −0.004 | — | 0.125 | — | −0.274 | — | — |
| FCGR2C | — | −0.004 | — | 0.125 | — | −0.274 | — | — |
| FCHSD2 | −0.001 | 0.075 | — | −0.322 | — | — | — | 0.077 |
| FCRL1 | — | −0.554 | — | 0.062 | — | — | — | 0.299 |
| FDX1 | — | 0.391 | — | — | — | 0.078 | — | — |
| FECH | — | −0.106 | — | −0.007 | — | — | — | 0.025 |
| FERMT3 | — | — | — | 0.010 | — | −0.029 | — | 0.006 |
| FEZ2 | — | 0.199 | 0.039 | 0.184 | — | — | — | 0.518 |
| FGF11 | — | 0.350 | — | — | 0.399 | — | — | — |
| FGFR1OP2 | — | 0.028 | — | — | — | — | — | 0.483 |
| FGGY | — | — | −0.258 | — | — | −0.131 | — | — |
| FHL1 | — | 0.018 | — | 0.003 | — | 0.336 | — | 0.004 |
| FKBP11 | — | — | −0.052 | — | — | 0.233 | — | — |
| FKBP4 | — | — | — | 0.472 | — | — | — | 0.228 |
| FKBP5 | — | −0.293 | — | 0.611 | — | −0.104 | — | — |
| FLNA | — | −0.358 | 0.007 | 0.339 | — | — | — | −0.009 |
| FMC1-LUC7L2 | — | 0.003 | — | 0.111 | — | 0.091 | — | — |
| FMO1 | — | −0.278 | — | — | — | 0.093 | — | — |
| FN1 | — | −0.134 | — | 0.342 | — | — | — | −0.212 |
| FNBP1 | — | — | — | −0.498 | — | — | — | 0.137 |
| FOXO3 | — | — | 0.001 | 0.087 | — | 0.104 | — | — |
| FOXP4 | — | 0.246 | — | 0.343 | — | −0.227 | — | — |
| FOXRED1 | — | 0.232 | — | — | — | — | — | 0.303 |
| FSIP1 | — | −0.296 | — | — | — | 0.373 | — | — |
| FTL | — | — | — | 0.218 | — | 0.027 | — | — |
| FUT8 | — | — | −0.457 | 0.099 | — | — | — | 0.362 |
| FUZ | — | 0.091 | — | — | — | — | — | −0.391 |
| FXYD1 | −0.041 | 0.051 | — | — | — | 0.092 | — | — |
| FYB1 | −0.004 | −0.017 | — | 0.013 | — | — | — | 0.033 |
| FYTTD1 | — | −0.215 | — | −0.284 | — | 0.043 | — | — |
| G3BP2 | −0.006 | — | — | 0.242 | — | — | — | 0.391 |
| GAB1 | — | — | — | 0.314 | — | −0.087 | — | — |
| GABBR1 | — | 0.675 | — | −0.287 | — | 0.123 | — | — |
| GABPB2 | — | 0.149 | — | — | — | — | — | 0.385 |
| GANC | — | −0.195 | — | 0.085 | — | — | — | 0.512 |
| GAPVD1 | 0.003 | — | — | — | — | — | — | 0.211 |
| GATD1 | — | −0.340 | — | −0.140 | — | — | — | 0.225 |
| GBF1 | — | 0.158 | — | 0.245 | — | — | — | 0.742 |
| GBP6 | — | 0.041 | — | 0.342 | — | 0.611 | — | — |
| GCC2 | — | 0.476 | — | 0.320 | — | — | — | 0.185 |
| GCNT1 | — | 0.036 | — | 0.121 | — | — | — | 0.084 |
| GDA | — | 0.011 | — | −0.202 | — | −0.056 | — | −0.150 |
| GDI1 | — | 0.135 | — | −0.358 | — | 0.068 | — | — |
| GDI2 | — | — | — | 0.036 | — | — | — | 0.026 |
| GDPD2 | 0.332 | — | — | — | — | −0.108 | — | — |
| GEN1 | — | 0.290 | — | −0.435 | — | — | — | 0.743 |
| GGA2 | — | — | — | −0.284 | — | — | — | 0.277 |
| GGCT | — | — | — | 0.367 | — | — | — | 0.627 |
| GGPS1 | — | 0.051 | — | — | — | 0.065 | — | — |
| GGT5 | — | 0.278 | — | −0.425 | — | 0.474 | −0.227 | 0.650 |
| GHR | −0.044 | — | — | −0.437 | — | 0.037 | — | — |
| GIGYF2 | — | 0.071 | — | −0.518 | — | — | — | 0.406 |
| GJA1 | — | −0.176 | −0.059 | 0.243 | — | 0.042 | — | — |
| GK | — | −0.106 | — | 0.368 | — | 0.127 | — | 0.072 |
| GK3P | — | −0.106 | — | 0.368 | — | 0.127 | — | 0.072 |
| GLG1 | — | — | — | 0.199 | — | 0.087 | — | — |
| GLO1 | — | 0.048 | — | −0.105 | — | — | — | −0.064 |
| GLOD4 | −0.003 | — | — | −0.333 | — | 0.056 | — | — |
| GLT8D1 | — | 0.393 | −0.238 | — | — | — | — | 0.618 |
| GLYR1 | — | — | — | −0.233 | — | 0.190 | — | — |
| GMFB | — | — | — | 0.009 | — | — | — | 0.384 |
| GMPR2 | −0.017 | — | — | 0.091 | — | — | — | 0.197 |
| GNAS | — | −0.462 | — | 0.273 | — | — | — | −0.088 |
| GNB4 | — | — | −0.018 | — | — | 0.114 | — | — |
| GNG5 | — | −0.059 | — | — | — | — | — | 0.122 |
| GNPDA2 | — | — | — | 0.072 | — | — | — | 0.270 |
| GOLGA1 | — | 0.063 | — | 0.521 | — | — | — | 0.469 |
| GOLGA2 | — | — | — | −0.554 | — | — | — | 0.280 |
| GOLGA3 | — | 0.200 | — | — | — | — | — | 0.229 |
| GOLGA4 | — | −0.171 | — | −0.297 | — | — | — | 0.622 |
| GOLGA6A | — | — | — | −0.554 | — | — | — | 0.280 |
| GOLGA6B | — | — | — | −0.554 | — | — | — | 0.280 |
| GOLGA6C | — | — | — | −0.554 | — | — | — | 0.280 |
| GOLGA6D | — | — | — | −0.554 | — | — | — | 0.280 |
| GOLGA7 | — | — | — | 0.120 | — | — | — | 0.320 |
| GOLGA8A | −0.004 | — | — | −0.554 | — | — | — | 0.280 |
| GOLGA8B | — | — | — | −0.554 | — | — | — | 0.280 |
| GOLGA8F | — | — | — | −0.554 | — | — | — | 0.280 |
| GOLGA8G | — | — | −0.110 | −0.554 | — | — | — | 0.280 |
| GOLGA8H | — | — | — | −0.554 | — | — | — | 0.280 |
| GOLGA8J | — | — | — | −0.554 | — | — | — | 0.280 |
| GOLGA8K | — | — | — | −0.554 | — | — | — | 0.280 |
| GOLGA8M | — | — | — | −0.554 | — | — | — | 0.280 |
| GOLGA8N | — | — | — | −0.554 | — | — | — | 0.280 |
| GOLGA8O | — | — | — | −0.554 | — | — | — | 0.280 |
| GOLGA8Q | — | — | — | −0.554 | — | — | — | 0.280 |
| GOLGA8R | — | — | — | −0.554 | — | — | — | 0.280 |
| GOLGA8S | — | — | — | −0.554 | — | — | — | 0.280 |
| GOLGA8T | — | — | — | −0.554 | — | — | — | 0.280 |
| GOLPH3 | — | — | — | 0.022 | — | — | — | 0.234 |
| GOPC | — | — | — | 0.332 | — | −0.153 | — | — |
| GORAB | — | — | — | 0.313 | — | −0.351 | — | — |
| GPATCH2 | — | — | — | 0.117 | — | — | — | −0.115 |
| GPATCH2L | — | — | — | −0.213 | — | — | — | 0.365 |
| GPHN | 0.029 | −0.496 | — | — | — | 0.081 | — | — |
| GPR35 | — | 0.154 | — | −0.185 | — | −0.352 | — | −0.593 |
| GPRASP1 | — | 0.310 | — | −0.283 | — | 0.545 | — | — |
| GPS1 | −0.047 | 0.502 | — | −0.596 | — | — | — | 0.668 |
| GPT | — | 0.187 | −0.254 | — | — | — | — | 0.588 |
| GPX2 | — | −0.487 | — | — | — | — | −0.062 | — |
| GRAMD2B | — | — | — | 0.125 | — | — | — | 0.397 |
| GRAMD4 | — | 0.687 | — | −0.569 | — | — | — | 0.725 |
| GRAP2 | — | −0.154 | — | 0.191 | — | −0.112 | — | — |
| GRB10 | −0.002 | — | — | — | — | 0.044 | — | — |
| GRK2 | — | 0.110 | — | 0.205 | — | — | — | 0.091 |
| GRK3 | — | 0.001 | — | 0.199 | — | — | — | 0.485 |
| GRPEL2 | — | — | — | 0.321 | — | — | — | 0.665 |
| GRSF1 | — | — | — | −0.277 | — | — | — | 0.363 |
| GSK3B | −0.005 | — | — | — | — | — | — | 0.064 |
| GSTP1 | — | −0.129 | — | −0.274 | — | — | — | 0.173 |
| GSTZ1 | — | — | −0.027 | −0.578 | — | — | — | 0.782 |
| GTF2A2 | — | — | — | −0.304 | — | — | — | 0.365 |
| GTF2I | — | 0.003 | — | 0.642 | — | 0.031 | — | 0.730 |
| GTPBP4 | — | — | — | −0.609 | — | — | — | 0.519 |
| GYG1 | — | −0.458 | — | 0.229 | — | 0.023 | — | — |
| H2AFZ | — | — | — | 0.117 | — | — | — | 0.133 |
| HAAO | — | — | — | 0.548 | — | — | — | 0.502 |
| HADH | −0.009 | — | 0.054 | — | — | — | — | 0.528 |
| HADHA | −0.003 | — | −0.008 | 0.086 | — | 0.035 | — | 0.214 |
| HAUS4 | — | 0.652 | — | — | — | 0.197 | — | 0.483 |
| HBA2 | — | — | 0.000 | — | — | — | 0.002 | — |
| HDAC1 | — | — | — | 0.185 | — | — | — | 0.286 |
| HDAC10 | — | −0.537 | — | 0.851 | — | — | — | 0.631 |
| HDAC7 | — | −0.005 | — | −0.182 | — | 0.077 | — | — |
| HDAC8 | −0.013 | — | 0.134 | — | — | 0.196 | — | — |
| HDDC2 | — | — | 0.162 | −0.662 | — | 0.557 | — | — |
| HEATR6 | — | 0.219 | — | −0.843 | — | — | — | 0.708 |
| HEATR9 | — | — | 0.404 | — | 0.366 | — | — | — |
| HERC4 | — | 0.192 | — | 0.275 | — | −0.185 | — | — |
| HES6 | — | 0.386 | — | −0.767 | — | 0.135 | — | −0.849 |
| HGFAC | — | −0.588 | — | — | — | 0.568 | — | — |
| HIC1 | — | — | −0.022 | 0.351 | — | −0.176 | — | — |
| HINT1 | — | — | — | −0.245 | — | — | — | 0.075 |
| HIPK1 | — | — | — | 0.031 | — | −0.017 | — | 0.050 |
| HIVEP2 | — | — | — | 0.343 | — | −0.328 | — | 0.840 |
| HIVEP3 | — | 0.125 | — | −0.103 | — | 0.513 | — | — |
| HK3 | — | — | — | 0.698 | — | — | — | 0.330 |
| HLA-DMA | — | 0.035 | — | 0.087 | — | 0.103 | — | 0.154 |
| HLA-DMB | — | −0.258 | — | 0.453 | — | — | — | 0.093 |
| HLA-DOB | 0.202 | 0.464 | — | 0.486 | — | — | — | 0.261 |
| HLA-DQB1 | — | −0.017 | — | 0.413 | — | 0.046 | — | — |
| HLA-DQB2 | — | −0.017 | — | 0.413 | — | 0.046 | — | — |
| HMBOX1 | −0.036 | 0.280 | — | — | — | — | — | −0.631 |
| HMBS | −0.014 | −0.293 | 0.026 | 0.602 | — | — | — | 0.310 |
| HMGA1 | — | −0.350 | −0.002 | 0.080 | — | −0.046 | — | — |
| HMGN2 | −0.002 | — | — | 0.034 | — | — | — | 0.311 |
| HMGN3 | — | — | — | −0.405 | — | — | — | 0.539 |
| HMGN4 | — | — | — | 0.034 | — | — | — | 0.311 |
| HNRNPA2B1 | — | −0.012 | 0.043 | 0.036 | — | −0.506 | — | — |
| HNRNPK | — | — | — | 0.006 | — | — | — | 0.033 |
| HNRNPL | — | — | — | 0.033 | — | — | — | 0.051 |
| HNRNPR | −0.002 | 0.094 | — | 0.411 | — | — | — | −0.164 |
| HOMEZ | — | — | — | 0.286 | — | — | — | −0.446 |
| HOOK3 | — | — | — | −0.149 | — | — | — | 0.177 |
| HPS3 | — | −0.096 | — | −0.555 | — | — | — | 0.223 |
| HPS5 | — | 0.039 | — | −0.319 | — | — | — | 0.652 |
| HRAS | — | — | — | 0.338 | — | 0.103 | — | — |
| HSBP1 | — | 0.003 | −0.058 | — | — | 0.010 | — | 0.015 |
| HSF1 | — | 0.011 | — | −0.210 | — | — | — | −0.280 |
| HSP90AA1 | — | — | — | 0.059 | — | — | — | 0.039 |
| HSP90B1 | — | — | — | 0.103 | — | — | — | 0.404 |
| HSPB1 | — | −0.081 | — | −0.271 | — | −0.035 | — | — |
| HYOU1 | — | −0.017 | — | — | — | — | — | 0.333 |
| IDH3A | — | — | — | 0.216 | — | — | — | −0.206 |
| IFI16 | — | 0.287 | — | −0.531 | — | — | — | 0.685 |
| IFRD1 | — | −0.330 | — | 0.414 | — | — | — | 0.194 |
| IFT46 | — | −0.088 | — | 0.261 | — | — | — | −0.363 |
| IGF2BP2 | — | 0.044 | — | 0.558 | — | −0.157 | — | — |
| IL12A | — | — | — | −0.416 | — | — | — | 0.476 |
| IL15 | — | — | — | 0.484 | — | — | — | 0.638 |
| IL16 | — | −0.526 | — | 0.188 | — | 0.337 | — | — |
| IL17RA | — | — | — | 0.096 | — | — | — | 0.382 |
| IL1R1 | — | — | — | −0.830 | — | −0.554 | — | — |
| IL27RA | — | — | — | 0.318 | — | — | — | 0.196 |
| IL33 | — | −0.225 | — | — | — | 0.071 | — | — |
| IL4R | — | −0.042 | −0.003 | — | — | −0.022 | — | — |
| ILF3 | — | 0.243 | — | −0.577 | — | — | — | 0.747 |
| ILK | — | 0.158 | — | 0.231 | — | — | — | 0.037 |
| ILVBL | — | 0.390 | — | 0.831 | — | — | — | 0.352 |
| IMMT | — | −0.450 | — | 0.322 | — | — | — | 0.338 |
| IMPA1 | −0.015 | — | — | −0.112 | — | 0.122 | — | −0.134 |
| IMPDH1 | — | — | — | −0.583 | — | 0.031 | — | — |
| ING4 | — | −0.056 | — | 0.297 | — | — | — | 0.247 |
| INPP5D | — | 0.060 | — | 0.165 | — | — | — | 0.066 |
| INPP5E | — | −0.053 | — | 0.249 | — | 0.238 | — | — |
| INPP5F | — | 0.169 | −0.254 | — | — | — | — | 0.787 |
| INSR | — | — | — | −0.267 | — | — | — | 0.661 |
| INTS10 | 0.070 | — | — | — | — | −0.237 | — | 0.441 |
| INTS2 | — | −0.390 | — | — | — | — | — | 0.597 |
| INVS | — | — | — | 0.644 | — | 0.068 | — | — |
| IP6K2 | — | 0.294 | — | −0.249 | — | — | — | 0.356 |
| IPMK | — | — | — | 0.283 | — | — | — | 0.666 |
| IQCC | — | 0.817 | — | — | — | — | — | 0.347 |
| IRAK1 | 0.083 | 0.329 | — | 0.307 | — | 0.299 | — | — |
| IRF2 | — | — | — | 0.081 | — | 0.022 | — | 0.090 |
| IRF7 | — | 0.054 | −0.007 | −0.845 | — | — | — | −0.139 |
| IRF9 | — | −0.418 | — | 0.327 | — | 0.308 | — | 0.426 |
| ISG20 | — | — | — | 0.024 | — | — | — | 0.019 |
| ITCH | — | −0.160 | — | 0.075 | — | — | — | 0.277 |
| ITFG2 | — | −0.081 | — | — | — | — | — | 0.552 |
| ITGA1 | — | 0.006 | — | — | — | 0.017 | — | — |
| ITGA4 | 0.068 | — | — | — | — | — | — | 0.042 |
| ITGA6 | −0.003 | −0.069 | — | — | — | 0.105 | — | 0.008 |
| ITGA8 | — | 0.041 | — | −0.877 | — | 0.025 | — | — |
| ITGB1 | −0.002 | — | — | 0.221 | — | 0.024 | — | 0.008 |
| ITGB1BP1 | — | 0.117 | — | — | — | — | — | 0.177 |
| ITGB3BP | — | −0.531 | — | — | — | — | — | −0.625 |
| ITGB5 | — | 0.616 | — | −0.234 | — | 0.072 | — | 0.029 |
| ITK | — | −0.429 | — | — | — | 0.380 | — | — |
| ITPR2 | — | −0.264 | — | 0.047 | — | 0.037 | — | 0.083 |
| ITSN1 | — | 0.317 | −0.292 | −0.087 | — | 0.185 | — | 0.438 |
| JAG2 | — | −0.296 | — | — | — | 0.283 | — | — |
| JARID2 | — | 0.170 | — | −0.471 | — | — | — | 0.033 |
| JKAMP | — | — | — | −0.251 | — | — | — | 0.765 |
| JMJD6 | — | −0.103 | — | −0.612 | — | — | — | 0.397 |
| JMJD8 | — | — | −0.028 | — | — | 0.389 | — | — |
| KANSL1 | −0.001 | 0.274 | — | 0.420 | — | — | — | 0.320 |
| KAT5 | — | — | — | −0.487 | — | — | — | 0.345 |
| KAT6B | 0.002 | 0.111 | −0.300 | 0.644 | — | — | — | 0.591 |
| KCNAB2 | 0.356 | −0.111 | −0.270 | 0.111 | — | — | — | −0.168 |
| KCNN4 | — | 0.367 | — | — | — | — | — | 0.366 |
| KCNQ1 | — | 0.064 | — | — | — | 0.238 | — | — |
| KCNQ5 | — | −0.184 | — | — | — | — | −0.371 | — |
| KCNT1 | — | −0.075 | — | — | — | −0.257 | — | — |
| KCTD2 | −0.001 | — | — | — | — | 0.448 | — | — |
| KDM2A | — | 0.113 | −0.005 | −0.555 | — | — | — | 0.091 |
| KDM2B | — | — | — | 0.244 | — | 0.269 | — | — |
| KDM3B | — | −0.167 | — | 0.397 | — | — | — | 0.094 |
| KEAP1 | — | 0.076 | — | 0.288 | — | — | — | 0.402 |
| KHNYN | −0.001 | — | 0.009 | −0.279 | — | 0.145 | — | — |
| KIAA0040 | 0.010 | — | 0.004 | 0.606 | — | 0.134 | — | — |
| KIAA0513 | 0.012 | — | — | — | — | 0.041 | — | — |
| KIAA1109 | — | 0.826 | — | 0.334 | — | — | — | 0.731 |
| KIAA1211 | — | −0.314 | — | 0.104 | — | — | — | 0.176 |
| KIF24 | — | — | — | −0.682 | — | 0.165 | — | — |
| KLC2 | — | — | 0.058 | 0.628 | — | 0.263 | — | — |
| KLHDC10 | — | — | — | −0.291 | — | — | — | 0.565 |
| KLHDC2 | — | −0.765 | | −0.264 | — | — | — | 0.312 |
| KLHL12 | — | 0.108 | −0.061 | 0.329 | — | — | — | −0.229 |
| KLHL13 | — | −0.246 | — | — | — | 0.146 | — | — |
| KLHL20 | — | −0.238 | — | — | — | — | — | 0.288 |
| KLHL5 | — | −0.263 | — | 0.036 | — | 0.083 | — | 0.503 |
| KLRB1 | — | — | — | 0.348 | — | −0.462 | — | — |
| KLRC1 | — | — | — | 0.294 | — | — | — | 0.464 |
| KLRC2 | — | — | — | 0.294 | — | — | — | 0.464 |
| KLRC3 | — | — | — | 0.294 | — | — | — | 0.464 |
| KLRC4 | — | — | — | 0.294 | — | — | — | 0.464 |
| KLRC4-KLRK1 | — | — | — | 0.294 | — | — | — | 0.464 |
| KMT5A | — | — | — | 0.148 | — | — | — | −0.659 |
| KNDC1 | — | 0.244 | — | — | — | −0.098 | — | — |
| KPNA3 | — | — | −0.018 | — | — | — | — | 0.641 |
| KPTN | — | 0.291 | — | −0.382 | — | −0.260 | — | — |
| KRI1 | — | 0.118 | −0.030 | — | — | — | — | −0.344 |
| KRIT1 | −0.014 | — | −0.192 | 0.788 | — | — | — | 0.269 |
| KTN1 | — | — | — | 0.459 | — | — | — | 0.518 |
| L3MBTL3 | — | 0.315 | — | −0.796 | — | 0.250 | — | 0.787 |
| LAPTM5 | — | — | — | 0.010 | — | — | — | 0.010 |
| LARP4 | — | −0.003 | — | −0.260 | — | — | — | 0.371 |
| LAS1L | −0.016 | −0.202 | — | — | — | — | — | 0.246 |
| LAT2 | — | — | — | 0.100 | — | — | — | −0.330 |
| LCORL | — | 0.150 | — | 0.390 | — | — | — | 0.275 |
| LDB1 | — | — | — | −0.540 | — | — | — | −0.197 |
| LDHA | 0.032 | — | — | 0.324 | — | — | — | 0.121 |
| LENG8 | — | — | — | 0.297 | — | — | — | 0.325 |
| LETMD1 | — | — | — | −0.330 | — | — | — | 0.580 |
| LGI3 | — | 0.029 | — | — | — | 0.066 | — | — |
| LHFPL6 | — | — | — | 0.732 | 0.207 | — | — | — |
| LIFR | — | −0.158 | — | — | — | 0.034 | — | — |
| LIMD1 | — | — | — | 0.280 | — | — | — | 0.458 |
| LIMS1 | −0.002 | −0.449 | — | — | — | 0.015 | — | 0.032 |
| LIMS2 | — | −0.510 | — | — | — | 0.037 | — | — |
| LIMS3 | — | −0.449 | — | — | — | 0.015 | — | 0.032 |
| LIMS4 | — | −0.449 | — | — | — | 0.015 | — | 0.032 |
| LIPA | — | −0.143 | — | — | 0.723 | — | — | — |
| LIPE | — | — | — | 0.857 | — | −0.133 | — | — |
| LMAN1 | — | 0.287 | — | 0.635 | — | 0.073 | — | 0.666 |
| LMBRD1 | — | — | −0.009 | — | — | −0.126 | — | — |
| LMF1 | — | — | — | 0.340 | — | 0.174 | — | — |
| LMF2 | — | −0.080 | — | 0.589 | — | — | — | −0.282 |
| LMNA | — | 0.064 | — | 0.065 | — | 0.227 | — | — |
| LMO2 | — | −0.068 | — | 0.009 | — | 0.046 | — | — |
| LMO7 | — | 0.025 | — | — | — | −0.371 | — | — |
| LPCAT1 | — | 0.068 | — | 0.019 | — | 0.014 | — | — |
| LPIN2 | — | −0.241 | — | 0.007 | — | — | — | 0.132 |
| LRBA | — | 0.327 | 0.166 | — | — | — | — | 0.604 |
| LRCH1 | — | — | — | 0.207 | — | — | — | 0.364 |
| LRIG2 | — | 0.115 | — | 0.309 | — | — | — | 0.271 |
| LRP6 | — | — | — | 0.129 | — | — | — | 0.677 |
| LRRFIP1 | — | −0.105 | −0.015 | 0.535 | — | 0.032 | — | — |
| LRRK1 | — | −0.180 | — | 0.759 | — | — | — | 0.390 |
| LRWD1 | — | — | — | 0.326 | — | — | — | 0.261 |
| LSM3 | — | 0.387 | — | 0.013 | — | — | — | 0.300 |
| LSP1 | 0.583 | 0.323 | — | 0.128 | — | — | — | 0.059 |
| LTB | — | — | — | −0.235 | — | 0.408 | — | 0.035 |
| LTBP1 | — | 0.023 | −0.305 | −0.064 | — | 0.088 | — | −0.172 |
| LUC7L2 | — | 0.003 | — | 0.111 | — | 0.091 | — | — |
| LY6G6C | — | 0.465 | — | 0.052 | — | — | — | 0.036 |
| LY9 | — | 0.619 | — | 0.203 | — | — | — | 0.404 |
| LZTR1 | −0.001 | 0.052 | — | 0.035 | — | 0.066 | — | — |
| MACO1 | — | — | — | 0.772 | — | — | — | 0.202 |
| MADD | — | — | — | 0.026 | — | 0.087 | — | 0.264 |
| MALT1 | — | −0.323 | — | −0.368 | — | −0.150 | — | −0.227 |
| MAN1A1 | — | — | — | 0.322 | — | 0.104 | — | 0.068 |
| MAP2K2 | — | 0.085 | — | — | — | 0.118 | — | −0.421 |
| MAP3K12 | — | −0.042 | −0.002 | 0.190 | — | 0.323 | — | — |
| MAP3K4 | — | 0.020 | — | −0.461 | — | — | — | 0.508 |
| MAP4K2 | — | 0.076 | — | 0.048 | — | −0.103 | — | 0.191 |
| MAP4K4 | — | — | — | −0.568 | — | — | — | −0.499 |
| MAP7D1 | — | — | — | −0.624 | — | — | — | −0.414 |
| MAPK1 | — | 0.029 | — | 0.045 | — | — | — | 0.021 |
| MAPK10 | 0.012 | — | — | — | — | −0.267 | — | — |
| MAPK11 | — | −0.476 | — | — | — | 0.262 | — | 0.472 |
| MAPK14 | — | 0.081 | — | −0.090 | — | 0.018 | — | 0.183 |
| MAPK1IP1L | — | −0.169 | — | −0.306 | — | −0.340 | — | — |
| MAPK8IP3 | — | −0.103 | — | 0.039 | — | — | — | 0.545 |
| MAPKAPK3 | — | 0.750 | −0.003 | 0.260 | — | — | — | −0.354 |
| MARCH7 | — | — | — | 0.022 | — | — | — | −0.393 |
| MARK2 | — | — | −0.003 | 0.020 | — | — | — | 0.518 |
| MARS | — | −0.506 | — | −0.224 | — | — | — | 0.716 |
| MATR3 | — | −0.074 | — | −0.076 | — | — | — | 0.167 |
| MAU2 | — | −0.184 | 0.001 | −0.202 | — | — | — | −0.139 |
| MBNL1 | — | — | — | 0.027 | — | −0.136 | — | 0.037 |
| MBNL2 | −0.002 | 0.063 | 0.082 | 0.064 | — | 0.189 | — | 0.036 |
| MBTD1 | — | 0.007 | — | 0.367 | — | — | — | 0.584 |
| MCF2L | — | 0.005 | — | — | — | 0.053 | — | 0.139 |
| MCM2 | −0.714 | — | — | — | — | — | — | 0.593 |
| MCM3 | −0.013 | −0.204 | 0.014 | — | — | — | — | 0.467 |
| MCM9 | — | −0.429 | — | 0.364 | — | — | — | 0.297 |
| MCRS1 | — | 0.747 | — | 0.617 | — | — | — | 0.222 |
| MDM1 | — | −0.170 | — | 0.045 | — | — | — | 0.121 |
| MDM4 | 0.005 | 0.022 | — | 0.005 | — | 0.033 | — | — |
| MECOM | 0.006 | 0.014 | — | 0.532 | — | 0.461 | — | — |
| MED20 | — | 0.313 | — | 0.350 | — | — | — | 0.328 |
| MEF2A | −0.003 | — | — | 0.290 | — | — | — | −0.256 |
| MEF2C | — | −0.118 | — | −0.113 | — | 0.063 | — | 0.083 |
| MEIS1 | 0.014 | 0.162 | — | −0.852 | — | — | — | 0.431 |
| MEST | — | 0.422 | — | −0.263 | — | — | — | 0.249 |
| METRNL | — | −0.269 | — | — | — | — | — | −0.165 |
| METTL14 | — | 0.157 | — | — | — | 0.211 | — | 0.613 |
| METTL16 | — | 0.061 | — | — | — | — | — | 0.604 |
| METTL22 | — | 0.508 | — | — | — | — | — | 0.591 |
| METTL23 | −0.012 | −0.353 | — | −0.225 | — | — | — | 0.473 |
| METTL25 | — | −0.402 | — | — | — | 0.517 | — | — |
| METTL3 | — | −0.032 | — | 0.089 | — | — | — | 0.812 |
| METTL4 | — | — | — | 0.529 | — | — | — | −0.464 |
| METTL7A | — | 0.003 | −0.029 | 0.273 | — | 0.004 | — | — |
| MFF | −0.127 | — | — | 0.010 | — | — | — | 0.071 |
| MFGE8 | — | 0.031 | −0.029 | — | — | 0.007 | — | 0.481 |
| MFSD2B | — | — | — | 0.248 | — | — | — | 0.011 |
| MGAT1 | — | −0.109 | 0.000 | −0.302 | — | — | — | 0.249 |
| MGLL | — | 0.004 | −0.014 | −0.115 | — | 0.031 | — | — |
| MIA2 | — | 0.104 | −0.009 | — | — | — | — | 0.236 |
| MICU2 | −0.026 | 0.065 | — | 0.177 | — | — | — | 0.039 |
| MIER1 | — | — | — | 0.007 | — | 0.049 | — | — |
| MIF | — | — | — | 0.049 | — | — | — | 0.062 |
| MILR1 | — | −0.032 | — | 0.358 | — | −0.351 | — | −0.272 |
| MINDY1 | — | — | — | 0.408 | — | — | — | 0.168 |
| MINDY3 | — | 0.082 | — | 0.323 | — | — | — | 0.108 |
| MIS18A | — | −0.557 | — | −0.574 | — | — | — | 0.347 |
| MLH1 | −0.013 | −0.387 | −0.038 | −0.797 | — | — | — | −0.556 |
| MME | — | — | — | −0.715 | — | 0.277 | — | — |
| MMS19 | — | 0.166 | — | 0.288 | — | — | — | 0.826 |
| MOB1B | −0.001 | 0.123 | — | −0.522 | — | — | — | 0.253 |
| MOB4 | −0.017 | — | — | — | — | — | — | 0.209 |
| MON1A | −0.471 | 0.479 | — | −0.264 | — | — | — | 0.193 |
| MPC1 | — | — | — | 0.492 | — | — | — | 0.202 |
| MPP6 | — | 0.443 | — | −0.308 | — | — | — | 0.316 |
| MPP7 | 0.020 | 0.049 | — | — | — | 0.117 | — | — |
| MRAS | 0.007 | 0.007 | — | 0.433 | — | — | 0.331 | — |
| MRGBP | — | −0.114 | — | −0.557 | — | — | — | 0.194 |
| MROH1 | −0.025 | — | — | — | — | — | — | −0.350 |
| MRPL28 | — | — | — | 0.275 | — | — | — | 0.155 |
| MRPL52 | −0.021 | — | — | — | — | — | — | 0.348 |
| MRPS18C | — | 0.564 | — | — | — | — | — | 0.242 |
| MRPS24 | — | — | — | −0.150 | — | 0.247 | — | — |
| MRPS5 | — | — | — | −0.457 | — | — | — | 0.451 |
| MS4A4A | — | 0.358 | — | −0.270 | — | — | — | 0.111 |
| MS4A4E | — | 0.358 | — | −0.270 | — | — | — | 0.111 |
| MSH3 | — | −0.551 | — | −0.769 | — | — | — | −0.356 |
| MSL1 | — | −0.026 | −0.002 | −0.114 | — | — | — | 0.338 |
| MSLN | — | 0.312 | −0.384 | — | — | 0.052 | — | — |
| MSMO1 | — | 0.443 | — | 0.623 | — | — | — | 0.415 |
| MSTO1 | — | — | −0.093 | — | — | — | — | 0.751 |
| MT2A | — | −0.196 | — | — | — | −0.070 | — | — |
| MTCH1 | — | −0.058 | — | — | — | — | — | 0.270 |
| MTCH2 | — | — | — | −0.161 | — | — | — | 0.244 |
| MTCL1 | — | −0.108 | — | — | — | 0.049 | — | — |
| MTDH | — | — | — | 0.006 | — | — | — | 0.059 |
| MTF2 | −0.009 | 0.032 | −0.009 | 0.021 | — | — | — | 0.510 |
| MTHFS | — | −0.114 | — | 0.147 | — | 0.127 | — | — |
| MTMR3 | — | — | — | −0.323 | — | — | — | −0.345 |
| MTREX | — | — | — | 0.348 | — | — | — | 0.297 |
| MTSS1 | — | 0.002 | 0.027 | 0.023 | — | 0.142 | — | 0.770 |
| MTSS2 | — | 0.031 | — | 0.210 | — | 0.103 | — | — |
| MTUS1 | 0.700 | — | — | 0.446 | — | −0.099 | — | — |
| MUS81 | — | 0.299 | — | −0.544 | — | — | — | 0.460 |
| MX1 | — | 0.194 | — | 0.285 | — | 0.248 | — | — |
| MXD3 | — | — | — | 0.172 | — | — | — | −0.736 |
| MXI1 | — | — | — | 0.025 | — | 0.173 | — | — |
| MYBBP1A | — | — | — | 0.232 | — | — | — | 0.538 |
| MYCBP2 | 0.080 | — | 0.143 | — | — | — | — | −0.394 |
| MYEF2 | −0.020 | −0.178 | −0.329 | 0.610 | — | — | — | 0.565 |
| MYH10 | — | — | −0.068 | — | — | 0.030 | — | — |
| MYH7 | — | 0.698 | — | — | — | 0.368 | — | — |
| MYH9 | — | 0.088 | — | — | — | — | — | −0.006 |
| MYL12A | — | — | — | 0.314 | — | 0.080 | — | — |
| MYL12B | — | — | — | 0.188 | — | 0.005 | — | 0.022 |
| MYLK | −0.001 | — | −0.351 | 0.010 | — | 0.016 | — | −0.626 |
| MYO1C | −0.001 | — | — | — | — | — | — | 0.197 |
| MYOF | — | −0.137 | — | 0.286 | — | — | — | 0.553 |
| NAA15 | — | — | — | 0.463 | — | — | — | 0.630 |
| NAA16 | — | — | — | 0.475 | — | — | — | 0.706 |
| NAA40 | −0.003 | −0.442 | — | −0.085 | — | — | — | −0.375 |
| NAA60 | — | — | −0.002 | 0.384 | — | — | — | 0.282 |
| NADK2 | — | — | — | 0.053 | — | — | — | 0.164 |
| NCALD | — | 0.173 | — | 0.282 | — | 0.355 | — | — |
| NCAPD3 | — | −0.296 | — | −0.358 | — | — | — | 0.461 |
| NCK2 | — | 0.269 | — | — | — | 0.074 | — | — |
| NCKAP1L | — | −0.222 | — | 0.025 | — | — | — | 0.287 |
| NCOA4 | — | — | 0.000 | — | — | — | — | 0.063 |
| NCOA7 | — | — | — | 0.171 | — | — | — | 0.498 |
| NCOR2 | −0.003 | 0.407 | — | 0.039 | — | — | — | −0.736 |
| NDFIP2 | — | −0.168 | — | 0.003 | — | — | — | 0.114 |
| NDRG1 | — | — | — | 0.087 | — | — | — | 0.192 |
| NDRG2 | −0.011 | — | — | −0.309 | — | 0.076 | 0.094 | — |
| NDUFA11 | — | — | — | −0.421 | — | — | — | 0.046 |
| NDUFA4 | — | — | — | 0.036 | — | — | — | 0.041 |
| NDUFAF2 | — | 0.521 | — | 0.400 | — | — | — | 0.705 |
| NDUFS1 | — | — | — | 0.325 | — | — | — | 0.469 |
| NDUFS4 | — | — | — | 0.332 | — | — | — | 0.024 |
| NDUFS6 | — | — | — | 0.415 | — | — | — | 0.141 |
| NDUFS7 | — | — | 0.181 | — | — | — | — | 0.197 |
| NEDD4L | — | −0.199 | −0.034 | 0.220 | — | 0.058 | — | — |
| NEK1 | — | 0.384 | — | 0.466 | — | −0.210 | — | — |
| NEK4 | — | 0.197 | — | −0.295 | — | 0.212 | — | — |
| NEMF | — | 0.118 | −0.033 | 0.354 | — | — | — | 0.400 |
| NEURL1 | — | 0.649 | — | — | — | — | — | 0.269 |
| NFATC3 | −0.279 | — | — | 0.328 | — | — | — | 0.109 |
| NFE2L1 | −0.003 | −0.043 | — | −0.446 | — | 0.026 | — | — |
| NFE2L2 | — | — | −0.003 | — | — | 0.415 | — | — |
| NFKB2 | — | — | — | −0.440 | — | — | — | 0.659 |
| NFU1 | — | — | — | 0.817 | — | — | — | 0.337 |
| NFX1 | — | — | — | 0.324 | — | — | — | 0.206 |
| NFYB | — | 0.184 | — | 0.322 | — | — | — | 0.241 |
| NGLY1 | — | — | −0.024 | — | — | — | — | 0.261 |
| NIPSNAP2 | — | — | −0.039 | — | — | — | — | 0.111 |
| NISCH | — | 0.054 | — | 0.377 | — | 0.052 | — | 0.778 |
| NKTR | — | −0.337 | — | — | — | — | — | −0.205 |
| NME2 | — | — | — | 0.389 | — | — | 0.030 | — |
| NMNAT3 | — | 0.476 | −0.287 | 0.464 | — | — | — | −0.645 |
| NMT2 | — | — | — | 0.368 | — | — | — | 0.204 |
| NOL4L | — | −0.227 | — | — | — | −0.363 | — | — |
| NOL7 | — | −0.299 | — | −0.770 | — | — | — | 0.099 |
| NOL8 | — | 0.072 | — | −0.085 | — | 0.188 | — | — |
| NOL9 | — | 0.256 | — | — | — | — | — | 0.469 |
| NOLC1 | — | 0.026 | — | — | — | — | — | 0.416 |
| NOP56 | — | — | — | 0.322 | — | — | — | 0.361 |
| NOX4 | — | 0.022 | — | — | — | 0.030 | — | — |
| NPNT | — | 0.000 | — | −0.096 | — | 0.066 | — | — |
| NPTN | — | — | — | 0.031 | — | — | — | 0.011 |
| NR3C1 | −0.016 | 0.207 | — | 0.555 | — | — | — | 0.179 |
| NRBP1 | — | 0.070 | — | 0.023 | — | — | — | 0.072 |
| NRF1 | — | −0.319 | — | 0.477 | — | 0.134 | — | — |
| NSG1 | — | — | — | −0.301 | — | 0.230 | — | — |
| NSMCE2 | — | 0.119 | — | — | — | — | — | −0.306 |
| NSUN2 | — | — | — | 0.203 | — | — | — | 0.599 |
| NSUN4 | — | — | — | −0.377 | — | — | — | 0.281 |
| NTMT1 | — | 0.341 | — | −0.299 | — | — | — | −0.232 |
| NUBP2 | — | 0.039 | — | — | — | 0.288 | — | — |
| NUCB2 | — | — | −0.132 | — | — | — | — | 0.231 |
| NUDCD1 | — | 0.057 | — | −0.050 | — | — | — | −0.554 |
| NUDT13 | — | −0.057 | — | −0.213 | — | — | — | 0.657 |
| NUDT16 | — | — | — | 0.180 | — | — | — | 0.671 |
| NUP88 | — | 0.078 | — | −0.311 | — | — | — | 0.551 |
| NUP98 | — | −0.068 | — | 0.301 | — | — | — | −0.124 |
| NXPE2 | — | — | — | 0.393 | — | — | — | −0.607 |
| ODC1 | −0.405 | — | — | — | — | — | — | 0.026 |
| OGDH | — | — | — | 0.467 | — | — | — | 0.125 |
| OGFOD2 | — | −0.474 | — | — | — | — | — | −0.606 |
| OLFML3 | — | — | −0.385 | — | — | 0.172 | — | — |
| ORC3 | — | — | −0.081 | — | — | — | — | 0.479 |
| OSBPL6 | — | — | — | 0.413 | — | 0.088 | — | — |
| OSGEPL1 | — | 0.546 | 0.599 | — | — | — | — | 0.827 |
| OTUD5 | — | −0.207 | — | — | — | — | — | 0.077 |
| OXNAD1 | — | — | — | 0.248 | — | — | — | 0.584 |
| P2RY14 | — | −0.676 | — | — | — | — | — | 0.728 |
| P4HA1 | — | 0.170 | — | 0.274 | — | −0.456 | — | — |
| P4HTM | — | 0.409 | — | — | — | 0.326 | — | — |
| PACS1 | — | — | — | 0.432 | — | 0.178 | — | 0.345 |
| PACSIN2 | — | — | — | 0.151 | — | — | — | 0.020 |
| PAIP2 | — | −0.003 | −0.524 | 0.012 | — | — | — | 0.008 |
| PAN2 | — | 0.239 | — | — | — | — | — | 0.284 |
| PAPOLA | — | 0.074 | 0.023 | — | — | — | — | 0.244 |
| PAQR7 | — | 0.107 | — | −0.582 | — | 0.181 | — | — |
| PAQR8 | — | 0.026 | — | −0.551 | — | 0.078 | — | — |
| PARD6A | — | 0.462 | — | 0.037 | — | — | — | −0.340 |
| PARN | — | — | −0.035 | — | — | — | — | 0.764 |
| PARP10 | −0.007 | — | — | — | — | — | — | 0.547 |
| PARP6 | — | −0.049 | — | −0.866 | — | — | — | 0.476 |
| PARP9 | — | — | −0.035 | — | — | — | — | 0.509 |
| PAXX | — | −0.127 | — | — | — | — | — | 0.474 |
| PBDC1 | — | — | −0.062 | −0.125 | — | 0.348 | — | −0.354 |
| PCBP2 | — | — | — | 0.010 | — | — | — | 0.053 |
| PCED1A | — | −0.069 | — | 0.372 | — | — | — | 0.352 |
| PCMT1 | — | −0.567 | — | 0.006 | — | 0.064 | — | 0.011 |
| PCNT | — | — | — | 0.599 | — | — | — | 0.587 |
| PCOLCE | — | 0.055 | — | — | — | 0.180 | — | — |
| PCSK7 | — | 0.298 | — | — | — | — | — | −0.259 |
| PCYT2 | — | — | — | −0.417 | — | — | — | 0.842 |
| PDE1B | — | — | 0.542 | — | — | −0.536 | — | — |
| PDGFA | — | — | — | 0.330 | — | — | — | 0.083 |
| PDGFRA | — | — | — | −0.626 | — | −0.012 | — | — |
| PDLIM5 | — | 0.084 | — | — | — | 0.181 | — | — |
| PDLIM7 | — | — | — | 0.308 | — | −0.156 | — | 0.044 |
| PDPR | 0.004 | — | −0.008 | — | — | — | — | −0.607 |
| PDRG1 | — | — | — | −0.306 | — | — | — | −0.118 |
| PDZD2 | — | 0.002 | — | — | — | 0.060 | — | — |
| PDZK1IP1 | — | 0.237 | — | 0.327 | — | — | — | 0.016 |
| PEX2 | — | 0.139 | — | −0.254 | — | — | — | 0.618 |
| PFKFB2 | — | 0.052 | — | — | — | −0.117 | — | — |
| PFKFB3 | 0.004 | −0.036 | — | — | — | −0.020 | — | — |
| PFN1 | — | 0.125 | — | −0.111 | — | — | — | −0.067 |
| PGAP2 | 0.005 | 0.160 | −0.022 | 0.192 | — | — | — | 0.621 |
| PGGT1B | — | 0.120 | — | 0.007 | — | — | — | −0.205 |
| PHF21A | — | — | — | −0.539 | — | 0.133 | — | — |
| PHF7 | — | −0.249 | −0.345 | −0.197 | — | −0.375 | — | — |
| PHIP | — | 0.129 | — | 0.571 | — | — | — | 0.590 |
| PHKB | — | 0.211 | — | 0.302 | — | 0.070 | — | — |
| PI16 | — | 0.299 | — | 0.271 | — | 0.213 | — | — |
| PI4K2A | — | 0.145 | — | — | — | — | — | 0.586 |
| PIEZO2 | — | −0.149 | — | — | — | 0.099 | — | — |
| PIGC | — | 0.099 | — | 0.135 | — | — | — | 0.502 |
| PIGH | — | −0.167 | −0.020 | — | — | — | — | −0.240 |
| PIGN | −0.266 | −0.206 | −0.053 | 0.228 | — | — | — | 0.034 |
| PIK3CD | — | −0.184 | −0.001 | −0.560 | — | −0.213 | — | — |
| PIK3CG | — | — | — | 0.316 | — | — | — | 0.139 |
| PIK3R4 | — | 0.105 | — | 0.319 | — | 0.127 | — | — |
| PIKFYVE | — | −0.070 | — | — | — | — | — | 0.738 |
| PIM1 | — | −0.145 | — | −0.018 | — | — | — | −0.112 |
| PJA1 | — | — | — | −0.398 | — | 0.240 | — | — |
| PKIG | — | −0.013 | — | 0.102 | — | — | — | −0.402 |
| PKN2 | — | −0.263 | −0.046 | −0.204 | — | — | — | 0.143 |
| PKN3 | — | −0.279 | — | 0.275 | — | 0.374 | — | — |
| PLA2G12A | 0.031 | −0.061 | — | 0.287 | — | — | — | 0.028 |
| PLA2G4F | 0.024 | −0.285 | — | — | — | 0.269 | — | — |
| PLA2G7 | — | −0.086 | — | −0.035 | — | −0.137 | — | −0.061 |
| PLCB2 | −0.043 | — | — | — | — | — | — | −0.204 |
| PLCB4 | — | −0.113 | — | 0.785 | — | 0.252 | — | — |
| PLCE1 | — | — | — | 0.542 | — | 0.071 | — | — |
| PLEKHA1 | — | — | −0.026 | 0.125 | — | — | — | −0.561 |
| PLEKHA6 | — | −0.060 | — | −0.295 | — | 0.444 | — | — |
| PLEKHG5 | — | 0.540 | — | — | — | 0.224 | — | — |
| PLK4 | — | −0.473 | 0.422 | — | — | — | — | 0.749 |
| PLP2 | — | — | — | 0.518 | — | — | — | 0.242 |
| PLPBP | — | −0.049 | — | −0.183 | — | — | — | 0.279 |
| PLS3 | — | −0.037 | — | — | — | 0.094 | — | — |
| PLSCR1 | — | −0.366 | — | 0.403 | — | — | — | −0.195 |
| PLTP | — | 0.064 | −0.008 | — | — | — | — | −0.400 |
| PLXNC1 | −0.004 | — | −0.002 | — | — | — | — | −0.191 |
| PM20D1 | −0.780 | — | −0.294 | — | — | — | 0.568 | — |
| PML | — | 0.370 | — | — | — | 0.122 | — | — |
| PMM1 | — | −0.495 | — | 0.061 | — | — | — | 0.464 |
| PNN | — | — | — | −0.675 | — | — | — | −0.295 |
| PNPLA6 | — | −0.390 | — | — | — | 0.178 | — | — |
| PNPLA8 | — | — | — | 0.101 | — | — | — | 0.026 |
| POC1A | — | −0.729 | — | — | — | −0.292 | — | — |
| PODN | — | 0.356 | — | — | — | 0.270 | — | — |
| POFUT2 | — | −0.681 | — | 0.307 | — | — | — | 0.453 |
| POLR2I | −0.059 | −0.552 | — | −0.170 | — | — | — | 0.200 |
| POLR3H | — | 0.608 | — | −0.241 | — | — | — | 0.753 |
| PON2 | −0.003 | — | — | 0.384 | — | — | — | 0.200 |
| POPDC3 | — | — | −0.457 | — | −0.171 | — | — | — |
| POSTN | — | 0.002 | −0.205 | — | — | 0.035 | — | — |
| PPAT | — | −0.190 | — | — | — | — | 0.649 | 0.606 |
| PPFIBP1 | — | −0.396 | — | — | — | — | — | 0.744 |
| PPHLN1 | — | 0.074 | — | −0.370 | — | — | — | 0.673 |
| PPIP5K1 | — | −0.517 | — | — | — | — | — | 0.581 |
| PPP1CA | — | — | — | 0.003 | — | 0.009 | — | — |
| PPP1CC | — | −0.077 | — | 0.179 | — | — | — | 0.196 |
| PPP1R18 | — | 0.063 | — | 0.221 | — | — | — | 0.122 |
| PPP2R1A | — | — | — | 0.326 | — | — | — | 0.095 |
| PPP2R2D | — | −0.369 | — | — | — | — | — | 0.571 |
| PPP6R2 | — | 0.119 | −0.007 | 0.438 | — | −0.296 | — | — |
| PQBP1 | — | — | — | −0.199 | — | 0.121 | — | 0.115 |
| PQLC1 | — | 0.486 | — | 0.326 | — | — | — | −0.304 |
| PQLC3 | — | 0.071 | — | — | — | — | — | −0.117 |
| PRC1 | — | −0.290 | — | — | — | — | — | 0.737 |
| PRDM6 | — | −0.441 | — | — | — | 0.076 | — | — |
| PRDX2 | — | 0.659 | — | — | — | 0.059 | — | — |
| PRDX5 | — | — | — | 0.016 | — | — | — | 0.010 |
| PRDX6 | — | 0.193 | — | 0.041 | — | — | — | 0.041 |
| PRICKLE2 | — | 0.095 | — | — | — | 0.053 | — | — |
| PRKCD | — | — | — | 0.009 | — | — | — | 0.041 |
| PRKDC | −0.019 | — | — | — | — | — | — | −0.618 |
| PRKG1 | — | −0.119 | −0.417 | 0.168 | — | 0.063 | — | — |
| PRMT2 | — | 0.450 | — | 0.069 | — | 0.065 | — | — |
| PRMT9 | — | — | — | −0.522 | — | — | — | 0.735 |
| PRPF19 | — | — | — | 0.320 | — | — | — | 0.357 |
| PRPS2 | — | — | — | −0.372 | — | 0.187 | — | — |
| PRPSAP2 | −0.005 | −0.323 | −0.016 | — | — | −0.528 | — | — |
| PRR14 | — | — | −0.036 | −0.820 | — | — | — | 0.544 |
| PRRG2 | — | −0.235 | — | — | — | — | — | 0.203 |
| PSAT1 | — | −0.663 | — | 0.347 | — | — | — | −0.295 |
| PSD3 | −0.008 | 0.069 | — | −0.517 | — | 0.059 | — | 0.092 |
| PSG1 | — | — | — | 0.077 | — | — | — | 0.370 |
| PSG11 | — | — | — | 0.077 | — | — | — | 0.370 |
| PSG2 | — | — | — | 0.077 | — | — | — | 0.370 |
| PSG3 | — | — | — | 0.077 | — | — | — | 0.370 |
| PSG4 | — | — | — | 0.077 | — | — | — | 0.370 |
| PSG5 | — | — | — | 0.077 | — | — | — | 0.370 |
| PSG6 | — | — | — | 0.077 | — | — | — | 0.370 |
| PSG7 | — | — | — | 0.077 | — | — | — | 0.370 |
| PSG8 | — | — | — | 0.077 | — | — | — | 0.370 |
| PSG9 | — | — | — | 0.077 | — | — | — | 0.370 |
| PSMB9 | — | 0.017 | — | −0.300 | — | — | — | 0.294 |
| PSMD11 | 0.020 | — | −0.029 | — | — | — | — | 0.398 |
| PSMD13 | — | −0.114 | — | 0.278 | — | 0.133 | — | 0.070 |
| PSMD5 | — | — | — | 0.175 | — | — | — | 0.675 |
| PSME1 | — | — | — | −0.135 | — | — | — | 0.037 |
| PSMG3 | — | −0.638 | — | −0.805 | — | — | — | 0.515 |
| PTGR2 | — | 0.050 | — | — | — | 0.074 | — | — |
| PTK2 | — | 0.157 | — | −0.262 | — | −0.085 | — | 0.130 |
| PTK2B | — | — | — | 0.373 | — | — | — | 0.204 |
| PTP4A3 | — | −0.308 | — | 0.320 | — | 0.150 | — | — |
| PTPN6 | — | −0.438 | — | 0.054 | — | — | — | 0.066 |
| PTPRA | — | −0.140 | — | −0.369 | — | — | — | 0.099 |
| PTPRC | — | 0.155 | — | 0.172 | — | — | — | 0.109 |
| PTPRG | — | −0.347 | — | −0.560 | — | 0.074 | — | — |
| PTPRK | — | −0.108 | 0.566 | — | — | 0.087 | — | — |
| PTPRM | — | 0.003 | — | — | — | 0.018 | — | — |
| PTPRS | — | 0.009 | — | 0.456 | — | 0.040 | — | 0.830 |
| PUM2 | −0.002 | −0.029 | 0.010 | 0.004 | — | — | — | 0.093 |
| PXYLP1 | — | 0.474 | — | 0.293 | — | 0.248 | — | — |
| PYHIN1 | — | 0.287 | — | −0.531 | — | — | — | 0.685 |
| PYROXD1 | −0.015 | 0.275 | −0.053 | — | — | — | — | 0.617 |
| QARS | −0.018 | 0.131 | — | — | — | — | — | 0.271 |
| R3HDM1 | — | 0.426 | — | −0.531 | — | −0.166 | — | 0.292 |
| R3HDM4 | — | — | — | 0.017 | — | — | — | 0.014 |
| RAB11B | — | 0.338 | — | — | — | — | — | 0.057 |
| RAB2B | — | −0.308 | — | — | — | — | — | 0.638 |
| RAB44 | — | −0.641 | — | — | — | — | — | −0.754 |
| RAB7A | — | 0.150 | — | 0.374 | — | 0.016 | — | — |
| RABEP1 | — | −0.121 | — | — | — | — | — | 0.552 |
| RABGGTA | — | — | — | 0.406 | — | — | — | 0.675 |
| RAC1 | −0.002 | — | −0.005 | 0.008 | — | — | — | 0.004 |
| RACK1 | — | — | 0.008 | 0.026 | — | — | — | 0.012 |
| RAD1 | — | 0.353 | — | — | — | — | — | 0.383 |
| RAD17 | — | 0.065 | — | −0.161 | — | — | −0.285 | 0.487 |
| RAD18 | — | 0.254 | −0.536 | −0.102 | — | −0.230 | — | — |
| RAD51 | — | — | 0.133 | 0.436 | — | — | — | 0.802 |
| RALBP1 | — | −0.077 | — | 0.236 | — | 0.031 | — | — |
| RALGAPA2 | — | −0.595 | — | — | — | — | — | 0.304 |
| RALGAPB | −0.007 | — | — | — | — | — | — | 0.595 |
| RALGPS1 | — | 0.229 | — | −0.117 | — | −0.541 | — | — |
| RALGPS2 | — | −0.129 | — | −0.052 | — | — | — | 0.364 |
| RAMAC | — | −0.286 | — | −0.266 | — | — | — | 0.029 |
| RAMACL | — | −0.286 | — | −0.266 | — | — | — | 0.029 |
| RAMP1 | — | — | — | 0.252 | — | — | — | 0.109 |
| RAMP2 | — | 0.024 | — | — | — | 0.242 | — | — |
| RANBP3 | — | 0.907 | — | — | — | — | — | 0.548 |
| RANGAP1 | — | — | — | 0.145 | — | 0.368 | — | 0.237 |
| RAPGEF2 | — | — | — | −0.323 | — | 0.055 | — | 0.718 |
| RARRES2 | — | 0.043 | — | — | — | 0.108 | — | — |
| RASA4 | — | −0.568 | 0.029 | — | — | — | — | 0.218 |
| RASA4B | 0.055 | −0.568 | — | — | — | — | — | 0.218 |
| RASAL3 | — | 0.144 | — | −0.208 | — | — | — | 0.450 |
| RASGRP3 | — | −0.092 | — | −0.370 | — | — | — | 0.145 |
| RB1CC1 | −0.169 | — | — | −0.387 | — | 0.086 | — | — |
| RBL2 | — | 0.078 | — | −0.384 | — | — | — | −0.184 |
| RBM25 | — | — | — | 0.109 | — | 0.448 | — | — |
| RBM3 | — | −0.648 | — | −0.603 | — | −0.142 | — | — |
| RBM5 | — | — | — | 0.060 | — | — | — | 0.186 |
| RBM6 | — | 0.211 | — | — | — | 0.199 | — | — |
| RBM7 | — | — | — | 0.506 | — | 0.421 | — | 0.193 |
| RBMS2 | 0.011 | 0.182 | 0.039 | — | — | — | — | 0.613 |
| RBX1 | — | 0.089 | — | — | — | — | — | 0.273 |
| RCBTB2 | — | 0.300 | — | −0.210 | — | — | — | 0.560 |
| RCC2 | — | — | −0.004 | — | — | — | — | 0.130 |
| RCOR3 | — | −0.235 | −0.005 | −0.815 | — | — | — | 0.568 |
| RDX | — | — | 0.799 | −0.432 | — | 0.040 | — | — |
| RELB | — | 0.036 | — | — | — | — | — | 0.337 |
| RELCH | — | 0.026 | — | 0.415 | — | — | — | 0.746 |
| RETREG1 | 0.189 | — | — | 0.158 | — | −0.054 | — | — |
| RETREG2 | — | −0.062 | — | 0.346 | — | — | — | 0.329 |
| REX1BD | — | 0.302 | — | — | — | — | — | 0.238 |
| REXO1 | — | 0.120 | — | — | — | — | — | −0.394 |
| REXO5 | — | 0.435 | −0.535 | — | — | −0.433 | — | 0.659 |
| RFC1 | — | −0.191 | — | 0.029 | — | — | — | 0.255 |
| RFFL | — | −0.367 | — | 0.475 | — | — | — | 0.517 |
| RFX3 | — | — | — | 0.014 | — | 0.173 | — | 0.593 |
| RFX5 | — | 0.263 | — | 0.600 | — | — | — | 0.596 |
| RFX7 | — | −0.300 | — | 0.854 | — | — | — | −0.418 |
| RGS1 | — | −0.749 | — | −0.072 | — | — | — | −0.449 |
| RGS12 | — | 0.003 | — | 0.759 | — | — | 0.189 | — |
| RHOJ | — | — | −0.211 | 0.271 | — | — | — | 0.046 |
| RHOT1 | −0.003 | 0.134 | — | −0.378 | — | — | — | 0.355 |
| RHOT2 | — | — | 0.049 | −0.335 | — | — | — | 0.742 |
| RIC8A | — | 0.129 | −0.007 | 0.033 | — | — | — | 0.228 |
| RIDA | — | 0.635 | — | 0.058 | — | — | — | 0.025 |
| RIF1 | — | 0.012 | — | −0.285 | — | — | — | 0.826 |
| RIN2 | −0.030 | — | — | — | — | −0.118 | — | — |
| RIOK1 | — | 0.212 | — | 0.048 | — | — | — | 0.394 |
| RMC1 | −0.095 | — | — | — | — | — | — | −0.310 |
| RMI1 | — | −0.071 | — | 0.328 | — | — | — | −0.362 |
| RNF103 | — | 0.185 | — | 0.311 | — | — | — | −0.305 |
| RNF123 | −0.017 | 0.435 | — | — | — | — | — | −0.408 |
| RNF13 | — | −0.064 | — | — | — | — | — | 0.119 |
| RNF138 | — | — | — | −0.192 | — | — | — | 0.092 |
| RNF14 | — | 0.026 | — | 0.282 | — | — | — | 0.063 |
| RNF141 | — | — | — | −0.552 | — | 0.019 | — | — |
| RNF167 | −0.003 | −0.008 | — | 0.050 | — | — | — | 0.509 |
| RNF181 | — | — | — | 0.005 | — | 0.128 | — | 0.126 |
| RNF38 | −0.001 | — | — | 0.138 | — | 0.200 | — | — |
| RNF4 | — | 0.311 | — | −0.273 | — | — | — | 0.103 |
| RNF44 | — | 0.062 | — | 0.050 | — | — | — | −0.056 |
| RNF5 | — | 0.520 | — | — | — | — | — | 0.166 |
| ROCK2 | −0.008 | — | — | 0.157 | — | 0.027 | — | 0.069 |
| RPA1 | — | — | — | 0.461 | — | 0.154 | — | — |
| RPIA | — | 0.277 | — | — | — | — | — | 0.516 |
| RPL10A | — | −0.156 | — | 0.138 | — | 0.052 | — | — |
| RPL11 | — | — | — | 0.016 | — | — | — | 0.046 |
| RPL22L1 | — | −0.086 | — | −0.516 | — | — | — | 0.053 |
| RPL23 | — | — | — | 0.010 | — | — | — | 0.138 |
| RPL28 | −0.003 | — | −0.011 | −0.103 | −0.030 | — | −0.028 | — |
| RPL30 | — | — | −0.004 | −0.317 | — | — | — | 0.069 |
| RPL35A | −0.060 | — | — | 0.017 | — | — | — | 0.059 |
| RPL37A | — | — | −0.005 | −0.244 | — | — | — | 0.032 |
| RPL8 | — | — | — | 0.055 | — | — | — | 0.053 |
| RPP30 | — | 0.141 | — | — | — | 0.225 | — | — |
| RPP40 | — | — | — | 0.284 | — | — | — | 0.357 |
| RPRD1B | — | — | — | 0.438 | — | 0.149 | — | — |
| RPS13 | — | 0.107 | — | 0.090 | — | — | — | 0.030 |
| RPS23 | — | 0.206 | — | 0.076 | — | — | — | 0.184 |
| RPS24 | — | — | — | 0.003 | — | — | — | 0.004 |
| RPS27L | — | −0.071 | — | 0.344 | — | — | — | 0.047 |
| RPS6 | — | — | — | 0.011 | — | — | — | 0.038 |
| RPS6KB1 | — | 0.146 | 0.039 | — | — | −0.109 | — | −0.139 |
| RRAGC | −0.009 | — | — | 0.324 | — | — | — | −0.087 |
| RREB1 | −0.008 | — | — | 0.194 | — | — | — | 0.169 |
| RRP36 | — | — | — | 0.261 | — | — | — | 0.619 |
| RRP8 | — | — | — | 0.297 | — | — | — | 0.431 |
| RSAD2 | — | 0.154 | — | 0.092 | — | −0.087 | — | −0.035 |
| RSBN1 | — | 0.029 | — | — | — | −0.159 | — | — |
| RSRC2 | — | 0.108 | — | −0.240 | — | 0.052 | — | — |
| RTKN2 | — | 0.009 | — | — | — | −0.775 | 0.387 | — |
| RTN3 | 0.012 | −0.033 | — | — | — | 0.030 | — | — |
| RUFY3 | — | −0.192 | — | −0.676 | — | — | — | 0.760 |
| RUSC2 | −0.006 | −0.340 | — | — | — | 0.127 | — | — |
| RYBP | — | 0.072 | — | — | — | — | — | 0.431 |
| S100A1 | — | −0.398 | — | 0.018 | — | — | — | 0.009 |
| SAA1 | — | −0.116 | — | — | — | −0.129 | — | — |
| SAA2 | — | −0.116 | — | — | — | −0.129 | — | — |
| SAAL1 | — | −0.621 | — | — | — | — | — | 0.634 |
| SAFB2 | — | — | — | −0.669 | — | — | — | −0.339 |
| SAMHD1 | −0.006 | — | 0.016 | 0.008 | — | 0.128 | — | 0.269 |
| SAP18 | — | — | — | 0.094 | — | — | — | 0.178 |
| SARS | — | — | — | 0.271 | — | — | — | 0.250 |
| SART1 | — | — | — | 0.298 | — | — | — | 0.280 |
| SART3 | — | 0.325 | — | — | — | — | — | −0.212 |
| SAXO2 | — | −0.613 | −0.259 | — | — | 0.249 | — | — |
| SBF1 | — | −0.212 | — | −0.267 | — | — | — | 0.243 |
| SCAF11 | — | 0.046 | — | — | — | 0.036 | | 0.132 |
| SCAMP1 | — | 0.656 | — | 0.253 | — | — | — | 0.052 |
| SCART1 | — | 0.724 | — | — | — | 0.591 | — | — |
| SCD | — | — | — | −0.126 | — | 0.011 | — | — |
| SDCBP | — | — | — | 0.022 | — | — | — | 0.006 |
| SDHAF2 | — | −0.016 | — | 0.017 | — | — | — | 0.081 |
| SEC11C | — | −0.436 | — | 0.190 | — | — | — | 0.097 |
| SEC31A | — | — | — | −0.588 | — | 0.371 | — | — |
| SEC61G | — | −0.178 | — | −0.118 | — | 0.310 | — | −0.063 |
| SELENOM | — | 0.074 | — | −0.072 | — | 0.150 | — | — |
| SELENOP | — | 0.003 | — | 0.051 | — | 0.017 | — | 0.055 |
| SEMA6D | — | 0.089 | — | 0.056 | — | — | — | 0.799 |
| SENP1 | −0.069 | −0.249 | −0.098 | 0.145 | — | 0.248 | — | — |
| SENP2 | — | 0.005 | — | −0.298 | — | — | — | 0.417 |
| SENP5 | — | — | 0.014 | — | — | 0.326 | — | — |
| SENP7 | — | −0.477 | — | 0.025 | — | — | — | −0.138 |
| SEPT4 | — | 0.016 | — | — | — | 0.131 | — | — |
| SERF2 | — | — | — | 0.007 | — | — | — | 0.010 |
| SERHL2 | — | 0.042 | −0.086 | −0.111 | — | — | — | −0.284 |
| SERPINB6 | — | — | 0.004 | 0.067 | 0.107 | — | — | — |
| SERPINF2 | — | — | — | 0.369 | — | — | 0.775 | — |
| SERPINH1 | — | −0.039 | — | 0.553 | — | 0.419 | — | — |
| SETD4 | — | — | — | −0.405 | — | −0.436 | — | — |
| SF3B3 | −0.250 | — | — | −0.177 | — | 0.156 | — | 0.465 |
| SFI1 | — | — | −0.011 | −0.071 | — | — | — | 0.716 |
| SFSWAP | — | 0.489 | — | 0.293 | — | −0.132 | — | — |
| SFTPA2 | — | — | — | −0.489 | 0.025 | — | — | — |
| SFXN5 | −0.403 | — | −0.003 | — | — | 0.166 | — | — |
| SGCE | — | 0.226 | 0.605 | 0.177 | — | 0.074 | — | — |
| SGK1 | — | — | — | 0.037 | — | — | — | 0.333 |
| SGK3 | — | −0.301 | — | 0.199 | — | — | — | 0.215 |
| SGMS1 | — | 0.198 | — | 0.039 | — | 0.055 | — | — |
| SH3KBP1 | — | −0.113 | — | −0.041 | — | 0.050 | — | — |
| SH3PXD2A | 0.000 | — | 0.036 | −0.077 | — | — | — | −0.674 |
| SHANK3 | — | −0.551 | — | — | — | 0.030 | — | — |
| SHARPIN | — | — | — | 0.419 | — | — | — | 0.238 |
| SHISA5 | — | −0.009 | — | 0.048 | — | — | — | 0.163 |
| SHKBP1 | — | — | — | 0.633 | — | — | — | 0.235 |
| SHLD2 | — | — | — | 0.743 | — | 0.198 | — | — |
| SHROOM3 | — | −0.010 | — | — | — | 0.099 | — | — |
| SHTN1 | — | 0.242 | — | — | — | — | — | 0.852 |
| SIKE1 | — | — | — | −0.413 | — | — | — | 0.231 |
| SIL1 | — | 0.177 | — | 0.113 | — | 0.113 | — | — |
| SIPA1L1 | −0.026 | 0.038 | — | 0.292 | — | 0.115 | — | — |
| SIRT7 | — | 0.250 | — | 0.351 | — | — | — | 0.192 |
| SIVA1 | — | — | — | 0.567 | — | — | — | 0.288 |
| SKIV2L | −0.155 | 0.378 | — | 0.619 | — | — | — | 0.175 |
| SKP2 | — | −0.123 | — | 0.809 | — | −0.659 | — | — |
| SLBP | — | −0.032 | — | 0.188 | — | — | — | 0.164 |
| SLC12A6 | — | −0.068 | — | −0.290 | — | — | — | 0.577 |
| SLC12A7 | — | −0.181 | — | 0.190 | — | — | — | 0.470 |
| SLC16A6 | — | — | −0.013 | — | — | −0.427 | — | — |
| SLC17A5 | — | 0.330 | — | 0.583 | — | — | — | −0.314 |
| SLC18A2 | — | — | — | 0.008 | — | — | — | 0.145 |
| SLC1A5 | −0.011 | 0.019 | — | — | — | −0.024 | — | 0.062 |
| SLC20A2 | −0.003 | −0.089 | — | 0.024 | — | — | — | 0.189 |
| SLC25A1 | — | 0.317 | — | — | — | — | — | 0.682 |
| SLC25A11 | — | −0.119 | — | — | — | 0.084 | — | — |
| SLC25A17 | — | 0.114 | — | 0.029 | — | — | — | 0.094 |
| SLC25A39 | — | — | — | 0.052 | — | — | — | 0.052 |
| SLC25A40 | — | 0.489 | — | 0.309 | — | — | — | 0.753 |
| SLC35A5 | — | −0.124 | — | — | — | — | — | 0.596 |
| SLC35B2 | — | — | −0.034 | — | — | — | — | 0.307 |
| SLC35B3 | — | −0.090 | — | 0.034 | — | — | — | −0.140 |
| SLC37A1 | — | 0.034 | — | −0.326 | — | 0.057 | — | — |
| SLC38A2 | — | — | −0.002 | 0.203 | — | — | — | 0.173 |
| SLC39A2 | −0.285 | — | — | — | — | −0.388 | — | — |
| SLC43A1 | — | −0.089 | — | — | — | −0.272 | — | — |
| SLC44A2 | — | — | — | −0.062 | — | — | — | 0.210 |
| SLC4A7 | 0.002 | — | −0.443 | 0.245 | — | — | — | −0.107 |
| SLC50A1 | −0.037 | −0.444 | — | — | — | — | — | 0.368 |
| SLC7A6OS | — | −0.299 | — | 0.811 | — | — | — | 0.337 |
| SLC7A7 | — | 0.312 | 0.122 | — | — | −0.453 | — | — |
| SLC9A7 | 0.008 | 0.762 | 0.027 | 0.086 | — | — | −0.404 | — |
| SLCO2B1 | — | — | — | −0.100 | — | — | −0.145 | — |
| SLIT2 | — | 0.010 | — | −0.845 | — | 0.038 | — | — |
| SLX4 | — | 0.324 | — | — | — | 0.365 | — | — |
| SMARCA2 | −0.002 | — | −0.030 | — | — | 0.036 | — | — |
| SMC4 | −0.008 | — | — | 0.050 | — | — | — | 0.240 |
| SMC6 | — | 0.379 | — | — | — | — | — | 0.263 |
| SMCHD1 | — | 0.507 | — | −0.263 | — | 0.179 | — | 0.421 |
| SMCO4 | — | — | −0.015 | 0.829 | — | 0.442 | — | — |
| SMIM1 | — | −0.017 | — | — | — | 0.316 | — | 0.141 |
| SMIM15 | — | −0.132 | — | 0.235 | — | — | — | 0.091 |
| SNAP23 | −0.037 | — | −0.008 | 0.171 | — | 0.023 | — | 0.006 |
| SNAP47 | — | — | 0.473 | — | — | 0.209 | — | — |
| SNRK | — | — | 0.005 | 0.210 | — | — | — | 0.791 |
| SNRNP27 | — | 0.360 | — | — | — | — | — | 0.235 |
| SNRNP40 | — | — | 0.026 | — | — | −0.054 | — | — |
| SNRPC | — | −0.298 | — | 0.602 | — | — | — | 0.224 |
| SNX13 | — | — | — | 0.356 | — | — | — | 0.373 |
| SNX32 | — | −0.512 | — | −0.299 | — | 0.334 | — | — |
| SOAT1 | — | — | — | 0.260 | — | — | — | 0.392 |
| SORBS1 | — | 0.089 | — | — | — | — | — | 0.497 |
| SORBS2 | — | −0.116 | — | −0.423 | — | −0.072 | — | — |
| SORT1 | — | 0.185 | — | 0.287 | — | — | — | 0.100 |
| SP110 | 0.020 | — | 0.018 | −0.737 | — | — | — | 0.637 |
| SP140 | — | −0.106 | — | −0.198 | — | 0.122 | — | 0.206 |
| SPAG1 | — | −0.179 | — | −0.554 | — | — | — | 0.709 |
| SPAG9 | — | — | — | 0.300 | — | — | — | 0.417 |
| SPATA5 | — | 0.422 | — | 0.280 | — | — | — | 0.752 |
| SPATS2L | −0.002 | — | — | — | — | 0.139 | — | — |
| SPC24 | — | — | — | 0.331 | — | — | — | 0.039 |
| SPCS1 | — | 0.076 | — | 0.006 | — | — | — | 0.043 |
| SPECC1L | — | — | −0.010 | — | — | 0.023 | — | — |
| SPIN3 | −0.018 | — | — | — | — | — | 0.280 | — |
| SPINT1 | 0.002 | 0.055 | — | — | — | −0.112 | — | — |
| SPINT2 | — | 0.012 | −0.007 | 0.575 | — | — | — | 0.486 |
| SPIRE1 | — | — | — | −0.085 | — | — | — | 0.399 |
| SPON2 | — | 0.148 | — | — | — | 0.152 | — | — |
| SPPL2B | — | — | — | 0.325 | — | — | — | 0.587 |
| SRCAP | — | 0.160 | — | — | — | — | — | 0.628 |
| SREBF1 | — | 0.259 | — | — | — | — | — | 0.212 |
| SRGAP2 | — | — | — | −0.406 | — | — | — | 0.731 |
| SRGAP2B | — | — | — | −0.406 | — | — | — | 0.731 |
| SRGAP2C | — | — | — | −0.406 | — | — | — | 0.731 |
| SRP14 | — | −0.065 | — | 0.482 | — | — | — | 0.033 |
| SRPK1 | — | — | — | −0.846 | — | — | — | −0.317 |
| SRPRB | 0.019 | −0.080 | — | 0.349 | — | — | — | 0.440 |
| SRPX2 | — | 0.376 | — | — | — | −0.490 | — | — |
| SSBP3 | −0.003 | 0.063 | — | 0.016 | — | — | — | 0.068 |
| SSBP4 | — | −0.534 | — | 0.684 | — | — | — | 0.105 |
| SSH2 | 0.001 | 0.028 | — | 0.123 | — | — | — | 0.078 |
| SSH3 | — | 0.294 | — | — | — | — | — | 0.718 |
| SSR1 | — | −0.814 | — | — | — | — | — | 0.046 |
| SSR3 | — | — | — | 0.002 | — | — | — | 0.131 |
| ST20-MTHFS | — | −0.114 | — | 0.147 | — | 0.127 | — | — |
| ST3GAL1 | — | — | 0.026 | 0.040 | — | — | — | 0.016 |
| ST3GAL5 | −0.015 | — | 0.009 | — | — | — | — | −0.523 |
| ST7 | — | 0.165 | — | −0.169 | — | — | — | 0.185 |
| STARD3 | — | — | — | 0.592 | — | — | — | −0.332 |
| STARD3NL | — | −0.321 | — | 0.325 | — | — | — | 0.218 |
| STAU1 | — | 0.197 | — | 0.015 | — | — | — | 0.375 |
| STBD1 | — | — | — | 0.225 | — | 0.169 | — | — |
| STK16 | — | 0.279 | — | −0.465 | — | — | — | 0.511 |
| STK26 | −0.005 | −0.057 | 0.018 | 0.018 | — | — | — | 0.101 |
| STK38 | — | — | — | 0.070 | — | — | — | 0.348 |
| STN1 | — | 0.635 | — | 0.289 | — | 0.207 | — | — |
| STRADA | — | 0.066 | — | −0.242 | — | — | — | −0.381 |
| STRADB | — | 0.263 | — | 0.286 | — | — | — | 0.085 |
| STRN4 | — | — | — | 0.032 | — | — | — | 0.063 |
| STX7 | — | — | — | 0.073 | — | 0.177 | — | — |
| STYX | — | −0.016 | — | 0.603 | — | — | — | 0.651 |
| SUCO | 0.003 | — | — | — | — | 0.464 | — | 0.765 |
| SUDS3 | — | — | — | −0.169 | — | — | — | 0.080 |
| SUGT1 | — | — | — | 0.330 | — | 0.034 | — | 0.318 |
| SUN2 | — | — | — | 0.143 | — | — | — | −0.149 |
| SUPT4H1 | −0.021 | — | — | — | — | 0.122 | — | — |
| SURF2 | — | — | — | 0.299 | — | — | — | 0.325 |
| SUV39H1 | — | — | — | 0.675 | — | — | — | 0.331 |
| SWT1 | — | −0.398 | — | 0.152 | — | — | — | 0.285 |
| SYNM | — | −0.031 | — | −0.588 | — | 0.050 | — | — |
| SYT7 | 0.014 | −0.308 | — | −0.082 | — | −0.267 | — | — |
| SYTL1 | — | 0.778 | — | — | — | 0.605 | — | — |
| SYTL3 | — | 0.222 | — | 0.357 | — | — | — | 0.643 |
| SYVN1 | — | −0.055 | — | — | — | — | — | 0.648 |
| TACC2 | — | 0.427 | — | — | — | 0.073 | — | — |
| TAF2 | — | — | — | 0.288 | — | — | — | 0.791 |
| TAGLN2 | — | — | — | 0.003 | 0.002 | — | — | 0.003 |
| TAP1 | — | 0.652 | — | −0.244 | — | — | — | 0.133 |
| TARBP2 | — | 0.274 | — | −0.152 | — | — | — | 0.168 |
| TARDBP | — | −0.118 | — | −0.301 | — | — | — | 0.332 |
| TARS | — | — | — | 0.448 | — | — | — | 0.645 |
| TARS2 | — | — | — | 0.405 | — | — | — | 0.638 |
| TAX1BP1 | −0.003 | — | — | — | — | — | — | 0.027 |
| TAX1BP3 | — | 0.059 | — | 0.189 | — | — | — | 0.209 |
| TBC1D10C | — | −0.205 | — | 0.269 | — | — | — | −0.260 |
| TBC1D5 | — | 0.061 | −0.002 | — | — | — | — | 0.094 |
| TBK1 | — | 0.286 | — | 0.049 | — | — | — | −0.745 |
| TBP | — | −0.254 | — | 0.339 | — | — | — | 0.423 |
| TBRG1 | — | — | — | 0.452 | — | — | — | 0.323 |
| TBX4 | −0.138 | 0.298 | — | — | — | 0.099 | — | — |
| TCERG1 | — | −0.137 | — | 0.202 | — | — | — | 0.639 |
| TCF12 | — | −0.111 | — | — | — | −0.200 | — | 0.087 |
| TCF19 | — | −0.204 | — | — | — | 0.504 | — | — |
| TCF4 | — | 0.233 | 0.030 | −0.084 | — | — | 0.430 | — |
| TEC | — | 0.105 | — | −0.495 | — | — | — | 0.044 |
| TECPR1 | — | — | — | −0.455 | — | — | — | 0.612 |
| TEDC2 | — | — | — | 0.250 | — | — | −0.188 | — |
| TEF | — | 0.091 | — | −0.353 | — | 0.143 | — | — |
| TERF1 | — | 0.138 | — | −0.238 | — | 0.171 | — | — |
| TERF2 | — | −0.423 | — | −0.406 | — | — | — | 0.535 |
| TFB1M | — | — | — | −0.394 | — | 0.367 | — | — |
| TFCP2 | — | — | — | 0.425 | — | 0.186 | — | — |
| TFDP1 | 0.703 | 0.337 | −0.196 | — | — | — | — | 0.648 |
| TFE3 | — | 0.285 | — | — | — | — | — | 0.400 |
| TFEC | — | — | — | 0.619 | — | — | — | 0.656 |
| TGFB1I1 | — | 0.003 | — | 0.102 | — | — | — | −0.104 |
| TGIF1 | 0.004 | 0.274 | — | — | — | 0.266 | — | 0.607 |
| THBS3 | — | 0.023 | — | — | — | 0.043 | — | — |
| TIA1 | — | 0.199 | — | 0.670 | — | — | — | 0.359 |
| TIAL1 | — | — | — | 0.197 | — | 0.236 | — | — |
| TIAM2 | — | 0.195 | — | −0.151 | — | — | — | 0.315 |
| TIFA | — | — | — | 0.657 | — | −0.056 | — | — |
| TIMM10B | — | −0.088 | — | — | — | — | — | 0.853 |
| TIMP3 | — | 0.155 | — | — | — | 0.034 | — | — |
| TJP2 | −0.014 | −0.475 | −0.011 | 0.253 | — | — | — | −0.087 |
| TLE3 | −0.044 | — | — | −0.094 | — | — | — | 0.270 |
| TLE5 | — | — | — | −0.100 | — | — | — | 0.006 |
| TLK1 | — | — | — | 0.011 | — | — | — | 0.445 |
| TMBIM1 | — | −0.018 | — | −0.260 | — | — | — | 0.047 |
| TMC6 | — | — | 0.014 | 0.298 | — | — | — | 0.326 |
| TMCC1 | — | 0.202 | — | −0.133 | — | 0.112 | — | — |
| TMCC2 | — | 0.334 | — | — | — | 0.267 | — | — |
| TMCC3 | — | — | — | 0.090 | — | 0.453 | — | — |
| TMEM123 | — | 0.339 | — | 0.272 | — | 0.087 | — | 0.027 |
| TMEM163 | — | 0.431 | — | 0.424 | — | 0.072 | — | — |
| TMEM176A | −0.010 | — | — | — | — | 0.061 | — | — |
| TMEM208 | −0.050 | 0.567 | — | 0.835 | — | — | — | −0.338 |
| TMEM229B | — | −0.521 | — | 0.306 | — | 0.126 | — | −0.376 |
| TMEM232 | — | −0.092 | — | — | — | 0.192 | — | — |
| TMEM241 | — | — | −0.211 | −0.188 | — | — | — | 0.149 |
| TMEM256 | — | 0.423 | — | 0.192 | — | — | — | 0.082 |
| TMEM256- | — | 0.423 | — | 0.192 | — | — | — | 0.082 |
| PLSCR3 | | | | | | | | |
| TMEM87B | — | — | — | 0.338 | — | — | — | 0.284 |
| TMSB4X | — | — | — | 0.133 | — | 0.002 | — | −0.001 |
| TMSB4Y | — | — | — | 0.133 | — | 0.002 | — | −0.001 |
| TNFRSF19 | 0.003 | 0.278 | — | — | — | 0.053 | — | — |
| TNFSF13B | — | −0.089 | — | 0.465 | — | 0.184 | — | — |
| TNIK | — | 0.100 | — | 0.052 | — | 0.149 | — | 0.152 |
| TNIP2 | — | — | −0.011 | −0.400 | — | −0.077 | — | — |
| TNPO3 | — | — | — | 0.272 | — | — | — | 0.797 |
| TNRC6C | — | 0.159 | — | — | — | — | — | −0.428 |
| TOE1 | — | −0.124 | — | — | — | — | — | 0.414 |
| TOM1 | — | −0.214 | — | — | — | −0.094 | — | — |
| TOR1A | — | −0.149 | — | — | — | — | — | 0.470 |
| TP53I11 | — | 0.110 | — | 0.029 | — | — | — | −0.259 |
| TPCN2 | — | — | — | 0.471 | — | — | — | 0.808 |
| TPD52 | 0.010 | — | — | — | — | −0.146 | — | 0.055 |
| TPI1 | −0.002 | — | −0.002 | 0.006 | — | — | — | 0.062 |
| TPM2 | — | 0.216 | — | — | — | 0.053 | — | −0.014 |
| TPP1 | 0.000 | 0.128 | — | — | — | — | — | −0.158 |
| TPRA1 | −0.019 | 0.153 | −0.052 | −0.663 | — | 0.356 | — | — |
| TRA2A | −0.002 | 0.026 | — | 0.455 | — | 0.558 | — | — |
| TRABD | — | 0.422 | — | 0.139 | — | — | — | −0.451 |
| TRAF3IP3 | — | 0.439 | −0.066 | −0.280 | — | — | — | −0.090 |
| TRAPPC11 | — | — | −0.021 | 0.055 | — | — | — | 0.251 |
| TRAPPC13 | — | −0.036 | — | −0.079 | — | 0.201 | — | 0.431 |
| TRAPPC4 | −0.015 | −0.325 | — | 0.584 | — | — | — | 0.182 |
| TRAPPC8 | — | — | — | −0.211 | — | — | — | 0.645 |
| TREM2 | — | — | — | 0.363 | — | — | 0.297 | — |
| TREML1 | — | −0.352 | — | −0.018 | — | — | — | 0.010 |
| TRIM28 | −0.012 | — | — | 0.566 | — | — | — | 0.190 |
| TRMT1 | — | −0.153 | — | 0.372 | — | — | — | 0.242 |
| TRMT112 | — | −0.680 | −0.010 | −0.626 | — | — | — | 0.384 |
| TRNT1 | — | — | — | 0.039 | — | — | — | −0.452 |
| TRPC1 | −0.008 | — | — | — | — | — | — | 0.268 |
| TRPC4AP | — | 0.089 | — | — | — | — | — | 0.075 |
| TRPS1 | — | −0.027 | — | 0.060 | — | 0.073 | — | — |
| TRPV2 | — | — | — | 0.564 | — | — | — | 0.344 |
| TRUB1 | — | — | — | −0.815 | — | 0.620 | — | — |
| TRUB2 | — | — | — | 0.434 | — | — | — | −0.616 |
| TSC2 | 0.021 | 0.021 | 0.095 | −0.247 | — | 0.084 | — | 0.246 |
| TSGA10 | — | — | — | 0.164 | — | — | — | 0.764 |
| TSN | −0.013 | 0.276 | — | — | — | 0.122 | — | — |
| TSPAN32 | — | 0.429 | — | 0.021 | — | −0.276 | — | — |
| TSPAN9 | — | 0.018 | — | 0.141 | — | 0.066 | — | — |
| TTC13 | — | 0.301 | — | — | — | — | — | 0.408 |
| TTC21A | 0.205 | — | 0.236 | — | — | 0.102 | — | — |
| TTC3 | — | 0.042 | — | 0.073 | — | 0.156 | — | — |
| TTC37 | — | — | — | −0.499 | — | — | — | 0.543 |
| TTPAL | — | — | — | 0.008 | — | — | — | 0.420 |
| TUBB | — | 0.047 | — | 0.124 | — | — | — | 0.091 |
| TUBGCP3 | — | −0.313 | — | 0.320 | — | — | — | −0.237 |
| TUBGCP5 | — | 0.079 | — | −0.337 | — | — | — | 0.840 |
| TUFM | — | 0.639 | — | — | — | 0.313 | — | — |
| TUT7 | — | 0.020 | — | −0.334 | — | — | — | 0.155 |
| TXN2 | — | — | — | 0.399 | — | 0.142 | — | 0.067 |
| TXNDC16 | — | — | — | −0.832 | — | 0.095 | — | — |
| TYRO3 | — | 0.530 | — | — | — | 0.143 | — | — |
| U2AF1L4 | — | 0.325 | — | −0.484 | — | — | — | 0.667 |
| U2SURP | — | −0.105 | — | — | — | — | — | 0.143 |
| UBA2 | — | 0.105 | — | — | — | — | — | 0.318 |
| UBA7 | — | −0.178 | — | 0.075 | — | — | — | −0.289 |
| UBAC2 | −0.020 | −0.593 | −0.002 | −0.268 | — | — | — | 0.241 |
| UBAP2L | — | 0.307 | — | −0.229 | — | — | — | 0.462 |
| UBASH3A | 0.074 | 0.515 | 0.054 | −0.769 | — | — | — | 0.503 |
| UBC | — | — | — | 0.177 | — | 0.009 | — | 0.027 |
| UBE2D2 | — | 0.327 | — | 0.186 | — | — | — | 0.212 |
| UBE2Q1 | — | — | — | 0.316 | — | — | — | −0.155 |
| UBFD1 | — | 0.086 | — | 0.345 | — | — | — | 0.518 |
| UBL4A | — | −0.175 | — | — | — | — | — | −0.200 |
| UBL5 | — | — | — | 0.021 | — | 0.064 | — | 0.021 |
| UBN1 | — | — | — | 0.146 | — | −0.157 | — | — |
| UBR2 | — | — | — | −0.421 | — | — | — | −0.545 |
| UBR5 | — | −0.161 | — | — | — | — | — | 0.272 |
| UBTF | — | 0.016 | — | 0.143 | — | 0.039 | — | — |
| UBXN1 | — | — | — | −0.432 | — | — | — | 0.100 |
| UEVLD | — | 0.382 | — | −0.701 | — | — | — | 0.340 |
| UFC1 | — | 0.189 | — | 0.258 | — | −0.335 | — | — |
| UNC119 | — | −0.163 | — | — | — | — | — | −0.074 |
| UPF2 | — | — | — | 0.324 | — | 0.111 | — | — |
| UQCR11 | — | — | — | 0.028 | — | — | — | 0.016 |
| UQCRH | — | — | — | 0.015 | — | — | — | 0.020 |
| UQCRHL | — | — | — | 0.015 | — | — | — | 0.020 |
| USE1 | — | −0.266 | — | 0.190 | — | — | — | 0.133 |
| USP21 | — | 0.298 | — | — | — | — | — | 0.748 |
| USP28 | — | −0.651 | — | 0.620 | — | 0.262 | — | — |
| USP33 | — | — | — | 0.274 | — | — | — | 0.607 |
| USP40 | — | — | — | −0.252 | — | — | — | 0.639 |
| USP49 | — | — | — | −0.617 | −0.331 | — | — | — |
| USP53 | — | — | −0.247 | — | — | — | — | 0.609 |
| USP7 | — | −0.096 | −0.018 | — | — | — | — | 0.256 |
| USP8 | — | — | — | 0.650 | — | — | — | 0.351 |
| USPL1 | — | 0.319 | — | 0.086 | — | — | — | −0.372 |
| UTRN | — | 0.012 | — | — | — | 0.047 | — | — |
| UTY | — | −0.091 | — | 0.079 | — | — | — | 0.424 |
| UVRAG | — | −0.324 | −0.004 | −0.289 | — | 0.036 | — | 0.079 |
| VAC14 | — | −0.250 | — | 0.411 | — | — | — | 0.282 |
| VAV1 | — | −0.035 | — | 0.164 | — | — | — | 0.352 |
| VCAN | — | −0.059 | — | −0.068 | — | −0.434 | — | — |
| VCL | — | — | −0.009 | — | — | — | — | 0.028 |
| VDAC3 | 0.009 | −0.079 | −0.017 | 0.159 | — | — | — | 0.044 |
| VEZT | −0.010 | — | — | 0.555 | — | — | — | 0.679 |
| VGLL4 | 0.003 | 0.014 | — | 0.321 | — | — | — | 0.163 |
| VIM | — | — | — | 0.006 | — | −0.021 | — | −0.111 |
| VLDLR | — | −0.379 | — | −0.408 | — | 0.183 | — | — |
| VPS11 | — | — | 0.107 | 0.431 | — | — | — | 0.355 |
| VPS13A | — | −0.026 | −0.354 | 0.187 | — | −0.364 | — | — |
| VPS13B | — | — | — | −0.585 | — | 0.038 | — | 0.293 |
| VPS13D | −0.006 | — | — | — | — | — | — | −0.781 |
| VPS26A | — | — | — | 0.325 | — | — | — | 0.240 |
| VPS28 | — | −0.020 | — | — | — | — | — | 0.070 |
| VPS53 | — | — | — | −0.862 | — | — | — | −0.468 |
| VPS8 | — | 0.444 | — | −0.490 | — | 0.104 | — | — |
| VRK1 | −0.337 | — | — | 0.387 | — | — | — | 0.305 |
| VRK2 | −0.010 | 0.472 | — | — | — | — | — | 0.122 |
| VTI1A | — | — | — | −0.445 | — | 0.029 | — | — |
| WDR1 | — | 0.178 | — | 0.196 | — | — | — | 0.074 |
| WDR11 | — | −0.319 | — | 0.325 | — | 0.264 | — | — |
| WDR74 | — | — | — | 0.411 | — | — | — | 0.259 |
| WDR75 | — | −0.254 | — | — | — | — | — | 0.781 |
| WDYHV1 | — | — | — | 0.044 | — | 0.106 | — | 0.581 |
| WFDC8 | — | −0.474 | — | — | — | −0.180 | — | — |
| WNK1 | — | 0.034 | — | 0.064 | — | 0.008 | — | — |
| WRB | — | 0.314 | — | 0.289 | — | 0.281 | — | — |
| WRNIP1 | — | −0.282 | — | 0.335 | — | — | — | 0.073 |
| WWP2 | — | — | −0.002 | 0.430 | — | — | — | 0.220 |
| XAB2 | — | — | — | 0.327 | — | — | — | 0.573 |
| XPO6 | −0.015 | — | — | 0.311 | — | — | — | 0.341 |
| XPO7 | — | 0.102 | — | — | — | — | — | 0.449 |
| XPR1 | — | — | — | −0.795 | — | 0.188 | — | 0.188 |
| YIPF4 | — | — | — | 0.418 | — | 0.147 | — | — |
| YJU2 | — | 0.194 | — | −0.385 | — | — | — | 0.487 |
| YPEL5 | — | — | — | 0.014 | — | — | — | 0.006 |
| YWHAQ | — | — | — | 0.304 | — | 0.036 | — | — |
| ZBP1 | — | −0.394 | — | 0.312 | — | 0.573 | — | — |
| ZBTB17 | — | — | — | 0.641 | — | — | — | 0.555 |
| ZBTB2 | — | −0.323 | — | −0.317 | — | — | — | −0.273 |
| ZBTB20 | 0.035 | −0.383 | −0.070 | −0.278 | −0.312 | — | — | — |
| ZBTB34 | — | — | — | −0.086 | — | — | — | −0.608 |
| ZBTB38 | −0.003 | −0.013 | — | 0.208 | — | — | — | −0.543 |
| ZBTB4 | — | — | — | 0.243 | — | — | — | 0.439 |
| ZBTB7A | — | — | — | −0.146 | — | — | — | −0.174 |
| ZC3H10 | — | — | — | 0.314 | — | 0.417 | — | — |
| ZC3H7B | — | — | — | 0.396 | — | 0.042 | — | — |
| ZC3HC1 | — | −0.199 | — | −0.816 | — | — | — | 0.211 |
| ZCRB1 | — | 0.143 | — | — | — | −0.115 | — | — |
| ZDHHC12 | — | 0.196 | — | 0.430 | — | — | — | −0.358 |
| ZDHHC20 | 0.011 | −0.006 | — | — | — | — | — | 0.138 |
| ZDHHC4 | — | −0.158 | — | 0.035 | — | — | — | 0.365 |
| ZDHHC6 | — | 0.208 | — | 0.300 | — | — | — | 0.659 |
| ZEB2 | −0.555 | 0.175 | 0.028 | −0.291 | — | — | — | 0.050 |
| ZFAND3 | — | 0.008 | — | 0.162 | — | — | — | 0.072 |
| ZFAND6 | — | — | — | 0.102 | — | 0.073 | — | — |
| ZFP1 | — | 0.213 | — | 0.239 | — | — | — | 0.148 |
| ZFY | — | 0.290 | — | — | — | — | — | 0.210 |
| ZFYVE16 | — | — | 0.122 | — | — | — | 0.340 | — |
| ZFYVE26 | — | 0.051 | — | −0.143 | — | — | — | 0.780 |
| ZGPAT | — | −0.697 | — | — | — | — | — | 0.071 |
| ZHX1 | — | 0.171 | — | — | — | — | — | 0.580 |
| ZMYND11 | −0.004 | 0.072 | — | −0.278 | — | — | — | −0.327 |
| ZMYND8 | — | — | — | 0.250 | — | 0.264 | — | −0.266 |
| ZNF131 | — | 0.095 | — | 0.270 | — | — | — | 0.123 |
| ZNF148 | — | — | −0.007 | 0.258 | — | 0.120 | — | — |
| ZNF160 | — | — | — | 0.576 | — | — | — | 0.448 |
| ZNF195 | — | 0.122 | — | −0.284 | — | −0.722 | — | — |
| ZNF236 | — | — | — | 0.329 | — | −0.122 | — | — |
| ZNF280D | — | −0.274 | — | 0.205 | — | 0.327 | — | 0.639 |
| ZNF287 | — | 0.658 | — | 0.279 | — | — | −0.417 | — |
| ZNF32 | — | 0.053 | — | 0.082 | — | 0.212 | — | 0.624 |
| ZNF330 | — | −0.087 | — | 0.460 | — | — | — | 0.480 |
| ZNF410 | — | −0.317 | — | 0.503 | — | — | — | 0.179 |
| ZNF429 | — | 0.122 | — | −0.284 | — | −0.722 | — | — |
| ZNF532 | — | −0.489 | — | −0.806 | — | 0.220 | — | — |
| ZNF644 | — | −0.076 | — | −0.024 | — | — | — | 0.053 |
| ZNF665 | — | — | — | 0.576 | — | — | — | 0.448 |
| ZNF667 | — | −0.186 | — | 0.156 | 0.187 | — | — | — |
| ZNF687 | — | 0.157 | — | −0.242 | — | — | — | 0.493 |
| ZNF76 | — | −0.383 | −0.466 | — | — | −0.207 | — | — |
| ZSWIM8 | 0.039 | — | — | 0.077 | — | — | — | 0.377 |
| ZWINT | — | — | — | −0.504 | — | — | — | 0.060 |

### LIST OF EMBODIMENTS

Specific compositions and methods of RNA sequencing to diagnose sepsis have been described. The detailed description in this specification is illustrative and not restrictive or exhaustive. The detailed description is not intended to limit the disclosure to the precise form disclosed. Other equivalents and modifications besides those already described are possible without departing from the inventive concepts described in this specification, as those skilled in the art will recognize. When the specification or claims recite method steps or functions in order, alternative embodiments may perform the tasks in a different order or substantially concurrently. The inventive subject matter is not to be restricted except in the spirit of the disclosure.

When interpreting the disclosure, all terms should be interpreted in the broadest possible manner consistent with the context. Unless otherwise defined, all technical and scientific terms used in this specification have the same meaning as commonly understood by one of ordinary skill in the art to which this invention belongs. This invention is not limited to the particular methodology, protocols, reagents, and the like described in this specification and, as such, can vary in practice. The terminology used in this specification is not intended to limit the scope of the invention, which is defined solely by the claims.

All patents and publications cited throughout this specification are expressly incorporated by reference to disclose and describe the materials and methods that might be used with the technologies described in this specification. The publications discussed are provided solely for their disclosure before the filing date. They should not be construed as an admission that the inventors may not antedate such disclosure under prior invention or for any other reason. If there is an apparent discrepancy between a previous patent or publication and the description provided in this specification, the present specification (including any definitions) and claims shall control. All statements as to the date or representation as to the contents of these documents are based on the information available to the applicants and constitute no admission as to the correctness of the dates or contents of these documents. The dates of publication provided in this specification may differ from the actual publication dates. If there is an apparent discrepancy between a publication date provided in this specification and the actual publication date supplied by the publisher, the actual publication date shall control.

The terms “comprises” and “comprising” should be interpreted as referring to elements, components, or steps in a non-exclusive manner, indicating that the referenced elements, components, or steps may be present, used, or combined with other elements, components, or steps. The singular terms “a,” “an,” and “the” include plural referents unless context indicates otherwise. Similarly, the word “or” should cover “and” unless the context indicates otherwise. The abbreviation “e.g.” is used to indicate a non-limiting example and is synonymous with the term “For example.”

When a range of values is provided, each intervening value, to the tenth of the unit of the lower limit, unless the context dictates otherwise, between the upper and lower limit of that range and any other stated or intervening value in that range of values.

Some embodiments of the technology described can be defined according to the following numbered paragraphs:

1. A method of using unmapped bacterial RNA reads to identify bacteria causing sepsis.

2. A method of using unmapped viral reads to identify sepsis or viral reactivation.

3. A method of using unmapped B/T V(D)J to identify sepsis.

4. A method of using a Principal Component Analysis of RNA splicing entropy to identify sepsis.

5. A method of using RNA lariats to identify sepsis.

6. A method of using a Principal Component Analysis of gene expression, alternative RNA splicing, or alternative transcription start and end to identify sepsis.

We claim:

1. A method of using unmapped bacterial RNA reads to identify bacteria causing sepsis, comprising the steps of:
(a) obtaining RNA sequencing from a body sample;
(b) aligning the RNA sequencing data (reads) to the genome of interest;
(c) selecting the un-mapped reads and analyzing the reads using a Read Origin Protocol (ROP); and
(d) identifying bacteria that are present in the sample;
wherein the bacteria that are present in the sample are identified as causing sepsis.

2. A method of using unmapped viral reads to identify sepsis or viral reactivation, comprising the steps of:
(a) obtaining RNA sequencing from a body sample;
(b) aligning the RNA sequencing data (reads) to the genome of interest;
(c) selecting the un-mapped reads and analyzing the reads using a Read Origin Protocol (ROP); and
(d) identifying the viruses present in the sample;
wherein the virus identified with Principal Component Analysis (A) is used to identify likely sepsis samples.

3. A method of using unmapped B/T V(D)J to identify sepsis, comprising the steps of:
(a) obtaining RNA sequencing from a body sample;
(b) aligning the RNA sequencing data (reads) to the genome of interest;
(c) selecting the un-mapped reads and analyzing the reads using a Read Origin Protocol (ROP); and
(d) identifying the T/B cell epitopes present in the samples;
wherein the he T/B cell epitopes identified with Principal Component Analysis (A) is are used to identify likely sepsis samples.

4. A method of using a Principal Component Analysis (PCA) of RNA splicing entropy to identify sepsis, comprising the steps of:
(a) obtaining RNA sequencing from a body sample;
(b) aligning the RNA sequencing data (reads) to the genome of interest;
(c) selecting the un-mapped reads and analyzing the reads using a Read Origin Protocol (ROP); and
(d) selecting the mapped reads and using a program that enables detection and quantification of alternative RNA splicing events to identity gene expression, RNA splicing events, alternative transcription start/end, or RNA splicing entropy;
wherein RNA splicing entropy identified by PCA identify likely sepsis samples.

5. A method of using RNA lariats to identify sepsis, comprising the steps of:
(a) obtaining RNA sequencing from a body sample;
(b) aligning the RNA sequencing data (reads) to the genome of interest;
(c) selecting the un-mapped reads and analyzing the reads using a Read Origin Protocol (ROP); and
(d) selecting the mapped reads and using a program that enables detection and quantification of alternative RNA splicing events to identity gene expression, RNA splicing events, alternative transcription start/end, or RNA splicing entropy;
wherein RNA lariats identified by PCA identify likely sepsis samples.

6. A method of using a Principal Component Analysis (PCA) of gene expression, alternative RNA splicing, or alternative transcription start and end to identify sepsis, comprising the steps of:
(a) obtaining RNA sequencing from a body sample;
(b) aligning the RNA sequencing data (reads) to the genome of interest;
(c) selecting the un-mapped reads and analyzing the reads using a Read Origin Protocol (ROP); and
(d) selecting the mapped reads and using a program that enables detection and quantification of alternative RNA splicing events to identity gene expression, RNA splicing events, alternative transcription start/end, or RNA splicing entropy;
wherein the gene expression changes, RNA splicing events, and alternative transcription start/end that are identified by PCA identify likely sepsis samples.
